SEQUENCE LISTING (25 sequences)

SEQ ID NO: 1 - protein, 422 aa, Homo Sapiens
MSVGAMKKGVGRAVGLGGGSGCQATEEDPLPDCGACAPGQGGRRWRLPQPAWVEGSSARLWEQATGTGWMDLEASLLPTGPNASNTSDGPDNLTSAGSPPRTGSISYINIIMPSVFGTICLLGIIGNSTVIFAVVKKSKLHWCNNVPDIFIINLSVVDLLFLLGMPFMIHQLMGNGVWHFGETMCTLITAMDANSQFTSTYILTAMAIDRYLATVHPISSTKFRKPSVATLVICLLWALSFISITPVWLYARLIPFPGGAVGCGIRLPNPDTDLYWFTLYQFFLAFALPFVVITAAYVRILQRMTSSVAPASQRSIRLRTKRVTRTAIAICLVFFVCWAPYYVLQLTQLSISRPTLTFVYLYNAAISLGYANSCLNPFVYIVLCETFRKRLVLSVKPAAQGQLRAVSNAQTADEERTESKGT

SEQ ID NO: 2 - protein, 30 aa, Homo Sapiens
SYINIIMPSVFGTICLLGIIGNSTVIFAVV

SEQ ID NO: 3 - protein, 30 aa, Homo Sapiens
VPDIFIINLSVVDLLFLLGMPFMIHQLMGN

SEQ ID NO: 4 - protein, 31 aa, Homo Sapiens
TMCTLITAMDANSQFTSTYILTAMAIDRYLA

SEQ ID NO: 5 - protein, 24 aa, Homo Sapiens
PSVATLVICLLWALSFISITPVWL

SEQ ID NO: 6 - protein, 29 aa, Homo Sapiens
LYWFTLYQFFLAFALPFVVITAAYVRILQ

SEQ ID NO: 7 - protein, 32 aa, Homo Sapiens
RTKRVTRTAIAICLVFFVCWAPYYVLQLTQLS

SEQ ID NO: 8 - protein, 24 aa, Homo Sapiens
YLYNAAISLGYANSCLNPFVYIVL

SEQ ID NO: 9 - protein, 7 aa, Homo Sapiens
GVWHFGE

SEQ ID NO: 10 - protein, 24 aa, Homo Sapiens
YARLIPFPGGAVGCGIRLPNPDTD

SEQ ID NO: 11 - protein, 9 aa, Homo Sapiens
ISRPTLTFV

SEQ ID NO: 12 - protein, 10 aa, Homo Sapiens
KKSKLHWCNN

SEQ ID NO: 13 - protein, 12 aa, Homo Sapiens
TVHPISSTKFRK

SEQ ID NO: 14 - protein, 16 aa, Homo Sapiens
RMTSSVAPASQRSIRL

SEQ ID NO: 15 - protein, 18 aa, Homo Sapiens
CETFRKRLVLSVKPAAQG

SEQ ID NO: 16 - protein, 15 aa, Homo Sapiens
SYINIIMPSVFGTIC

SEQ ID NO: 17 - protein, 37 aa, Homo Sapiens
FLLGMPFMIHQLMGNGVWHFGETMCTLITAMDANSQF

SEQ ID NO: 18 - protein, 54 aa, Homo Sapiens
LLWALSFISITPVWLYARLIPFPGGAVGCGIRLPNPDTDLYWFTLYQFFLAFAL

SEQ ID NO: 19 - protein, 39 aa, Homo Sapiens
VCWAPYYVLQLTQLSISRPTLTFVYLYNAAISLGYANSC

SEQ ID NO: 20 - protein, 15 aa, Homo Sapiens
FLLGMPFMIHQLMGN

SEQ ID NO: 21 - protein, 15 aa, Homo Sapiens
TMCTLITAMDANSQF

SEQ ID NO: 22 - protein, 15 aa, Homo Sapiens
LLWALSFISITPVWL

SEQ ID NO: 23 - protein, 15 aa, Homo Sapiens
LYWFTLYQFFLAFAL

SEQ ID NO: 24 - protein, 15 aa, Homo Sapiens
VCWAPYYVLQLTQLS

SEQ ID NO: 25 - protein, 15 aa, Homo Sapiens
YLYNAAISLGYANSC

(19) United States
(12) Patent Application Publication
(10) Pub. No.: US 2005/0170433 A1
(43) Pub. Date: Aug. 4, 2005

(54) Three dimensional coordinates of melanin-concentrating hormone receptors

(75) Inventors: Glen Edward Mieling, West Chester, OH (US); Katherine King Mieling, West Chester, OH (US); Rodney Dean Bush, Fairfield, OH (US); Anny-Odile Colson, Loveland, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/414,522

(22) Filed: Apr. 15, 2003

(51) Int. Cl. G01N 33/53; G01N 33/567; G06F 19/00; G01N 33/48; G01N 33/50

(52) U.S. Cl. 435/7.2; 702/19

(57) ABSTRACT

Three-dimensional coordinates of melanin-concentrating hormone receptors are useful in computer aided drug design.

### FIELD OF THE INVENTION

The present invention relates to three-dimensional structures of G protein-coupled receptors, especially melanin-concentrating hormone receptor 1, and structures derived therefrom.

### BACKGROUND OF THE INVENTION

I. GPCRs

Most eukaryotic organisms utilize guanine nucleotide-binding regulatory protein (hereinafter, referred to as “G protein”)-coupled receptors (G protein-coupled receptors; hereinafter, frequently referred to as “GPCRs”) to respond to a variety of stimuli such as Ca²⁺, amines, hormones, peptides, and even large proteins. Each GPCR is composed of an extracellular region, an intracellular region and a transmembrane bundle of seven α-helices. The binding of specific ligands to the extracellular or transmembrane domain evokes conformational changes that are transmitted to the intracellular region. The conformational switch from inactive to active state in the intracellular region produces a catalytically competent receptor that is responsible for binding and nucleotide exchange (GDP [symbol] GTP) on the α-subunit of several hundreds of G proteins of a specific subtype. The α-and/or A′-subunit of the G proteins activates the next target protein(s) to evoke further intracellular responses. Because all of several hundreds of members in the GPCR family appear to share a common motif in their transmembrane structure and a common topology, it is generally accepted that a similar switching mechanism functions in all of these receptors.

II. MCH-R1

Neuropeptides present in the hypothalamus play a major role in mediating the control of body weight. (Flier, et al. 1998. Cell, 92, 437-440). Melanin-concentrating hormone (MCH) produced in mammals is a cyclic 19-amino acid neuropeptide synthesized as part of a larger pre-prohormone precursor in the hypothalamus which also encodes neuropeptide EI (NEI) and neuropeptide GE (NGE). (Nahon, et al., 1990. Mol. Endocrinol. 4,632-637; Vaughan, et al., U.S. Pat. No 5,049,655; and Vaughan, et al., 1989. Endocrinology 125, 1660-1665.) MCH was first identified in salmon pituitary. In fish, MCH affects melanin aggregation thus affecting skin pigmentation. In trout and eels MCH has also been shown to be involved in stress induced or corticotropin-releasing-factor (CRF)-stimulated adrenocorticotropin (ACTH) release. (Kawauchi, et al., 1983. Nature 305, 321-323.)

In humans two genes encoding MCH have been identified that are expressed in the brain. (Breton, et al., 1993. Mol. Brain Res. 18, 297-310.) In mammals MCH has been localized primarily to neuronal cell bodies of the hypothalamus which are implicated in the control of food intake, including perikarya of the lateral hypothalamus and zona inertia. (Knigge, et al., 1996. Peptides 17, 1063-1073.)

Pharmacological and genetic evidence suggest that the primary mode of MCH action is to promote feeding (orexigenic). MCH mRNA is up-regulated in fasted mice and rats, in the ob/ob mouse and in mice with targeted disruption in the gene for neuropeptide Y (NPY). (Qu, et al., 1996. Nature 380, 243-247 and Erickson, et al., 1996. Nature 381, 415-418.) Injection of MCH centrally (ICV) stimulates food intake and MCH antagonizes the hypophagic effects seen with the α melanocyte stimulating hormone (αMSH). (Qu, et al., 1996. Nature 380, 243-247.) MCH deficient mice are lean, hypophagic and have increased metabolic rate. (Shimada, et al., 1998. Nature 396, 670-673.) The administration of MCH has been indicated to be useful for promoting eating, appetite or the gain or maintenance of weight. (Maratos-Flier, U.S. Pat. No. 5,849,708.)

MCH action is not limited to modulation of food intake as effects on the hypothalamic-pituitary-axis have been reported. (Nahon, 1994. Critical Rev. in Neurobiol. 8, 221-262.) MCH may be involved in the body response to stress as MCH can modulate the stress-induced release of CRF from the hypothalamus and ACTH from the pituitary. In addition, MCH neuronal systems may be involved in reproductive or maternal function.

MCH can bind at least two different receptors: MCH-R1 and MCH-R2. (Chambers, et al. 1999. Nature 400, 262-265; Saito, et al., 1999. Nature 400, 265-269; Bäichner, et al., 1999. FEBS Letters 457:522-524; Shimomura, et al., 1999. Biochemical and Biophysical Research Communications 261, 622-626; Sailer, et al., Proc. Natl. Acad. Sci. 98:7564-7569, 2001.) The amino acid identity between MCH-R2 and MCH-R1 is about 38%. (Sailer, et al., Proc. Natl. Acad. Sci. 98:7564-7569, 2001). MCH-R2 is detected specifically in most regions of the brain, with an expression pattern similar to that of MCH-R1.

Because MCH is an important regulator of food intake and energy balance, agents capable of modulating MCH receptor activity, especially MCH-R1, are highly desirable for the treatment of obesity, eating disorders (e.g., bulimia and anorexia), sexual disorders (e.g., anorgasmic or psychogenic impotence) and metabolic disorders, such as diabetes. Small molecule, non-peptide antagonists of MCH receptors would be of particular value as therapeutic agents for the diagnosis, treatment or prevention of diseases and disorders mediated through the MCH receptor, more particularly eating disorders related to obesity of any cause in humans. Thus there is a need to provide for potent and selective antagonists of the MCH-R1 receptor.

III. Three-Dimensional Structural Information

The biological properties of a protein depend directly on the protein's three-dimensional (3D) conformation. In turn, the 3D conformation determines the activity of enzymes, the capacity and specificity of binding proteins, and the potential for small molecules to interact with the receptor. Models at the atomic level are needed to obtain insight into the mechanisms of MCH-R1 activation, and the source of interactions between specific ligands and MCH-R1. Thus, there is a need for 3D structure models for the MCH-R1 receptor.

### SUMMARY OF THE INVENTION

The present invention attempts to address this need by providing a 3D structure of human MCH-R1 receptor that is useful in identifying potent and selective agonists or antagonist of the MCH-R1 receptor for the treatment of MCH-R1 mediated disorders.

One aspect of the invention provides for a method of identifying a drug candidate useful for the treatment of a melanin concentrating hormone receptor 1 (MCH-R1) mediated disorder comprising the steps of:
(a) using a three-dimensional (3D) structure of MCH-R1 according to Table 1;
(b) employing said 3D structure to design or select in silico said drug candidate.

Another aspect of the invention provides a G-protein-coupled receptor selected from the group consisting of the following (a) or (b):
(a) a G protein-coupled receptor having three-dimensional structure I defined by the atomic coordinates as shown in Table 1;
(b) a G protein-coupled receptor having three-dimensional structure II defined by derived coordinates from the atomic coordinates as shown in Table 1, wherein the mean residual of the discrepancies between the positions of the α carbon atoms in the amino acid residues of seven helix sites SEQ ID NOS: 2-8 of the three-dimensional structure I and the positions of the corresponding α carbon atoms in the amino acid residues of the corresponding seven helix sites of the three-dimensional structure II is 1.5 Å or less when an image of the three-dimensional structure I obtained by computer-processing the atomic coordinates of Table 1 and an image of the three-dimensional structure II obtained by computer-processing said derived coordinates are superimposed.

These and other objects, features, and advantages will become apparent to those of ordinary skill in the art from a reading of the following detailed description and the appended claims.

### SEQUENCE LISTING DESCRIPTION

Each of the protein sequences in the sequence listing is shown in Table A.

TABLE A

| | | |
|---|---|---|
| | | Position on SEQ ID |
| Sequence Description | SEQ ID NO: | NO:1 |

| | | |
|---|---|---|
| MCH-R1^{a} | 1 | — |
| HI | 2 | 106-135 |
| HII | 3 | 146-175 |
| HIII | 4 | 183-213 |
| HIV | 5 | 226-249 |
| HV | 6 | 274-302 |
| HVI | 7 | 319-350 |
| HVII | 8 | 360-383 |
| ECI | 9 | 176-182 |
| ECII | 10 | 250-273 |
| ECIII | 11 | 351-359 |
| ICI | 12 | 136-145 |
| IC2 | 13 | 214-225 |
| IC3 | 14 | 303-318 |
| CTERM | 15 | 384-401 |
| THI | 16 | 106-120 |
| THII-ECI-THIII | 17 | 161-197 |
| THIV-ECII-THV | 18 | 235-288 |
| THVI-ECIII-THVII | 19 | 336-374 |
| THII | 20 | 161-175 |
| THIII | 21 | 183-197 |
| THIV | 22 | 235-249 |
| THV | 23 | 274-288 |
| THVI | 24 | 336-350 |
| THVII | 25 | 360-374 |

^{a}The corresponding human MCH-R1 accession number is NP_005288

### DETAILED DESCRIPTION OF THE INVENTION

I. 3D Coordinates of MCH-R1

G protein-coupled receptors (GPCRs) function in a number of signaling pathways, converting a variety of external stimuli to activate multiple copies of G protein subtypes specific to the relevant G protein-coupled receptor. GPCRs, including MCH-R1, share many structural features, including a bundle of seven transmembrane α-helices connected by six loops of varying lengths. In the present invention, in silico 3D coordinates of the transmembrane intra- and extra-cellular regions of MCH-R1 are provided. These coordinates were obtained by homology from the 3D structure of bacterial rhodopsin and in-house SAR data. The model was later refined using the 3D coordinates of mammalian rhodopsin (Palczewski, K.; Kumasaka, T.; Hori, T.; Behnke, C. A.; Motoshima, H.; Fox, B. A.; Trong, I. L.; Teller, D. C.; Okada, T.; Stenkamp, R. E.; Yamamoto, M.; Miyano, M. Science, 2000, 289, 739). Further refinement, such as coordinates modification in view of SAR data generated in-house, was also performed.

As a result of the above analysis, the 3D structure of human MCH-R1 receptor can be obtained as 3D coordinates. The atomic coordinates obtained by the invention are shown in Table 1 below.

TABLE 1

| | | | | | | | |
|---|---|---|---|---|---|---|---|
| ATOM | 1 | N | ILE | 105 | −40.873 | 24.697 | 19.527 |
| ATOM | 2 | CA | ILE | 105 | −40.26 | 23.523 | 20.146 |
| ATOM | 3 | C | ILE | 105 | −41.295 | 22.42 | 20.21 |
| ATOM | 4 | O | ILE | 105 | −41.087 | 21.377 | 19.609 |
| ATOM | 5 | CB | ILE | 105 | −39.654 | 23.854 | 21.544 |
| ATOM | 6 | CG1 | ILE | 105 | −38.606 | 25.001 | 21.407 |
| ATOM | 7 | CG2 | ILE | 105 | −39.038 | 22.574 | 22.18 |
| ATOM | 8 | CD1 | ILE | 105 | −38.033 | 25.492 | 22.765 |
| ATOM | 9 | HA | ILE | 105 | −39.43 | 23.17 | 19.508 |
| ATOM | 10 | HB | ILE | 105 | −40.46 | 24.211 | 22.205 |
| ATOM | 11 | HG11 | ILE | 105 | −37.774 | 24.675 | 20.762 |
| ATOM | 12 | HG12 | ILE | 105 | −39.086 | 25.868 | 20.924 |
| ATOM | 13 | HD11 | ILE | 105 | −37.396 | 24.728 | 23.234 |
| ATOM | 14 | HD12 | ILE | 105 | −37.415 | 26.39 | 22.607 |
| ATOM | 15 | HD13 | ILE | 105 | −38.849 | 25.751 | 23.457 |
| ATOM | 16 | HG21 | ILE | 105 | −38.179 | 22.226 | 21.586 |
| ATOM | 17 | HG22 | ILE | 105 | −39.779 | 21.762 | 22.227 |
| ATOM | 18 | HG23 | ILE | 105 | −38.702 | 22.763 | 23.21 |
| ATOM | 19 | H | ILE | 105 | −41.501 | 25.27 | 20.055 |
| ATOM | 20 | HNCA | ILE | 105 | −40.694 | 24.898 | 18.563 |
| ATOM | 21 | N | SER | 106 | −42.427 | 22.608 | 20.925 |
| ATOM | 22 | CA | SER | 106 | −43.437 | 21.553 | 20.948 |
| ATOM | 23 | C | SER | 106 | −43.939 | 21.352 | 19.539 |
| ATOM | 24 | O | SER | 106 | −43.916 | 20.23 | 19.06 |
| ATOM | 25 | CB | SER | 106 | −44.63 | 21.864 | 21.888 |
| ATOM | 26 | OG | SER | 106 | −45.569 | 20.776 | 21.819 |
| ATOM | 27 | HA | SER | 106 | −42.972 | 20.617 | 21.304 |
| ATOM | 28 | H | SER | 106 | −42.608 | 23.462 | 21.415 |
| ATOM | 29 | HB1 | SER | 106 | −44.256 | 21.98 | 22.919 |
| ATOM | 30 | HB2 | SER | 106 | −45.118 | 22.804 | 21.582 |
| ATOM | 31 | HG | SER | 106 | −46.329 | 20.901 | 22.38 |
| ATOM | 32 | N | TYR | 107 | −44.382 | 22.423 | 18.842 |
| ATOM | 33 | CA | TYR | 107 | −44.881 | 22.238 | 17.481 |
| ATOM | 34 | C | TYR | 107 | −43.857 | 21.508 | 16.64 |
| ATOM | 35 | O | TYR | 107 | −44.239 | 20.669 | 15.84 |
| ATOM | 36 | CB | TYR | 107 | −45.305 | 23.562 | 16.79 |
| ATOM | 37 | CG | TYR | 107 | −44.099 | 24.423 | 16.387 |
| ATOM | 38 | CD1 | TYR | 107 | −43.604 | 25.416 | 17.24 |
| ATOM | 39 | CD2 | TYR | 107 | −43.491 | 24.23 | 15.142 |
| ATOM | 40 | CE1 | TYR | 107 | −42.53 | 26.22 | 16.845 |
| ATOM | 41 | CE2 | TYR | 107 | −42.435 | 25.05 | 14.735 |
| ATOM | 42 | CZ | TYR | 107 | −41.941 | 26.042 | 15.587 |
| ATOM | 43 | OH | TYR | 107 | −40.867 | 26.831 | 15.165 |
| ATOM | 44 | HA | TYR | 107 | −45.781 | 21.608 | 17.559 |
| ATOM | 45 | H | TYR | 107 | −44.381 | 23.338 | 19.244 |
| ATOM | 46 | HB2 | TYR | 107 | −45.878 | 23.323 | 15.878 |
| ATOM | 47 | HB1 | TYR | 107 | −45.976 | 24.13 | 17.453 |
| ATOM | 48 | HD2 | TYR | 107 | −43.836 | 23.441 | 14.481 |
| ATOM | 49 | HE2 | TYR | 107 | −41.992 | 24.918 | 13.754 |
| ATOM | 50 | HE1 | TYR | 107 | −42.161 | 26.982 | 17.523 |
| ATOM | 51 | HD1 | TYR | 107 | −44.052 | 25.575 | 18.215 |
| ATOM | 52 | HH | TYR | 107 | −40.605 | 27.502 | 15.785 |
| ATOM | 53 | N | ILE | 108 | −42.545 | 21.786 | 16.815 |
| ATOM | 54 | CA | ILE | 108 | −41.542 | 21.036 | 16.059 |
| ATOM | 55 | C | ILE | 108 | −41.648 | 19.586 | 16.482 |
| ATOM | 56 | O | ILE | 108 | −41.731 | 18.716 | 15.627 |
| ATOM | 57 | CB | ILE | 108 | −40.087 | 21.572 | 16.255 |
| ATOM | 58 | CG1 | ILE | 108 | −39.975 | 23.043 | 15.75 |
| ATOM | 59 | CG2 | ILE | 108 | −39.064 | 20.648 | 15.534 |
| ATOM | 60 | CD1 | ILE | 108 | −38.596 | 23.707 | 16.008 |
| ATOM | 61 | HA | ILE | 108 | −41.785 | 21.104 | 14.986 |
| ATOM | 62 | H | ILE | 108 | −42.252 | 22.445 | 17.508 |
| ATOM | 63 | HB | ILE | 108 | −39.853 | 21.552 | 17.332 |
| ATOM | 64 | HG11 | ILE | 108 | −40.201 | 23.087 | 14.673 |
| ATOM | 65 | HG12 | ILE | 108 | −40.716 | 23.659 | 16.277 |
| ATOM | 66 | HD11 | ILE | 108 | −37.811 | 23.271 | 15.373 |
| ATOM | 67 | HD12 | ILE | 108 | −38.652 | 24.783 | 15.776 |
| ATOM | 68 | HD13 | ILE | 108 | −38.305 | 23.596 | 17.064 |
| ATOM | 69 | HG21 | ILE | 108 | −39.24 | 20.655 | 14.447 |
| ATOM | 70 | HG22 | ILE | 108 | −38.029 | 20.962 | 15.728 |
| ATOM | 71 | HG23 | ILE | 108 | −39.153 | 19.614 | 15.895 |
| ATOM | 72 | N | ASN | 109 | −41.653 | 19.299 | 17.803 |
| ATOM | 73 | CA | ASN | 109 | −41.743 | 17.907 | 18.244 |
| ATOM | 74 | C | ASN | 109 | −43.096 | 17.296 | 17.937 |
| ATOM | 75 | O | ASN | 109 | −43.193 | 16.085 | 18.026 |
| ATOM | 76 | CB | ASN | 109 | −41.43 | 17.759 | 19.758 |
| ATOM | 77 | CG | ASN | 109 | −41.147 | 16.333 | 20.179 |
| ATOM | 78 | OD1 | ASN | 109 | −40.861 | 15.493 | 19.341 |
| ATOM | 79 | ND2 | ASN | 109 | −41.198 | 16.019 | 21.49 |
| ATOM | 80 | HA | ASN | 109 | −40.981 | 17.34 | 17.682 |
| ATOM | 81 | H | ASN | 109 | −41.622 | 20.028 | 18.493 |
| ATOM | 82 | HB1 | ASN | 109 | −40.527 | 18.343 | 19.995 |
| ATOM | 83 | HB2 | ASN | 109 | −42.272 | 18.172 | 20.334 |
| ATOM | 84 | HD22 | ASN | 109 | −41.006 | 15.078 | 21.773 |
| ATOM | 85 | HD21 | ASN | 109 | −41.418 | 16.704 | 22.185 |
| ATOM | 86 | N | ILE | 110 | −44.152 | 18.061 | 17.577 |
| ATOM | 87 | CA | ILE | 110 | −45.382 | 17.447 | 17.06 |
| ATOM | 88 | C | ILE | 110 | −45.199 | 17.126 | 15.588 |
| ATOM | 89 | O | ILE | 110 | −45.728 | 16.121 | 15.139 |
| ATOM | 90 | CB | ILE | 110 | −46.627 | 18.364 | 17.283 |
| ATOM | 91 | CG1 | ILE | 110 | −46.893 | 18.527 | 18.813 |
| ATOM | 92 | CG2 | ILE | 110 | −47.882 | 17.806 | 16.548 |
| ATOM | 93 | CD1 | ILE | 110 | −47.977 | 19.581 | 19.17 |
| ATOM | 94 | HA | ILE | 110 | −45.583 | 16.499 | 17.586 |
| ATOM | 95 | H | ILE | 110 | −44.09 | 19.059 | 17.601 |
| ATOM | 96 | HB | ILE | 110 | −46.407 | 19.348 | 16.842 |
| ATOM | 97 | HG11 | ILE | 110 | −47.188 | 17.555 | 19.243 |
| ATOM | 98 | HG12 | ILE | 110 | −45.963 | 18.836 | 19.312 |
| ATOM | 99 | HD11 | ILE | 110 | −48.982 | 19.247 | 18.876 |
| ATOM | 100 | HD12 | ILE | 110 | −47.989 | 19.748 | 20.259 |
| ATOM | 101 | HD13 | ILE | 110 | −47.763 | 20.541 | 18.678 |
| ATOM | 102 | HG21 | ILE | 110 | −48.131 | 16.803 | 16.926 |
| ATOM | 103 | HG22 | ILE | 110 | −48.753 | 18.461 | 16.688 |
| ATOM | 104 | HG23 | ILE | 110 | −47.712 | 17.739 | 15.463 |
| ATOM | 105 | N | ILE | 111 | −44.47 | 17.941 | 14.793 |
| ATOM | 106 | CA | ILE | 111 | −44.342 | 17.631 | 13.365 |
| ATOM | 107 | C | ILE | 111 | −43.666 | 16.288 | 13.173 |
| ATOM | 108 | O | ILE | 111 | −44.235 | 15.43 | 12.517 |
| ATOM | 109 | CB | ILE | 111 | −43.61 | 18.762 | 12.571 |
| ATOM | 110 | CG1 | ILE | 111 | −44.517 | 20.032 | 12.49 |
| ATOM | 111 | CG2 | ILE | 111 | −43.205 | 18.277 | 11.148 |
| ATOM | 112 | CD1 | ILE | 111 | −43.768 | 21.317 | 12.041 |
| ATOM | 113 | HA | ILE | 111 | −45.357 | 17.545 | 12.942 |
| ATOM | 114 | H | ILE | 111 | −44.02 | 18.758 | 15.161 |
| ATOM | 115 | HB | ILE | 111 | −42.683 | 19.014 | 13.113 |
| ATOM | 116 | HG11 | ILE | 111 | −45.355 | 19.844 | 11.799 |
| ATOM | 117 | HG12 | ILE | 111 | −44.953 | 20.241 | 13.478 |
| ATOM | 118 | HD11 | ILE | 111 | −43.425 | 21.248 | 10.999 |
| ATOM | 119 | HD12 | ILE | 111 | −44.443 | 22.184 | 12.113 |
| ATOM | 120 | HD13 | ILE | 111 | −42.898 | 21.502 | 12.688 |
| ATOM | 121 | HG21 | ILE | 111 | −44.1 | 17.975 | 10.583 |
| ATOM | 122 | HG22 | ILE | 111 | −42.688 | 19.067 | 10.586 |
| ATOM | 123 | HG23 | ILE | 111 | −42.515 | 17.421 | 11.196 |
| ATOM | 124 | N | MET | 112 | −42.442 | 16.068 | 13.704 |
| ATOM | 125 | CA | MET | 112 | −41.75 | 14.813 | 13.397 |
| ATOM | 126 | C | MET | 112 | −42.571 | 13.561 | 13.668 |
| ATOM | 127 | O | MET | 112 | −42.664 | 12.764 | 12.748 |
| ATOM | 128 | CB | MET | 112 | −40.332 | 14.694 | 14.022 |
| ATOM | 129 | CG | MET | 112 | −39.649 | 13.368 | 13.585 |
| ATOM | 130 | SD | MET | 112 | −37.912 | 13.33 | 14.13 |
| ATOM | 131 | CE | MET | 112 | −37.437 | 11.674 | 13.55 |
| ATOM | 132 | HA | MET | 112 | −41.582 | 14.852 | 12.307 |
| ATOM | 133 | H | MET | 112 | −41.982 | 16.768 | 14.257 |
| ATOM | 134 | HB1 | MET | 112 | −39.719 | 15.544 | 13.68 |
| ATOM | 135 | HB2 | MET | 112 | −40.388 | 14.739 | 15.12 |
| ATOM | 136 | HG1 | MET | 112 | −40.169 | 12.502 | 14.025 |
| ATOM | 137 | HG2 | MET | 112 | −39.66 | 13.27 | 12.488 |
| ATOM | 138 | LPD1 | MET | 112 | −37.544 | 13.817 | 13.833 |
| ATOM | 139 | LPD2 | MET | 112 | −37.859 | 13.386 | 14.805 |
| ATOM | 140 | HE1 | MET | 112 | −37.553 | 11.606 | 12.459 |
| ATOM | 141 | HE2 | MET | 112 | −36.388 | 11.483 | 13.818 |
| ATOM | 142 | HE3 | MET | 112 | −38.079 | 10.928 | 14.04 |
| ATOM | 143 | N | PRO | 113 | −43.183 | 13.276 | 14.845 |
| ATOM | 144 | CA | PRO | 113 | −43.927 | 12.033 | 14.98 |
| ATOM | 145 | C | PRO | 113 | −45.09 | 11.98 | 14.016 |
| ATOM | 146 | O | PRO | 113 | −45.422 | 10.888 | 13.582 |
| ATOM | 147 | CB | PRO | 113 | −44.354 | 12.093 | 16.467 |
| ATOM | 148 | CG | PRO | 113 | −44.337 | 13.597 | 16.812 |
| ATOM | 149 | CD | PRO | 113 | −43.135 | 14.149 | 16.005 |
| ATOM | 150 | HA | PRO | 113 | −43.263 | 11.171 | 14.809 |
| ATOM | 151 | HD2 | PRO | 113 | −42.198 | 13.995 | 16.561 |
| ATOM | 152 | HD1 | PRO | 113 | −43.273 | 15.21 | 15.772 |
| ATOM | 153 | HG2 | PRO | 113 | −44.26 | 13.778 | 17.896 |
| ATOM | 154 | HG1 | PRO | 113 | −45.278 | 14.043 | 16.453 |
| ATOM | 155 | HB1 | PRO | 113 | −45.336 | 11.633 | 16.649 |
| ATOM | 156 | HB2 | PRO | 113 | −43.6 | 11.585 | 17.093 |
| ATOM | 157 | N | SER | 114 | −45.72 | 13.116 | 13.637 |
| ATOM | 158 | CA | SER | 114 | −46.755 | 13.049 | 12.606 |
| ATOM | 159 | C | SER | 114 | −46.127 | 12.61 | 11.301 |
| ATOM | 160 | O | SER | 114 | −46.705 | 11.792 | 10.602 |
| ATOM | 161 | CB | SER | 114 | −47.46 | 14.416 | 12.407 |
| ATOM | 162 | OG | SER | 114 | −48.039 | 14.818 | 13.661 |
| ATOM | 163 | HA | SER | 114 | −47.521 | 12.316 | 12.912 |
| ATOM | 164 | H | SER | 114 | −45.459 | 14.016 | 13.997 |
| ATOM | 165 | HB1 | SER | 114 | −46.731 | 15.168 | 12.064 |
| ATOM | 166 | HB2 | SER | 114 | −48.246 | 14.316 | 11.638 |
| ATOM | 167 | HG | SER | 114 | −48.47 | 15.666 | 13.617 |
| ATOM | 168 | N | VAL | 115 | −44.929 | 13.139 | 10.957 |
| ATOM | 169 | CA | VAL | 115 | −44.262 | 12.684 | 9.738 |
| ATOM | 170 | C | VAL | 115 | −43.977 | 11.207 | 9.882 |
| ATOM | 171 | O | VAL | 115 | −44.283 | 10.459 | 8.967 |
| ATOM | 172 | CB | VAL | 115 | −42.952 | 13.471 | 9.427 |
| ATOM | 173 | CG1 | VAL | 115 | −42.158 | 12.814 | 8.263 |
| ATOM | 174 | CG2 | VAL | 115 | −43.268 | 14.953 | 9.078 |
| ATOM | 175 | HA | VAL | 115 | −44.95 | 12.823 | 8.888 |
| ATOM | 176 | H | VAL | 115 | −44.467 | 13.802 | 11.552 |
| ATOM | 177 | HB | VAL | 115 | −42.303 | 13.451 | 10.317 |
| ATOM | 178 | HG11 | VAL | 115 | −42.787 | 12.736 | 7.362 |
| ATOM | 179 | HG12 | VAL | 115 | −41.266 | 13.411 | 8.018 |
| ATOM | 180 | HG13 | VAL | 115 | −41.82 | 11.806 | 8.544 |
| ATOM | 181 | HG21 | VAL | 115 | −43.842 | 15.437 | 9.88 |
| ATOM | 182 | HG22 | VAL | 115 | −42.335 | 15.521 | 8.936 |
| ATOM | 183 | HG23 | VAL | 115 | −43.857 | 15.011 | 8.15 |
| ATOM | 184 | N | PHE | 116 | −43.398 | 10.749 | 11.015 |
| ATOM | 185 | CA | PHE | 116 | −43.148 | 9.317 | 11.156 |
| ATOM | 186 | C | PHE | 116 | −44.44 | 8.553 | 10.983 |
| ATOM | 187 | O | PHE | 116 | −44.444 | 7.548 | 10.291 |
| ATOM | 188 | CB | PHE | 116 | −42.541 | 8.909 | 12.522 |
| ATOM | 189 | CG | PHE | 116 | −42.41 | 7.379 | 12.547 |
| ATOM | 190 | CD1 | PHE | 116 | −41.398 | 6.758 | 11.807 |
| ATOM | 191 | CD2 | PHE | 116 | −43.308 | 6.591 | 13.278 |
| ATOM | 192 | CE1 | PHE | 116 | −41.344 | 5.365 | 11.716 |
| ATOM | 193 | CE2 | PHE | 116 | −43.24 | 5.196 | 13.208 |
| ATOM | 194 | CZ | PHE | 116 | −42.281 | 4.584 | 12.396 |
| ATOM | 195 | HA | PHE | 116 | −42.446 | 9.018 | 10.361 |
| ATOM | 196 | H | PHE | 116 | −43.145 | 11.37 | 11.761 |
| ATOM | 197 | HB1 | PHE | 116 | −41.554 | 9.379 | 12.661 |
| ATOM | 198 | HB2 | PHE | 116 | −43.2 | 9.248 | 13.334 |
| ATOM | 199 | HD2 | PHE | 116 | −44.067 | 7.056 | 13.899 |
| ATOM | 200 | HE2 | PHE | 116 | −43.933 | 4.59 | 13.782 |
| ATOM | 201 | HZ | PHE | 116 | −42.266 | 3.505 | 12.293 |
| ATOM | 202 | HE1 | PHE | 116 | −40.574 | 4.895 | 11.115 |
| ATOM | 203 | HD1 | PHE | 116 | −40.653 | 7.356 | 11.292 |
| ATOM | 204 | N | GLY | 117 | −45.553 | 9.003 | 11.601 |
| ATOM | 205 | CA | GLY | 117 | −46.816 | 8.297 | 11.412 |
| ATOM | 206 | C | GLY | 117 | −47.161 | 8.218 | 9.944 |
| ATOM | 207 | O | GLY | 117 | −47.586 | 7.171 | 9.482 |
| ATOM | 208 | HA2 | GLY | 117 | −47.642 | 8.819 | 11.921 |
| ATOM | 209 | HA1 | GLY | 117 | −46.734 | 7.286 | 11.841 |
| ATOM | 210 | H | GLY | 117 | −45.537 | 9.823 | 12.179 |
| ATOM | 211 | N | THR | 118 | −46.989 | 9.322 | 9.185 |
| ATOM | 212 | CA | THR | 118 | −47.309 | 9.275 | 7.758 |
| ATOM | 213 | C | THR | 118 | −46.394 | 8.284 | 7.074 |
| ATOM | 214 | O | THR | 118 | −46.872 | 7.443 | 6.328 |
| ATOM | 215 | CB | THR | 118 | −47.189 | 10.671 | 7.078 |
| ATOM | 216 | OG1 | THR | 118 | −48.156 | 11.593 | 7.616 |
| ATOM | 217 | CG2 | THR | 118 | −47.452 | 10.576 | 5.552 |
| ATOM | 218 | HA | THR | 118 | −48.353 | 8.939 | 7.642 |
| ATOM | 219 | H | THR | 118 | −46.619 | 10.165 | 9.585 |
| ATOM | 220 | HB | THR | 118 | −46.172 | 11.074 | 7.224 |
| ATOM | 221 | HG1 | THR | 118 | −48.042 | 11.772 | 8.545 |
| ATOM | 222 | HG23 | THR | 118 | −46.685 | 9.964 | 5.055 |
| ATOM | 223 | HG21 | THR | 118 | −48.441 | 10.133 | 5.36 |
| ATOM | 224 | HG22 | THR | 118 | −47.428 | 11.584 | 5.109 |
| ATOM | 225 | N | ILE | 119 | −45.066 | 8.365 | 7.319 |
| ATOM | 226 | CA | ILE | 119 | −44.146 | 7.409 | 6.698 |
| ATOM | 227 | C | ILE | 119 | −44.62 | 6.019 | 7.06 |
| ATOM | 228 | O | ILE | 119 | −44.647 | 5.153 | 6.201 |
| ATOM | 229 | CB | ILE | 119 | −42.666 | 7.612 | 7.158 |
| ATOM | 230 | CG1 | ILE | 119 | −42.093 | 8.961 | 6.615 |
| ATOM | 231 | CG2 | ILE | 119 | −41.766 | 6.418 | 6.721 |
| ATOM | 232 | CD1 | ILE | 119 | −40.723 | 9.358 | 7.231 |
| ATOM | 233 | HA | ILE | 119 | −44.197 | 7.52 | 5.602 |
| ATOM | 234 | H | ILE | 119 | −44.713 | 9.045 | 7.966 |
| ATOM | 235 | HB | ILE | 119 | −42.662 | 7.637 | 8.26 |
| ATOM | 236 | HG11 | ILE | 119 | −41.992 | 8.901 | 5.519 |
| ATOM | 237 | HG12 | ILE | 119 | −42.792 | 9.783 | 6.833 |
| ATOM | 238 | HD11 | ILE | 119 | −39.927 | 8.658 | 6.946 |
| ATOM | 239 | HD12 | ILE | 119 | −40.424 | 10.356 | 6.873 |
| ATOM | 240 | HD13 | ILE | 119 | −40.786 | 9.385 | 8.329 |
| ATOM | 241 | HG21 | ILE | 119 | −41.695 | 6.369 | 5.624 |
| ATOM | 242 | HG22 | ILE | 119 | −40.755 | 6.519 | 7.139 |
| ATOM | 243 | HG23 | ILE | 119 | −42.156 | 5.457 | 7.085 |
| ATOM | 244 | N | CYS | 120 | −44.991 | 5.786 | 8.336 |
| ATOM | 245 | CA | CYS | 120 | −45.392 | 4.446 | 8.749 |
| ATOM | 246 | C | CYS | 120 | −46.619 | 3.976 | 8.004 |
| ATOM | 247 | O | CYS | 120 | −46.604 | 2.885 | 7.455 |
| ATOM | 248 | CB | CYS | 120 | −45.648 | 4.39 | 10.276 |
| ATOM | 249 | SG | CYS | 120 | −46.046 | 2.68 | 10.75 |
| ATOM | 250 | HA | CYS | 120 | −44.567 | 3.763 | 8.514 |
| ATOM | 251 | H | CYS | 120 | −44.977 | 6.518 | 9.019 |
| ATOM | 252 | HB1 | CYS | 120 | −44.75 | 4.714 | 10.825 |
| ATOM | 253 | HB2 | CYS | 120 | −46.487 | 5.046 | 10.55 |
| ATOM | 254 | HG | CYS | 120 | −46.353 | 2.856 | 11.693 |
| ATOM | 255 | LPG1 | CYS | 120 | −46.539 | 2.416 | 10.365 |
| ATOM | 256 | LPG2 | CYS | 120 | −45.502 | 2.275 | 10.729 |
| ATOM | 257 | N | LEU | 121 | −47.702 | 4.783 | 7.978 |
| ATOM | 258 | CA | LEU | 121 | −48.923 | 4.328 | 7.315 |
| ATOM | 259 | C | LEU | 121 | −48.625 | 3.987 | 5.872 |
| ATOM | 260 | O | LEU | 121 | −49.063 | 2.952 | 5.393 |
| ATOM | 261 | CB | LEU | 121 | −50.038 | 5.415 | 7.324 |
| ATOM | 262 | CG | LEU | 121 | −50.637 | 5.732 | 8.729 |
| ATOM | 263 | CD1 | LEU | 121 | −51.515 | 7.013 | 8.644 |
| ATOM | 264 | CD2 | LEU | 121 | −51.485 | 4.556 | 9.293 |
| ATOM | 265 | HA | LEU | 121 | −49.276 | 3.408 | 7.805 |
| ATOM | 266 | H | LEU | 121 | −47.671 | 5.693 | 8.4 |
| ATOM | 267 | HB1 | LEU | 121 | −49.604 | 6.332 | 6.895 |
| ATOM | 268 | HB2 | LEU | 121 | −50.861 | 5.096 | 6.662 |
| ATOM | 269 | HG | LEU | 121 | −49.826 | 5.935 | 9.447 |
| ATOM | 270 | HD21 | LEU | 121 | −50.868 | 3.668 | 9.489 |
| ATOM | 271 | HD22 | LEU | 121 | −51.953 | 4.847 | 10.246 |
| ATOM | 272 | HD23 | LEU | 121 | −52.283 | 4.282 | 8.586 |
| ATOM | 273 | HD11 | LEU | 121 | −52.347 | 6.861 | 7.939 |
| ATOM | 274 | HD12 | LEU | 121 | −51.934 | 7.266 | 9.631 |
| ATOM | 275 | HD13 | LEU | 121 | −50.917 | 7.872 | 8.301 |
| ATOM | 276 | N | LEU | 122 | −47.884 | 4.857 | 5.154 |
| ATOM | 277 | CA | LEU | 122 | −47.649 | 4.599 | 3.736 |
| ATOM | 278 | C | LEU | 122 | −46.677 | 3.45 | 3.564 |
| ATOM | 279 | O | LEU | 122 | −46.929 | 2.561 | 2.766 |
| ATOM | 280 | CB | LEU | 122 | −47.075 | 5.854 | 3.021 |
| ATOM | 281 | CG | LEU | 122 | −47.974 | 7.128 | 3.063 |
| ATOM | 282 | CD1 | LEU | 122 | −47.205 | 8.32 | 2.425 |
| ATOM | 283 | CD2 | LEU | 122 | −49.33 | 6.921 | 2.332 |
| ATOM | 284 | HA | LEU | 122 | −48.593 | 4.308 | 3.249 |
| ATOM | 285 | H | LEU | 122 | −47.476 | 5.666 | 5.587 |
| ATOM | 286 | HB1 | LEU | 122 | −46.112 | 6.094 | 3.499 |
| ATOM | 287 | HB2 | LEU | 122 | −46.887 | 5.596 | 1.966 |
| ATOM | 288 | HG | LEU | 122 | −48.195 | 7.402 | 4.108 |
| ATOM | 289 | HD21 | LEU | 122 | −49.958 | 6.186 | 2.855 |
| ATOM | 290 | HD22 | LEU | 122 | −49.891 | 7.868 | 2.295 |
| ATOM | 291 | HD23 | LEU | 122 | −49.161 | 6.577 | 1.3 |
| ATOM | 292 | HD11 | LEU | 122 | −46.99 | 8.113 | 1.365 |
| ATOM | 293 | HD12 | LEU | 122 | −47.8 | 9.245 | 2.487 |
| ATOM | 294 | HD13 | LEU | 122 | −46.251 | 8.492 | 2.946 |
| ATOM | 295 | N | GLY | 123 | −45.54 | 3.454 | 4.294 |
| ATOM | 296 | CA | GLY | 123 | −44.525 | 2.43 | 4.066 |
| ATOM | 297 | C | GLY | 123 | −44.979 | 1.056 | 4.495 |
| ATOM | 298 | O | GLY | 123 | −44.606 | 0.104 | 3.826 |
| ATOM | 299 | HA2 | GLY | 123 | −43.599 | 2.672 | 4.606 |
| ATOM | 300 | HA1 | GLY | 123 | −44.275 | 2.405 | 2.993 |
| ATOM | 301 | H | GLY | 123 | −45.362 | 4.161 | 4.982 |
| ATOM | 302 | N | ILE | 124 | −45.759 | 0.894 | 5.59 |
| ATOM | 303 | CA | ILE | 124 | −46.226 | −0.453 | 5.926 |
| ATOM | 304 | C | ILE | 124 | −47.066 | −0.936 | 4.77 |
| ATOM | 305 | O | ILE | 124 | −46.85 | −2.039 | 4.293 |
| ATOM | 306 | CB | ILE | 124 | −47.041 | −0.578 | 7.254 |
| ATOM | 307 | CG1 | ILE | 124 | −46.092 | −0.359 | 8.47 |
| ATOM | 308 | CG2 | ILE | 124 | −47.75 | −1.963 | 7.351 |
| ATOM | 309 | CD1 | ILE | 124 | −46.78 | −0.457 | 9.858 |
| ATOM | 310 | HA | ILE | 124 | −45.351 | −1.115 | 6.002 |
| ATOM | 311 | H | ILE | 124 | −46.062 | 1.677 | 6.141 |
| ATOM | 312 | HB | ILE | 124 | −47.822 | 0.202 | 7.257 |
| ATOM | 313 | HG11 | ILE | 124 | −45.276 | −1.099 | 8.444 |
| ATOM | 314 | HG12 | ILE | 124 | −45.652 | 0.645 | 8.375 |
| ATOM | 315 | HD11 | ILE | 124 | −47.092 | −1.486 | 10.089 |
| ATOM | 316 | HD12 | ILE | 124 | −46.073 | −0.155 | 10.646 |
| ATOM | 317 | HD13 | ILE | 124 | −47.658 | 0.205 | 9.899 |
| ATOM | 318 | HG21 | ILE | 124 | −47 | −2.767 | 7.387 |
| ATOM | 319 | HG22 | ILE | 124 | −48.382 | −2.027 | 8.247 |
| ATOM | 320 | HG23 | ILE | 124 | −48.419 | −2.146 | 6.497 |
| ATOM | 321 | N | ILE | 125 | −48.044 | −0.123 | 4.32 |
| ATOM | 322 | CA | ILE | 125 | −48.936 | −0.596 | 3.265 |
| ATOM | 323 | C | ILE | 125 | −48.097 | −0.909 | 2.048 |
| ATOM | 324 | O | ILE | 125 | −48.209 | −2.004 | 1.52 |
| ATOM | 325 | CB | ILE | 125 | −50.079 | 0.421 | 2.957 |
| ATOM | 326 | CG1 | ILE | 125 | −51.049 | 0.514 | 4.179 |
| ATOM | 327 | CG2 | ILE | 125 | −50.839 | 0.011 | 1.662 |
| ATOM | 328 | CD1 | ILE | 125 | −52.043 | 1.705 | 4.103 |
| ATOM | 329 | HA | ILE | 125 | −49.405 | −1.537 | 3.599 |
| ATOM | 330 | H | ILE | 125 | −48.156 | 0.803 | 4.69 |
| ATOM | 331 | HB | ILE | 125 | −49.619 | 1.408 | 2.784 |
| ATOM | 332 | HG11 | ILE | 125 | −51.617 | −0.426 | 4.274 |
| ATOM | 333 | HG12 | ILE | 125 | −50.472 | 0.641 | 5.109 |
| ATOM | 334 | HD11 | ILE | 125 | −52.768 | 1.578 | 3.287 |
| ATOM | 335 | HD12 | ILE | 125 | −52.608 | 1.778 | 5.045 |
| ATOM | 336 | HD13 | ILE | 125 | −51.498 | 2.65 | 3.953 |
| ATOM | 337 | HG21 | ILE | 125 | −51.291 | −0.985 | 1.786 |
| ATOM | 338 | HG22 | ILE | 125 | −51.635 | 0.73 | 1.419 |
| ATOM | 339 | HG23 | ILE | 125 | −50.16 | −0.018 | 0.796 |
| ATOM | 340 | N | GLY | 126 | −47.249 | 0.03 | 1.577 |
| ATOM | 341 | CA | GLY | 126 | −46.479 | −0.244 | 0.366 |
| ATOM | 342 | C | GLY | 126 | −45.643 | −1.493 | 0.508 |
| ATOM | 343 | O | GLY | 126 | −45.741 | −2.385 | −0.319 |
| ATOM | 344 | HA2 | GLY | 126 | −45.793 | 0.588 | 0.14 |
| ATOM | 345 | HA1 | GLY | 126 | −47.171 | −0.352 | −0.484 |
| ATOM | 346 | H | GLY | 126 | −47.15 | 0.921 | 2.03 |
| ATOM | 347 | N | ASN | 127 | −44.791 | −1.579 | 1.551 |
| ATOM | 348 | CA | ASN | 127 | −43.893 | −2.73 | 1.652 |
| ATOM | 349 | C | ASN | 127 | −44.662 | −4.015 | 1.883 |
| ATOM | 350 | O | ASN | 127 | −44.339 | −5.013 | 1.256 |
| ATOM | 351 | CB | ASN | 127 | −42.836 | −2.496 | 2.76 |
| ATOM | 352 | CG | ASN | 127 | −41.959 | −1.326 | 2.383 |
| ATOM | 353 | OD1 | ASN | 127 | −41.217 | −1.453 | 1.424 |
| ATOM | 354 | ND2 | ASN | 127 | −42.005 | −0.175 | 3.086 |
| ATOM | 355 | HA | ASN | 127 | −43.35 | −2.833 | 0.697 |
| ATOM | 356 | H | ASN | 127 | −44.762 | −0.856 | 2.248 |
| ATOM | 357 | HB1 | ASN | 127 | −43.353 | −2.326 | 3.712 |
| ATOM | 358 | HB2 | ASN | 127 | −42.198 | −3.383 | 2.875 |
| ATOM | 359 | HD22 | ASN | 127 | −41.416 | 0.592 | 2.816 |
| ATOM | 360 | HD21 | ASN | 127 | −42.614 | −0.064 | 3.87 |
| ATOM | 361 | N | SER | 128 | −45.691 | −4.04 | 2.761 |
| ATOM | 362 | CA | SER | 128 | −46.468 | −5.272 | 2.924 |
| ATOM | 363 | C | SER | 128 | −47.094 | −5.642 | 1.6 |
| ATOM | 364 | O | SER | 128 | −46.996 | −6.786 | 1.182 |
| ATOM | 365 | CB | SER | 128 | −47.607 | −5.152 | 3.972 |
| ATOM | 366 | OG | SER | 128 | −47.048 | −5.009 | 5.289 |
| ATOM | 367 | HA | SER | 128 | −45.797 | −6.088 | 3.234 |
| ATOM | 368 | H | SER | 128 | −45.96 | −3.221 | 3.275 |
| ATOM | 369 | HB1 | SER | 128 | −48.249 | −4.288 | 3.731 |
| ATOM | 370 | HB2 | SER | 128 | −48.223 | −6.068 | 3.942 |
| ATOM | 371 | HG | SER | 128 | −47.711 | −4.929 | 5.969 |
| ATOM | 372 | N | THR | 129 | −47.749 | −4.669 | 0.931 |
| ATOM | 373 | CA | THR | 129 | −48.36 | −4.956 | −0.363 |
| ATOM | 374 | C | THR | 129 | −47.338 | −5.555 | −1.3 |
| ATOM | 375 | O | THR | 129 | −47.644 | −6.537 | −1.96 |
| ATOM | 376 | CB | THR | 129 | −48.97 | −3.66 | −0.963 |
| ATOM | 377 | OG1 | THR | 129 | −50.069 | −3.27 | −0.118 |
| ATOM | 378 | CG2 | THR | 129 | −49.464 | −3.876 | −2.413 |
| ATOM | 379 | HA | THR | 129 | −49.175 | −5.682 | −0.213 |
| ATOM | 380 | H | THR | 129 | −47.824 | −3.739 | 1.304 |
| ATOM | 381 | HB | THR | 129 | −48.201 | −2.868 | −0.978 |
| ATOM | 382 | HG1 | THR | 129 | −50.469 | −2.449 | −0.382 |
| ATOM | 383 | HG23 | THR | 129 | −48.611 | −4.09 | −3.074 |
| ATOM | 384 | HG21 | THR | 129 | −50.173 | −4.718 | −2.444 |
| ATOM | 385 | HG22 | THR | 129 | −49.968 | −2.969 | −2.783 |
| ATOM | 386 | N | VAL | 130 | −46.117 | −4.982 | −1.378 |
| ATOM | 387 | CA | VAL | 130 | −45.117 | −5.543 | −2.285 |
| ATOM | 388 | C | VAL | 130 | −44.836 | −6.983 | −1.921 |
| ATOM | 389 | O | VAL | 130 | −44.83 | −7.824 | −2.806 |
| ATOM | 390 | CB | VAL | 130 | −43.79 | −4.726 | −2.304 |
| ATOM | 391 | CG1 | VAL | 130 | −42.648 | −5.488 | −3.032 |
| ATOM | 392 | CG2 | VAL | 130 | −43.994 | −3.355 | −3.008 |
| ATOM | 393 | HA | VAL | 130 | −45.538 | −5.524 | −3.3 |
| ATOM | 394 | H | VAL | 130 | −45.883 | −4.184 | −0.815 |
| ATOM | 395 | HB | VAL | 130 | −43.469 | −4.555 | −1.262 |
| ATOM | 396 | HG11 | VAL | 130 | −42.958 | −5.753 | −4.053 |
| ATOM | 397 | HG12 | VAL | 130 | −41.764 | −4.839 | −3.096 |
| ATOM | 398 | HG13 | VAL | 130 | −42.358 | −6.405 | −2.496 |
| ATOM | 399 | HG21 | VAL | 130 | −44.836 | −2.798 | −2.579 |
| ATOM | 400 | HG22 | VAL | 130 | −43.084 | −2.747 | −2.901 |
| ATOM | 401 | HG23 | VAL | 130 | −44.191 | −3.498 | −4.08 |
| ATOM | 402 | N | ILE | 131 | −44.586 | −7.301 | −0.633 |
| ATOM | 403 | CA | ILE | 131 | −44.219 | −8.679 | −0.293 |
| ATOM | 404 | C | ILE | 131 | −45.377 | −9.574 | −0.644 |
| ATOM | 405 | O | ILE | 131 | −45.185 | −10.553 | −1.349 |
| ATOM | 406 | CB | ILE | 131 | −43.862 | −8.871 | 1.211 |
| ATOM | 407 | CG1 | ILE | 131 | −42.571 | −8.071 | 1.544 |
| ATOM | 408 | CG2 | ILE | 131 | −43.679 | −10.374 | 1.572 |
| ATOM | 409 | CD1 | ILE | 131 | −42.457 | −7.705 | 3.042 |
| ATOM | 410 | HA | ILE | 131 | −43.344 | −8.977 | −0.896 |
| ATOM | 411 | H | ILE | 131 | −44.668 | −6.603 | 0.086 |
| ATOM | 412 | HB | ILE | 131 | −44.698 | −8.472 | 1.809 |
| ATOM | 413 | HG11 | ILE | 131 | −41.674 | −8.618 | 1.213 |
| ATOM | 414 | HG12 | ILE | 131 | −42.603 | −7.128 | 0.99 |
| ATOM | 415 | HD11 | ILE | 131 | −42.391 | −8.597 | 3.68 |
| ATOM | 416 | HD12 | ILE | 131 | −41.564 | −7.087 | 3.204 |
| ATOM | 417 | HD13 | ILE | 131 | −43.336 | −7.116 | 3.337 |
| ATOM | 418 | HG21 | ILE | 131 | −42.875 | −10.813 | 0.962 |
| ATOM | 419 | HG22 | ILE | 131 | −43.415 | −10.497 | 2.632 |
| ATOM | 420 | HG23 | ILE | 131 | −44.603 | −10.947 | 1.395 |
| ATOM | 421 | N | PHE | 132 | −46.59 | −9.249 | −0.15 |
| ATOM | 422 | CA | PHE | 132 | −47.734 | −10.104 | −0.439 |
| ATOM | 423 | C | PHE | 132 | −47.839 | −10.323 | −1.927 |
| ATOM | 424 | O | PHE | 132 | −48.001 | −11.452 | −2.363 |
| ATOM | 425 | CB | PHE | 132 | −49.049 | −9.447 | 0.056 |
| ATOM | 426 | CG | PHE | 132 | −50.257 | −10.296 | −0.362 |
| ATOM | 427 | CD1 | PHE | 132 | −51.009 | −9.962 | −1.494 |
| ATOM | 428 | CD2 | PHE | 132 | −50.615 | −11.416 | 0.396 |
| ATOM | 429 | CE1 | PHE | 132 | −52.151 | −10.698 | −1.824 |
| ATOM | 430 | CE2 | PHE | 132 | −51.746 | −12.165 | 0.057 |
| ATOM | 431 | CZ | PHE | 132 | −52.525 | −11.794 | −1.041 |
| ATOM | 432 | HA | PHE | 132 | −47.595 | −11.075 | 0.064 |
| ATOM | 433 | H | PHE | 132 | −46.72 | −8.426 | 0.411 |
| ATOM | 434 | HB1 | PHE | 132 | −49.026 | −9.351 | 1.153 |
| ATOM | 435 | HB2 | PHE | 132 | −49.144 | −8.432 | −0.361 |
| ATOM | 436 | HD2 | PHE | 132 | −50.016 | −11.708 | 1.253 |
| ATOM | 437 | HE2 | PHE | 132 | −52.019 | −13.034 | 0.649 |
| ATOM | 438 | HZ | PHE | 132 | −53.421 | −12.358 | −1.284 |
| ATOM | 439 | HE1 | PHE | 132 | −52.747 | −10.417 | −2.687 |
| ATOM | 440 | HD1 | PHE | 132 | −50.711 | −9.128 | −2.123 |
| ATOM | 441 | N | ALA | 133 | −47.757 | −9.242 | −2.729 |
| ATOM | 442 | CA | ALA | 133 | −47.975 | −9.396 | −4.162 |
| ATOM | 443 | C | ALA | 133 | −46.844 | −10.121 | −4.857 |
| ATOM | 444 | O | ALA | 133 | −47.134 | −10.928 | −5.727 |
| ATOM | 445 | CB | ALA | 133 | −48.209 | −8.016 | −4.819 |
| ATOM | 446 | HA | ALA | 133 | −48.894 | −9.991 | −4.291 |
| ATOM | 447 | H | ALA | 133 | −47.567 | −8.33 | −2.353 |
| ATOM | 448 | HB1 | ALA | 133 | −48.262 | −8.108 | −5.913 |
| ATOM | 449 | HB2 | ALA | 133 | −49.15 | −7.589 | −4.442 |
| ATOM | 450 | HB3 | ALA | 133 | −47.387 | −7.337 | −4.564 |
| ATOM | 451 | N | VAL | 134 | −45.56 | −9.87 | −4.514 |
| ATOM | 452 | CA | VAL | 134 | −44.486 | −10.641 | −5.145 |
| ATOM | 453 | C | VAL | 134 | −44.653 | −12.082 | −4.718 |
| ATOM | 454 | O | VAL | 134 | −44.691 | −12.958 | −5.567 |
| ATOM | 455 | CB | VAL | 134 | −43.046 | −10.154 | −4.791 |
| ATOM | 456 | CG1 | VAL | 134 | −41.977 | −11.128 | −5.366 |
| ATOM | 457 | CG2 | VAL | 134 | −42.764 | −8.727 | −5.339 |
| ATOM | 458 | HA | VAL | 134 | −44.597 | −10.589 | −6.241 |
| ATOM | 459 | H | VAL | 134 | −45.337 | −9.208 | −3.793 |
| ATOM | 460 | HB | VAL | 134 | −42.942 | −10.127 | −3.692 |
| ATOM | 461 | HG11 | VAL | 134 | −42.107 | −11.246 | −6.452 |
| ATOM | 462 | HG12 | VAL | 134 | −40.964 | −10.74 | −5.184 |
| ATOM | 463 | HG13 | VAL | 134 | −42.053 | −12.122 | −4.897 |
| ATOM | 464 | HG21 | VAL | 134 | −43.532 | −8.019 | −5.003 |
| ATOM | 465 | HG22 | VAL | 134 | −41.787 | −8.364 | −4.984 |
| ATOM | 466 | HG23 | VAL | 134 | −42.748 | −8.735 | −6.439 |
| ATOM | 467 | N | VAL | 135 | −44.754 | −12.347 | −3.397 |
| ATOM | 468 | CA | VAL | 135 | −44.849 | −13.731 | −2.93 |
| ATOM | 469 | C | VAL | 135 | −46.077 | −14.395 | −3.514 |
| ATOM | 470 | O | VAL | 135 | −46.029 | −15.586 | −3.778 |
| ATOM | 471 | CB | VAL | 135 | −44.843 | −13.813 | −1.372 |
| ATOM | 472 | CG1 | VAL | 135 | −45.164 | −15.248 | −0.865 |
| ATOM | 473 | CG2 | VAL | 135 | −43.464 | −13.365 | −0.804 |
| ATOM | 474 | HA | VAL | 135 | −43.972 | −14.284 | −3.306 |
| ATOM | 475 | H | VAL | 135 | −44.787 | −11.599 | −2.731 |
| ATOM | 476 | HB | VAL | 135 | −45.629 | −13.141 | −0.984 |
| ATOM | 477 | HG11 | VAL | 135 | −44.455 | −15.972 | −1.294 |
| ATOM | 478 | HG12 | VAL | 135 | −45.09 | −15.293 | 0.232 |
| ATOM | 479 | HG13 | VAL | 135 | −46.186 | −15.548 | −1.142 |
| ATOM | 480 | HG21 | VAL | 135 | −43.187 | −12.355 | −1.142 |
| ATOM | 481 | HG22 | VAL | 135 | −43.489 | −13.36 | 0.297 |
| ATOM | 482 | HG23 | VAL | 135 | −42.674 | −14.059 | −1.128 |
| ATOM | 483 | N | LYS | 136 | −47.193 | −13.666 | −3.738 |
| ATOM | 484 | CA | LYS | 136 | −48.38 | −14.311 | −4.303 |
| ATOM | 485 | C | LYS | 136 | −48.119 | −14.994 | −5.631 |
| ATOM | 486 | O | LYS | 136 | −48.946 | −15.817 | −5.992 |
| ATOM | 487 | CB | LYS | 136 | −49.567 | −13.308 | −4.41 |
| ATOM | 488 | CG | LYS | 136 | −50.884 | −13.942 | −4.945 |
| ATOM | 489 | CD | LYS | 136 | −52.107 | −13.027 | −4.661 |
| ATOM | 490 | CE | LYS | 136 | −53.436 | −13.622 | −5.202 |
| ATOM | 491 | NZ | LYS | 136 | −54.611 | −12.819 | −4.777 |
| ATOM | 492 | HA | LYS | 136 | −48.679 | −15.103 | −3.596 |
| ATOM | 493 | H | LYS | 136 | −47.231 | −12.693 | −3.498 |
| ATOM | 494 | HB1 | LYS | 136 | −49.758 | −12.915 | −3.399 |
| ATOM | 495 | HB2 | LYS | 136 | −49.278 | −12.466 | −5.059 |
| ATOM | 496 | HG1 | LYS | 136 | −50.805 | −14.113 | −6.031 |
| ATOM | 497 | HG2 | LYS | 136 | −51.049 | −14.912 | −4.449 |
| ATOM | 498 | HD1 | LYS | 136 | −52.201 | −12.905 | −3.572 |
| ATOM | 499 | HD2 | LYS | 136 | −51.936 | −12.035 | −5.109 |
| ATOM | 500 | HE1 | LYS | 136 | −53.386 | −13.667 | −6.304 |
| ATOM | 501 | HE2 | LYS | 136 | −53.548 | −14.654 | −4.827 |
| ATOM | 502 | HZ1 | LYS | 136 | −54.554 | −11.786 | −5.093 |
| ATOM | 503 | HZ2 | LYS | 136 | −55.534 | −13.217 | −5.18 |
| ATOM | 504 | HZ3 | LYS | 136 | −54.728 | −12.808 | −3.701 |
| ATOM | 505 | N | LYS | 137 | −47.025 | −14.713 | −6.384 |
| ATOM | 506 | CA | LYS | 137 | −46.802 | −15.406 | −7.657 |
| ATOM | 507 | C | LYS | 137 | −45.378 | −15.905 | −7.774 |
| ATOM | 508 | O | LYS | 137 | −44.519 | −15.373 | −7.091 |
| ATOM | 509 | CB | LYS | 137 | −47.153 | −14.459 | −8.835 |
| ATOM | 510 | CG | LYS | 137 | −48.605 | −13.918 | −8.705 |
| ATOM | 511 | CD | LYS | 137 | −49.017 | −13.102 | −9.961 |
| ATOM | 512 | CE | LYS | 137 | −50.469 | −12.549 | −9.895 |
| ATOM | 513 | NZ | LYS | 137 | −50.667 | −11.485 | −8.88 |
| ATOM | 514 | HA | LYS | 137 | −47.436 | −16.305 | −7.734 |
| ATOM | 515 | H | LYS | 137 | −46.345 | −14.028 | −6.108 |
| ATOM | 516 | HB1 | LYS | 137 | −46.445 | −13.614 | −8.837 |
| ATOM | 517 | HB2 | LYS | 137 | −47.037 | −15.008 | −9.784 |
| ATOM | 518 | HG1 | LYS | 137 | −49.303 | −14.761 | −8.581 |
| ATOM | 519 | HG2 | LYS | 137 | −48.661 | −13.283 | −7.807 |
| ATOM | 520 | HD1 | LYS | 137 | −48.314 | −12.27 | −10.096 |
| ATOM | 521 | HD2 | LYS | 137 | −48.934 | −13.76 | −10.843 |
| ATOM | 522 | HE1 | LYS | 137 | −50.714 | −12.135 | −10.888 |
| ATOM | 523 | HE2 | LYS | 137 | −51.163 | −13.386 | −9.704 |
| ATOM | 524 | HZ1 | LYS | 137 | −50.061 | −10.602 | −9.034 |
| ATOM | 525 | HZ2 | LYS | 137 | −51.687 | −11.136 | −8.878 |
| ATOM | 526 | HZ3 | LYS | 137 | −50.508 | −11.817 | −7.865 |
| ATOM | 527 | N | SER | 138 | −45.101 | −16.928 | −8.622 |
| ATOM | 528 | CA | SER | 138 | −43.777 | −17.553 | −8.63 |
| ATOM | 529 | C | SER | 138 | −43.327 | −17.857 | −10.042 |
| ATOM | 530 | O | SER | 138 | −43.119 | −19.012 | −10.378 |
| ATOM | 531 | CB | SER | 138 | −43.842 | −18.836 | −7.762 |
| ATOM | 532 | OG | SER | 138 | −44.23 | −18.475 | −6.425 |
| ATOM | 533 | HA | SER | 138 | −43.004 | −16.897 | −8.197 |
| ATOM | 534 | H | SER | 138 | −45.809 | −17.34 | −9.197 |
| ATOM | 535 | HB1 | SER | 138 | −44.582 | −19.53 | −8.195 |
| ATOM | 536 | HB2 | SER | 138 | −42.855 | −19.33 | −7.749 |
| ATOM | 537 | HG | SER | 138 | −44.295 | −19.223 | −5.838 |
| ATOM | 538 | N | LYS | 139 | −43.151 | −16.817 | −10.887 |
| ATOM | 539 | CA | LYS | 139 | −42.596 | −17.029 | −12.226 |
| ATOM | 540 | C | LYS | 139 | −41.706 | −15.845 | −12.534 |
| ATOM | 541 | O | LYS | 139 | −42.192 | −14.868 | −13.086 |
| ATOM | 542 | CB | LYS | 139 | −43.721 | −17.217 | −13.279 |
| ATOM | 543 | CG | LYS | 139 | −43.126 | −17.45 | −14.699 |
| ATOM | 544 | CD | LYS | 139 | −44.223 | −17.779 | −15.749 |
| ATOM | 545 | CE | LYS | 139 | −43.607 | −17.983 | −17.162 |
| ATOM | 546 | NZ | LYS | 139 | −44.628 | −18.386 | −18.161 |
| ATOM | 547 | HA | LYS | 139 | −41.972 | −17.938 | −12.251 |
| ATOM | 548 | H | LYS | 139 | −43.386 | −15.887 | −10.6 |
| ATOM | 549 | HB1 | LYS | 139 | −44.323 | −18.092 | −12.984 |
| ATOM | 550 | HB2 | LYS | 139 | −44.382 | −16.336 | −13.279 |
| ATOM | 551 | HG1 | LYS | 139 | −42.576 | −16.553 | −15.028 |
| ATOM | 552 | HG2 | LYS | 139 | −42.414 | −18.29 | −14.66 |
| ATOM | 553 | HD1 | LYS | 139 | −44.748 | −18.699 | −15.444 |
| ATOM | 554 | HD2 | LYS | 139 | −44.957 | −16.957 | −15.783 |
| ATOM | 555 | HE1 | LYS | 139 | −43.127 | −17.042 | −17.482 |
| ATOM | 556 | HE2 | LYS | 139 | −42.824 | −18.76 | −17.109 |
| ATOM | 557 | HZ1 | LYS | 139 | −45.414 | −17.649 | −18.258 |
| ATOM | 558 | HZ2 | LYS | 139 | −44.197 | −18.514 | −19.146 |
| ATOM | 559 | HZ3 | LYS | 139 | −45.098 | −19.326 | −17.904 |
| ATOM | 560 | N | LEU | 140 | −40.408 | −15.911 | −12.157 |
| ATOM | 561 | CA | LEU | 140 | −39.484 | −14.806 | −12.4 |
| ATOM | 562 | C | LEU | 140 | −38.466 | −15.342 | −13.392 |
| ATOM | 563 | O | LEU | 140 | −37.993 | −16.447 | −13.175 |
| ATOM | 564 | CB | LEU | 140 | −38.828 | −14.387 | −11.05 |
| ATOM | 565 | CG | LEU | 140 | −39.84 | −14.149 | −9.884 |
| ATOM | 566 | CD1 | LEU | 140 | −39.115 | −14.123 | −8.507 |
| ATOM | 567 | CD2 | LEU | 140 | −40.635 | −12.827 | −10.055 |
| ATOM | 568 | HA | LEU | 140 | −39.99 | −13.927 | −12.825 |
| ATOM | 569 | H | LEU | 140 | −40.03 | −16.724 | −11.707 |
| ATOM | 570 | HB1 | LEU | 140 | −38.184 | −15.219 | −10.741 |
| ATOM | 571 | HB2 | LEU | 140 | −38.197 | −13.498 | −11.201 |
| ATOM | 572 | HG | LEU | 140 | −40.559 | −14.986 | −9.85 |
| ATOM | 573 | HD21 | LEU | 140 | −41.11 | −12.779 | −11.04 |
| ATOM | 574 | HD22 | LEU | 140 | −41.42 | −12.745 | −9.286 |
| ATOM | 575 | HD23 | LEU | 140 | −39.964 | −11.964 | −9.956 |
| ATOM | 576 | HD11 | LEU | 140 | −38.414 | −13.279 | −8.458 |
| ATOM | 577 | HD12 | LEU | 140 | −39.845 | −14.012 | −7.69 |
| ATOM | 578 | HD13 | LEU | 140 | −38.553 | −15.055 | −8.337 |
| ATOM | 579 | N | HIS | 141 | −38.118 | −14.625 | −14.486 |
| ATOM | 580 | CA | HIS | 141 | −37.131 | −15.151 | −15.438 |
| ATOM | 581 | C | HIS | 141 | −35.719 | −14.827 | −14.995 |
| ATOM | 582 | O | HIS | 141 | −35.546 | −14.14 | −13.999 |
| ATOM | 583 | CB | HIS | 141 | −37.42 | −14.581 | −16.855 |
| ATOM | 584 | CG | HIS | 141 | −38.75 | −15.144 | −17.355 |
| ATOM | 585 | CD2 | HIS | 141 | −39.982 | −14.517 | −17.461 |
| ATOM | 586 | CE1 | HIS | 141 | −40.19 | −16.584 | −18.158 |
| ATOM | 587 | HA | HIS | 141 | −37.204 | −16.25 | −15.504 |
| ATOM | 588 | H | HIS | 141 | −38.511 | −13.72 | −14.651 |
| ATOM | 589 | HB1 | HIS | 141 | −37.45 | −13.481 | −16.84 |
| ATOM | 590 | HB2 | HIS | 141 | −36.656 | −14.889 | −17.583 |
| ATOM | 591 | HD2 | HIS | 141 | −40.211 | −13.49 | −17.208 |
| ATOM | 592 | N2 | HIS | 141 | −40.847 | −15.428 | −17.944 |
| ATOM | 593 | HE1 | HIS | 141 | −40.616 | −17.497 | −18.561 |
| ATOM | 594 | N1 | HIS | 141 | −38.909 | −16.408 | −17.787 |
| ATOM | 595 | N | TRP | 142 | −34.675 | −15.314 | −15.709 |
| ATOM | 596 | CA | TRP | 142 | −33.314 | −14.88 | −15.385 |
| ATOM | 597 | C | TRP | 142 | −33.207 | −13.391 | −15.652 |
| ATOM | 598 | O | TRP | 142 | −34.051 | −12.869 | −16.362 |
| ATOM | 599 | CB | TRP | 142 | −32.204 | −15.677 | −16.127 |
| ATOM | 600 | CG | TRP | 142 | −31.927 | −15.171 | −17.521 |
| ATOM | 601 | CD1 | TRP | 142 | −32.759 | −15.153 | −18.577 |
| ATOM | 602 | CD2 | TRP | 142 | −30.619 | −14.584 | −17.991 |
| ATOM | 603 | NE1 | TRP | 142 | −32.145 | −14.636 | −19.61 |
| ATOM | 604 | CE2 | TRP | 142 | −30.88 | −14.283 | −19.314 |
| ATOM | 605 | CE3 | TRP | 142 | −29.376 | −14.332 | −17.412 |
| ATOM | 606 | CZ2 | TRP | 142 | −29.916 | −13.706 | −20.141 |
| ATOM | 607 | CZ3 | TRP | 142 | −28.394 | −13.76 | −18.232 |
| ATOM | 608 | CH2 | TRP | 142 | −28.663 | −13.444 | −19.573 |
| ATOM | 609 | HA | TRP | 142 | −33.175 | −15.053 | −14.303 |
| ATOM | 610 | HB1 | TRP | 142 | −31.265 | −15.599 | −15.557 |
| ATOM | 611 | HB2 | TRP | 142 | −32.471 | −16.744 | −16.169 |
| ATOM | 612 | HE1 | TRP | 142 | −32.575 | −14.516 | −20.54 |
| ATOM | 613 | HD1 | TRP | 142 | −33.788 | −15.509 | −18.59 |
| ATOM | 614 | HZ2 | TRP | 142 | −30.132 | −13.471 | −21.178 |
| ATOM | 615 | HH2 | TRP | 142 | −27.887 | −12.988 | −20.18 |
| ATOM | 616 | HZ3 | TRP | 142 | −27.41 | −13.556 | −17.82 |
| ATOM | 617 | HE3 | TRP | 142 | −29.175 | −14.565 | −16.374 |
| ATOM | 618 | H | TRP | 142 | −34.814 | −15.939 | −16.477 |
| ATOM | 619 | N | CYS | 143 | −32.214 | −12.682 | −15.065 |
| ATOM | 620 | CA | CYS | 143 | −32.197 | −11.214 | −15.119 |
| ATOM | 621 | C | CYS | 143 | −33.421 | −10.589 | −14.475 |
| ATOM | 622 | O | CYS | 143 | −33.652 | −9.411 | −14.702 |
| ATOM | 623 | CB | CYS | 143 | −31.982 | −10.668 | −16.557 |
| ATOM | 624 | SG | CYS | 143 | −30.466 | −11.407 | −17.246 |
| ATOM | 625 | HA | CYS | 143 | −31.347 | −10.846 | −14.516 |
| ATOM | 626 | HB1 | CYS | 143 | −32.838 | −10.911 | −17.204 |
| ATOM | 627 | HB2 | CYS | 143 | −31.856 | −9.573 | −16.544 |
| ATOM | 628 | HG | CYS | 143 | −30.51 | −11.008 | −18.171 |
| ATOM | 629 | LPG1 | CYS | 143 | −29.91 | −11.211 | −16.909 |
| ATOM | 630 | LPG2 | CYS | 143 | −30.496 | −12.085 | −17.258 |
| ATOM | 631 | H | CYS | 143 | −31.517 | −13.145 | −14.516 |
| ATOM | 632 | N | ASN | 144 | −34.206 | −11.341 | −13.665 |
| ATOM | 633 | CA | ASN | 144 | −35.392 | −10.789 | −13.003 |
| ATOM | 634 | C | ASN | 144 | −35.478 | −11.371 | −11.603 |
| ATOM | 635 | O | ASN | 144 | −35.521 | −10.601 | −10.658 |
| ATOM | 636 | CB | ASN | 144 | −36.691 | −11.031 | −13.807 |
| ATOM | 637 | CG | ASN | 144 | −36.622 | −10.352 | −15.153 |
| ATOM | 638 | OD1 | ASN | 144 | −37.177 | −9.275 | −15.307 |
| ATOM | 639 | ND2 | ASN | 144 | −35.945 | −10.947 | −16.157 |
| ATOM | 640 | HA | ASN | 144 | −35.298 | −9.697 | −12.91 |
| ATOM | 641 | HB1 | ASN | 144 | −36.865 | −12.102 | −13.949 |
| ATOM | 642 | HB2 | ASN | 144 | −37.541 | −10.635 | −13.234 |
| ATOM | 643 | HD22 | ASN | 144 | −35.886 | −10.493 | −17.045 |
| ATOM | 644 | HD21 | ASN | 144 | −35.481 | −11.826 | −16.03 |
| ATOM | 645 | H | ASN | 144 | −33.989 | −12.3 | −13.502 |
| ATOM | 646 | N | ASN | 145 | −35.467 | −12.717 | −11.434 |
| ATOM | 647 | CA | ASN | 145 | −35.277 | −13.308 | −10.104 |
| ATOM | 648 | C | ASN | 145 | −34.37 | −12.461 | −9.242 |
| ATOM | 649 | O | ASN | 145 | −34.793 | −12.043 | −8.178 |
| ATOM | 650 | CB | ASN | 145 | −34.606 | −14.713 | −10.158 |
| ATOM | 651 | CG | ASN | 145 | −35.599 | −15.81 | −10.439 |
| ATOM | 652 | OD1 | ASN | 145 | −36.093 | −16.417 | −9.502 |
| ATOM | 653 | ND2 | ASN | 145 | −35.933 | −16.098 | −11.71 |
| ATOM | 654 | HA | ASN | 145 | −36.25 | −13.39 | −9.595 |
| ATOM | 655 | HB1 | ASN | 145 | −33.812 | −14.71 | −10.919 |
| ATOM | 656 | HB2 | ASN | 145 | −34.129 | −14.963 | −9.199 |
| ATOM | 657 | HD22 | ASN | 145 | −36.626 | −16.799 | −11.882 |
| ATOM | 658 | HD21 | ASN | 145 | −35.528 | −15.624 | −12.488 |
| ATOM | 659 | H | ASN | 145 | −35.58 | −13.329 | −12.215 |
| ATOM | 660 | N | VAL | 146 | −33.115 | −12.208 | −9.67 |
| ATOM | 661 | CA | VAL | 146 | −32.177 | −11.513 | −8.79 |
| ATOM | 662 | C | VAL | 146 | −32.732 | −10.166 | −8.349 |
| ATOM | 663 | O | VAL | 146 | −32.933 | −10.01 | −7.152 |
| ATOM | 664 | CB | VAL | 146 | −30.727 | −11.501 | −9.372 |
| ATOM | 665 | CG1 | VAL | 146 | −29.809 | −10.566 | −8.542 |
| ATOM | 666 | CG2 | VAL | 146 | −30.106 | −12.929 | −9.419 |
| ATOM | 667 | HA | VAL | 146 | −32.107 | −12.114 | −7.869 |
| ATOM | 668 | H | VAL | 146 | −32.811 | −12.51 | −10.574 |
| ATOM | 669 | HB | VAL | 146 | −30.75 | −11.135 | −10.41 |
| ATOM | 670 | HG11 | VAL | 146 | −29.834 | −10.854 | −7.479 |
| ATOM | 671 | HG12 | VAL | 146 | −28.77 | −10.629 | −8.898 |
| ATOM | 672 | HG13 | VAL | 146 | −30.144 | −9.524 | −8.64 |
| ATOM | 673 | HG21 | VAL | 146 | −30.753 | −13.635 | −9.961 |
| ATOM | 674 | HG22 | VAL | 146 | −29.131 | −12.902 | −9.931 |
| ATOM | 675 | HG23 | VAL | 146 | −29.945 | −13.315 | −8.402 |
| ATOM | 676 | N | PRO | 147 | −33.012 | −9.151 | −9.205 |
| ATOM | 677 | CA | PRO | 147 | −33.523 | −7.891 | −8.681 |
| ATOM | 678 | C | PRO | 147 | −34.892 | −8.007 | −8.042 |
| ATOM | 679 | O | PRO | 147 | −35.1 | −7.371 | −7.02 |
| ATOM | 680 | CB | PRO | 147 | −33.464 | −6.994 | −9.943 |
| ATOM | 681 | CG | PRO | 147 | −33.573 | −8.001 | −11.11 |
| ATOM | 682 | CD | PRO | 147 | −32.744 | −9.214 | −10.632 |
| ATOM | 683 | HA | PRO | 147 | −32.828 | −7.506 | −7.928 |
| ATOM | 684 | HD2 | PRO | 147 | −31.678 | −9.015 | −10.813 |
| ATOM | 685 | HD1 | PRO | 147 | −33.048 | −10.138 | −11.14 |
| ATOM | 686 | HG2 | PRO | 147 | −33.2 | −7.626 | −12.072 |
| ATOM | 687 | HG1 | PRO | 147 | −34.635 | −8.252 | −11.222 |
| ATOM | 688 | HB1 | PRO | 147 | −34.246 | −6.217 | −9.958 |
| ATOM | 689 | HB2 | PRO | 147 | −32.477 | −6.503 | −10.011 |
| ATOM | 690 | N | ASP | 148 | −35.845 | −8.801 | −8.581 |
| ATOM | 691 | CA | ASP | 148 | −37.138 | −8.936 | −7.906 |
| ATOM | 692 | C | ASP | 148 | −36.917 | −9.463 | −6.504 |
| ATOM | 693 | O | ASP | 148 | −37.553 | −8.999 | −5.571 |
| ATOM | 694 | CB | ASP | 148 | −38.092 | −9.92 | −8.637 |
| ATOM | 695 | CG | ASP | 148 | −38.594 | −9.383 | −9.953 |
| ATOM | 696 | OD1 | ASP | 148 | −38.601 | −10.134 | −10.966 |
| ATOM | 697 | OD2 | ASP | 148 | −39.028 | −8.201 | −10.005 |
| ATOM | 698 | HA | ASP | 148 | −37.625 | −7.949 | −7.842 |
| ATOM | 699 | H | ASP | 148 | −35.673 | −9.345 | −9.404 |
| ATOM | 700 | HB1 | ASP | 148 | −37.579 | −10.881 | −8.793 |
| ATOM | 701 | HB2 | ASP | 148 | −38.982 | −10.103 | −8.014 |
| ATOM | 702 | N | ILE | 149 | −36.012 | −10.451 | −6.336 |
| ATOM | 703 | CA | ILE | 149 | −35.801 | −11.038 | −5.014 |
| ATOM | 704 | C | ILE | 149 | −35.164 | −10.028 | −4.079 |
| ATOM | 705 | O | ILE | 149 | −35.58 | −9.983 | −2.931 |
| ATOM | 706 | CB | ILE | 149 | −35.065 | −12.41 | −5.131 |
| ATOM | 707 | CG1 | ILE | 149 | −36.039 | −13.473 | −5.733 |
| ATOM | 708 | CG2 | ILE | 149 | −34.495 | −12.861 | −3.762 |
| ATOM | 709 | CD1 | ILE | 149 | −35.341 | −14.776 | −6.206 |
| ATOM | 710 | HA | ILE | 149 | −36.792 | −11.257 | −4.579 |
| ATOM | 711 | H | ILE | 149 | −35.473 | −10.787 | −7.113 |
| ATOM | 712 | HB | ILE | 149 | −34.2 | −12.274 | −5.799 |
| ATOM | 713 | HG11 | ILE | 149 | −36.814 | −13.73 | −4.992 |
| ATOM | 714 | HG12 | ILE | 149 | −36.55 | −13.041 | −6.608 |
| ATOM | 715 | HD11 | ILE | 149 | −34.955 | −15.369 | −5.365 |
| ATOM | 716 | HD12 | ILE | 149 | −36.055 | −15.406 | −6.76 |
| ATOM | 717 | HD13 | ILE | 149 | −34.507 | −14.529 | −6.876 |
| ATOM | 718 | HG21 | ILE | 149 | −35.276 | −12.815 | −2.987 |
| ATOM | 719 | HG22 | ILE | 149 | −34.089 | −13.881 | −3.785 |
| ATOM | 720 | HG23 | ILE | 149 | −33.665 | −12.19 | −3.497 |
| ATOM | 721 | N | PHE | 150 | −34.19 | −9.178 | −4.486 |
| ATOM | 722 | CA | PHE | 150 | −33.745 | −8.144 | −3.545 |
| ATOM | 723 | C | PHE | 150 | −34.932 | −7.33 | −3.09 |
| ATOM | 724 | O | PHE | 150 | −34.998 | −6.966 | −1.929 |
| ATOM | 725 | CB | PHE | 150 | −32.833 | −7.014 | −4.093 |
| ATOM | 726 | CG | PHE | 150 | −31.503 | −7.476 | −4.686 |
| ATOM | 727 | CD1 | PHE | 150 | −31.127 | −7.006 | −5.947 |
| ATOM | 728 | CD2 | PHE | 150 | −30.648 | −8.337 | −3.994 |
| ATOM | 729 | CE1 | PHE | 150 | −30.022 | −7.545 | −6.608 |
| ATOM | 730 | CE2 | PHE | 150 | −29.478 | −8.787 | −4.611 |
| ATOM | 731 | CZ | PHE | 150 | −29.204 | −8.457 | −5.942 |
| ATOM | 732 | HA | PHE | 150 | −33.277 | −8.631 | −2.674 |
| ATOM | 733 | H | PHE | 150 | −33.807 | −9.211 | −5.414 |
| ATOM | 734 | HB1 | PHE | 150 | −33.393 | −6.443 | −4.852 |
| ATOM | 735 | HB2 | PHE | 150 | −32.578 | −6.325 | −3.273 |
| ATOM | 736 | HD2 | PHE | 150 | −30.875 | −8.652 | −2.982 |
| ATOM | 737 | HE2 | PHE | 150 | −28.787 | −9.391 | −4.046 |
| ATOM | 738 | HZ | PHE | 150 | −28.362 | −8.9 | −6.463 |
| ATOM | 739 | HE1 | PHE | 150 | −29.804 | −7.259 | −7.63 |
| ATOM | 740 | HD1 | PHE | 150 | −31.699 | −6.213 | −6.411 |
| ATOM | 741 | N | ILE | 151 | −35.861 | −6.996 | −4.01 |
| ATOM | 742 | CA | ILE | 151 | −36.937 | −6.081 | −3.64 |
| ATOM | 743 | C | ILE | 151 | −37.789 | −6.704 | −2.552 |
| ATOM | 744 | O | ILE | 151 | −38.339 | −5.969 | −1.744 |
| ATOM | 745 | CB | ILE | 151 | −37.681 | −5.568 | −4.909 |
| ATOM | 746 | CG1 | ILE | 151 | −36.706 | −4.698 | −5.77 |
| ATOM | 747 | CG2 | ILE | 151 | −38.946 | −4.769 | −4.501 |
| ATOM | 748 | CD1 | ILE | 151 | −37.179 | −4.47 | −7.228 |
| ATOM | 749 | HA | ILE | 151 | −36.473 | −5.198 | −3.175 |
| ATOM | 750 | H | ILE | 151 | −35.822 | −7.361 | −4.945 |
| ATOM | 751 | HB | ILE | 151 | −38.009 | −6.442 | −5.496 |
| ATOM | 752 | HG11 | ILE | 151 | −36.57 | −3.714 | −5.298 |
| ATOM | 753 | HG12 | ILE | 151 | −35.713 | −5.169 | −5.83 |
| ATOM | 754 | HD11 | ILE | 151 | −38.069 | −3.833 | −7.235 |
| ATOM | 755 | HD12 | ILE | 151 | −36.393 | −3.958 | −7.808 |
| ATOM | 756 | HD13 | ILE | 151 | −37.412 | −5.423 | −7.724 |
| ATOM | 757 | HG21 | ILE | 151 | −38.687 | −3.94 | −3.825 |
| ATOM | 758 | HG22 | ILE | 151 | −39.455 | −4.358 | −5.38 |
| ATOM | 759 | HG23 | ILE | 151 | −39.651 | −5.44 | −3.989 |
| ATOM | 760 | N | ILE | 152 | −37.881 | −8.052 | −2.467 |
| ATOM | 761 | CA | ILE | 152 | −38.504 | −8.656 | −1.288 |
| ATOM | 762 | C | ILE | 152 | −37.66 | −8.239 | −0.098 |
| ATOM | 763 | O | ILE | 152 | −38.209 | −7.768 | 0.886 |
| ATOM | 764 | CB | ILE | 152 | −38.598 | −10.215 | −1.34 |
| ATOM | 765 | CG1 | ILE | 152 | −39.452 | −10.7 | −2.553 |
| ATOM | 766 | CG2 | ILE | 152 | −39.164 | −10.774 | −0.001 |
| ATOM | 767 | CD1 | ILE | 152 | −39.422 | −12.243 | −2.744 |
| ATOM | 768 | HA | ILE | 152 | −39.524 | −8.252 | −1.174 |
| ATOM | 769 | H | ILE | 152 | −37.437 | −8.643 | −3.143 |
| ATOM | 770 | HB | ILE | 152 | −37.576 | −10.612 | −1.457 |
| ATOM | 771 | HG11 | ILE | 152 | −40.495 | −10.363 | −2.436 |
| ATOM | 772 | HG12 | ILE | 152 | −39.062 | −10.256 | −3.481 |
| ATOM | 773 | HD11 | ILE | 152 | −40.014 | −12.756 | −1.971 |
| ATOM | 774 | HD12 | ILE | 152 | −39.847 | −12.519 | −3.721 |
| ATOM | 775 | HD13 | ILE | 152 | −38.387 | −12.616 | −2.71 |
| ATOM | 776 | HG21 | ILE | 152 | −40.201 | −10.436 | 0.144 |
| ATOM | 777 | HG22 | ILE | 152 | −39.149 | −11.873 | 0.015 |
| ATOM | 778 | HG23 | ILE | 152 | −38.565 | −10.441 | 0.859 |
| ATOM | 779 | N | ASN | 153 | −36.318 | −8.405 | −0.166 |
| ATOM | 780 | CA | ASN | 153 | −35.472 | −8.049 | 0.976 |
| ATOM | 781 | C | ASN | 153 | −35.652 | −6.589 | 1.327 |
| ATOM | 782 | O | ASN | 153 | −35.829 | −6.294 | 2.497 |
| ATOM | 783 | CB | ASN | 153 | −33.964 | −8.331 | 0.711 |
| ATOM | 784 | CG | ASN | 153 | −33.095 | −8.346 | 1.948 |
| ATOM | 785 | OD1 | ASN | 153 | −33.607 | −8.406 | 3.053 |
| ATOM | 786 | ND2 | ASN | 153 | −31.753 | −8.314 | 1.799 |
| ATOM | 787 | HA | ASN | 153 | −35.799 | −8.675 | 1.823 |
| ATOM | 788 | H | ASN | 153 | −35.889 | −8.771 | −0.995 |
| ATOM | 789 | HB1 | ASN | 153 | −33.87 | −9.328 | 0.264 |
| ATOM | 790 | HB2 | ASN | 153 | −33.561 | −7.596 | −0.002 |
| ATOM | 791 | HD22 | ASN | 153 | −31.17 | −8.313 | 2.612 |
| ATOM | 792 | HD21 | ASN | 153 | −31.324 | −8.294 | 0.896 |
| ATOM | 793 | N | LEU | 154 | −35.623 | −5.655 | 0.346 |
| ATOM | 794 | CA | LEU | 154 | −35.834 | −4.241 | 0.674 |
| ATOM | 795 | C | LEU | 154 | −37.148 | −4.108 | 1.404 |
| ATOM | 796 | O | LEU | 154 | −37.226 | −3.398 | 2.393 |
| ATOM | 797 | CB | LEU | 154 | −35.919 | −3.288 | −0.559 |
| ATOM | 798 | CG | LEU | 154 | −34.587 | −3.137 | −1.355 |
| ATOM | 799 | CD1 | LEU | 154 | −34.808 | −2.434 | −2.723 |
| ATOM | 800 | CD2 | LEU | 154 | −33.524 | −2.322 | −0.563 |
| ATOM | 801 | HA | LEU | 154 | −35.034 | −3.913 | 1.351 |
| ATOM | 802 | H | LEU | 154 | −35.497 | −5.925 | −0.609 |
| ATOM | 803 | HB1 | LEU | 154 | −36.708 | −3.679 | −1.22 |
| ATOM | 804 | HB2 | LEU | 154 | −36.235 | −2.285 | −0.229 |
| ATOM | 805 | HG | LEU | 154 | −34.218 | −4.147 | −1.584 |
| ATOM | 806 | HD21 | LEU | 154 | −33.278 | −2.783 | 0.402 |
| ATOM | 807 | HD22 | LEU | 154 | −32.596 | −2.241 | −1.15 |
| ATOM | 808 | HD23 | LEU | 154 | −33.894 | −1.305 | −0.363 |
| ATOM | 809 | HD11 | LEU | 154 | −35.036 | −1.373 | −2.574 |
| ATOM | 810 | HD12 | LEU | 154 | −33.902 | −2.504 | −3.345 |
| ATOM | 811 | HD13 | LEU | 154 | −35.64 | −2.89 | −3.273 |
| ATOM | 812 | N | SER | 155 | −38.211 | −4.781 | 0.923 |
| ATOM | 813 | CA | SER | 155 | −39.513 | −4.592 | 1.554 |
| ATOM | 814 | C | SER | 155 | −39.527 | −5.193 | 2.946 |
| ATOM | 815 | O | SER | 155 | −40.066 | −4.572 | 3.849 |
| ATOM | 816 | CB | SER | 155 | −40.633 | −5.184 | 0.664 |
| ATOM | 817 | OG | SER | 155 | −40.736 | −4.516 | −0.607 |
| ATOM | 818 | HA | SER | 155 | −39.708 | −3.511 | 1.634 |
| ATOM | 819 | H | SER | 155 | −38.122 | −5.406 | 0.143 |
| ATOM | 820 | HB1 | SER | 155 | −40.456 | −6.261 | 0.519 |
| ATOM | 821 | HB2 | SER | 155 | −41.596 | −5.037 | 1.172 |
| ATOM | 822 | HG | SER | 155 | −39.962 | −4.621 | −1.153 |
| ATOM | 823 | N | VAL | 156 | −38.942 | −6.393 | 3.165 |
| ATOM | 824 | CA | VAL | 156 | −38.882 | −6.944 | 4.525 |
| ATOM | 825 | C | VAL | 156 | −38.073 | −6.004 | 5.388 |
| ATOM | 826 | O | VAL | 156 | −38.521 | −5.624 | 6.458 |
| ATOM | 827 | CB | VAL | 156 | −38.266 | −8.376 | 4.559 |
| ATOM | 828 | CG1 | VAL | 156 | −37.94 | −8.824 | 6.014 |
| ATOM | 829 | CG2 | VAL | 156 | −39.229 | −9.401 | 3.896 |
| ATOM | 830 | HA | VAL | 156 | −39.896 | −6.998 | 4.952 |
| ATOM | 831 | H | VAL | 156 | −38.501 | −6.884 | 2.411 |
| ATOM | 832 | HB | VAL | 156 | −37.319 | −8.371 | 3.992 |
| ATOM | 833 | HG11 | VAL | 156 | −38.842 | −8.769 | 6.642 |
| ATOM | 834 | HG12 | VAL | 156 | −37.57 | −9.861 | 6.021 |
| ATOM | 835 | HG13 | VAL | 156 | −37.161 | −8.188 | 6.463 |
| ATOM | 836 | HG21 | VAL | 156 | −39.519 | −9.08 | 2.885 |
| ATOM | 837 | HG22 | VAL | 156 | −38.745 | −10.387 | 3.818 |
| ATOM | 838 | HG23 | VAL | 156 | −40.143 | −9.511 | 4.5 |
| ATOM | 839 | N | VAL | 157 | −36.868 | −5.617 | 4.923 |
| ATOM | 840 | CA | VAL | 157 | −36.055 | −4.649 | 5.66 |
| ATOM | 841 | C | VAL | 157 | −36.907 | −3.458 | 6.04 |
| ATOM | 842 | O | VAL | 157 | −36.975 | −3.098 | 7.206 |
| ATOM | 843 | CB | VAL | 157 | −34.85 | −4.201 | 4.776 |
| ATOM | 844 | CG1 | VAL | 157 | −34.257 | −2.836 | 5.199 |
| ATOM | 845 | CG2 | VAL | 157 | −33.731 | −5.279 | 4.747 |
| ATOM | 846 | HA | VAL | 157 | −35.678 | −5.112 | 6.585 |
| ATOM | 847 | H | VAL | 157 | −36.537 | −5.975 | 4.048 |
| ATOM | 848 | HB | VAL | 157 | −35.208 | −4.054 | 3.748 |
| ATOM | 849 | HG11 | VAL | 157 | −33.894 | −2.879 | 6.236 |
| ATOM | 850 | HG12 | VAL | 157 | −34.998 | −2.028 | 5.113 |
| ATOM | 851 | HG13 | VAL | 157 | −33.425 | −2.598 | 4.519 |
| ATOM | 852 | HG21 | VAL | 157 | −34.138 | −6.27 | 4.503 |
| ATOM | 853 | HG22 | VAL | 157 | −32.983 | −5.019 | 3.984 |
| ATOM | 854 | HG23 | VAL | 157 | −33.231 | −5.335 | 5.725 |
| ATOM | 855 | N | ASP | 158 | −37.564 | −2.821 | 5.049 |
| ATOM | 856 | CA | ASP | 158 | −38.346 | −1.626 | 5.346 |
| ATOM | 857 | C | ASP | 158 | −39.519 | −1.966 | 6.245 |
| ATOM | 858 | O | ASP | 158 | −39.89 | −1.123 | 7.047 |
| ATOM | 859 | CB | ASP | 158 | −38.755 | −0.891 | 4.042 |
| ATOM | 860 | CG | ASP | 158 | −37.56 | −0.291 | 3.33 |
| ATOM | 861 | OD1 | ASP | 158 | −36.605 | 0.159 | 4.026 |
| ATOM | 862 | OD2 | ASP | 158 | −37.565 | −0.24 | 2.068 |
| ATOM | 863 | HA | ASP | 158 | −37.707 | −0.937 | 5.923 |
| ATOM | 864 | H | ASP | 158 | −37.516 | −3.146 | 4.101 |
| ATOM | 865 | HB1 | ASP | 158 | −39.288 | −1.588 | 3.379 |
| ATOM | 866 | HB2 | ASP | 158 | −39.438 | −0.07 | 4.293 |
| ATOM | 867 | N | LEU | 159 | −40.125 | −3.175 | 6.189 |
| ATOM | 868 | CA | LEU | 159 | −41.112 | −3.518 | 7.215 |
| ATOM | 869 | C | LEU | 159 | −40.446 | −3.579 | 8.57 |
| ATOM | 870 | O | LEU | 159 | −40.995 | −3.027 | 9.511 |
| ATOM | 871 | CB | LEU | 159 | −41.849 | −4.875 | 7.016 |
| ATOM | 872 | CG | LEU | 159 | −42.995 | −4.897 | 5.962 |
| ATOM | 873 | CD1 | LEU | 159 | −43.721 | −6.27 | 6.053 |
| ATOM | 874 | CD2 | LEU | 159 | −44.042 | −3.769 | 6.181 |
| ATOM | 875 | HA | LEU | 159 | −41.835 | −2.696 | 7.303 |
| ATOM | 876 | H | LEU | 159 | −39.865 | −3.862 | 5.508 |
| ATOM | 877 | HB1 | LEU | 159 | −41.108 | −5.653 | 6.783 |
| ATOM | 878 | HB2 | LEU | 159 | −42.315 | −5.154 | 7.97 |
| ATOM | 879 | HG | LEU | 159 | −42.556 | −4.787 | 4.957 |
| ATOM | 880 | HD21 | LEU | 159 | −43.618 | −2.775 | 6.002 |
| ATOM | 881 | HD22 | LEU | 159 | −44.873 | −3.884 | 5.474 |
| ATOM | 882 | HD23 | LEU | 159 | −44.444 | −3.801 | 7.205 |
| ATOM | 883 | HD11 | LEU | 159 | −44.187 | −6.402 | 7.042 |
| ATOM | 884 | HD12 | LEU | 159 | −44.519 | −6.341 | 5.302 |
| ATOM | 885 | HD13 | LEU | 159 | −43.012 | −7.096 | 5.892 |
| ATOM | 886 | N | LEU | 160 | −39.274 | −4.225 | 8.732 |
| ATOM | 887 | CA | LEU | 160 | −38.667 | −4.262 | 10.064 |
| ATOM | 888 | C | LEU | 160 | −38.485 | −2.845 | 10.574 |
| ATOM | 889 | O | LEU | 160 | −38.683 | −2.608 | 11.756 |
| ATOM | 890 | CB | LEU | 160 | −37.326 | −5.046 | 10.075 |
| ATOM | 891 | CG | LEU | 160 | −37.479 | −6.569 | 9.761 |
| ATOM | 892 | CD1 | LEU | 160 | −36.088 | −7.187 | 9.45 |
| ATOM | 893 | CD2 | LEU | 160 | −38.147 | −7.353 | 10.927 |
| ATOM | 894 | HA | LEU | 160 | −39.377 | −4.746 | 10.752 |
| ATOM | 895 | H | LEU | 160 | −38.809 | −4.64 | 7.946 |
| ATOM | 896 | HB1 | LEU | 160 | −36.663 | −4.571 | 9.334 |
| ATOM | 897 | HB2 | LEU | 160 | −36.852 | −4.936 | 11.063 |
| ATOM | 898 | HG | LEU | 160 | −38.102 | −6.711 | 8.862 |
| ATOM | 899 | HD21 | LEU | 160 | −39.184 | −7.031 | 11.097 |
| ATOM | 900 | HD22 | LEU | 160 | −38.173 | −8.429 | 10.697 |
| ATOM | 901 | HD23 | LEU | 160 | −37.578 | −7.214 | 11.859 |
| ATOM | 902 | HD11 | LEU | 160 | −35.407 | −7.052 | 10.304 |
| ATOM | 903 | HD12 | LEU | 160 | −36.173 | −8.263 | 9.232 |
| ATOM | 904 | HD13 | LEU | 160 | −35.65 | −6.699 | 8.566 |
| ATOM | 905 | N | PHE | 161 | −38.139 | −1.877 | 9.696 |
| ATOM | 906 | CA | PHE | 161 | −38.089 | −0.473 | 10.123 |
| ATOM | 907 | C | PHE | 161 | −39.423 | 0.039 | 10.629 |
| ATOM | 908 | O | PHE | 161 | −39.405 | 0.954 | 11.436 |
| ATOM | 909 | CB | PHE | 161 | −37.521 | 0.401 | 8.968 |
| ATOM | 910 | CG | PHE | 161 | −37.397 | 1.892 | 9.319 |
| ATOM | 911 | CD1 | PHE | 161 | −36.313 | 2.365 | 10.064 |
| ATOM | 912 | CD2 | PHE | 161 | −38.357 | 2.807 | 8.877 |
| ATOM | 913 | CE1 | PHE | 161 | −36.221 | 3.72 | 10.391 |
| ATOM | 914 | CE2 | PHE | 161 | −38.283 | 4.16 | 9.215 |
| ATOM | 915 | CZ | PHE | 161 | −37.216 | 4.614 | 9.991 |
| ATOM | 916 | HA | PHE | 161 | −37.423 | −0.393 | 10.99 |
| ATOM | 917 | H | PHE | 161 | −37.951 | −2.107 | 8.736 |
| ATOM | 918 | HB1 | PHE | 161 | −36.524 | 0.026 | 8.684 |
| ATOM | 919 | HB2 | PHE | 161 | −38.171 | 0.3 | 8.09 |
| ATOM | 920 | HD2 | PHE | 161 | −39.169 | 2.469 | 8.253 |
| ATOM | 921 | HE2 | PHE | 161 | −39.047 | 4.853 | 8.877 |
| ATOM | 922 | HZ | PHE | 161 | −37.158 | 5.654 | 10.282 |
| ATOM | 923 | HE1 | PHE | 161 | −35.375 | 4.084 | 10.958 |
| ATOM | 924 | HD1 | PHE | 161 | −35.535 | 1.685 | 10.383 |
| ATOM | 925 | N | LEU | 162 | −40.579 | −0.515 | 10.193 |
| ATOM | 926 | CA | LEU | 162 | −41.886 | −0.006 | 10.619 |
| ATOM | 927 | C | LEU | 162 | −42.744 | −0.953 | 11.434 |
| ATOM | 928 | O | LEU | 162 | −43.766 | −0.48 | 11.903 |
| ATOM | 929 | CB | LEU | 162 | −42.679 | 0.381 | 9.344 |
| ATOM | 930 | CG | LEU | 162 | −41.919 | 1.398 | 8.449 |
| ATOM | 931 | CD1 | LEU | 162 | −42.656 | 1.592 | 7.097 |
| ATOM | 932 | CD2 | LEU | 162 | −41.716 | 2.753 | 9.187 |
| ATOM | 933 | HA | LEU | 162 | −41.788 | 0.886 | 11.255 |
| ATOM | 934 | H | LEU | 162 | −40.565 | −1.253 | 9.521 |
| ATOM | 935 | HB1 | LEU | 162 | −42.87 | −0.54 | 8.771 |
| ATOM | 936 | HB2 | LEU | 162 | −43.646 | 0.818 | 9.638 |
| ATOM | 937 | HG | LEU | 162 | −40.937 | 0.97 | 8.193 |
| ATOM | 938 | HD21 | LEU | 162 | −40.958 | 2.659 | 9.978 |
| ATOM | 939 | HD22 | LEU | 162 | −41.377 | 3.534 | 8.491 |
| ATOM | 940 | HD23 | LEU | 162 | −42.647 | 3.094 | 9.657 |
| ATOM | 941 | HD11 | LEU | 162 | −43.668 | 1.985 | 7.253 |
| ATOM | 942 | HD12 | LEU | 162 | −42.092 | 2.297 | 6.467 |
| ATOM | 943 | HD13 | LEU | 162 | −42.736 | 0.635 | 6.556 |
| ATOM | 944 | N | LEU | 163 | −42.429 | −2.251 | 11.645 |
| ATOM | 945 | CA | LEU | 163 | −43.296 | −3.068 | 12.503 |
| ATOM | 946 | C | LEU | 163 | −42.974 | −2.811 | 13.958 |
| ATOM | 947 | O | LEU | 163 | −43.889 | −2.62 | 14.744 |
| ATOM | 948 | CB | LEU | 163 | −43.173 | −4.596 | 12.252 |
| ATOM | 949 | CG | LEU | 163 | −43.53 | −5.03 | 10.801 |
| ATOM | 950 | CD1 | LEU | 163 | −43.334 | −6.565 | 10.651 |
| ATOM | 951 | CD2 | LEU | 163 | −44.967 | −4.626 | 10.368 |
| ATOM | 952 | HA | LEU | 163 | −44.353 | −2.792 | 12.358 |
| ATOM | 953 | H | LEU | 163 | −41.597 | −2.653 | 11.257 |
| ATOM | 954 | HB1 | LEU | 163 | −42.136 | −4.901 | 12.472 |
| ATOM | 955 | HB2 | LEU | 163 | −43.837 | −5.127 | 12.954 |
| ATOM | 956 | HG | LEU | 163 | −42.827 | −4.528 | 10.126 |
| ATOM | 957 | HD21 | LEU | 163 | −45.062 | −3.535 | 10.258 |
| ATOM | 958 | HD22 | LEU | 163 | −45.213 | −5.074 | 9.393 |
| ATOM | 959 | HD23 | LEU | 163 | −45.702 | −4.977 | 11.108 |
| ATOM | 960 | HD11 | LEU | 163 | −44.032 | −7.104 | 11.31 |
| ATOM | 961 | HD12 | LEU | 163 | −43.519 | −6.884 | 9.613 |
| ATOM | 962 | HD13 | LEU | 163 | −42.305 | −6.854 | 10.917 |
| ATOM | 963 | N | GLY | 164 | −41.682 | −2.814 | 14.353 |
| ATOM | 964 | CA | GLY | 164 | −41.355 | −2.581 | 15.758 |
| ATOM | 965 | C | GLY | 164 | −41.366 | −1.11 | 16.102 |
| ATOM | 966 | O | GLY | 164 | −41.744 | −0.783 | 17.216 |
| ATOM | 967 | HA2 | GLY | 164 | −40.357 | −2.985 | 15.985 |
| ATOM | 968 | HA1 | GLY | 164 | −42.069 | −3.112 | 16.409 |
| ATOM | 969 | H | GLY | 164 | −40.933 | −2.958 | 13.7 |
| ATOM | 970 | N | MET | 165 | −40.933 | −0.202 | 15.193 |
| ATOM | 971 | CA | MET | 165 | −40.866 | 1.213 | 15.56 |
| ATOM | 972 | C | MET | 165 | −42.148 | 1.685 | 16.221 |
| ATOM | 973 | O | MET | 165 | −42.028 | 2.217 | 17.312 |
| ATOM | 974 | CB | MET | 165 | −40.454 | 2.174 | 14.405 |
| ATOM | 975 | CG | MET | 165 | −38.929 | 2.438 | 14.313 |
| ATOM | 976 | SD | MET | 165 | −38.675 | 3.643 | 12.963 |
| ATOM | 977 | CE | MET | 165 | −37.057 | 4.334 | 13.409 |
| ATOM | 978 | HA | MET | 165 | −40.084 | 1.291 | 16.333 |
| ATOM | 979 | H | MET | 165 | −40.609 | −0.482 | 14.288 |
| ATOM | 980 | HB1 | MET | 165 | −40.781 | 1.783 | 13.433 |
| ATOM | 981 | HB2 | MET | 165 | −40.942 | 3.148 | 14.564 |
| ATOM | 982 | HG1 | MET | 165 | −38.373 | 1.513 | 14.095 |
| ATOM | 983 | HG2 | MET | 165 | −38.562 | 2.866 | 15.259 |
| ATOM | 984 | LPD1 | MET | 165 | −38.656 | 3.338 | 12.357 |
| ATOM | 985 | LPD2 | MET | 165 | −39.157 | 4.121 | 12.961 |
| ATOM | 986 | HE1 | MET | 165 | −36.284 | 3.559 | 13.309 |
| ATOM | 987 | HE2 | MET | 165 | −36.817 | 5.188 | 12.76 |
| ATOM | 988 | HE3 | MET | 165 | −37.097 | 4.691 | 14.443 |
| ATOM | 989 | N | PRO | 166 | −43.38 | 1.541 | 15.677 |
| ATOM | 990 | CA | PRO | 166 | −44.554 | 1.983 | 16.414 |
| ATOM | 991 | C | PRO | 166 | −44.574 | 1.539 | 17.855 |
| ATOM | 992 | O | PRO | 166 | −44.943 | 2.335 | 18.701 |
| ATOM | 993 | CB | PRO | 166 | −45.67 | 1.291 | 15.597 |
| ATOM | 994 | CG | PRO | 166 | −45.094 | 1.289 | 14.165 |
| ATOM | 995 | CD | PRO | 166 | −43.604 | 0.93 | 14.378 |
| ATOM | 996 | HA | PRO | 166 | −44.63 | 3.081 | 16.335 |
| ATOM | 997 | HD2 | PRO | 166 | −43 | 1.345 | 13.564 |
| ATOM | 998 | HD1 | PRO | 166 | −43.465 | −0.156 | 14.45 |
| ATOM | 999 | HG2 | PRO | 166 | −45.165 | 2.314 | 13.767 |
| ATOM | 1000 | HG1 | PRO | 166 | −45.619 | 0.6 | 13.483 |
| ATOM | 1001 | HB1 | PRO | 166 | −46.641 | 1.807 | 15.677 |
| ATOM | 1002 | HB2 | PRO | 166 | −45.795 | 0.244 | 15.922 |
| ATOM | 1003 | N | PHE | 167 | −44.18 | 0.289 | 18.178 |
| ATOM | 1004 | CA | PHE | 167 | −44.225 | −0.127 | 19.579 |
| ATOM | 1005 | C | PHE | 167 | −43.36 | 0.793 | 20.412 |
| ATOM | 1006 | O | PHE | 167 | −43.784 | 1.214 | 21.476 |
| ATOM | 1007 | CB | PHE | 167 | −43.743 | −1.59 | 19.762 |
| ATOM | 1008 | CG | PHE | 167 | −43.93 | −2.024 | 21.224 |
| ATOM | 1009 | CD1 | PHE | 167 | −45.112 | −2.652 | 21.631 |
| ATOM | 1010 | CD2 | PHE | 167 | −42.919 | −1.795 | 22.164 |
| ATOM | 1011 | CE1 | PHE | 167 | −45.275 | −3.06 | 22.958 |
| ATOM | 1012 | CE2 | PHE | 167 | −43.081 | −2.195 | 23.493 |
| ATOM | 1013 | CZ | PHE | 167 | −44.26 | −2.832 | 23.891 |
| ATOM | 1014 | HA | PHE | 167 | −45.269 | −0.059 | 19.93 |
| ATOM | 1015 | H | PHE | 167 | −43.836 | −0.345 | 17.481 |
| ATOM | 1016 | HB1 | PHE | 167 | −44.323 | −2.254 | 19.102 |
| ATOM | 1017 | HB2 | PHE | 167 | −42.684 | −1.682 | 19.479 |
| ATOM | 1018 | HD2 | PHE | 167 | −42.001 | −1.303 | 21.865 |
| ATOM | 1019 | HE2 | PHE | 167 | −42.292 | −2.011 | 24.216 |
| ATOM | 1020 | HZ | PHE | 167 | −44.386 | −3.148 | 24.922 |
| ATOM | 1021 | HE1 | PHE | 167 | −46.192 | −3.555 | 23.265 |
| ATOM | 1022 | HD1 | PHE | 167 | −45.911 | −2.827 | 20.918 |
| ATOM | 1023 | N | MET | 168 | −42.133 | 1.109 | 19.939 |
| ATOM | 1024 | CA | MET | 168 | −41.23 | 1.944 | 20.73 |
| ATOM | 1025 | C | MET | 168 | −41.489 | 3.412 | 20.475 |
| ATOM | 1026 | O | MET | 168 | −41.676 | 4.147 | 21.43 |
| ATOM | 1027 | CB | MET | 168 | −39.756 | 1.545 | 20.45 |
| ATOM | 1028 | CG | MET | 168 | −39.495 | 0.09 | 20.925 |
| ATOM | 1029 | SD | MET | 168 | −37.715 | −0.277 | 20.907 |
| ATOM | 1030 | CE | MET | 168 | −37.748 | −2.034 | 21.382 |
| ATOM | 1031 | HA | MET | 168 | −41.406 | 1.773 | 21.806 |
| ATOM | 1032 | H | MET | 168 | −41.837 | 0.798 | 19.031 |
| ATOM | 1033 | HB1 | MET | 168 | −39.524 | 1.648 | 19.379 |
| ATOM | 1034 | HB2 | MET | 168 | −39.09 | 2.224 | 20.999 |
| ATOM | 1035 | HG1 | MET | 168 | −39.824 | −0.043 | 21.967 |
| ATOM | 1036 | HG2 | MET | 168 | −40.026 | −0.629 | 20.282 |
| ATOM | 1037 | LPD1 | MET | 168 | −37.446 | −0.188 | 20.29 |
| ATOM | 1038 | LPD2 | MET | 168 | −37.376 | 0.099 | 21.359 |
| ATOM | 1039 | HE1 | MET | 168 | −38.216 | −2.63 | 20.586 |
| ATOM | 1040 | HE2 | MET | 168 | −36.716 | −2.382 | 21.534 |
| ATOM | 1041 | HE3 | MET | 168 | −38.32 | −2.165 | 22.311 |
| ATOM | 1042 | N | ILE | 169 | −41.527 | 3.892 | 19.214 |
| ATOM | 1043 | CA | ILE | 169 | −41.855 | 5.299 | 18.98 |
| ATOM | 1044 | C | ILE | 169 | −43.169 | 5.627 | 19.66 |
| ATOM | 1045 | O | ILE | 169 | −43.288 | 6.728 | 20.175 |
| ATOM | 1046 | CB | ILE | 169 | −41.852 | 5.694 | 17.466 |
| ATOM | 1047 | CG1 | ILE | 169 | −40.391 | 5.656 | 16.91 |
| ATOM | 1048 | CG2 | ILE | 169 | −42.504 | 7.092 | 17.252 |
| ATOM | 1049 | CD1 | ILE | 169 | −40.253 | 6.096 | 15.427 |
| ATOM | 1050 | HA | ILE | 169 | −41.089 | 5.909 | 19.487 |
| ATOM | 1051 | H | ILE | 169 | −41.376 | 3.283 | 18.438 |
| ATOM | 1052 | HB | ILE | 169 | −42.468 | 4.962 | 16.916 |
| ATOM | 1053 | HG11 | ILE | 169 | −39.739 | 6.308 | 17.514 |
| ATOM | 1054 | HG12 | ILE | 169 | −40.004 | 4.627 | 16.993 |
| ATOM | 1055 | HD11 | ILE | 169 | −40.391 | 7.181 | 15.31 |
| ATOM | 1056 | HD12 | ILE | 169 | −39.245 | 5.854 | 15.057 |
| ATOM | 1057 | HD13 | ILE | 169 | −40.988 | 5.575 | 14.801 |
| ATOM | 1058 | HG21 | ILE | 169 | −41.935 | 7.866 | 17.79 |
| ATOM | 1059 | HG22 | ILE | 169 | −42.549 | 7.359 | 16.187 |
| ATOM | 1060 | HG23 | ILE | 169 | −43.543 | 7.101 | 17.614 |
| ATOM | 1061 | N | HIS | 170 | −44.178 | 4.728 | 19.718 |
| ATOM | 1062 | CA | HIS | 170 | −45.396 | 5.1 | 20.437 |
| ATOM | 1063 | C | HIS | 170 | −45.065 | 5.439 | 21.874 |
| ATOM | 1064 | O | HIS | 170 | −45.715 | 6.319 | 22.412 |
| ATOM | 1065 | CB | HIS | 170 | −46.54 | 4.056 | 20.402 |
| ATOM | 1066 | CG | HIS | 170 | −47.788 | 4.65 | 21.01 |
| ATOM | 1067 | ND1 | HIS | 170 | −47.975 | 4.828 | 22.299 |
| ATOM | 1068 | CD2 | HIS | 170 | −48.866 | 5.085 | 20.326 |
| ATOM | 1069 | CE1 | HIS | 170 | −49.134 | 5.369 | 22.502 |
| ATOM | 1070 | NE2 | HIS | 170 | −49.705 | 5.544 | 21.399 |
| ATOM | 1071 | HA | HIS | 170 | −45.785 | 6.003 | 19.94 |
| ATOM | 1072 | H | HIS | 170 | −44.11 | 3.819 | 19.303 |
| ATOM | 1073 | HB1 | HIS | 170 | −46.765 | 3.804 | 19.354 |
| ATOM | 1074 | HB2 | HIS | 170 | −46.249 | 3.138 | 20.936 |
| ATOM | 1075 | HD2 | HIS | 170 | −49.057 | 5.094 | 19.253 |
| ATOM | 1076 | HE1 | HIS | 170 | −49.563 | 5.638 | 23.467 |
| ATOM | 1077 | HD1 | HIS | 170 | −47.301 | 4.584 | 23.039 |
| ATOM | 1078 | N | GLN | 171 | −44.072 | 4.796 | 22.533 |
| ATOM | 1079 | CA | GLN | 171 | −43.689 | 5.258 | 23.87 |
| ATOM | 1080 | C | GLN | 171 | −43.352 | 6.73 | 23.775 |
| ATOM | 1081 | O | GLN | 171 | −43.778 | 7.496 | 24.623 |
| ATOM | 1082 | CB | GLN | 171 | −42.451 | 4.535 | 24.476 |
| ATOM | 1083 | CG | GLN | 171 | −42.7 | 3.044 | 24.824 |
| ATOM | 1084 | CD | GLN | 171 | −43.599 | 2.937 | 26.033 |
| ATOM | 1085 | OE1 | GLN | 171 | −43.09 | 2.998 | 27.142 |
| ATOM | 1086 | NE2 | GLN | 171 | −44.93 | 2.792 | 25.869 |
| ATOM | 1087 | HA | GLN | 171 | −44.549 | 5.142 | 24.547 |
| ATOM | 1088 | H | GLN | 171 | −43.549 | 4.061 | 22.099 |
| ATOM | 1089 | HB1 | GLN | 171 | −41.598 | 4.617 | 23.788 |
| ATOM | 1090 | HB2 | GLN | 171 | −42.158 | 5.047 | 25.405 |
| ATOM | 1091 | HG1 | GLN | 171 | −43.121 | 2.502 | 23.963 |
| ATOM | 1092 | HG2 | GLN | 171 | −41.738 | 2.566 | 25.075 |
| ATOM | 1093 | HE22 | GLN | 171 | −45.514 | 2.739 | 26.68 |
| ATOM | 1094 | HE21 | GLN | 171 | −45.348 | 2.735 | 24.962 |
| ATOM | 1095 | N | LEU | 172 | −42.594 | 7.16 | 22.743 |
| ATOM | 1096 | CA | LEU | 172 | −42.293 | 8.584 | 22.623 |
| ATOM | 1097 | C | LEU | 172 | −43.585 | 9.363 | 22.533 |
| ATOM | 1098 | O | LEU | 172 | −43.731 | 10.365 | 23.213 |
| ATOM | 1099 | CB | LEU | 172 | −41.448 | 8.923 | 21.357 |
| ATOM | 1100 | CG | LEU | 172 | −40.783 | 10.329 | 21.444 |
| ATOM | 1101 | CD1 | LEU | 172 | −39.5 | 10.286 | 22.321 |
| ATOM | 1102 | CD2 | LEU | 172 | −40.433 | 10.862 | 20.027 |
| ATOM | 1103 | HA | LEU | 172 | −41.761 | 8.88 | 23.54 |
| ATOM | 1104 | H | LEU | 172 | −42.264 | 6.526 | 22.041 |
| ATOM | 1105 | HB1 | LEU | 172 | −42.107 | 8.887 | 20.477 |
| ATOM | 1106 | HB2 | LEU | 172 | −40.661 | 8.172 | 21.196 |
| ATOM | 1107 | HG | LEU | 172 | −41.5 | 11.04 | 21.89 |
| ATOM | 1108 | HD21 | LEU | 172 | −41.334 | 10.924 | 19.397 |
| ATOM | 1109 | HD22 | LEU | 172 | −40.007 | 11.874 | 20.102 |
| ATOM | 1110 | HD23 | LEU | 172 | −39.705 | 10.199 | 19.535 |
| ATOM | 1111 | HD11 | LEU | 172 | −38.737 | 9.643 | 21.856 |
| ATOM | 1112 | HD12 | LEU | 172 | −39.085 | 11.297 | 22.429 |
| ATOM | 1113 | HD13 | LEU | 172 | −39.703 | 9.896 | 23.327 |
| ATOM | 1114 | N | MET | 173 | −44.536 | 8.918 | 21.683 |
| ATOM | 1115 | CA | MET | 173 | −45.762 | 9.691 | 21.487 |
| ATOM | 1116 | C | MET | 173 | −46.577 | 9.735 | 22.762 |
| ATOM | 1117 | O | MET | 173 | −47.031 | 10.804 | 23.139 |
| ATOM | 1118 | CB | MET | 173 | −46.642 | 9.093 | 20.353 |
| ATOM | 1119 | CG | MET | 173 | −45.952 | 9.186 | 18.964 |
| ATOM | 1120 | SD | MET | 173 | −46.861 | 8.152 | 17.768 |
| ATOM | 1121 | CE | MET | 173 | −46.108 | 8.663 | 16.189 |
| ATOM | 1122 | HA | MET | 173 | −45.491 | 10.723 | 21.206 |
| ATOM | 1123 | H | MET | 173 | −44.406 | 8.056 | 21.187 |
| ATOM | 1124 | HB1 | MET | 173 | −46.87 | 8.045 | 20.598 |
| ATOM | 1125 | HB2 | MET | 173 | −47.601 | 9.635 | 20.301 |
| ATOM | 1126 | HG1 | MET | 173 | −45.942 | 10.232 | 18.624 |
| ATOM | 1127 | HG2 | MET | 173 | −44.916 | 8.819 | 18.995 |
| ATOM | 1128 | LPD1 | MET | 173 | −46.762 | 7.491 | 17.887 |
| ATOM | 1129 | LPD2 | MET | 173 | −47.527 | 8.286 | 17.78 |
| ATOM | 1130 | HE1 | MET | 173 | −46.327 | 7.904 | 15.424 |
| ATOM | 1131 | HE2 | MET | 173 | −46.545 | 9.621 | 15.873 |
| ATOM | 1132 | HE3 | MET | 173 | −45.019 | 8.772 | 16.289 |
| ATOM | 1133 | N | GLY | 174 | −46.794 | 8.584 | 23.434 |
| ATOM | 1134 | CA | GLY | 174 | −47.64 | 8.578 | 24.624 |
| ATOM | 1135 | C | GLY | 174 | −46.914 | 9.117 | 25.831 |
| ATOM | 1136 | O | GLY | 174 | −47.457 | 9.965 | 26.521 |
| ATOM | 1137 | HA2 | GLY | 174 | −47.955 | 7.55 | 24.866 |
| ATOM | 1138 | HA1 | GLY | 174 | −48.554 | 9.166 | 24.44 |
| ATOM | 1139 | H | GLY | 174 | −46.381 | 7.723 | 23.131 |
| ATOM | 1140 | N | ASN | 175 | −45.691 | 8.624 | 26.127 |
| ATOM | 1141 | CA | ASN | 175 | −44.987 | 9.093 | 27.32 |
| ATOM | 1142 | C | ASN | 175 | −44.405 | 10.47 | 27.092 |
| ATOM | 1143 | O | ASN | 175 | −44.528 | 11.303 | 27.975 |
| ATOM | 1144 | CB | ASN | 175 | −43.839 | 8.135 | 27.719 |
| ATOM | 1145 | CG | ASN | 175 | −44.364 | 6.73 | 27.888 |
| ATOM | 1146 | OD1 | ASN | 175 | −44.158 | 5.909 | 27.009 |
| ATOM | 1147 | ND2 | ASN | 175 | −45.053 | 6.412 | 29.002 |
| ATOM | 1148 | HA | ASN | 175 | −45.697 | 9.137 | 28.164 |
| ATOM | 1149 | H | ASN | 175 | −45.249 | 7.947 | 25.533 |
| ATOM | 1150 | HB1 | ASN | 175 | −43.064 | 8.156 | 26.939 |
| ATOM | 1151 | HB2 | ASN | 175 | −43.382 | 8.476 | 28.661 |
| ATOM | 1152 | HD22 | ASN | 175 | −45.404 | 5.481 | 29.107 |
| ATOM | 1153 | HD21 | ASN | 175 | −45.21 | 7.077 | 29.732 |
| ATOM | 1154 | N | GLY | 176 | −43.762 | 10.726 | 25.928 |
| ATOM | 1155 | CA | GLY | 176 | −43.219 | 12.055 | 25.644 |
| ATOM | 1156 | C | GLY | 176 | −41.748 | 11.997 | 25.307 |
| ATOM | 1157 | O | GLY | 176 | −41.376 | 12.438 | 24.232 |
| ATOM | 1158 | HA2 | GLY | 176 | −43.317 | 12.752 | 26.49 |
| ATOM | 1159 | HA1 | GLY | 176 | −43.778 | 12.489 | 24.799 |
| ATOM | 1160 | H | GLY | 176 | −43.676 | 10.032 | 25.208 |
| ATOM | 1161 | N | VAL | 177 | −40.891 | 11.484 | 26.223 |
| ATOM | 1162 | CA | VAL | 177 | −39.442 | 11.528 | 26.002 |
| ATOM | 1163 | C | VAL | 177 | −38.798 | 10.237 | 26.469 |
| ATOM | 1164 | O | VAL | 177 | −39.262 | 9.714 | 27.469 |
| ATOM | 1165 | CB | VAL | 177 | −38.819 | 12.736 | 26.762 |
| ATOM | 1166 | CG1 | VAL | 177 | −37.279 | 12.809 | 26.555 |
| ATOM | 1167 | CG2 | VAL | 177 | −39.473 | 14.072 | 26.312 |
| ATOM | 1168 | HA | VAL | 177 | −39.22 | 11.671 | 24.936 |
| ATOM | 1169 | H | VAL | 177 | −41.221 | 11.118 | 27.096 |
| ATOM | 1170 | HB | VAL | 177 | −39.017 | 12.604 | 27.84 |
| ATOM | 1171 | HG11 | VAL | 177 | −37.035 | 12.884 | 25.484 |
| ATOM | 1172 | HG12 | VAL | 177 | −36.862 | 13.688 | 27.069 |
| ATOM | 1173 | HG13 | VAL | 177 | −36.785 | 11.918 | 26.969 |
| ATOM | 1174 | HG21 | VAL | 177 | −40.55 | 14.078 | 26.538 |
| ATOM | 1175 | HG22 | VAL | 177 | −39.016 | 14.923 | 26.84 |
| ATOM | 1176 | HG23 | VAL | 177 | −39.338 | 14.22 | 25.229 |
| ATOM | 1177 | N | TRP | 178 | −37.746 | 9.738 | 25.77 |
| ATOM | 1178 | CA | TRP | 178 | −36.988 | 8.552 | 26.191 |
| ATOM | 1179 | C | TRP | 178 | −37.562 | 7.753 | 27.342 |
| ATOM | 1180 | O | TRP | 178 | −36.96 | 7.706 | 28.403 |
| ATOM | 1181 | CB | TRP | 178 | −35.51 | 8.963 | 26.455 |
| ATOM | 1182 | CG | TRP | 178 | −34.923 | 9.511 | 25.181 |
| ATOM | 1183 | CD1 | TRP | 178 | −34.483 | 10.753 | 24.913 |
| ATOM | 1184 | CD2 | TRP | 178 | −34.756 | 8.708 | 23.918 |
| ATOM | 1185 | NE1 | TRP | 178 | −34.109 | 10.819 | 23.659 |
| ATOM | 1186 | CE2 | TRP | 178 | −34.286 | 9.645 | 23.022 |
| ATOM | 1187 | CE3 | TRP | 178 | −34.972 | 7.379 | 23.566 |
| ATOM | 1188 | CZ2 | TRP | 178 | −34.076 | 9.32 | 21.682 |
| ATOM | 1189 | CZ3 | TRP | 178 | −34.859 | 7.067 | 22.208 |
| ATOM | 1190 | CH2 | TRP | 178 | −34.464 | 8.037 | 21.274 |
| ATOM | 1191 | HA | TRP | 178 | −36.998 | 7.849 | 25.347 |
| ATOM | 1192 | H | TRP | 178 | −37.433 | 10.195 | 24.935 |
| ATOM | 1193 | HB1 | TRP | 178 | −35.472 | 9.712 | 27.261 |
| ATOM | 1194 | HB2 | TRP | 178 | −34.913 | 8.089 | 26.758 |
| ATOM | 1195 | HE1 | TRP | 178 | −33.745 | 11.671 | 23.207 |
| ATOM | 1196 | HD1 | TRP | 178 | −34.455 | 11.576 | 25.628 |
| ATOM | 1197 | HZ2 | TRP | 178 | −33.638 | 10.027 | 20.99 |
| ATOM | 1198 | HH2 | TRP | 178 | −34.462 | 7.794 | 20.218 |
| ATOM | 1199 | HZ3 | TRP | 178 | −35.082 | 6.06 | 21.88 |
| ATOM | 1200 | HE3 | TRP | 178 | −35.213 | 6.634 | 24.313 |
| ATOM | 1201 | N | HIS | 179 | −38.729 | 7.1 | 27.135 |
| ATOM | 1202 | CA | HIS | 179 | −39.367 | 6.333 | 28.209 |
| ATOM | 1203 | C | HIS | 179 | −39.061 | 4.852 | 28.129 |
| ATOM | 1204 | O | HIS | 179 | −39.805 | 4.074 | 28.701 |
| ATOM | 1205 | CB | HIS | 179 | −40.883 | 6.66 | 28.17 |
| ATOM | 1206 | CG | HIS | 179 | −41.713 | 5.993 | 29.236 |
| ATOM | 1207 | ND1 | HIS | 179 | −42.265 | 4.807 | 29.103 |
| ATOM | 1208 | CD2 | HIS | 179 | −42.012 | 6.508 | 30.445 |
| ATOM | 1209 | CE1 | HIS | 179 | −42.918 | 4.508 | 30.179 |
| ATOM | 1210 | NE2 | HIS | 179 | −42.816 | 5.451 | 31 |
| ATOM | 1211 | HA | HIS | 179 | −39.008 | 6.655 | 29.201 |
| ATOM | 1212 | H | HIS | 179 | −39.18 | 7.136 | 26.241 |
| ATOM | 1213 | HB1 | HIS | 179 | −40.977 | 7.752 | 28.284 |
| ATOM | 1214 | HB2 | HIS | 179 | −41.296 | 6.384 | 27.191 |
| ATOM | 1215 | HD2 | HIS | 179 | −41.727 | 7.461 | 30.886 |
| ATOM | 1216 | HE1 | HIS | 179 | −43.475 | 3.587 | 30.354 |
| ATOM | 1217 | HD1 | HIS | 179 | −42.191 | 4.202 | 28.268 |
| ATOM | 1218 | N | PHE | 180 | −37.971 | 4.403 | 27.463 |
| ATOM | 1219 | CA | PHE | 180 | −37.626 | 2.98 | 27.515 |
| ATOM | 1220 | C | PHE | 180 | −36.135 | 2.774 | 27.311 |
| ATOM | 1221 | O | PHE | 180 | −35.715 | 1.921 | 26.546 |
| ATOM | 1222 | CB | PHE | 180 | −38.579 | 2.173 | 26.587 |
| ATOM | 1223 | CG | PHE | 180 | −38.141 | 0.707 | 26.46 |
| ATOM | 1224 | CD1 | PHE | 180 | −38.183 | −0.132 | 27.579 |
| ATOM | 1225 | CD2 | PHE | 180 | −37.693 | 0.194 | 25.237 |
| ATOM | 1226 | CE1 | PHE | 180 | −37.719 | −1.448 | 27.496 |
| ATOM | 1227 | CE2 | PHE | 180 | −37.223 | −1.12 | 25.154 |
| ATOM | 1228 | CZ | PHE | 180 | −37.229 | −1.941 | 26.284 |
| ATOM | 1229 | HA | PHE | 180 | −37.777 | 2.61 | 28.543 |
| ATOM | 1230 | H | PHE | 180 | −37.382 | 5.018 | 26.935 |
| ATOM | 1231 | HB1 | PHE | 180 | −39.601 | 2.182 | 26.997 |
| ATOM | 1232 | HB2 | PHE | 180 | −38.635 | 2.659 | 25.603 |
| ATOM | 1233 | HD2 | PHE | 180 | −37.704 | 0.809 | 24.345 |
| ATOM | 1234 | HE2 | PHE | 180 | −36.852 | −1.501 | 24.21 |
| ATOM | 1235 | HZ | PHE | 180 | −36.855 | −2.958 | 26.22 |
| ATOM | 1236 | HE1 | PHE | 180 | −37.741 | −2.088 | 28.373 |
| ATOM | 1237 | HD1 | PHE | 180 | −38.575 | 0.235 | 28.522 |
| ATOM | 1238 | N | GLY | 181 | −35.302 | 3.558 | 28.038 |
| ATOM | 1239 | CA | GLY | 181 | −33.855 | 3.336 | 28.01 |
| ATOM | 1240 | C | GLY | 181 | −33.218 | 3.534 | 26.656 |
| ATOM | 1241 | O | GLY | 181 | −33.903 | 3.834 | 25.692 |
| ATOM | 1242 | HA2 | GLY | 181 | −33.652 | 2.304 | 28.333 |
| ATOM | 1243 | HA1 | GLY | 181 | −33.349 | 4.017 | 28.714 |
| ATOM | 1244 | H | GLY | 181 | −35.66 | 4.266 | 28.652 |
| ATOM | 1245 | N | GLU | 182 | −31.88 | 3.35 | 26.558 |
| ATOM | 1246 | CA | GLU | 182 | −31.237 | 3.4 | 25.245 |
| ATOM | 1247 | C | GLU | 182 | −31.673 | 2.208 | 24.418 |
| ATOM | 1248 | O | GLU | 182 | −31.503 | 2.256 | 23.211 |
| ATOM | 1249 | CB | GLU | 182 | −29.688 | 3.512 | 25.338 |
| ATOM | 1250 | CG | GLU | 182 | −29.271 | 4.885 | 25.933 |
| ATOM | 1251 | CD | GLU | 182 | −27.786 | 4.964 | 26.168 |
| ATOM | 1252 | OE1 | GLU | 182 | −27.023 | 5.342 | 25.234 |
| ATOM | 1253 | OE2 | GLU | 182 | −27.338 | 4.671 | 27.311 |
| ATOM | 1254 | HA | GLU | 182 | −31.592 | 4.3 | 24.716 |
| ATOM | 1255 | H | GLU | 182 | −31.329 | 3.105 | 27.358 |
| ATOM | 1256 | HB1 | GLU | 182 | −29.299 | 2.691 | 25.961 |
| ATOM | 1257 | HB2 | GLU | 182 | −29.244 | 3.419 | 24.333 |
| ATOM | 1258 | HG1 | GLU | 182 | −29.563 | 5.698 | 25.249 |
| ATOM | 1259 | HG2 | GLU | 182 | −29.78 | 5.053 | 26.895 |
| ATOM | 1260 | N | THR | 183 | −32.263 | 1.134 | 24.995 |
| ATOM | 1261 | CA | THR | 183 | −32.852 | 0.096 | 24.149 |
| ATOM | 1262 | C | THR | 183 | −33.861 | 0.727 | 23.207 |
| ATOM | 1263 | O | THR | 183 | −33.923 | 0.363 | 22.043 |
| ATOM | 1264 | CB | THR | 183 | −33.545 | −0.997 | 25.006 |
| ATOM | 1265 | OG1 | THR | 183 | −32.624 | −1.38 | 26.043 |
| ATOM | 1266 | CG2 | THR | 183 | −33.917 | −2.232 | 24.142 |
| ATOM | 1267 | HA | THR | 183 | −32.028 | −0.377 | 23.596 |
| ATOM | 1268 | H | THR | 183 | −32.345 | 1.031 | 25.987 |
| ATOM | 1269 | HB | THR | 183 | −34.451 | −0.564 | 25.467 |
| ATOM | 1270 | HG1 | THR | 183 | −32.982 | −2.034 | 26.635 |
| ATOM | 1271 | HG23 | THR | 183 | −34.558 | −1.944 | 23.295 |
| ATOM | 1272 | HG21 | THR | 183 | −33.004 | −2.702 | 23.745 |
| ATOM | 1273 | HG22 | THR | 183 | −34.455 | −2.973 | 24.752 |
| ATOM | 1274 | N | MET | 184 | −34.66 | 1.703 | 23.69 |
| ATOM | 1275 | CA | MET | 184 | −35.57 | 2.427 | 22.795 |
| ATOM | 1276 | C | MET | 184 | −34.797 | 3.041 | 21.643 |
| ATOM | 1277 | O | MET | 184 | −35.201 | 2.935 | 20.495 |
| ATOM | 1278 | CB | MET | 184 | −36.26 | 3.566 | 23.597 |
| ATOM | 1279 | CG | MET | 184 | −37.447 | 4.235 | 22.869 |
| ATOM | 1280 | SD | MET | 184 | −38.071 | 5.547 | 23.968 |
| ATOM | 1281 | CE | MET | 184 | −39.095 | 6.483 | 22.801 |
| ATOM | 1282 | HA | MET | 184 | −36.328 | 1.73 | 22.409 |
| ATOM | 1283 | H | MET | 184 | −34.614 | 1.961 | 24.661 |
| ATOM | 1284 | HB1 | MET | 184 | −36.642 | 3.162 | 24.541 |
| ATOM | 1285 | HB2 | MET | 184 | −35.517 | 4.333 | 23.846 |
| ATOM | 1286 | HG1 | MET | 184 | −37.141 | 4.649 | 21.896 |
| ATOM | 1287 | HG2 | MET | 184 | −38.263 | 3.516 | 22.724 |
| ATOM | 1288 | LPD1 | MET | 184 | −38.44 | 5.293 | 24.478 |
| ATOM | 1289 | LPD2 | MET | 184 | −37.567 | 5.925 | 24.219 |
| ATOM | 1290 | HE1 | MET | 184 | −39.785 | 5.805 | 22.279 |
| ATOM | 1291 | HE2 | MET | 184 | −39.667 | 7.231 | 23.367 |
| ATOM | 1292 | HE3 | MET | 184 | −38.448 | 6.986 | 22.068 |
| ATOM | 1293 | N | CYS | 185 | −33.662 | 3.699 | 21.963 |
| ATOM | 1294 | CA | CYS | 185 | −32.825 | 4.329 | 20.938 |
| ATOM | 1295 | C | CYS | 185 | −32.197 | 3.266 | 20.058 |
| ATOM | 1296 | O | CYS | 185 | −31.971 | 3.516 | 18.884 |
| ATOM | 1297 | CB | CYS | 185 | −31.773 | 5.2 | 21.683 |
| ATOM | 1298 | SG | CYS | 185 | −30.765 | 6.383 | 20.746 |
| ATOM | 1299 | HA | CYS | 185 | −33.454 | 4.982 | 20.317 |
| ATOM | 1300 | H | CYS | 185 | −33.361 | 3.723 | 22.918 |
| ATOM | 1301 | HB1 | CYS | 185 | −32.292 | 5.828 | 22.424 |
| ATOM | 1302 | HB2 | CYS | 185 | −31.059 | 4.554 | 22.214 |
| ATOM | 1303 | LPG1 | CYS | 185 | −30.547 | 6.88 | 21.153 |
| ATOM | 1304 | LPG2 | CYS | 185 | −30.232 | 6.068 | 20.467 |
| ATOM | 1305 | N | THR | 186 | −31.914 | 2.061 | 20.605 |
| ATOM | 1306 | CA | THR | 186 | −31.334 | 0.992 | 19.795 |
| ATOM | 1307 | C | THR | 186 | −32.239 | 0.618 | 18.654 |
| ATOM | 1308 | O | THR | 186 | −31.791 | 0.687 | 17.522 |
| ATOM | 1309 | CB | THR | 186 | −30.986 | −0.287 | 20.614 |
| ATOM | 1310 | OG1 | THR | 186 | −29.827 | −0.036 | 21.429 |
| ATOM | 1311 | CG2 | THR | 186 | −30.742 | −1.549 | 19.74 |
| ATOM | 1312 | HA | THR | 186 | −30.403 | 1.384 | 19.369 |
| ATOM | 1313 | H | THR | 186 | −32.086 | 1.882 | 21.574 |
| ATOM | 1314 | HB | THR | 186 | −31.833 | −0.557 | 21.259 |
| ATOM | 1315 | HG1 | THR | 186 | −29.94 | 0.691 | 22.034 |
| ATOM | 1316 | HG23 | THR | 186 | −31.671 | −1.88 | 19.25 |
| ATOM | 1317 | HG21 | THR | 186 | −29.995 | −1.37 | 18.957 |
| ATOM | 1318 | HG22 | THR | 186 | −30.385 | −2.373 | 20.376 |
| ATOM | 1319 | N | LEU | 187 | −33.498 | 0.199 | 18.898 |
| ATOM | 1320 | CA | LEU | 187 | −34.321 | −0.227 | 17.766 |
| ATOM | 1321 | C | LEU | 187 | −34.403 | 0.902 | 16.764 |
| ATOM | 1322 | O | LEU | 187 | −34.232 | 0.67 | 15.576 |
| ATOM | 1323 | CB | LEU | 187 | −35.764 | −0.596 | 18.203 |
| ATOM | 1324 | CG | LEU | 187 | −36.67 | −1.068 | 17.025 |
| ATOM | 1325 | CD1 | LEU | 187 | −36.326 | −2.513 | 16.564 |
| ATOM | 1326 | CD2 | LEU | 187 | −38.165 | −0.998 | 17.435 |
| ATOM | 1327 | HA | LEU | 187 | −33.831 | −1.097 | 17.303 |
| ATOM | 1328 | H | LEU | 187 | −33.864 | 0.167 | 19.833 |
| ATOM | 1329 | HB1 | LEU | 187 | −35.73 | −1.377 | 18.978 |
| ATOM | 1330 | HB2 | LEU | 187 | −36.195 | 0.312 | 18.651 |
| ATOM | 1331 | HG | LEU | 187 | −36.552 | −0.386 | 16.166 |
| ATOM | 1332 | HD21 | LEU | 187 | −38.436 | 0.013 | 17.773 |
| ATOM | 1333 | HD22 | LEU | 187 | −38.781 | −1.241 | 16.561 |
| ATOM | 1334 | HD23 | LEU | 187 | −38.39 | −1.712 | 18.239 |
| ATOM | 1335 | HD11 | LEU | 187 | −35.284 | −2.597 | 16.229 |
| ATOM | 1336 | HD12 | LEU | 187 | −36.975 | −2.809 | 15.724 |
| ATOM | 1337 | HD13 | LEU | 187 | −36.481 | −3.224 | 17.39 |
| ATOM | 1338 | N | ILE | 188 | −34.671 | 2.135 | 17.251 |
| ATOM | 1339 | CA | ILE | 188 | −34.8 | 3.275 | 16.343 |
| ATOM | 1340 | C | ILE | 188 | −33.593 | 3.335 | 15.433 |
| ATOM | 1341 | O | ILE | 188 | −33.751 | 3.575 | 14.246 |
| ATOM | 1342 | CB | ILE | 188 | −34.985 | 4.611 | 17.139 |
| ATOM | 1343 | CG1 | ILE | 188 | −36.432 | 4.677 | 17.728 |
| ATOM | 1344 | CG2 | ILE | 188 | −34.648 | 5.873 | 16.288 |
| ATOM | 1345 | CD1 | ILE | 188 | −36.638 | 5.79 | 18.79 |
| ATOM | 1346 | HA | ILE | 188 | −35.685 | 3.102 | 15.713 |
| ATOM | 1347 | H | ILE | 188 | −34.77 | 2.281 | 18.238 |
| ATOM | 1348 | HB | ILE | 188 | −34.257 | 4.604 | 17.966 |
| ATOM | 1349 | HG11 | ILE | 188 | −37.161 | 4.826 | 16.917 |
| ATOM | 1350 | HG12 | ILE | 188 | −36.677 | 3.72 | 18.217 |
| ATOM | 1351 | HD11 | ILE | 188 | −36.56 | 6.796 | 18.353 |
| ATOM | 1352 | HD12 | ILE | 188 | −37.638 | 5.697 | 19.245 |
| ATOM | 1353 | HD13 | ILE | 188 | −35.888 | 5.685 | 19.585 |
| ATOM | 1354 | HG21 | ILE | 188 | −35.327 | 5.961 | 15.431 |
| ATOM | 1355 | HG22 | ILE | 188 | −34.726 | 6.789 | 16.89 |
| ATOM | 1356 | HG23 | ILE | 188 | −33.617 | 5.836 | 15.907 |
| ATOM | 1357 | N | THR | 189 | −32.373 | 3.132 | 15.973 |
| ATOM | 1358 | CA | THR | 189 | −31.177 | 3.314 | 15.151 |
| ATOM | 1359 | C | THR | 189 | −30.861 | 2.059 | 14.377 |
| ATOM | 1360 | O | THR | 189 | −30.63 | 2.135 | 13.182 |
| ATOM | 1361 | CB | THR | 189 | −29.898 | 3.621 | 15.979 |
| ATOM | 1362 | OG1 | THR | 189 | −30.137 | 4.527 | 17.058 |
| ATOM | 1363 | CG2 | THR | 189 | −28.795 | 4.268 | 15.099 |
| ATOM | 1364 | HA | THR | 189 | −31.346 | 4.161 | 14.467 |
| ATOM | 1365 | H | THR | 189 | −32.284 | 2.845 | 16.928 |
| ATOM | 1366 | HB | THR | 189 | −29.522 | 2.686 | 16.419 |
| ATOM | 1367 | HG1 | THR | 189 | −30.695 | 4.195 | 17.756 |
| ATOM | 1368 | HG23 | THR | 189 | −28.474 | 3.581 | 14.301 |
| ATOM | 1369 | HG21 | THR | 189 | −29.163 | 5.199 | 14.641 |
| ATOM | 1370 | HG22 | THR | 189 | −27.924 | 4.525 | 15.722 |
| ATOM | 1371 | N | ALA | 190 | −30.815 | 0.895 | 15.064 |
| ATOM | 1372 | CA | ALA | 190 | −30.397 | −0.339 | 14.412 |
| ATOM | 1373 | C | ALA | 190 | −31.162 | −0.517 | 13.129 |
| ATOM | 1374 | O | ALA | 190 | −30.562 | −0.895 | 12.136 |
| ATOM | 1375 | CB | ALA | 190 | −30.658 | −1.568 | 15.323 |
| ATOM | 1376 | HA | ALA | 190 | −29.318 | −0.272 | 14.189 |
| ATOM | 1377 | H | ALA | 190 | −31.035 | 0.861 | 16.038 |
| ATOM | 1378 | HB1 | ALA | 190 | −30.367 | −2.497 | 14.81 |
| ATOM | 1379 | HB2 | ALA | 190 | −30.071 | −1.482 | 16.251 |
| ATOM | 1380 | HB3 | ALA | 190 | −31.726 | −1.628 | 15.585 |
| ATOM | 1381 | N | MET | 191 | −32.485 | −0.245 | 13.121 |
| ATOM | 1382 | CA | MET | 191 | −33.23 | −0.409 | 11.879 |
| ATOM | 1383 | C | MET | 191 | −32.912 | 0.72 | 10.92 |
| ATOM | 1384 | O | MET | 191 | −32.69 | 0.441 | 9.752 |
| ATOM | 1385 | CB | MET | 191 | −34.757 | −0.495 | 12.108 |
| ATOM | 1386 | CG | MET | 191 | −35.195 | −1.644 | 13.055 |
| ATOM | 1387 | SD | MET | 191 | −34.768 | −3.273 | 12.352 |
| ATOM | 1388 | CE | MET | 191 | −33.318 | −3.752 | 13.344 |
| ATOM | 1389 | HA | MET | 191 | −32.929 | −1.354 | 11.403 |
| ATOM | 1390 | H | MET | 191 | −32.956 | 0.106 | 13.936 |
| ATOM | 1391 | HB1 | MET | 191 | −35.126 | 0.462 | 12.514 |
| ATOM | 1392 | HB2 | MET | 191 | −35.199 | −0.668 | 11.116 |
| ATOM | 1393 | HG1 | MET | 191 | −34.748 | −1.528 | 14.052 |
| ATOM | 1394 | HG2 | MET | 191 | −36.29 | −1.622 | 13.167 |
| ATOM | 1395 | LPD1 | MET | 191 | −35.277 | −3.716 | 12.43 |
| ATOM | 1396 | LPD2 | MET | 191 | −34.61 | −3.223 | 11.693 |
| ATOM | 1397 | HE1 | MET | 191 | −33.595 | −3.815 | 14.407 |
| ATOM | 1398 | HE2 | MET | 191 | −32.966 | −4.737 | 13.005 |
| ATOM | 1399 | HE3 | MET | 191 | −32.507 | −3.022 | 13.221 |
| ATOM | 1400 | N | ASP | 192 | −32.885 | 1.994 | 11.379 |
| ATOM | 1401 | CA | ASP | 192 | −32.549 | 3.099 | 10.472 |
| ATOM | 1402 | C | ASP | 192 | −31.258 | 2.78 | 9.75 |
| ATOM | 1403 | O | ASP | 192 | −31.187 | 2.836 | 8.531 |
| ATOM | 1404 | CB | ASP | 192 | −32.378 | 4.434 | 11.258 |
| ATOM | 1405 | CG | ASP | 192 | −32.205 | 5.625 | 10.356 |
| ATOM | 1406 | OD1 | ASP | 192 | −31.258 | 6.43 | 10.58 |
| ATOM | 1407 | OD2 | ASP | 192 | −33.032 | 5.777 | 9.422 |
| ATOM | 1408 | HA | ASP | 192 | −33.355 | 3.209 | 9.728 |
| ATOM | 1409 | H | ASP | 192 | −33.084 | 2.197 | 12.341 |
| ATOM | 1410 | HB1 | ASP | 192 | −33.266 | 4.638 | 11.872 |
| ATOM | 1411 | HB2 | ASP | 192 | −31.518 | 4.371 | 11.939 |
| ATOM | 1412 | N | ALA | 193 | −30.21 | 2.424 | 10.523 |
| ATOM | 1413 | CA | ALA | 193 | −28.914 | 2.132 | 9.925 |
| ATOM | 1414 | C | ALA | 193 | −28.981 | 0.833 | 9.151 |
| ATOM | 1415 | O | ALA | 193 | −28.474 | 0.78 | 8.042 |
| ATOM | 1416 | CB | ALA | 193 | −27.832 | 2.064 | 11.033 |
| ATOM | 1417 | HA | ALA | 193 | −28.641 | 2.958 | 9.249 |
| ATOM | 1418 | H | ALA | 193 | −30.316 | 2.33 | 11.514 |
| ATOM | 1419 | HB1 | ALA | 193 | −26.85 | 1.806 | 10.608 |
| ATOM | 1420 | HB2 | ALA | 193 | −27.752 | 3.041 | 11.536 |
| ATOM | 1421 | HB3 | ALA | 193 | −28.113 | 1.311 | 11.783 |
| ATOM | 1422 | N | ASN | 194 | −29.595 | −0.241 | 9.698 |
| ATOM | 1423 | CA | ASN | 194 | −29.638 | −1.498 | 8.953 |
| ATOM | 1424 | C | ASN | 194 | −30.258 | −1.266 | 7.599 |
| ATOM | 1425 | O | ASN | 194 | −29.748 | −1.795 | 6.625 |
| ATOM | 1426 | CB | ASN | 194 | −30.407 | −2.635 | 9.677 |
| ATOM | 1427 | CG | ASN | 194 | −30.315 | −3.918 | 8.888 |
| ATOM | 1428 | OD1 | ASN | 194 | −29.432 | −4.713 | 9.168 |
| ATOM | 1429 | ND2 | ASN | 194 | −31.193 | −4.151 | 7.89 |
| ATOM | 1430 | HA | ASN | 194 | −28.603 | −1.838 | 8.799 |
| ATOM | 1431 | H | ASN | 194 | −30.02 | −0.194 | 10.604 |
| ATOM | 1432 | HB1 | ASN | 194 | −29.974 | −2.815 | 10.672 |
| ATOM | 1433 | HB2 | ASN | 194 | −31.46 | −2.349 | 9.804 |
| ATOM | 1434 | HD22 | ASN | 194 | −31.101 | −4.984 | 7.344 |
| ATOM | 1435 | HD21 | ASN | 194 | −31.917 | −3.501 | 7.665 |
| ATOM | 1436 | N | SER | 195 | −31.356 | −0.485 | 7.501 |
| ATOM | 1437 | CA | SER | 195 | −31.933 | −0.246 | 6.182 |
| ATOM | 1438 | C | SER | 195 | −30.988 | 0.599 | 5.362 |
| ATOM | 1439 | O | SER | 195 | −30.706 | 0.247 | 4.227 |
| ATOM | 1440 | CB | SER | 195 | −33.357 | 0.365 | 6.242 |
| ATOM | 1441 | OG | SER | 195 | −33.914 | 0.359 | 4.914 |
| ATOM | 1442 | HA | SER | 195 | −32.014 | −1.217 | 5.676 |
| ATOM | 1443 | H | SER | 195 | −31.763 | −0.041 | 8.304 |
| ATOM | 1444 | HB1 | SER | 195 | −33.974 | −0.257 | 6.914 |
| ATOM | 1445 | HB2 | SER | 195 | −33.32 | 1.39 | 6.647 |
| ATOM | 1446 | HG | SER | 195 | −34.84 | 0.592 | 4.882 |
| ATOM | 1447 | N | GLN | 196 | −30.471 | 1.713 | 5.929 |
| ATOM | 1448 | CA | GLN | 196 | −29.528 | 2.542 | 5.179 |
| ATOM | 1449 | C | GLN | 196 | −28.441 | 1.695 | 4.553 |
| ATOM | 1450 | O | GLN | 196 | −28.178 | 1.827 | 3.366 |
| ATOM | 1451 | CB | GLN | 196 | −28.847 | 3.576 | 6.119 |
| ATOM | 1452 | CG | GLN | 196 | −27.851 | 4.512 | 5.389 |
| ATOM | 1453 | CD | GLN | 196 | −27.254 | 5.527 | 6.338 |
| ATOM | 1454 | OE1 | GLN | 196 | −27.325 | 5.353 | 7.544 |
| ATOM | 1455 | NE2 | GLN | 196 | −26.641 | 6.611 | 5.824 |
| ATOM | 1456 | HA | GLN | 196 | −30.085 | 3.076 | 4.391 |
| ATOM | 1457 | H | GLN | 196 | −30.731 | 1.985 | 6.861 |
| ATOM | 1458 | HB1 | GLN | 196 | −29.622 | 4.186 | 6.607 |
| ATOM | 1459 | HB2 | GLN | 196 | −28.291 | 3.042 | 6.902 |
| ATOM | 1460 | HG1 | GLN | 196 | −27.02 | 3.929 | 4.961 |
| ATOM | 1461 | HG2 | GLN | 196 | −28.375 | 5.023 | 4.569 |
| ATOM | 1462 | HE22 | GLN | 196 | −26.227 | 7.284 | 6.437 |
| ATOM | 1463 | HE21 | GLN | 196 | −26.599 | 6.751 | 4.839 |
| ATOM | 1464 | N | PHE | 197 | −27.776 | 0.827 | 5.348 |
| ATOM | 1465 | CA | PHE | 197 | −26.635 | 0.083 | 4.821 |
| ATOM | 1466 | C | PHE | 197 | −27.051 | −1.164 | 4.073 |
| ATOM | 1467 | O | PHE | 197 | −26.392 | −1.492 | 3.099 |
| ATOM | 1468 | CB | PHE | 197 | −25.592 | −0.207 | 5.932 |
| ATOM | 1469 | CG | PHE | 197 | −24.934 | 1.118 | 6.36 |
| ATOM | 1470 | CD1 | PHE | 197 | −24.002 | 1.734 | 5.517 |
| ATOM | 1471 | CD2 | PHE | 197 | −25.246 | 1.73 | 7.579 |
| ATOM | 1472 | CE1 | PHE | 197 | −23.338 | 2.898 | 5.914 |
| ATOM | 1473 | CE2 | PHE | 197 | −24.602 | 2.906 | 7.973 |
| ATOM | 1474 | CZ | PHE | 197 | −23.641 | 3.489 | 7.144 |
| ATOM | 1475 | HA | PHE | 197 | −26.115 | 0.717 | 4.091 |
| ATOM | 1476 | H | PHE | 197 | −28.048 | 0.684 | 6.303 |
| ATOM | 1477 | HB1 | PHE | 197 | −26.067 | −0.727 | 6.779 |
| ATOM | 1478 | HB2 | PHE | 197 | −24.803 | −0.865 | 5.536 |
| ATOM | 1479 | HD2 | PHE | 197 | −25.993 | 1.296 | 8.229 |
| ATOM | 1480 | HE2 | PHE | 197 | −24.849 | 3.368 | 8.924 |
| ATOM | 1481 | HZ | PHE | 197 | −23.133 | 4.397 | 7.454 |
| ATOM | 1482 | HE1 | PHE | 197 | −22.588 | 3.342 | 5.267 |
| ATOM | 1483 | HD1 | PHE | 197 | −23.786 | 1.309 | 4.542 |
| ATOM | 1484 | N | THR | 198 | −28.129 | −1.88 | 4.464 |
| ATOM | 1485 | CA | THR | 198 | −28.527 | −3.054 | 3.684 |
| ATOM | 1486 | C | THR | 198 | −28.9 | −2.565 | 2.304 |
| ATOM | 1487 | O | THR | 198 | −28.469 | −3.152 | 1.323 |
| ATOM | 1488 | CB | THR | 198 | −29.69 | −3.9 | 4.288 |
| ATOM | 1489 | OG1 | THR | 198 | −29.436 | −4.309 | 5.64 |
| ATOM | 1490 | CG2 | THR | 198 | −29.866 | −5.229 | 3.508 |
| ATOM | 1491 | HA | THR | 198 | −27.65 | −3.712 | 3.584 |
| ATOM | 1492 | H | THR | 198 | −28.688 | −1.584 | 5.239 |
| ATOM | 1493 | HB | THR | 198 | −30.628 | −3.318 | 4.256 |
| ATOM | 1494 | HG1 | THR | 198 | −29.298 | −3.581 | 6.238 |
| ATOM | 1495 | HG23 | THR | 198 | −30.112 | −5.032 | 2.458 |
| ATOM | 1496 | HG21 | THR | 198 | −28.939 | −5.824 | 3.545 |
| ATOM | 1497 | HG22 | THR | 198 | −30.671 | −5.832 | 3.953 |
| ATOM | 1498 | N | SER | 199 | −29.688 | −1.471 | 2.206 |
| ATOM | 1499 | CA | SER | 199 | −29.974 | −0.909 | 0.89 |
| ATOM | 1500 | C | SER | 199 | −28.687 | −0.519 | 0.199 |
| ATOM | 1501 | O | SER | 199 | −28.528 | −0.835 | −0.97 |
| ATOM | 1502 | CB | SER | 199 | −30.926 | 0.307 | 0.981 |
| ATOM | 1503 | OG | SER | 199 | −32.165 | −0.171 | 1.532 |
| ATOM | 1504 | HA | SER | 199 | −30.496 | −1.669 | 0.293 |
| ATOM | 1505 | H | SER | 199 | −30.06 | −1.009 | 3.018 |
| ATOM | 1506 | HB1 | SER | 199 | −30.483 | 1.084 | 1.625 |
| ATOM | 1507 | HB2 | SER | 199 | −31.092 | 0.728 | −0.026 |
| ATOM | 1508 | HG | SER | 199 | −32.816 | 0.516 | 1.627 |
| ATOM | 1509 | N | THR | 200 | −27.749 | 0.163 | 0.897 |
| ATOM | 1510 | CA | THR | 200 | −26.505 | 0.564 | 0.236 |
| ATOM | 1511 | C | THR | 200 | −25.854 | −0.657 | −0.374 |
| ATOM | 1512 | O | THR | 200 | −25.469 | −0.616 | −1.532 |
| ATOM | 1513 | CB | THR | 200 | −25.461 | 1.255 | 1.165 |
| ATOM | 1514 | OG1 | THR | 200 | −25.972 | 2.467 | 1.75 |
| ATOM | 1515 | CG2 | THR | 200 | −24.182 | 1.663 | 0.388 |
| ATOM | 1516 | HA | THR | 200 | −26.766 | 1.279 | −0.559 |
| ATOM | 1517 | H | THR | 200 | −27.9 | 0.395 | 1.86 |
| ATOM | 1518 | HB | THR | 200 | −25.15 | 0.552 | 1.954 |
| ATOM | 1519 | HG1 | THR | 200 | −26.725 | 2.332 | 2.317 |
| ATOM | 1520 | HG23 | THR | 200 | −23.66 | 0.78 | −0.008 |
| ATOM | 1521 | HG21 | THR | 200 | −24.438 | 2.333 | −0.448 |
| ATOM | 1522 | HG22 | THR | 200 | −23.493 | 2.196 | 1.062 |
| ATOM | 1523 | N | TYR | 201 | −25.704 | −1.762 | 0.389 |
| ATOM | 1524 | CA | TYR | 201 | −25.041 | −2.932 | −0.178 |
| ATOM | 1525 | C | TYR | 201 | −25.896 | −3.567 | −1.261 |
| ATOM | 1526 | O | TYR | 201 | −25.343 | −3.964 | −2.275 |
| ATOM | 1527 | CB | TYR | 201 | −24.672 | −3.978 | 0.907 |
| ATOM | 1528 | CG | TYR | 201 | −23.68 | −3.47 | 1.968 |
| ATOM | 1529 | CD1 | TYR | 201 | −24.039 | −3.407 | 3.32 |
| ATOM | 1530 | CD2 | TYR | 201 | −22.384 | −3.094 | 1.593 |
| ATOM | 1531 | CE1 | TYR | 201 | −23.095 | −3.046 | 4.287 |
| ATOM | 1532 | CE2 | TYR | 201 | −21.439 | −2.736 | 2.559 |
| ATOM | 1533 | CZ | TYR | 201 | −21.776 | −2.768 | 3.914 |
| ATOM | 1534 | OH | TYR | 201 | −20.793 | −2.532 | 4.877 |
| ATOM | 1535 | HA | TYR | 201 | −24.106 | −2.595 | −0.657 |
| ATOM | 1536 | H | TYR | 201 | −26.045 | −1.799 | 1.333 |
| ATOM | 1537 | HB2 | TYR | 201 | −25.591 | −4.363 | 1.375 |
| ATOM | 1538 | HB1 | TYR | 201 | −24.156 | −4.81 | 0.411 |
| ATOM | 1539 | HD2 | TYR | 201 | −22.094 | −3.089 | 0.548 |
| ATOM | 1540 | HE2 | TYR | 201 | −20.438 | −2.443 | 2.26 |
| ATOM | 1541 | HE1 | TYR | 201 | −23.382 | −2.989 | 5.329 |
| ATOM | 1542 | HD1 | TYR | 201 | −25.048 | −3.648 | 3.634 |
| ATOM | 1543 | HH | TYR | 201 | −20.912 | −3.068 | 5.653 |
| ATOM | 1544 | N | ILE | 202 | −27.235 | −3.683 | −1.098 |
| ATOM | 1545 | CA | ILE | 202 | −28.067 | −4.24 | −2.176 |
| ATOM | 1546 | C | ILE | 202 | −27.738 | −3.558 | −3.483 |
| ATOM | 1547 | O | ILE | 202 | −27.602 | −4.232 | −4.492 |
| ATOM | 1548 | CB | ILE | 202 | −29.596 | −4.12 | −1.871 |
| ATOM | 1549 | CG1 | ILE | 202 | −30.032 | −5.248 | −0.885 |
| ATOM | 1550 | CG2 | ILE | 202 | −30.464 | −4.14 | −3.166 |
| ATOM | 1551 | CD1 | ILE | 202 | −31.335 | −4.96 | −0.102 |
| ATOM | 1552 | HA | ILE | 202 | −27.815 | −5.303 | −2.314 |
| ATOM | 1553 | H | ILE | 202 | −27.668 | −3.375 | −0.247 |
| ATOM | 1554 | HB | ILE | 202 | −29.763 | −3.139 | −1.406 |
| ATOM | 1555 | HG11 | ILE | 202 | −30.204 | −6.172 | −1.458 |
| ATOM | 1556 | HG12 | ILE | 202 | −29.238 | −5.427 | −0.142 |
| ATOM | 1557 | HD11 | ILE | 202 | −32.183 | −4.872 | −0.787 |
| ATOM | 1558 | HD12 | ILE | 202 | −31.56 | −5.781 | 0.595 |
| ATOM | 1559 | HD13 | ILE | 202 | −31.228 | −4.032 | 0.476 |
| ATOM | 1560 | HG21 | ILE | 202 | −30.265 | −5.058 | −3.739 |
| ATOM | 1561 | HG22 | ILE | 202 | −31.535 | −4.095 | −2.927 |
| ATOM | 1562 | HG23 | ILE | 202 | −30.248 | −3.272 | −3.809 |
| ATOM | 1563 | N | LEU | 203 | −27.609 | −2.215 | −3.492 |
| ATOM | 1564 | CA | LEU | 203 | −27.299 | −1.538 | −4.75 |
| ATOM | 1565 | C | LEU | 203 | −26.03 | −2.091 | −5.359 |
| ATOM | 1566 | O | LEU | 203 | −25.955 | −2.182 | −6.574 |
| ATOM | 1567 | CB | LEU | 203 | −27.079 | −0.017 | −4.562 |
| ATOM | 1568 | CG | LEU | 203 | −28.33 | 0.779 | −4.098 |
| ATOM | 1569 | CD1 | LEU | 203 | −27.875 | 2.216 | −3.727 |
| ATOM | 1570 | CD2 | LEU | 203 | −29.448 | 0.801 | −5.177 |
| ATOM | 1571 | HA | LEU | 203 | −28.121 | −1.716 | −5.459 |
| ATOM | 1572 | H | LEU | 203 | −27.711 | −1.688 | −2.644 |
| ATOM | 1573 | HB1 | LEU | 203 | −26.298 | 0.095 | −3.8 |
| ATOM | 1574 | HB2 | LEU | 203 | −26.709 | 0.42 | −5.504 |
| ATOM | 1575 | HG | LEU | 203 | −28.765 | 0.336 | −3.189 |
| ATOM | 1576 | HD21 | LEU | 203 | −29.902 | −0.191 | −5.313 |
| ATOM | 1577 | HD22 | LEU | 203 | −30.246 | 1.488 | −4.868 |
| ATOM | 1578 | HD23 | LEU | 203 | −29.059 | 1.144 | −6.143 |
| ATOM | 1579 | HD11 | LEU | 203 | −27.379 | 2.703 | −4.579 |
| ATOM | 1580 | HD12 | LEU | 203 | −28.748 | 2.805 | −3.425 |
| ATOM | 1581 | HD13 | LEU | 203 | −27.168 | 2.203 | −2.884 |
| ATOM | 1582 | N | THR | 204 | −25.015 | −2.47 | −4.551 |
| ATOM | 1583 | CA | THR | 204 | −23.815 | −3.064 | −5.137 |
| ATOM | 1584 | C | THR | 204 | −24.215 | −4.342 | −5.832 |
| ATOM | 1585 | O | THR | 204 | −23.84 | −4.544 | −6.976 |
| ATOM | 1586 | CB | THR | 204 | −22.689 | −3.344 | −4.099 |
| ATOM | 1587 | OG1 | THR | 204 | −22.224 | −2.119 | −3.506 |
| ATOM | 1588 | CG2 | THR | 204 | −21.462 | −4.007 | −4.773 |
| ATOM | 1589 | HA | THR | 204 | −23.405 | −2.379 | −5.894 |
| ATOM | 1590 | H | THR | 204 | −25.096 | −2.403 | −3.556 |
| ATOM | 1591 | HB | THR | 204 | −23.052 | −4.025 | −3.311 |
| ATOM | 1592 | HG1 | THR | 204 | −22.905 | −1.663 | −3.026 |
| ATOM | 1593 | HG23 | THR | 204 | −21.717 | −5 | −5.169 |
| ATOM | 1594 | HG21 | THR | 204 | −21.11 | −3.373 | −5.598 |
| ATOM | 1595 | HG22 | THR | 204 | −20.645 | −4.121 | −4.043 |
| ATOM | 1596 | N | ALA | 205 | −24.983 | −5.226 | −5.16 |
| ATOM | 1597 | CA | ALA | 205 | −25.378 | −6.463 | −5.825 |
| ATOM | 1598 | C | ALA | 205 | −26.227 | −6.162 | −7.039 |
| ATOM | 1599 | O | ALA | 205 | −26.106 | −6.887 | −8.013 |
| ATOM | 1600 | CB | ALA | 205 | −26.119 | −7.426 | −4.872 |
| ATOM | 1601 | HA | ALA | 205 | −24.459 | −6.977 | −6.155 |
| ATOM | 1602 | H | ALA | 205 | −25.309 | −5.038 | −4.229 |
| ATOM | 1603 | HB1 | ALA | 205 | −26.325 | −8.381 | −5.375 |
| ATOM | 1604 | HB2 | ALA | 205 | −25.478 | −7.635 | −4.004 |
| ATOM | 1605 | HB3 | ALA | 205 | −27.064 | −6.971 | −4.538 |
| ATOM | 1606 | N | MET | 206 | −27.078 | −5.109 | −7.036 |
| ATOM | 1607 | CA | MET | 206 | −27.773 | −4.756 | −8.273 |
| ATOM | 1608 | C | MET | 206 | −26.744 | −4.386 | −9.311 |
| ATOM | 1609 | O | MET | 206 | −26.754 | −4.95 | −10.392 |
| ATOM | 1610 | CB | MET | 206 | −28.768 | −3.566 | −8.174 |
| ATOM | 1611 | CG | MET | 206 | −30.008 | −3.917 | −7.321 |
| ATOM | 1612 | SD | MET | 206 | −31.181 | −2.518 | −7.33 |
| ATOM | 1613 | CE | MET | 206 | −32.456 | −3.147 | −8.472 |
| ATOM | 1614 | HA | MET | 206 | −28.327 | −5.635 | −8.632 |
| ATOM | 1615 | H | MET | 206 | −27.199 | −4.535 | −6.223 |
| ATOM | 1616 | HB1 | MET | 206 | −28.273 | −2.675 | −7.761 |
| ATOM | 1617 | HB2 | MET | 206 | −29.121 | −3.32 | −9.189 |
| ATOM | 1618 | HG1 | MET | 206 | −30.493 | −4.796 | −7.763 |
| ATOM | 1619 | HG2 | MET | 206 | −29.722 | −4.143 | −6.283 |
| ATOM | 1620 | LPD1 | MET | 206 | −31.436 | −2.402 | −6.711 |
| ATOM | 1621 | LPD2 | MET | 206 | −30.892 | −1.951 | −7.566 |
| ATOM | 1622 | HE1 | MET | 206 | −32.972 | −4.004 | −8.014 |
| ATOM | 1623 | HE2 | MET | 206 | −33.19 | −2.355 | −8.678 |
| ATOM | 1624 | HE3 | MET | 206 | −31.995 | −3.465 | −9.418 |
| ATOM | 1625 | N | ALA | 207 | −25.842 | −3.43 | −9.014 |
| ATOM | 1626 | CA | ALA | 207 | −24.89 | −3.005 | −10.034 |
| ATOM | 1627 | C | ALA | 207 | −24.123 | −4.198 | −10.561 |
| ATOM | 1628 | O | ALA | 207 | −23.868 | −4.262 | −11.754 |
| ATOM | 1629 | CB | ALA | 207 | −23.923 | −1.936 | −9.471 |
| ATOM | 1630 | HA | ALA | 207 | −25.462 | −2.555 | −10.865 |
| ATOM | 1631 | H | ALA | 207 | −25.818 | −3.005 | −8.106 |
| ATOM | 1632 | HB1 | ALA | 207 | −23.324 | −1.532 | −10.296 |
| ATOM | 1633 | HB2 | ALA | 207 | −24.487 | −1.107 | −9.017 |
| ATOM | 1634 | HB3 | ALA | 207 | −23.257 | −2.374 | −8.712 |
| ATOM | 1635 | N | ILE | 208 | −23.762 | −5.168 | −9.69 |
| ATOM | 1636 | CA | ILE | 208 | −23.122 | −6.386 | −10.186 |
| ATOM | 1637 | C | ILE | 208 | −24.118 | −7.128 | −11.055 |
| ATOM | 1638 | O | ILE | 208 | −23.765 | −7.493 | −12.164 |
| ATOM | 1639 | CB | ILE | 208 | −22.563 | −7.283 | −9.038 |
| ATOM | 1640 | CG1 | ILE | 208 | −21.379 | −6.556 | −8.324 |
| ATOM | 1641 | CG2 | ILE | 208 | −22.129 | −8.672 | −9.589 |
| ATOM | 1642 | CD1 | ILE | 208 | −20.905 | −7.252 | −7.02 |
| ATOM | 1643 | HA | ILE | 208 | −22.263 | −6.098 | −10.812 |
| ATOM | 1644 | H | ILE | 208 | −23.984 | −5.091 | −8.715 |
| ATOM | 1645 | HB | ILE | 208 | −23.375 | −7.455 | −8.315 |
| ATOM | 1646 | HG11 | ILE | 208 | −20.524 | −6.474 | −9.015 |
| ATOM | 1647 | HG12 | ILE | 208 | −21.682 | −5.533 | −8.055 |
| ATOM | 1648 | HD11 | ILE | 208 | −20.432 | −8.223 | −7.223 |
| ATOM | 1649 | HD12 | ILE | 208 | −20.158 | −6.621 | −6.512 |
| ATOM | 1650 | HD13 | ILE | 208 | −21.755 | −7.406 | −6.336 |
| ATOM | 1651 | HG21 | ILE | 208 | −21.338 | −8.556 | −10.346 |
| ATOM | 1652 | HG22 | ILE | 208 | −21.758 | −9.329 | −8.79 |
| ATOM | 1653 | HG23 | ILE | 208 | −22.983 | −9.188 | −10.05 |
| ATOM | 1654 | N | ASP | 209 | −25.363 | −7.368 | −10.587 |
| ATOM | 1655 | CA | ASP | 209 | −26.348 | −8.071 | −11.417 |
| ATOM | 1656 | C | ASP | 209 | −26.444 | −7.43 | −12.783 |
| ATOM | 1657 | O | ASP | 209 | −26.468 | −8.128 | −13.786 |
| ATOM | 1658 | CB | ASP | 209 | −27.757 | −8.053 | −10.763 |
| ATOM | 1659 | CG | ASP | 209 | −28.81 | −8.727 | −11.605 |
| ATOM | 1660 | OD1 | ASP | 209 | −29.707 | −8.017 | −12.136 |
| ATOM | 1661 | OD2 | ASP | 209 | −28.759 | −9.977 | −11.734 |
| ATOM | 1662 | HA | ASP | 209 | −26.026 | −9.117 | −11.528 |
| ATOM | 1663 | H | ASP | 209 | −25.629 | −7.063 | −9.672 |
| ATOM | 1664 | HB1 | ASP | 209 | −27.713 | −8.547 | −9.782 |
| ATOM | 1665 | HB2 | ASP | 209 | −28.077 | −7.016 | −10.602 |
| ATOM | 1666 | N | ARG | 210 | −26.503 | −6.083 | −12.844 |
| ATOM | 1667 | CA | ARG | 210 | −26.683 | −5.428 | −14.135 |
| ATOM | 1668 | C | ARG | 210 | −25.407 | −5.554 | −14.933 |
| ATOM | 1669 | O | ARG | 210 | −25.481 | −5.843 | −16.116 |
| ATOM | 1670 | CB | ARG | 210 | −27.106 | −3.939 | −14 |
| ATOM | 1671 | CG | ARG | 210 | −28.309 | −3.683 | −13.042 |
| ATOM | 1672 | CD | ARG | 210 | −29.417 | −4.772 | −13.104 |
| ATOM | 1673 | NE | ARG | 210 | −29.992 | −4.924 | −14.444 |
| ATOM | 1674 | CZ | ARG | 210 | −30.731 | −5.949 | −14.812 |
| ATOM | 1675 | NH1 | ARG | 210 | −31.034 | −6.954 | −14.024 |
| ATOM | 1676 | NH2 | ARG | 210 | −31.2 | −5.97 | −16.036 |
| ATOM | 1677 | HA | ARG | 210 | −27.476 | −5.954 | −14.693 |
| ATOM | 1678 | H | ARG | 210 | −26.414 | −5.526 | −12.016 |
| ATOM | 1679 | HB1 | ARG | 210 | −26.252 | −3.364 | −13.617 |
| ATOM | 1680 | HB2 | ARG | 210 | −27.335 | −3.567 | −15.013 |
| ATOM | 1681 | HG1 | ARG | 210 | −27.958 | −3.615 | −12.002 |
| ATOM | 1682 | HG2 | ARG | 210 | −28.733 | −2.697 | −13.274 |
| ATOM | 1683 | HD1 | ARG | 210 | −28.947 | −5.695 | −12.742 |
| ATOM | 1684 | HD2 | ARG | 210 | −30.224 | −4.505 | −12.401 |
| ATOM | 1685 | HE | ARG | 210 | −29.791 | −4.179 | −15.131 |
| ATOM | 1686 | HH12 | ARG | 210 | −31.617 | −7.738 | −14.352 |
| ATOM | 1687 | HH11 | ARG | 210 | −30.694 | −7.012 | −13.053 |
| ATOM | 1688 | HH22 | ARG | 210 | −31.783 | −6.753 | −16.36 |
| ATOM | 1689 | HH21 | ARG | 210 | −31.017 | −5.198 | −16.699 |
| ATOM | 1690 | N | TYR | 211 | −24.222 | −5.36 | −14.313 |
| ATOM | 1691 | CA | TYR | 211 | −22.979 | −5.6 | −15.045 |
| ATOM | 1692 | C | TYR | 211 | −23.001 | −7 | −15.617 |
| ATOM | 1693 | O | TYR | 211 | −22.715 | −7.162 | −16.794 |
| ATOM | 1694 | CB | TYR | 211 | −21.731 | −5.451 | −14.134 |
| ATOM | 1695 | CG | TYR | 211 | −20.494 | −6.023 | −14.841 |
| ATOM | 1696 | CD1 | TYR | 211 | −19.759 | −5.228 | −15.724 |
| ATOM | 1697 | CD2 | TYR | 211 | −20.097 | −7.347 | −14.616 |
| ATOM | 1698 | CE1 | TYR | 211 | −18.658 | −5.76 | −16.401 |
| ATOM | 1699 | CE2 | TYR | 211 | −18.996 | −7.879 | −15.293 |
| ATOM | 1700 | CZ | TYR | 211 | −18.28 | −7.092 | −16.198 |
| ATOM | 1701 | OH | TYR | 211 | −17.197 | −7.645 | −16.884 |
| ATOM | 1702 | HA | TYR | 211 | −22.898 | −4.882 | −15.877 |
| ATOM | 1703 | H | TYR | 211 | −24.179 | −5.103 | −13.344 |
| ATOM | 1704 | HB2 | TYR | 211 | −21.58 | −4.392 | −13.869 |
| ATOM | 1705 | HB1 | TYR | 211 | −21.884 | −6.007 | −13.199 |
| ATOM | 1706 | HD2 | TYR | 211 | −20.643 | −7.972 | −13.918 |
| ATOM | 1707 | HE2 | TYR | 211 | −18.695 | −8.907 | −15.119 |
| ATOM | 1708 | HE1 | TYR | 211 | −18.102 | −5.139 | −17.093 |
| ATOM | 1709 | HD1 | TYR | 211 | −20.05 | −4.197 | −15.89 |
| ATOM | 1710 | HH | TYR | 211 | −17.085 | −7.276 | −17.754 |
| ATOM | 1711 | N | LEU | 212 | −23.338 | −8.015 | −14.79 |
| ATOM | 1712 | CA | LEU | 212 | −23.344 | −9.392 | −15.276 |
| ATOM | 1713 | C | LEU | 212 | −24.317 | −9.526 | −16.425 |
| ATOM | 1714 | O | LEU | 212 | −23.98 | −10.127 | −17.433 |
| ATOM | 1715 | CB | LEU | 212 | −23.719 | −10.425 | −14.178 |
| ATOM | 1716 | CG | LEU | 212 | −22.646 | −10.604 | −13.059 |
| ATOM | 1717 | CD1 | LEU | 212 | −23.244 | −11.455 | −11.904 |
| ATOM | 1718 | CD2 | LEU | 212 | −21.347 | −11.291 | −13.574 |
| ATOM | 1719 | HA | LEU | 212 | −22.34 | −9.628 | −15.639 |
| ATOM | 1720 | H | LEU | 212 | −23.601 | −7.836 | −13.841 |
| ATOM | 1721 | HB1 | LEU | 212 | −24.668 | −10.093 | −13.731 |
| ATOM | 1722 | HB2 | LEU | 212 | −23.897 | −11.407 | −14.646 |
| ATOM | 1723 | HG | LEU | 212 | −22.371 | −9.619 | −12.649 |
| ATOM | 1724 | HD21 | LEU | 212 | −20.803 | −10.675 | −14.303 |
| ATOM | 1725 | HD22 | LEU | 212 | −20.66 | −11.481 | −12.735 |
| ATOM | 1726 | HD23 | LEU | 212 | −21.596 | −12.254 | −14.044 |
| ATOM | 1727 | HD11 | LEU | 212 | −23.536 | −12.449 | −12.274 |
| ATOM | 1728 | HD12 | LEU | 212 | −22.512 | −11.586 | −11.091 |
| ATOM | 1729 | HD13 | LEU | 212 | −24.133 | −10.961 | −11.484 |
| ATOM | 1730 | N | ALA | 213 | −25.545 | −8.981 | −16.299 |
| ATOM | 1731 | CA | ALA | 213 | −26.503 | −9.111 | −17.395 |
| ATOM | 1732 | C | ALA | 213 | −26.132 | −8.263 | −18.598 |
| ATOM | 1733 | O | ALA | 213 | −26.551 | −8.607 | −19.692 |
| ATOM | 1734 | CB | ALA | 213 | −27.924 | −8.715 | −16.914 |
| ATOM | 1735 | HA | ALA | 213 | −26.542 | −10.17 | −17.709 |
| ATOM | 1736 | H | ALA | 213 | −25.807 | −8.479 | −15.469 |
| ATOM | 1737 | HB1 | ALA | 213 | −28.656 | −8.931 | −17.706 |
| ATOM | 1738 | HB2 | ALA | 213 | −28.2 | −9.293 | −16.017 |
| ATOM | 1739 | HB3 | ALA | 213 | −27.962 | −7.642 | −16.672 |
| ATOM | 1740 | N | THR | 214 | −25.38 | −7.148 | −18.434 |
| ATOM | 1741 | CA | THR | 214 | −25.14 | −6.226 | −19.549 |
| ATOM | 1742 | C | THR | 214 | −23.813 | −6.401 | −20.258 |
| ATOM | 1743 | O | THR | 214 | −23.813 | −6.527 | −21.474 |
| ATOM | 1744 | CB | THR | 214 | −25.217 | −4.757 | −19.036 |
| ATOM | 1745 | OG1 | THR | 214 | −26.563 | −4.506 | −18.587 |
| ATOM | 1746 | CG2 | THR | 214 | −24.821 | −3.728 | −20.132 |
| ATOM | 1747 | HA | THR | 214 | −25.945 | −6.317 | −20.298 |
| ATOM | 1748 | H | THR | 214 | −25.017 | −6.908 | −17.531 |
| ATOM | 1749 | HB | THR | 214 | −24.52 | −4.632 | −18.188 |
| ATOM | 1750 | HG1 | THR | 214 | −26.656 | −3.649 | −18.184 |
| ATOM | 1751 | HG23 | THR | 214 | −23.755 | −3.812 | −20.387 |
| ATOM | 1752 | HG21 | THR | 214 | −25.407 | −3.894 | −21.047 |
| ATOM | 1753 | HG22 | THR | 214 | −24.999 | −2.703 | −19.778 |
| ATOM | 1754 | N | VAL | 215 | −22.662 | −6.346 | −19.55 |
| ATOM | 1755 | CA | VAL | 215 | −21.389 | −6.1 | −20.24 |
| ATOM | 1756 | C | VAL | 215 | −20.769 | −7.401 | −20.698 |
| ATOM | 1757 | O | VAL | 215 | −19.726 | −7.775 | −20.192 |
| ATOM | 1758 | CB | VAL | 215 | −20.439 | −5.23 | −19.36 |
| ATOM | 1759 | CG1 | VAL | 215 | −19.123 | −4.843 | −20.099 |
| ATOM | 1760 | CG2 | VAL | 215 | −21.133 | −3.916 | −18.902 |
| ATOM | 1761 | HA | VAL | 215 | −21.559 | −5.502 | −21.148 |
| ATOM | 1762 | H | VAL | 215 | −22.668 | −6.427 | −18.55 |
| ATOM | 1763 | HB | VAL | 215 | −20.197 | −5.829 | −18.47 |
| ATOM | 1764 | HG11 | VAL | 215 | −19.353 | −4.301 | −21.03 |
| ATOM | 1765 | HG12 | VAL | 215 | −18.506 | −4.19 | −19.465 |
| ATOM | 1766 | HG13 | VAL | 215 | −18.512 | −5.72 | −20.348 |
| ATOM | 1767 | HG21 | VAL | 215 | −22.048 | −4.114 | −18.327 |
| ATOM | 1768 | HG22 | VAL | 215 | −20.46 | −3.33 | −18.258 |
| ATOM | 1769 | HG23 | VAL | 215 | −21.388 | −3.3 | −19.778 |
| ATOM | 1770 | N | HIS | 216 | −21.407 | −8.094 | −21.672 |
| ATOM | 1771 | CA | HIS | 216 | −20.821 | −9.293 | −22.285 |
| ATOM | 1772 | C | HIS | 216 | −19.813 | −10.037 | −21.432 |
| ATOM | 1773 | O | HIS | 216 | −18.687 | −10.186 | −21.882 |
| ATOM | 1774 | CB | HIS | 216 | −20.193 | −8.796 | −23.616 |
| ATOM | 1775 | CG | HIS | 216 | −19.238 | −7.658 | −23.354 |
| ATOM | 1776 | ND1 | HIS | 216 | −18.065 | −7.81 | −22.783 |
| ATOM | 1777 | CD2 | HIS | 216 | −19.44 | −6.358 | −23.65 |
| ATOM | 1778 | CE1 | HIS | 216 | −17.471 | −6.663 | −22.691 |
| ATOM | 1779 | NE2 | HIS | 216 | −18.214 | −5.775 | −23.175 |
| ATOM | 1780 | HA | HIS | 216 | −21.612 | −10.025 | −22.519 |
| ATOM | 1781 | H | HIS | 216 | −22.28 | −7.76 | −22.035 |
| ATOM | 1782 | HB1 | HIS | 216 | −19.674 | −9.61 | −24.147 |
| ATOM | 1783 | HB2 | HIS | 216 | −20.998 | −8.433 | −24.269 |
| ATOM | 1784 | HD2 | HIS | 216 | −20.292 | −5.87 | −24.117 |
| ATOM | 1785 | HE1 | HIS | 216 | −16.486 | −6.48 | −22.261 |
| ATOM | 1786 | HD1 | HIS | 216 | −17.68 | −8.71 | −22.453 |
| ATOM | 1787 | N | PRO | 217 | −20.133 | −10.505 | −20.2 |
| ATOM | 1788 | CA | PRO | 217 | −19.102 | −11.056 | −19.333 |
| ATOM | 1789 | C | PRO | 217 | −19.245 | −12.55 | −19.148 |
| ATOM | 1790 | O | PRO | 217 | −20.095 | −13.163 | −19.774 |
| ATOM | 1791 | CB | PRO | 217 | −19.546 | −10.291 | −18.066 |
| ATOM | 1792 | CG | PRO | 217 | −21.095 | −10.282 | −18.14 |
| ATOM | 1793 | CD | PRO | 217 | −21.473 | −10.421 | −19.637 |
| ATOM | 1794 | HA | PRO | 217 | −18.067 | −10.8 | −19.614 |
| ATOM | 1795 | HD2 | PRO | 217 | −22.098 | −9.577 | −19.956 |
| ATOM | 1796 | HD1 | PRO | 217 | −22.019 | −11.356 | −19.841 |
| ATOM | 1797 | HG2 | PRO | 217 | −21.473 | −9.329 | −17.737 |
| ATOM | 1798 | HG1 | PRO | 217 | −21.513 | −11.123 | −17.562 |
| ATOM | 1799 | HB1 | PRO | 217 | −19.166 | −10.718 | −17.125 |
| ATOM | 1800 | HB2 | PRO | 217 | −19.175 | −9.255 | −18.144 |
| ATOM | 1801 | N | ILE | 218 | −18.405 | −13.15 | −18.275 |
| ATOM | 1802 | CA | ILE | 218 | −18.507 | −14.582 | −17.995 |
| ATOM | 1803 | C | ILE | 218 | −18.226 | −15.406 | −19.239 |
| ATOM | 1804 | O | ILE | 218 | −17.109 | −15.886 | −19.343 |
| ATOM | 1805 | CB | ILE | 218 | −19.824 | −14.948 | −17.242 |
| ATOM | 1806 | CG1 | ILE | 218 | −20.069 | −14.042 | −15.994 |
| ATOM | 1807 | CG2 | ILE | 218 | −19.859 | −16.462 | −16.886 |
| ATOM | 1808 | CD1 | ILE | 218 | −18.959 | −14.082 | −14.908 |
| ATOM | 1809 | HA | ILE | 218 | −17.696 | −14.856 | −17.298 |
| ATOM | 1810 | H | ILE | 218 | −17.7 | −12.619 | −17.798 |
| ATOM | 1811 | HB | ILE | 218 | −20.685 | −14.764 | −17.905 |
| ATOM | 1812 | HG11 | ILE | 218 | −20.184 | −12.997 | −16.324 |
| ATOM | 1813 | HG12 | ILE | 218 | −21.02 | −14.345 | −15.525 |
| ATOM | 1814 | HD11 | ILE | 218 | −18.011 | −13.673 | −15.285 |
| ATOM | 1815 | HD12 | ILE | 218 | −19.263 | −13.467 | −14.046 |
| ATOM | 1816 | HD13 | ILE | 218 | −18.79 | −15.106 | −14.546 |
| ATOM | 1817 | HG21 | ILE | 218 | −18.994 | −16.754 | −16.274 |
| ATOM | 1818 | HG22 | ILE | 218 | −20.779 | −16.704 | −16.331 |
| ATOM | 1819 | HG23 | ILE | 218 | −19.845 | −17.069 | −17.802 |
| ATOM | 1820 | N | SER | 219 | −19.184 | −15.588 | −20.179 |
| ATOM | 1821 | CA | SER | 219 | −18.971 | −16.444 | −21.353 |
| ATOM | 1822 | C | SER | 219 | −20.308 | −16.929 | −21.875 |
| ATOM | 1823 | O | SER | 219 | −20.628 | −16.703 | −23.031 |
| ATOM | 1824 | CB | SER | 219 | −18.181 | −17.762 | −21.1 |
| ATOM | 1825 | OG | SER | 219 | −18.749 | −18.424 | −19.953 |
| ATOM | 1826 | HA | SER | 219 | −18.463 | −15.857 | −22.136 |
| ATOM | 1827 | H | SER | 219 | −20.049 | −15.084 | −20.119 |
| ATOM | 1828 | HB1 | SER | 219 | −18.263 | −18.418 | −21.985 |
| ATOM | 1829 | HB2 | SER | 219 | −17.107 | −17.568 | −20.953 |
| ATOM | 1830 | HG | SER | 219 | −18.312 | −19.244 | −19.744 |
| ATOM | 1831 | N | SER | 220 | −21.084 | −17.639 | −21.026 |
| ATOM | 1832 | CA | SER | 220 | −22.261 | −18.353 | −21.523 |
| ATOM | 1833 | C | SER | 220 | −23.242 | −18.698 | −20.421 |
| ATOM | 1834 | O | SER | 220 | −24.404 | −18.337 | −20.542 |
| ATOM | 1835 | CB | SER | 220 | −21.767 | −19.676 | −22.16 |
| ATOM | 1836 | OG | SER | 220 | −21.087 | −20.447 | −21.152 |
| ATOM | 1837 | HA | SER | 220 | −22.785 | −17.749 | −22.283 |
| ATOM | 1838 | H | SER | 220 | −20.784 | −17.781 | −20.082 |
| ATOM | 1839 | HB1 | SER | 220 | −22.628 | −20.24 | −22.559 |
| ATOM | 1840 | HB2 | SER | 220 | −21.078 | −19.447 | −22.991 |
| ATOM | 1841 | HG | SER | 220 | −20.746 | −21.273 | −21.484 |
| ATOM | 1842 | N | THR | 221 | −22.795 | −19.411 | −19.359 |
| ATOM | 1843 | CA | THR | 221 | −23.698 | −19.776 | −18.265 |
| ATOM | 1844 | C | THR | 221 | −24.408 | −18.586 | −17.658 |
| ATOM | 1845 | O | THR | 221 | −24.04 | −17.456 | −17.936 |
| ATOM | 1846 | CB | THR | 221 | −22.999 | −20.571 | −17.126 |
| ATOM | 1847 | OG1 | THR | 221 | −24.02 | −21.053 | −16.232 |
| ATOM | 1848 | CG2 | THR | 221 | −21.978 | −19.695 | −16.35 |
| ATOM | 1849 | HA | THR | 221 | −24.471 | −20.433 | −18.697 |
| ATOM | 1850 | H | THR | 221 | −21.853 | −19.753 | −19.343 |
| ATOM | 1851 | HB | THR | 221 | −22.476 | −21.435 | −17.576 |
| ATOM | 1852 | HG1 | THR | 221 | −23.673 | −21.573 | −15.514 |
| ATOM | 1853 | HG23 | THR | 221 | −21.218 | −19.279 | −17.028 |
| ATOM | 1854 | HG21 | THR | 221 | −22.489 | −18.865 | −15.84 |
| ATOM | 1855 | HG22 | THR | 221 | −21.464 | −20.302 | −15.589 |
| ATOM | 1856 | N | LYS | 222 | −25.45 | −18.836 | −16.829 |
| ATOM | 1857 | CA | LYS | 222 | −26.31 | −17.757 | −16.339 |
| ATOM | 1858 | C | LYS | 222 | −26.718 | −18.03 | −14.908 |
| ATOM | 1859 | O | LYS | 222 | −26.381 | −19.083 | −14.392 |
| ATOM | 1860 | CB | LYS | 222 | −27.547 | −17.66 | −17.278 |
| ATOM | 1861 | CG | LYS | 222 | −27.099 | −17.328 | −18.726 |
| ATOM | 1862 | CD | LYS | 222 | −28.265 | −17.204 | −19.738 |
| ATOM | 1863 | CE | LYS | 222 | −27.759 | −16.688 | −21.118 |
| ATOM | 1864 | NZ | LYS | 222 | −26.61 | −17.458 | −21.66 |
| ATOM | 1865 | HA | LYS | 222 | −25.773 | −16.794 | −16.324 |
| ATOM | 1866 | H | LYS | 222 | −25.694 | −19.776 | −16.575 |
| ATOM | 1867 | HB1 | LYS | 222 | −28.087 | −18.62 | −17.265 |
| ATOM | 1868 | HB2 | LYS | 222 | −28.236 | −16.877 | −16.93 |
| ATOM | 1869 | HG1 | LYS | 222 | −26.516 | −16.392 | −18.72 |
| ATOM | 1870 | HG2 | LYS | 222 | −26.463 | −18.151 | −19.075 |
| ATOM | 1871 | HD1 | LYS | 222 | −28.745 | −18.19 | −19.859 |
| ATOM | 1872 | HD2 | LYS | 222 | −29.024 | −16.507 | −19.348 |
| ATOM | 1873 | HE1 | LYS | 222 | −28.605 | −16.726 | −21.826 |
| ATOM | 1874 | HE2 | LYS | 222 | −27.46 | −15.63 | −21.017 |
| ATOM | 1875 | HZ1 | LYS | 222 | −26.782 | −18.526 | −21.643 |
| ATOM | 1876 | HZ2 | LYS | 222 | −26.397 | −17.192 | −22.686 |
| ATOM | 1877 | HZ3 | LYS | 222 | −25.679 | −17.28 | −21.136 |
| ATOM | 1878 | N | PHE | 223 | −27.429 | −17.096 | −14.237 |
| ATOM | 1879 | CA | PHE | 223 | −27.738 | −17.306 | −12.825 |
| ATOM | 1880 | C | PHE | 223 | −28.447 | −18.622 | −12.586 |
| ATOM | 1881 | O | PHE | 223 | −29.419 | −18.892 | −13.274 |
| ATOM | 1882 | CB | PHE | 223 | −28.687 | −16.226 | −12.238 |
| ATOM | 1883 | CG | PHE | 223 | −28.093 | −14.818 | −12.348 |
| ATOM | 1884 | CD1 | PHE | 223 | −27.059 | −14.434 | −11.488 |
| ATOM | 1885 | CD2 | PHE | 223 | −28.577 | −13.905 | −13.292 |
| ATOM | 1886 | CE1 | PHE | 223 | −26.528 | −13.143 | −11.556 |
| ATOM | 1887 | CE2 | PHE | 223 | −28.031 | −12.622 | −13.382 |
| ATOM | 1888 | CZ | PHE | 223 | −27.001 | −12.245 | −12.516 |
| ATOM | 1889 | HA | PHE | 223 | −26.784 | −17.264 | −12.274 |
| ATOM | 1890 | H | PHE | 223 | −27.69 | −16.231 | −14.665 |
| ATOM | 1891 | HB1 | PHE | 223 | −29.656 | −16.267 | −12.761 |
| ATOM | 1892 | HB2 | PHE | 223 | −28.87 | −16.429 | −11.17 |
| ATOM | 1893 | HD2 | PHE | 223 | −29.38 | −14.189 | −13.96 |
| ATOM | 1894 | HE2 | PHE | 223 | −28.405 | −11.919 | −14.12 |
| ATOM | 1895 | HZ | PHE | 223 | −26.575 | −11.253 | −12.592 |
| ATOM | 1896 | HE1 | PHE | 223 | −25.752 | −12.842 | −10.86 |
| ATOM | 1897 | HD1 | PHE | 223 | −26.668 | −15.138 | −10.764 |
| ATOM | 1898 | N | ARG | 224 | −28.024 | −19.429 | −11.586 |
| ATOM | 1899 | CA | ARG | 224 | −28.896 | −20.503 | −11.113 |
| ATOM | 1900 | C | ARG | 224 | −30.006 | −19.726 | −10.434 |
| ATOM | 1901 | O | ARG | 224 | −29.897 | −19.41 | −9.259 |
| ATOM | 1902 | CB | ARG | 224 | −28.147 | −21.487 | −10.176 |
| ATOM | 1903 | CG | ARG | 224 | −27.112 | −22.329 | −10.976 |
| ATOM | 1904 | CD | ARG | 224 | −26.359 | −23.349 | −10.078 |
| ATOM | 1905 | NE | ARG | 224 | −25.404 | −24.149 | −10.855 |
| ATOM | 1906 | CZ | ARG | 224 | −24.666 | −25.11 | −10.343 |
| ATOM | 1907 | NH1 | ARG | 224 | −24.71 | −25.438 | −9.074 |
| ATOM | 1908 | NH2 | ARG | 224 | −23.849 | −25.772 | −11.13 |
| ATOM | 1909 | HA | ARG | 224 | −29.288 | −21.091 | −11.96 |
| ATOM | 1910 | H | ARG | 224 | −27.201 | −19.214 | −11.058 |
| ATOM | 1911 | HB1 | ARG | 224 | −27.63 | −20.915 | −9.393 |
| ATOM | 1912 | HB2 | ARG | 224 | −28.882 | −22.155 | −9.699 |
| ATOM | 1913 | HG1 | ARG | 224 | −27.632 | −22.887 | −11.773 |
| ATOM | 1914 | HG2 | ARG | 224 | −26.38 | −21.655 | −11.451 |
| ATOM | 1915 | HD1 | ARG | 224 | −25.828 | −22.787 | −9.291 |
| ATOM | 1916 | HD2 | ARG | 224 | −27.114 | −24.009 | −9.617 |
| ATOM | 1917 | HE | ARG | 224 | −25.321 | −23.937 | −11.863 |
| ATOM | 1918 | HH12 | ARG | 224 | −24.121 | −26.197 | −8.701 |
| ATOM | 1919 | HH11 | ARG | 224 | −25.327 | −24.953 | −8.411 |
| ATOM | 1920 | HH22 | ARG | 224 | −23.255 | −26.531 | −10.766 |
| ATOM | 1921 | HH21 | ARG | 224 | −23.778 | −25.552 | −12.135 |
| ATOM | 1922 | N | LYS | 225 | −31.051 | −19.359 | −11.213 |
| ATOM | 1923 | CA | LYS | 225 | −31.883 | −18.209 | −10.866 |
| ATOM | 1924 | C | LYS | 225 | −32.312 | −18.032 | −9.415 |
| ATOM | 1925 | O | LYS | 225 | −32.187 | −16.901 | −8.968 |
| ATOM | 1926 | CB | LYS | 225 | −32.967 | −17.93 | −11.957 |
| ATOM | 1927 | CG | LYS | 225 | −34.11 | −18.977 | −12.035 |
| ATOM | 1928 | CD | LYS | 225 | −34.922 | −18.875 | −13.359 |
| ATOM | 1929 | CE | LYS | 225 | −36.27 | −19.653 | −13.301 |
| ATOM | 1930 | NZ | LYS | 225 | −36.106 | −21.103 | −13.033 |
| ATOM | 1931 | HA | LYS | 225 | −31.179 | −17.372 | −11.013 |
| ATOM | 1932 | HB1 | LYS | 225 | −33.404 | −16.938 | −11.785 |
| ATOM | 1933 | HB2 | LYS | 225 | −32.47 | −17.893 | −12.942 |
| ATOM | 1934 | HG1 | LYS | 225 | −33.673 | −19.986 | −11.988 |
| ATOM | 1935 | HG2 | LYS | 225 | −34.782 | −18.819 | −11.176 |
| ATOM | 1936 | HD1 | LYS | 225 | −35.138 | −17.819 | −13.587 |
| ATOM | 1937 | HD2 | LYS | 225 | −34.31 | −19.264 | −14.191 |
| ATOM | 1938 | HE1 | LYS | 225 | −36.915 | −19.211 | −12.522 |
| ATOM | 1939 | HE2 | LYS | 225 | −36.785 | −19.516 | −14.268 |
| ATOM | 1940 | HZ1 | LYS | 225 | −35.738 | −21.308 | −12.037 |
| ATOM | 1941 | HZ2 | LYS | 225 | −37.049 | −21.629 | −13.106 |
| ATOM | 1942 | HZ3 | LYS | 225 | −35.432 | −21.571 | −13.737 |
| ATOM | 1943 | H | LYS | 225 | −31.125 | −19.711 | −12.148 |
| ATOM | 1944 | N | PRO | 226 | −32.802 | −19 | −8.594 |
| ATOM | 1945 | CA | PRO | 226 | −33.15 | −18.655 | −7.217 |
| ATOM | 1946 | C | PRO | 226 | −31.987 | −18.735 | −6.248 |
| ATOM | 1947 | O | PRO | 226 | −32.089 | −18.139 | −5.186 |
| ATOM | 1948 | CB | PRO | 226 | −34.121 | −19.824 | −6.922 |
| ATOM | 1949 | CG | PRO | 226 | −33.493 | −21.016 | −7.681 |
| ATOM | 1950 | CD | PRO | 226 | −33.044 | −20.375 | −9.017 |
| ATOM | 1951 | HA | PRO | 226 | −33.668 | −17.685 | −7.123 |
| ATOM | 1952 | HD2 | PRO | 226 | −33.896 | −20.425 | −9.704 |
| ATOM | 1953 | HD1 | PRO | 226 | −32.175 | −20.892 | −9.449 |
| ATOM | 1954 | HG2 | PRO | 226 | −34.2 | −21.851 | −7.817 |
| ATOM | 1955 | HG1 | PRO | 226 | −32.624 | −21.392 | −7.118 |
| ATOM | 1956 | HB1 | PRO | 226 | −34.255 | −20.018 | −5.845 |
| ATOM | 1957 | HB2 | PRO | 226 | −35.106 | −19.599 | −7.366 |
| ATOM | 1958 | N | SER | 227 | −30.889 | −19.473 | −6.537 |
| ATOM | 1959 | CA | SER | 227 | −29.894 | −19.714 | −5.493 |
| ATOM | 1960 | C | SER | 227 | −28.952 | −18.544 | −5.394 |
| ATOM | 1961 | O | SER | 227 | −28.763 | −18.048 | −4.295 |
| ATOM | 1962 | CB | SER | 227 | −29.072 | −21.005 | −5.727 |
| ATOM | 1963 | CG | SER | 227 | −28.275 | −20.873 | −6.915 |
| ATOM | 1964 | HA | SER | 227 | −30.408 | −19.846 | −4.524 |
| ATOM | 1965 | H | SER | 227 | −30.729 | −19.867 | −7.445 |
| ATOM | 1966 | HB1 | SER | 227 | −28.415 | −21.174 | −4.856 |
| ATOM | 1967 | HB2 | SER | 227 | −29.763 | −21.86 | −5.823 |
| ATOM | 1968 | HG | SER | 227 | −27.727 | −21.638 | −7.062 |
| ATOM | 1969 | N | VAL | 228 | −28.352 | −18.076 | −6.512 |
| ATOM | 1970 | CA | VAL | 228 | −27.463 | −16.919 | −6.408 |
| ATOM | 1971 | C | VAL | 228 | −28.234 | −15.823 | −5.71 |
| ATOM | 1972 | O | VAL | 228 | −27.73 | −15.273 | −4.743 |
| ATOM | 1973 | CB | VAL | 228 | −26.907 | −16.411 | −7.773 |
| ATOM | 1974 | CG1 | VAL | 228 | −26.159 | −15.057 | −7.587 |
| ATOM | 1975 | CG2 | VAL | 228 | −25.947 | −17.458 | −8.406 |
| ATOM | 1976 | HA | VAL | 228 | −26.608 | −17.199 | −5.771 |
| ATOM | 1977 | H | VAL | 228 | −28.531 | −18.501 | −7.402 |
| ATOM | 1978 | HB | VAL | 228 | −27.751 | −16.247 | −8.464 |
| ATOM | 1979 | HG11 | VAL | 228 | −25.374 | −15.158 | −6.821 |
| ATOM | 1980 | HG12 | VAL | 228 | −25.682 | −14.737 | −8.525 |
| ATOM | 1981 | HG13 | VAL | 228 | −26.851 | −14.259 | −7.276 |
| ATOM | 1982 | HG21 | VAL | 228 | −26.45 | −18.424 | −8.56 |
| ATOM | 1983 | HG22 | VAL | 228 | −25.585 | −17.107 | −9.385 |
| ATOM | 1984 | HG23 | VAL | 228 | −25.076 | −17.622 | −7.753 |
| ATOM | 1985 | N | ALA | 229 | −29.465 | −15.52 | −6.18 |
| ATOM | 1986 | CA | ALA | 229 | −30.284 | −14.507 | −5.515 |
| ATOM | 1987 | C | ALA | 229 | −30.296 | −14.706 | −4.013 |
| ATOM | 1988 | O | ALA | 229 | −30.153 | −13.75 | −3.267 |
| ATOM | 1989 | CB | ALA | 229 | −31.749 | −14.581 | −6.018 |
| ATOM | 1990 | HA | ALA | 229 | −29.889 | −13.509 | −5.76 |
| ATOM | 1991 | H | ALA | 229 | −29.834 | −15.989 | −6.985 |
| ATOM | 1992 | HB1 | ALA | 229 | −32.324 | −13.766 | −5.56 |
| ATOM | 1993 | HB2 | ALA | 229 | −31.8 | −14.477 | −7.112 |
| ATOM | 1994 | HB3 | ALA | 229 | −32.21 | −15.541 | −5.742 |
| ATOM | 1995 | N | THR | 230 | −30.471 | −15.962 | −3.548 |
| ATOM | 1996 | CA | THR | 230 | −30.499 | −16.209 | −2.107 |
| ATOM | 1997 | C | THR | 230 | −29.125 | −15.967 | −1.52 |
| ATOM | 1998 | O | THR | 230 | −29.023 | −15.334 | −0.479 |
| ATOM | 1999 | CB | THR | 230 | −30.966 | −17.656 | −1.773 |
| ATOM | 2000 | OG1 | THR | 230 | −32.306 | −17.894 | −2.249 |
| ATOM | 2001 | CG2 | THR | 230 | −30.976 | −17.904 | −0.242 |
| ATOM | 2002 | HA | THR | 230 | −31.222 | −15.516 | −1.647 |
| ATOM | 2003 | H | THR | 230 | −30.549 | −16.736 | −4.179 |
| ATOM | 2004 | HB | THR | 230 | −30.278 | −18.388 | −2.23 |
| ATOM | 2005 | HG1 | THR | 230 | −32.397 | −17.847 | −3.197 |
| ATOM | 2006 | HG23 | THR | 230 | −29.963 | −17.833 | 0.182 |
| ATOM | 2007 | HG21 | THR | 230 | −31.625 | −17.17 | 0.259 |
| ATOM | 2008 | HG22 | THR | 230 | −31.366 | −18.913 | −0.034 |
| ATOM | 2009 | N | LEU | 231 | −28.046 | −16.467 | −2.163 |
| ATOM | 2010 | CA | LEU | 231 | −26.712 | −16.287 | −1.595 |
| ATOM | 2011 | C | LEU | 231 | −26.438 | −14.81 | −1.42 |
| ATOM | 2012 | O | LEU | 231 | −25.967 | −14.411 | −0.366 |
| ATOM | 2013 | CB | LEU | 231 | −25.569 | −16.872 | −2.476 |
| ATOM | 2014 | CG | LEU | 231 | −25.617 | −18.413 | −2.716 |
| ATOM | 2015 | CD1 | LEU | 231 | −24.434 | −18.834 | −3.634 |
| ATOM | 2016 | CD2 | LEU | 231 | −25.569 | −19.222 | −1.389 |
| ATOM | 2017 | HA | LEU | 231 | −26.687 | −16.745 | −0.594 |
| ATOM | 2018 | H | LEU | 231 | −28.149 | −16.947 | −3.036 |
| ATOM | 2019 | HB1 | LEU | 231 | −25.593 | −16.363 | −3.453 |
| ATOM | 2020 | HB2 | LEU | 231 | −24.607 | −16.628 | −1.997 |
| ATOM | 2021 | HG | LEU | 231 | −26.544 | −18.686 | −3.244 |
| ATOM | 2022 | HD21 | LEU | 231 | −26.486 | −19.074 | −0.799 |
| ATOM | 2023 | HD22 | LEU | 231 | −25.485 | −20.299 | −1.6 |
| ATOM | 2024 | HD23 | LEU | 231 | −24.702 | −18.917 | −0.784 |
| ATOM | 2025 | HD11 | LEU | 231 | −23.472 | −18.62 | −3.143 |
| ATOM | 2026 | HD12 | LEU | 231 | −24.479 | −19.911 | −3.857 |
| ATOM | 2027 | HD13 | LEU | 231 | −24.468 | −18.287 | −4.589 |
| ATOM | 2028 | N | VAL | 232 | −26.721 | −13.975 | −2.445 |
| ATOM | 2029 | CA | VAL | 232 | −26.462 | −12.547 | −2.283 |
| ATOM | 2030 | C | VAL | 232 | −27.332 | −12.007 | −1.165 |
| ATOM | 2031 | O | VAL | 232 | −26.804 | −11.27 | −0.35 |
| ATOM | 2032 | CB | VAL | 232 | −26.468 | −11.687 | −3.587 |
| ATOM | 2033 | CG1 | VAL | 232 | −25.206 | −11.976 | −4.451 |
| ATOM | 2034 | CG2 | VAL | 232 | −27.729 | −11.873 | −4.471 |
| ATOM | 2035 | HA | VAL | 232 | −25.43 | −12.466 | −1.904 |
| ATOM | 2036 | H | VAL | 232 | −27.126 | −14.323 | −3.292 |
| ATOM | 2037 | HB | VAL | 232 | −26.421 | −10.628 | −3.279 |
| ATOM | 2038 | HG11 | VAL | 232 | −25.216 | −11.364 | −5.368 |
| ATOM | 2039 | HG12 | VAL | 232 | −25.179 | −13.039 | −4.739 |
| ATOM | 2040 | HG13 | VAL | 232 | −24.283 | −11.74 | −3.898 |
| ATOM | 2041 | HG21 | VAL | 232 | −28.647 | −11.772 | −3.875 |
| ATOM | 2042 | HG22 | VAL | 232 | −27.741 | −11.125 | −5.279 |
| ATOM | 2043 | HG23 | VAL | 232 | −27.726 | −12.85 | −4.963 |
| ATOM | 2044 | N | ILE | 233 | −28.635 | −12.355 | −1.037 |
| ATOM | 2045 | CA | ILE | 233 | −29.384 | −11.892 | 0.142 |
| ATOM | 2046 | C | ILE | 233 | −28.6 | −12.232 | 1.394 |
| ATOM | 2047 | O | ILE | 233 | −28.435 | −11.372 | 2.249 |
| ATOM | 2048 | CB | ILE | 233 | −30.852 | −12.434 | 0.22 |
| ATOM | 2049 | CG1 | ILE | 233 | −31.769 | −11.616 | −0.746 |
| ATOM | 2050 | CG2 | ILE | 233 | −31.424 | −12.395 | 1.67 |
| ATOM | 2051 | CD1 | ILE | 233 | −33.274 | −11.994 | −0.68 |
| ATOM | 2052 | HA | ILE | 233 | −29.431 | −10.793 | 0.117 |
| ATOM | 2053 | H | ILE | 233 | −29.079 | −12.96 | −1.703 |
| ATOM | 2054 | HB | ILE | 233 | −30.846 | −13.493 | −0.083 |
| ATOM | 2055 | HG11 | ILE | 233 | −31.697 | −10.542 | −0.505 |
| ATOM | 2056 | HG12 | ILE | 233 | −31.412 | −11.75 | −1.781 |
| ATOM | 2057 | HD11 | ILE | 233 | −33.713 | −11.828 | 0.313 |
| ATOM | 2058 | HD12 | ILE | 233 | −33.843 | −11.352 | −1.367 |
| ATOM | 2059 | HD13 | ILE | 233 | −33.415 | −13.049 | −0.96 |
| ATOM | 2060 | HG21 | ILE | 233 | −31.615 | −11.351 | 1.952 |
| ATOM | 2061 | HG22 | ILE | 233 | −32.363 | −12.962 | 1.744 |
| ATOM | 2062 | HG23 | ILE | 233 | −30.753 | −12.853 | 2.41 |
| ATOM | 2063 | N | CYS | 234 | −28.117 | −13.485 | 1.536 |
| ATOM | 2064 | CA | CYS | 234 | −27.415 | −13.848 | 2.767 |
| ATOM | 2065 | C | CYS | 234 | −26.203 | −12.962 | 2.967 |
| ATOM | 2066 | O | CYS | 234 | −26.037 | −12.403 | 4.041 |
| ATOM | 2067 | CB | CYS | 234 | −26.98 | −15.338 | 2.769 |
| ATOM | 2068 | SG | CYS | 234 | −28.457 | −16.395 | 2.59 |
| ATOM | 2069 | HA | CYS | 234 | −28.105 | −13.698 | 3.615 |
| ATOM | 2070 | H | CYS | 234 | −28.247 | −14.17 | 0.813 |
| ATOM | 2071 | HB1 | CYS | 234 | −26.288 | −15.543 | 1.938 |
| ATOM | 2072 | HB2 | CYS | 234 | −26.474 | −15.582 | 3.717 |
| ATOM | 2073 | HG | CYS | 234 | −27.996 | −17.292 | 2.564 |
| ATOM | 2074 | LPG1 | CYS | 234 | −28.878 | −16.339 | 3.12 |
| ATOM | 2075 | LPG2 | CYS | 234 | −28.797 | −16.266 | 2.017 |
| ATOM | 2076 | N | LEU | 235 | −25.34 | −12.816 | 1.937 |
| ATOM | 2077 | CA | LEU | 235 | −24.174 | −11.94 | 2.08 |
| ATOM | 2078 | C | LEU | 235 | −24.612 | −10.549 | 2.478 |
| ATOM | 2079 | O | LEU | 235 | −24.003 | −9.933 | 3.338 |
| ATOM | 2080 | CB | LEU | 235 | −23.401 | −11.823 | 0.734 |
| ATOM | 2081 | CG | LEU | 235 | −22.263 | −10.756 | 0.711 |
| ATOM | 2082 | CD1 | LEU | 235 | −21.158 | −11.055 | 1.761 |
| ATOM | 2083 | CD2 | LEU | 235 | −21.637 | −10.683 | −0.71 |
| ATOM | 2084 | HA | LEU | 235 | −23.52 | −12.362 | 2.859 |
| ATOM | 2085 | H | LEU | 235 | −25.502 | −13.288 | 1.064 |
| ATOM | 2086 | HB1 | LEU | 235 | −22.992 | −12.812 | 0.472 |
| ATOM | 2087 | HB2 | LEU | 235 | −24.127 | −11.541 | −0.044 |
| ATOM | 2088 | HG | LEU | 235 | −22.689 | −9.759 | 0.922 |
| ATOM | 2089 | HD21 | LEU | 235 | −22.404 | −10.435 | −1.461 |
| ATOM | 2090 | HD22 | LEU | 235 | −20.858 | −9.905 | −0.75 |
| ATOM | 2091 | HD23 | LEU | 235 | −21.182 | −11.648 | −0.98 |
| ATOM | 2092 | HD11 | LEU | 235 | −20.742 | −12.061 | 1.601 |
| ATOM | 2093 | HD12 | LEU | 235 | −20.341 | −10.322 | 1.674 |
| ATOM | 2094 | HD13 | LEU | 235 | −21.55 | −10.994 | 2.786 |
| ATOM | 2095 | N | LEU | 236 | −25.67 | −10.021 | 1.834 |
| ATOM | 2096 | CA | LEU | 236 | −26.039 | −8.626 | 2.062 |
| ATOM | 2097 | C | LEU | 236 | −26.486 | −8.43 | 3.492 |
| ATOM | 2098 | O | LEU | 236 | −26.058 | −7.476 | 4.125 |
| ATOM | 2099 | CB | LEU | 236 | −27.125 | −8.211 | 1.034 |
| ATOM | 2100 | CG | LEU | 236 | −26.557 | −8.211 | −0.424 |
| ATOM | 2101 | CD1 | LEU | 236 | −27.703 | −8.349 | −1.455 |
| ATOM | 2102 | CD2 | LEU | 236 | −25.727 | −6.932 | −0.703 |
| ATOM | 2103 | HA | LEU | 236 | −25.147 | −7.997 | 1.923 |
| ATOM | 2104 | H | LEU | 236 | −26.2 | −10.58 | 1.191 |
| ATOM | 2105 | HB1 | LEU | 236 | −27.961 | −8.921 | 1.122 |
| ATOM | 2106 | HB2 | LEU | 236 | −27.522 | −7.216 | 1.29 |
| ATOM | 2107 | HG | LEU | 236 | −25.873 | −9.056 | −0.599 |
| ATOM | 2108 | HD21 | LEU | 236 | −24.788 | −6.967 | −0.138 |
| ATOM | 2109 | HD22 | LEU | 236 | −25.465 | −6.831 | −1.764 |
| ATOM | 2110 | HD23 | LEU | 236 | −26.303 | −6.048 | −0.404 |
| ATOM | 2111 | HD11 | LEU | 236 | −28.413 | −7.533 | −1.316 |
| ATOM | 2112 | HD12 | LEU | 236 | −27.307 | −8.302 | −2.475 |
| ATOM | 2113 | HD13 | LEU | 236 | −28.241 | −9.3 | −1.346 |
| ATOM | 2114 | N | TRP | 237 | −27.33 | −9.332 | 4.039 |
| ATOM | 2115 | CA | TRP | 237 | −27.636 | −9.237 | 5.466 |
| ATOM | 2116 | C | TRP | 237 | −26.358 | −9.349 | 6.266 |
| ATOM | 2117 | O | TRP | 237 | −26.216 | −8.611 | 7.227 |
| ATOM | 2118 | CB | TRP | 237 | −28.639 | −10.317 | 5.958 |
| ATOM | 2119 | CG | TRP | 237 | −30.042 | −9.785 | 5.849 |
| ATOM | 2120 | CD1 | TRP | 237 | −30.958 | −10.032 | 4.901 |
| ATOM | 2121 | CD2 | TRP | 237 | −30.675 | −8.837 | 6.835 |
| ATOM | 2122 | NE1 | TRP | 237 | −32.057 | −9.372 | 5.173 |
| ATOM | 2123 | CE2 | TRP | 237 | −31.945 | −8.669 | 6.317 |
| ATOM | 2124 | CE3 | TRP | 237 | −30.251 | −8.206 | 8.003 |
| ATOM | 2125 | CZ2 | TRP | 237 | −32.894 | −7.879 | 6.965 |
| ATOM | 2126 | CZ3 | TRP | 237 | −31.193 | −7.407 | 8.661 |
| ATOM | 2127 | CH2 | TRP | 237 | −32.488 | −7.236 | 8.143 |
| ATOM | 2128 | HA | TRP | 237 | −28.055 | −8.236 | 5.668 |
| ATOM | 2129 | H | TRP | 237 | −27.694 | −10.095 | 3.496 |
| ATOM | 2130 | HB1 | TRP | 237 | −28.515 | −11.258 | 5.401 |
| ATOM | 2131 | HB2 | TRP | 237 | −28.48 | −10.54 | 7.026 |
| ATOM | 2132 | HE1 | TRP | 237 | −32.905 | −9.383 | 4.581 |
| ATOM | 2133 | HD1 | TRP | 237 | −30.779 | −10.681 | 4.049 |
| ATOM | 2134 | HZ2 | TRP | 237 | −33.9 | −7.772 | 6.574 |
| ATOM | 2135 | HH2 | TRP | 237 | −33.182 | −6.589 | 8.667 |
| ATOM | 2136 | HZ3 | TRP | 237 | −30.918 | −6.912 | 9.586 |
| ATOM | 2137 | HE3 | TRP | 237 | −29.241 | −8.331 | 8.379 |
| ATOM | 2138 | N | ALA | 238 | −25.412 | −10.247 | 5.914 |
| ATOM | 2139 | CA | ALA | 238 | −24.179 | −10.319 | 6.697 |
| ATOM | 2140 | C | ALA | 238 | −23.466 | −8.983 | 6.681 |
| ATOM | 2141 | O | ALA | 238 | −23.053 | −8.516 | 7.731 |
| ATOM | 2142 | CB | ALA | 238 | −23.211 | −11.42 | 6.191 |
| ATOM | 2143 | HA | ALA | 238 | −24.45 | −10.569 | 7.736 |
| ATOM | 2144 | H | ALA | 238 | −25.538 | −10.862 | 5.129 |
| ATOM | 2145 | HB1 | ALA | 238 | −22.333 | −11.482 | 6.852 |
| ATOM | 2146 | HB2 | ALA | 238 | −23.72 | −12.397 | 6.186 |
| ATOM | 2147 | HB3 | ALA | 238 | −22.861 | −11.201 | 5.173 |
| ATOM | 2148 | N | LEU | 239 | −23.305 | −8.341 | 5.502 |
| ATOM | 2149 | CA | LEU | 239 | −22.592 | −7.062 | 5.461 |
| ATOM | 2150 | C | LEU | 239 | −23.313 | −6.036 | 6.309 |
| ATOM | 2151 | O | LEU | 239 | −22.67 | −5.297 | 7.04 |
| ATOM | 2152 | CB | LEU | 239 | −22.443 | −6.505 | 4.017 |
| ATOM | 2153 | CG | LEU | 239 | −21.569 | −7.374 | 3.06 |
| ATOM | 2154 | CD1 | LEU | 239 | −21.653 | −6.805 | 1.616 |
| ATOM | 2155 | CD2 | LEU | 239 | −20.081 | −7.442 | 3.506 |
| ATOM | 2156 | HA | LEU | 239 | −21.595 | −7.195 | 5.904 |
| ATOM | 2157 | H | LEU | 239 | −23.688 | −8.727 | 4.659 |
| ATOM | 2158 | HB1 | LEU | 239 | −23.455 | −6.391 | 3.594 |
| ATOM | 2159 | HB2 | LEU | 239 | −21.984 | −5.508 | 4.072 |
| ATOM | 2160 | HG | LEU | 239 | −21.962 | −8.402 | 3.032 |
| ATOM | 2161 | HD21 | LEU | 239 | −19.966 | −7.992 | 4.451 |
| ATOM | 2162 | HD22 | LEU | 239 | −19.479 | −7.968 | 2.748 |
| ATOM | 2163 | HD23 | LEU | 239 | −19.67 | −6.429 | 3.632 |
| ATOM | 2164 | HD11 | LEU | 239 | −21.258 | −5.778 | 1.583 |
| ATOM | 2165 | HD12 | LEU | 239 | −21.073 | −7.427 | 0.916 |
| ATOM | 2166 | HD13 | LEU | 239 | −22.697 | −6.791 | 1.271 |
| ATOM | 2167 | N | SER | 240 | −24.659 | −5.963 | 6.235 |
| ATOM | 2168 | CA | SER | 240 | −25.368 | −4.999 | 7.076 |
| ATOM | 2169 | C | SER | 240 | −25.21 | −5.355 | 8.535 |
| ATOM | 2170 | O | SER | 240 | −24.871 | −4.5 | 9.337 |
| ATOM | 2171 | CB | SER | 240 | −26.868 | −4.932 | 6.707 |
| ATOM | 2172 | OG | SER | 240 | −27.527 | −3.888 | 7.443 |
| ATOM | 2173 | HA | SER | 240 | −24.954 | −3.992 | 6.909 |
| ATOM | 2174 | H | SER | 240 | −25.18 | −6.576 | 5.631 |
| ATOM | 2175 | HB1 | SER | 240 | −26.925 | −4.685 | 5.638 |
| ATOM | 2176 | HB2 | SER | 240 | −27.371 | −5.902 | 6.861 |
| ATOM | 2177 | HG | SER | 240 | −27.591 | −4.092 | 8.372 |
| ATOM | 2178 | N | PHE | 241 | −25.445 | −6.63 | 8.905 |
| ATOM | 2179 | CA | PHE | 241 | −25.27 | −7.03 | 10.299 |
| ATOM | 2180 | C | PHE | 241 | −23.888 | −6.642 | 10.775 |
| ATOM | 2181 | O | PHE | 241 | −23.763 | −6.123 | 11.873 |
| ATOM | 2182 | CB | PHE | 241 | −25.467 | −8.561 | 10.44 |
| ATOM | 2183 | CG | PHE | 241 | −25.273 | −9.014 | 11.891 |
| ATOM | 2184 | CD1 | PHE | 241 | −26.302 | −8.839 | 12.823 |
| ATOM | 2185 | CD2 | PHE | 241 | −24.075 | −9.617 | 12.293 |
| ATOM | 2186 | CE1 | PHE | 241 | −26.161 | −9.321 | 14.128 |
| ATOM | 2187 | CE2 | PHE | 241 | −23.945 | −10.126 | 13.588 |
| ATOM | 2188 | CZ | PHE | 241 | −24.988 | −9.979 | 14.506 |
| ATOM | 2189 | HA | PHE | 241 | −26.024 | −6.509 | 10.913 |
| ATOM | 2190 | H | PHE | 241 | −25.723 | −7.313 | 8.227 |
| ATOM | 2191 | HB1 | PHE | 241 | −26.482 | −8.836 | 10.113 |
| ATOM | 2192 | HB2 | PHE | 241 | −24.755 | −9.084 | 9.784 |
| ATOM | 2193 | HD2 | PHE | 241 | −23.242 | −9.696 | 11.601 |
| ATOM | 2194 | HE2 | PHE | 241 | −23.031 | −10.634 | 13.88 |
| ATOM | 2195 | HZ | PHE | 241 | −24.886 | −10.374 | 15.512 |
| ATOM | 2196 | HE1 | PHE | 241 | −26.963 | −9.184 | 14.847 |
| ATOM | 2197 | HD1 | PHE | 241 | −27.216 | −8.33 | 12.535 |
| ATOM | 2198 | N | ILE | 242 | −22.838 | −6.871 | 9.954 |
| ATOM | 2199 | CA | ILE | 242 | −21.49 | −6.453 | 10.347 |
| ATOM | 2200 | C | ILE | 242 | −21.455 | −4.948 | 10.501 |
| ATOM | 2201 | O | ILE | 242 | −20.829 | −4.467 | 11.431 |
| ATOM | 2202 | CB | ILE | 242 | −20.417 | −6.936 | 9.32 |
| ATOM | 2203 | CG1 | ILE | 242 | −20.27 | −8.488 | 9.403 |
| ATOM | 2204 | CG2 | ILE | 242 | −19.05 | −6.225 | 9.553 |
| ATOM | 2205 | CD1 | ILE | 242 | −19.607 | −9.118 | 8.147 |
| ATOM | 2206 | HA | ILE | 242 | −21.252 | −6.895 | 11.328 |
| ATOM | 2207 | H | ILE | 242 | −22.982 | −7.288 | 9.053 |
| ATOM | 2208 | HB | ILE | 242 | −20.763 | −6.654 | 8.313 |
| ATOM | 2209 | HG11 | ILE | 242 | −19.686 | −8.754 | 10.299 |
| ATOM | 2210 | HG12 | ILE | 242 | −21.26 | −8.958 | 9.512 |
| ATOM | 2211 | HD11 | ILE | 242 | −18.574 | −8.768 | 8.01 |
| ATOM | 2212 | HD12 | ILE | 242 | −19.582 | −10.215 | 8.251 |
| ATOM | 2213 | HD13 | ILE | 242 | −20.186 | −8.874 | 7.244 |
| ATOM | 2214 | HG21 | ILE | 242 | −18.7 | −6.403 | 10.582 |
| ATOM | 2215 | HG22 | ILE | 242 | −18.283 | −6.59 | 8.856 |
| ATOM | 2216 | HG23 | ILE | 242 | −19.132 | −5.138 | 9.397 |
| ATOM | 2217 | N | SER | 243 | −22.103 | −4.167 | 9.612 |
| ATOM | 2218 | CA | SER | 243 | −22.092 | −2.715 | 9.795 |
| ATOM | 2219 | C | SER | 243 | −22.776 | −2.304 | 11.086 |
| ATOM | 2220 | O | SER | 243 | −22.282 | −1.398 | 11.738 |
| ATOM | 2221 | CB | SER | 243 | −22.764 | −1.99 | 8.595 |
| ATOM | 2222 | OG | SER | 243 | −22.837 | −0.566 | 8.783 |
| ATOM | 2223 | HA | SER | 243 | −21.042 | −2.378 | 9.838 |
| ATOM | 2224 | H | SER | 243 | −22.596 | −4.567 | 8.835 |
| ATOM | 2225 | HB1 | SER | 243 | −22.216 | −2.218 | 7.667 |
| ATOM | 2226 | HB2 | SER | 243 | −23.796 | −2.345 | 8.473 |
| ATOM | 2227 | HG | SER | 243 | −21.983 | −0.163 | 8.899 |
| ATOM | 2228 | N | ILE | 244 | −23.914 | −2.93 | 11.473 |
| ATOM | 2229 | CA | ILE | 244 | −24.678 | −2.453 | 12.637 |
| ATOM | 2230 | C | ILE | 244 | −24.275 | −3.112 | 13.94 |
| ATOM | 2231 | O | ILE | 244 | −24.13 | −2.407 | 14.928 |
| ATOM | 2232 | CB | ILE | 244 | −26.218 | −2.62 | 12.409 |
| ATOM | 2233 | CG1 | ILE | 244 | −26.782 | −1.468 | 11.526 |
| ATOM | 2234 | CG2 | ILE | 244 | −27.024 | −2.642 | 13.743 |
| ATOM | 2235 | CD1 | ILE | 244 | −26.093 | −1.335 | 10.146 |
| ATOM | 2236 | HA | ILE | 244 | −24.496 | −1.376 | 12.787 |
| ATOM | 2237 | H | ILE | 244 | −24.258 | −3.71 | 10.944 |
| ATOM | 2238 | HB | ILE | 244 | −26.412 | −3.584 | 11.91 |
| ATOM | 2239 | HG11 | ILE | 244 | −27.857 | −1.632 | 11.36 |
| ATOM | 2240 | HG12 | ILE | 244 | −26.663 | −0.52 | 12.07 |
| ATOM | 2241 | HD11 | ILE | 244 | −26.115 | −2.292 | 9.609 |
| ATOM | 2242 | HD12 | ILE | 244 | −26.623 | −0.589 | 9.541 |
| ATOM | 2243 | HD13 | ILE | 244 | −25.054 | −0.994 | 10.254 |
| ATOM | 2244 | HG21 | ILE | 244 | −26.82 | −1.734 | 14.332 |
| ATOM | 2245 | HG22 | ILE | 244 | −28.104 | −2.688 | 13.538 |
| ATOM | 2246 | HG23 | ILE | 244 | −26.773 | −3.523 | 14.354 |
| ATOM | 2247 | N | THR | 245 | −24.117 | −4.452 | 14.012 |
| ATOM | 2248 | CA | THR | 245 | −23.98 | −5.075 | 15.33 |
| ATOM | 2249 | C | THR | 245 | −22.861 | −4.527 | 16.196 |
| ATOM | 2250 | O | THR | 245 | −23.101 | −4.441 | 17.392 |
| ATOM | 2251 | CB | THR | 245 | −23.988 | −6.634 | 15.347 |
| ATOM | 2252 | OG1 | THR | 245 | −24.333 | −7.046 | 16.685 |
| ATOM | 2253 | CG2 | THR | 245 | −22.638 | −7.28 | 14.935 |
| ATOM | 2254 | HA | THR | 245 | −24.916 | −4.804 | 15.849 |
| ATOM | 2255 | H | THR | 245 | −24.185 | −5.031 | 13.197 |
| ATOM | 2256 | HB | THR | 245 | −24.782 | −6.979 | 14.662 |
| ATOM | 2257 | HG1 | THR | 245 | −24.433 | −7.987 | 16.782 |
| ATOM | 2258 | HG23 | THR | 245 | −22.652 | −8.357 | 15.152 |
| ATOM | 2259 | HG21 | THR | 245 | −22.458 | −7.162 | 13.857 |
| ATOM | 2260 | HG22 | THR | 245 | −21.8 | −6.844 | 15.497 |
| ATOM | 2261 | N | PRO | 246 | −21.648 | −4.139 | 15.719 |
| ATOM | 2262 | CA | PRO | 246 | −20.603 | −3.715 | 16.645 |
| ATOM | 2263 | C | PRO | 246 | −21.047 | −2.748 | 17.712 |
| ATOM | 2264 | O | PRO | 246 | −20.523 | −2.787 | 18.813 |
| ATOM | 2265 | CB | PRO | 246 | −19.64 | −3.031 | 15.644 |
| ATOM | 2266 | CG | PRO | 246 | −19.793 | −3.899 | 14.375 |
| ATOM | 2267 | CD | PRO | 246 | −21.324 | −4.097 | 14.298 |
| ATOM | 2268 | HA | PRO | 246 | −20.134 | −4.606 | 17.092 |
| ATOM | 2269 | HD2 | PRO | 246 | −21.593 | −4.997 | 13.733 |
| ATOM | 2270 | HD1 | PRO | 246 | −21.757 | −3.204 | 13.822 |
| ATOM | 2271 | HG2 | PRO | 246 | −19.299 | −4.872 | 14.535 |
| ATOM | 2272 | HG1 | PRO | 246 | −19.368 | −3.425 | 13.474 |
| ATOM | 2273 | HB1 | PRO | 246 | −19.991 | −2.009 | 15.419 |
| ATOM | 2274 | HB2 | PRO | 246 | −18.603 | −2.973 | 16.004 |
| ATOM | 2275 | N | VAL | 247 | −22.017 | −1.861 | 17.411 |
| ATOM | 2276 | CA | VAL | 247 | −22.409 | −0.88 | 18.413 |
| ATOM | 2277 | C | VAL | 247 | −23.195 | −1.592 | 19.484 |
| ATOM | 2278 | O | VAL | 247 | −22.865 | −1.458 | 20.651 |
| ATOM | 2279 | CB | VAL | 247 | −23.292 | 0.278 | 17.878 |
| ATOM | 2280 | CG1 | VAL | 247 | −23.385 | 1.398 | 18.953 |
| ATOM | 2281 | CG2 | VAL | 247 | −22.774 | 0.875 | 16.544 |
| ATOM | 2282 | HA | VAL | 247 | −21.496 | −0.444 | 18.849 |
| ATOM | 2283 | H | VAL | 247 | −22.496 | −1.906 | 16.534 |
| ATOM | 2284 | HB | VAL | 247 | −24.301 | −0.118 | 17.686 |
| ATOM | 2285 | HG11 | VAL | 247 | −22.394 | 1.816 | 19.164 |
| ATOM | 2286 | HG12 | VAL | 247 | −24.007 | 2.22 | 18.587 |
| ATOM | 2287 | HG13 | VAL | 247 | −23.813 | 1.022 | 19.895 |
| ATOM | 2288 | HG21 | VAL | 247 | −22.777 | 0.167 | 15.704 |
| ATOM | 2289 | HG22 | VAL | 247 | −23.453 | 1.687 | 16.271 |
| ATOM | 2290 | HG23 | VAL | 247 | −21.758 | 1.274 | 16.663 |
| ATOM | 2291 | N | TRP | 248 | −24.245 | −2.355 | 19.108 |
| ATOM | 2292 | CA | TRP | 248 | −25.029 | −3.049 | 20.125 |
| ATOM | 2293 | C | TRP | 248 | −24.138 | −4.014 | 20.87 |
| ATOM | 2294 | O | TRP | 248 | −24.355 | −4.216 | 22.054 |
| ATOM | 2295 | CB | TRP | 248 | −26.232 | −3.796 | 19.495 |
| ATOM | 2296 | CG | TRP | 248 | −27.048 | −4.503 | 20.547 |
| ATOM | 2297 | CD1 | TRP | 248 | −27.198 | −5.828 | 20.693 |
| ATOM | 2298 | CD2 | TRP | 248 | −27.855 | −3.844 | 21.636 |
| ATOM | 2299 | NE1 | TRP | 248 | −27.987 | −6.067 | 21.71 |
| ATOM | 2300 | CE2 | TRP | 248 | −28.402 | −4.928 | 22.297 |
| ATOM | 2301 | CE3 | TRP | 248 | −28.113 | −2.532 | 22.039 |
| ATOM | 2302 | CZ2 | TRP | 248 | −29.245 | −4.772 | 23.397 |
| ATOM | 2303 | CZ3 | TRP | 248 | −28.933 | −2.363 | 23.162 |
| ATOM | 2304 | CH2 | TRP | 248 | −29.505 | −3.462 | 23.819 |
| ATOM | 2305 | HA | TRP | 248 | −25.403 | −2.301 | 20.841 |
| ATOM | 2306 | H | TRP | 248 | −24.475 | −2.492 | 18.141 |
| ATOM | 2307 | HB1 | TRP | 248 | −26.884 | −3.079 | 18.971 |
| ATOM | 2308 | HB2 | TRP | 248 | −25.855 | −4.524 | 18.759 |
| ATOM | 2309 | HE1 | TRP | 248 | −28.253 | −7.011 | 22.025 |
| ATOM | 2310 | HD1 | TRP | 248 | −26.734 | −6.584 | 20.059 |
| ATOM | 2311 | HZ2 | TRP | 248 | −29.678 | −5.629 | 23.901 |
| ATOM | 2312 | HH2 | TRP | 248 | −30.159 | −3.295 | 24.669 |
| ATOM | 2313 | HZ3 | TRP | 248 | −29.13 | −1.362 | 23.534 |
| ATOM | 2314 | HE3 | TRP | 248 | −27.696 | −1.686 | 21.505 |
| ATOM | 2315 | N | LEU | 249 | −23.12 | −4.615 | 20.213 |
| ATOM | 2316 | CA | LEU | 249 | −22.182 | −5.444 | 20.963 |
| ATOM | 2317 | C | LEU | 249 | −21.392 | −4.591 | 21.935 |
| ATOM | 2318 | O | LEU | 249 | −21.154 | −5.06 | 23.036 |
| ATOM | 2319 | CB | LEU | 249 | −21.142 | −6.176 | 20.066 |
| ATOM | 2320 | CG | LEU | 249 | −21.725 | −7.158 | 19.005 |
| ATOM | 2321 | CD1 | LEU | 249 | −20.561 | −7.845 | 18.236 |
| ATOM | 2322 | CD2 | LEU | 249 | −22.66 | −8.23 | 19.629 |
| ATOM | 2323 | HA | LEU | 249 | −22.745 | −6.187 | 21.552 |
| ATOM | 2324 | H | LEU | 249 | −22.966 | −4.465 | 19.234 |
| ATOM | 2325 | HB1 | LEU | 249 | −20.541 | −5.413 | 19.547 |
| ATOM | 2326 | HB2 | LEU | 249 | −20.471 | −6.747 | 20.728 |
| ATOM | 2327 | HG | LEU | 249 | −22.312 | −6.595 | 18.265 |
| ATOM | 2328 | HD21 | LEU | 249 | −23.584 | −7.773 | 20.015 |
| ATOM | 2329 | HD22 | LEU | 249 | −22.954 | −8.971 | 18.87 |
| ATOM | 2330 | HD23 | LEU | 249 | −22.147 | −8.755 | 20.449 |
| ATOM | 2331 | HD11 | LEU | 249 | −19.95 | −8.449 | 18.925 |
| ATOM | 2332 | HD12 | LEU | 249 | −20.952 | −8.506 | 17.447 |
| ATOM | 2333 | HD13 | LEU | 249 | −19.912 | −7.094 | 17.761 |
| ATOM | 2334 | N | TYR | 250 | −20.948 | −3.367 | 21.554 |
| ATOM | 2335 | CA | TYR | 250 | −19.992 | −2.626 | 22.383 |
| ATOM | 2336 | C | TYR | 250 | −20.536 | −1.421 | 23.126 |
| ATOM | 2337 | O | TYR | 250 | −20.208 | −1.306 | 24.297 |
| ATOM | 2338 | CB | TYR | 250 | −18.79 | −2.205 | 21.493 |
| ATOM | 2339 | CG | TYR | 250 | −18.234 | −3.364 | 20.639 |
| ATOM | 2340 | CD1 | TYR | 250 | −17.788 | −3.11 | 19.336 |
| ATOM | 2341 | CD2 | TYR | 250 | −18.16 | −4.677 | 21.121 |
| ATOM | 2342 | CE1 | TYR | 250 | −17.354 | −4.152 | 18.51 |
| ATOM | 2343 | CE2 | TYR | 250 | −17.777 | −5.727 | 20.281 |
| ATOM | 2344 | CZ | TYR | 250 | −17.403 | −5.476 | 18.961 |
| ATOM | 2345 | OH | TYR | 250 | −17.082 | −6.548 | 18.123 |
| ATOM | 2346 | HA | TYR | 250 | −19.586 | −3.274 | 23.175 |
| ATOM | 2347 | H | TYR | 250 | −21.199 | −2.979 | 20.665 |
| ATOM | 2348 | HB2 | TYR | 250 | −19.137 | −1.396 | 20.83 |
| ATOM | 2349 | HB1 | TYR | 250 | −17.972 | −1.812 | 22.116 |
| ATOM | 2350 | HD2 | TYR | 250 | −18.393 | −4.913 | 22.151 |
| ATOM | 2351 | HE2 | TYR | 250 | −17.77 | −6.746 | 20.653 |
| ATOM | 2352 | HE1 | TYR | 250 | −16.975 | −3.927 | 17.52 |
| ATOM | 2353 | HD1 | TYR | 250 | −17.777 | −2.096 | 18.952 |
| ATOM | 2354 | HH | TYR | 250 | −16.937 | −6.302 | 17.216 |
| ATOM | 2355 | N | ALA | 251 | −21.321 | −0.484 | 22.537 |
| ATOM | 2356 | CA | ALA | 251 | −21.668 | 0.722 | 23.295 |
| ATOM | 2357 | C | ALA | 251 | −22.928 | 1.456 | 22.877 |
| ATOM | 2358 | O | ALA | 251 | −23.987 | 0.941 | 23.201 |
| ATOM | 2359 | CB | ALA | 251 | −20.425 | 1.64 | 23.378 |
| ATOM | 2360 | HA | ALA | 251 | −21.897 | 0.433 | 24.335 |
| ATOM | 2361 | H | ALA | 251 | −21.623 | −0.561 | 21.586 |
| ATOM | 2362 | HB1 | ALA | 251 | −20.109 | 1.891 | 22.362 |
| ATOM | 2363 | HB2 | ALA | 251 | −20.64 | 2.57 | 23.924 |
| ATOM | 2364 | HB3 | ALA | 251 | −19.598 | 1.116 | 23.874 |
| ATOM | 2365 | N | ARG | 252 | −22.9 | 2.656 | 22.237 |
| ATOM | 2366 | CA | ARG | 252 | −24.087 | 3.507 | 22.24 |
| ATOM | 2367 | C | ARG | 252 | −24.674 | 3.717 | 20.865 |
| ATOM | 2368 | O | ARG | 252 | −24.006 | 4.242 | 19.988 |
| ATOM | 2369 | CB | ARG | 252 | −23.771 | 4.917 | 22.81 |
| ATOM | 2370 | CG | ARG | 252 | −23.3 | 4.847 | 24.286 |
| ATOM | 2371 | CD | ARG | 252 | −23.129 | 6.265 | 24.898 |
| ATOM | 2372 | NE | ARG | 252 | −22.867 | 6.148 | 26.337 |
| ATOM | 2373 | CZ | ARG | 252 | −23.792 | 5.873 | 27.235 |
| ATOM | 2374 | NH1 | ARG | 252 | −25.055 | 5.69 | 26.935 |
| ATOM | 2375 | NH2 | ARG | 252 | −23.44 | 5.771 | 28.496 |
| ATOM | 2376 | HA | ARG | 252 | −24.859 | 3.108 | 22.917 |
| ATOM | 2377 | H | ARG | 252 | −22.091 | 3.059 | 21.808 |
| ATOM | 2378 | HB1 | ARG | 252 | −22.993 | 5.403 | 22.204 |
| ATOM | 2379 | HB2 | ARG | 252 | −24.683 | 5.534 | 22.756 |
| ATOM | 2380 | HG1 | ARG | 252 | −24.028 | 4.265 | 24.87 |
| ATOM | 2381 | HG2 | ARG | 252 | −22.338 | 4.316 | 24.346 |
| ATOM | 2382 | HD1 | ARG | 252 | −22.265 | 6.74 | 24.4 |
| ATOM | 2383 | HD2 | ARG | 252 | −23.986 | 6.926 | 24.699 |
| ATOM | 2384 | HE | ARG | 252 | −21.893 | 6.291 | 26.647 |
| ATOM | 2385 | HH12 | ARG | 252 | −25.753 | 5.447 | 27.659 |
| ATOM | 2386 | HH11 | ARG | 252 | −25.413 | 5.763 | 25.973 |
| ATOM | 2387 | HH22 | ARG | 252 | −24.135 | 5.554 | 29.226 |
| ATOM | 2388 | HH21 | ARG | 252 | −22.462 | 5.9 | 28.792 |
| ATOM | 2389 | N | LEU | 253 | −25.961 | 3.361 | 20.68 |
| ATOM | 2390 | CA | LEU | 253 | −26.729 | 3.951 | 19.592 |
| ATOM | 2391 | C | LEU | 253 | −27.26 | 5.195 | 20.29 |
| ATOM | 2392 | O | LEU | 253 | −28.229 | 5.091 | 21.024 |
| ATOM | 2393 | CB | LEU | 253 | −27.744 | 2.881 | 19.103 |
| ATOM | 2394 | CG | LEU | 253 | −27.027 | 1.641 | 18.467 |
| ATOM | 2395 | CD1 | LEU | 253 | −27.943 | 0.397 | 18.401 |
| ATOM | 2396 | CD2 | LEU | 253 | −26.472 | 1.917 | 17.042 |
| ATOM | 2397 | HA | LEU | 253 | −26.104 | 4.237 | 18.734 |
| ATOM | 2398 | H | LEU | 253 | −26.475 | 2.853 | 21.376 |
| ATOM | 2399 | HB1 | LEU | 253 | −28.358 | 2.573 | 19.963 |
| ATOM | 2400 | HB2 | LEU | 253 | −28.409 | 3.357 | 18.377 |
| ATOM | 2401 | HG | LEU | 253 | −26.195 | 1.351 | 19.128 |
| ATOM | 2402 | HD21 | LEU | 253 | −27.269 | 2.23 | 16.361 |
| ATOM | 2403 | HD22 | LEU | 253 | −26.02 | 1.019 | 16.602 |
| ATOM | 2404 | HD23 | LEU | 253 | −25.711 | 2.7 | 17.066 |
| ATOM | 2405 | HD11 | LEU | 253 | −28.818 | 0.592 | 17.764 |
| ATOM | 2406 | HD12 | LEU | 253 | −28.262 | 0.143 | 19.42 |
| ATOM | 2407 | HD13 | LEU | 253 | −27.404 | −0.472 | 17.993 |
| ATOM | 2408 | N | ILE | 254 | −26.581 | 6.361 | 20.135 |
| ATOM | 2409 | CA | ILE | 254 | −26.773 | 7.511 | 21.033 |
| ATOM | 2410 | C | ILE | 254 | −27.984 | 8.348 | 20.665 |
| ATOM | 2411 | O | ILE | 254 | −28.157 | 8.541 | 19.473 |
| ATOM | 2412 | CB | ILE | 254 | −25.481 | 8.406 | 21.05 |
| ATOM | 2413 | CG1 | ILE | 254 | −25.395 | 9.303 | 22.324 |
| ATOM | 2414 | CG2 | ILE | 254 | −25.342 | 9.292 | 19.775 |
| ATOM | 2415 | CD1 | ILE | 254 | −24.076 | 10.117 | 22.439 |
| ATOM | 2416 | HA | ILE | 254 | −26.893 | 7.075 | 22.036 |
| ATOM | 2417 | H | ILE | 254 | −25.872 | 6.412 | 19.432 |
| ATOM | 2418 | HB | ILE | 254 | −24.618 | 7.718 | 21.088 |
| ATOM | 2419 | HG11 | ILE | 254 | −26.236 | 10.017 | 22.334 |
| ATOM | 2420 | HG12 | ILE | 254 | −25.452 | 8.66 | 23.217 |
| ATOM | 2421 | HD11 | ILE | 254 | −24.013 | 10.907 | 21.672 |
| ATOM | 2422 | HD12 | ILE | 254 | −24.02 | 10.601 | 23.428 |
| ATOM | 2423 | HD13 | ILE | 254 | −23.214 | 9.445 | 22.338 |
| ATOM | 2424 | HG21 | ILE | 254 | −26.051 | 10.133 | 19.812 |
| ATOM | 2425 | HG22 | ILE | 254 | −24.321 | 9.69 | 19.684 |
| ATOM | 2426 | HG23 | ILE | 254 | −25.54 | 8.731 | 18.857 |
| ATOM | 2427 | N | PRO | 255 | −28.848 | 8.876 | 21.577 |
| ATOM | 2428 | CA | PRO | 255 | −29.993 | 9.666 | 21.126 |
| ATOM | 2429 | C | PRO | 255 | −29.719 | 11.06 | 20.594 |
| ATOM | 2430 | O | PRO | 255 | −28.608 | 11.557 | 20.68 |
| ATOM | 2431 | CB | PRO | 255 | −30.842 | 9.699 | 22.423 |
| ATOM | 2432 | CG | PRO | 255 | −29.78 | 9.627 | 23.54 |
| ATOM | 2433 | CD | PRO | 255 | −28.786 | 8.571 | 23.003 |
| ATOM | 2434 | HA | PRO | 255 | −30.543 | 9.128 | 20.351 |
| ATOM | 2435 | HD2 | PRO | 255 | −29.166 | 7.552 | 23.182 |
| ATOM | 2436 | HD1 | PRO | 255 | −27.816 | 8.693 | 23.499 |
| ATOM | 2437 | HG2 | PRO | 255 | −30.204 | 9.361 | 24.523 |
| ATOM | 2438 | HG1 | PRO | 255 | −29.277 | 10.606 | 23.616 |
| ATOM | 2439 | HB1 | PRO | 255 | −31.489 | 10.586 | 22.507 |
| ATOM | 2440 | HB2 | PRO | 255 | −31.468 | 8.793 | 22.489 |
| ATOM | 2441 | N | PHE | 256 | −30.782 | 11.69 | 20.032 |
| ATOM | 2442 | CA | PHE | 256 | −30.706 | 13.035 | 19.457 |
| ATOM | 2443 | C | PHE | 256 | −31.818 | 13.857 | 20.074 |
| ATOM | 2444 | O | PHE | 256 | −32.807 | 14.082 | 19.398 |
| ATOM | 2445 | CB | PHE | 256 | −30.87 | 12.893 | 17.917 |
| ATOM | 2446 | CG | PHE | 256 | −31.068 | 14.225 | 17.171 |
| ATOM | 2447 | CD1 | PHE | 256 | −29.973 | 15.051 | 16.898 |
| ATOM | 2448 | CD2 | PHE | 256 | −32.339 | 14.624 | 16.737 |
| ATOM | 2449 | CE1 | PHE | 256 | −30.146 | 16.247 | 16.197 |
| ATOM | 2450 | CE2 | PHE | 256 | −32.524 | 15.85 | 16.093 |
| ATOM | 2451 | CZ | PHE | 256 | −31.423 | 16.662 | 15.812 |
| ATOM | 2452 | HA | PHE | 256 | −29.738 | 13.522 | 19.637 |
| ATOM | 2453 | H | PHE | 256 | −31.668 | 11.224 | 19.966 |
| ATOM | 2454 | HB1 | PHE | 256 | −29.987 | 12.369 | 17.531 |
| ATOM | 2455 | HB2 | PHE | 256 | −31.733 | 12.261 | 17.691 |
| ATOM | 2456 | HD2 | PHE | 256 | −33.202 | 13.987 | 16.894 |
| ATOM | 2457 | HE2 | PHE | 256 | −33.522 | 16.169 | 15.809 |
| ATOM | 2458 | HZ | PHE | 256 | −31.559 | 17.608 | 15.3 |
| ATOM | 2459 | HE1 | PHE | 256 | −29.286 | 16.853 | 15.949 |
| ATOM | 2460 | HD1 | PHE | 256 | −28.984 | 14.766 | 17.227 |
| ATOM | 2461 | N | PRO | 257 | −31.761 | 14.329 | 21.343 |
| ATOM | 2462 | CA | PRO | 257 | −32.89 | 15.083 | 21.881 |
| ATOM | 2463 | C | PRO | 257 | −32.874 | 16.496 | 21.338 |
| ATOM | 2464 | O | PRO | 257 | −32.538 | 17.414 | 22.069 |
| ATOM | 2465 | CB | PRO | 257 | −32.552 | 14.984 | 23.391 |
| ATOM | 2466 | CG | PRO | 257 | −31.006 | 14.984 | 23.419 |
| ATOM | 2467 | CD | PRO | 257 | −30.623 | 14.086 | 22.219 |
| ATOM | 2468 | HA | PRO | 257 | −33.868 | 14.617 | 21.675 |
| ATOM | 2469 | HD2 | PRO | 257 | −30.594 | 13.025 | 22.511 |
| ATOM | 2470 | HD1 | PRO | 257 | −29.65 | 14.391 | 21.804 |
| ATOM | 2471 | HG2 | PRO | 257 | −30.589 | 14.624 | 24.374 |
| ATOM | 2472 | HG1 | PRO | 257 | −30.644 | 16.011 | 23.241 |
| ATOM | 2473 | HB1 | PRO | 257 | −33.001 | 15.787 | 23.997 |
| ATOM | 2474 | HB2 | PRO | 257 | −32.902 | 14.015 | 23.783 |
| ATOM | 2475 | N | GLY | 258 | −33.243 | 16.702 | 20.052 |
| ATOM | 2476 | CA | GLY | 258 | −33.225 | 18.046 | 19.468 |
| ATOM | 2477 | C | GLY | 258 | −34.627 | 18.596 | 19.361 |
| ATOM | 2478 | O | GLY | 258 | −34.998 | 19.058 | 18.293 |
| ATOM | 2479 | HA2 | GLY | 258 | −32.781 | 17.987 | 18.461 |
| ATOM | 2480 | HA1 | GLY | 258 | −32.621 | 18.761 | 20.047 |
| ATOM | 2481 | H | GLY | 258 | −33.555 | 15.942 | 19.471 |
| ATOM | 2482 | N | GLY | 259 | −35.436 | 18.561 | 20.445 |
| ATOM | 2483 | CA | GLY | 259 | −36.844 | 18.939 | 20.316 |
| ATOM | 2484 | C | GLY | 259 | −37.509 | 17.812 | 19.565 |
| ATOM | 2485 | O | GLY | 259 | −38.196 | 17.011 | 20.178 |
| ATOM | 2486 | HA2 | GLY | 259 | −36.96 | 19.89 | 19.773 |
| ATOM | 2487 | HA1 | GLY | 259 | −37.317 | 19.057 | 21.305 |
| ATOM | 2488 | H | GLY | 259 | −35.111 | 18.193 | 21.319 |
| ATOM | 2489 | N | ALA | 260 | −37.264 | 17.708 | 18.24 |
| ATOM | 2490 | CA | ALA | 260 | −37.568 | 16.46 | 17.544 |
| ATOM | 2491 | C | ALA | 260 | −36.582 | 15.423 | 18.035 |
| ATOM | 2492 | O | ALA | 260 | −35.611 | 15.805 | 18.67 |
| ATOM | 2493 | CB | ALA | 260 | −37.438 | 16.636 | 16.013 |
| ATOM | 2494 | HA | ALA | 260 | −38.592 | 16.131 | 17.787 |
| ATOM | 2495 | HB1 | ALA | 260 | −38.145 | 17.402 | 15.668 |
| ATOM | 2496 | HB2 | ALA | 260 | −36.413 | 16.946 | 15.757 |
| ATOM | 2497 | HB3 | ALA | 260 | −37.661 | 15.698 | 15.492 |
| ATOM | 2498 | H | ALA | 260 | −36.723 | 18.404 | 17.763 |
| ATOM | 2499 | N | VAL | 261 | −36.812 | 14.111 | 17.784 |
| ATOM | 2500 | CA | VAL | 261 | −36.022 | 13.084 | 18.466 |
| ATOM | 2501 | C | VAL | 261 | −35.515 | 12.025 | 17.506 |
| ATOM | 2502 | O | VAL | 261 | −36.147 | 11.79 | 16.489 |
| ATOM | 2503 | CB | VAL | 261 | −36.9 | 12.49 | 19.605 |
| ATOM | 2504 | CG1 | VAL | 261 | −36.115 | 11.465 | 20.466 |
| ATOM | 2505 | CG2 | VAL | 261 | −37.458 | 13.626 | 20.511 |
| ATOM | 2506 | HA | VAL | 261 | −35.137 | 13.529 | 18.939 |
| ATOM | 2507 | HB | VAL | 261 | −37.748 | 11.965 | 19.134 |
| ATOM | 2508 | HG11 | VAL | 261 | −35.215 | 11.94 | 20.884 |
| ATOM | 2509 | HG12 | VAL | 261 | −36.727 | 11.096 | 21.301 |
| ATOM | 2510 | HG13 | VAL | 261 | −35.827 | 10.595 | 19.856 |
| ATOM | 2511 | HG21 | VAL | 261 | −38.149 | 14.274 | 19.952 |
| ATOM | 2512 | HG22 | VAL | 261 | −38.015 | 13.214 | 21.364 |
| ATOM | 2513 | HG23 | VAL | 261 | −36.635 | 14.242 | 20.904 |
| ATOM | 2514 | H | VAL | 261 | −37.58 | 13.806 | 17.218 |
| ATOM | 2515 | N | GLY | 262 | −34.367 | 11.373 | 17.805 |
| ATOM | 2516 | CA | GLY | 262 | −33.827 | 10.356 | 16.902 |
| ATOM | 2517 | C | GLY | 262 | −32.552 | 9.773 | 17.467 |
| ATOM | 2518 | O | GLY | 262 | −32.345 | 9.955 | 18.656 |
| ATOM | 2519 | HA2 | GLY | 262 | −33.622 | 10.806 | 15.92 |
| ATOM | 2520 | HA1 | GLY | 262 | −34.554 | 9.54 | 16.782 |
| ATOM | 2521 | H | GLY | 262 | −33.863 | 11.566 | 18.649 |
| ATOM | 2522 | N | CYS | 263 | −31.688 | 9.086 | 16.68 |
| ATOM | 2523 | CA | CYS | 263 | −30.496 | 8.474 | 17.272 |
| ATOM | 2524 | C | CYS | 263 | −29.377 | 8.187 | 16.289 |
| ATOM | 2525 | O | CYS | 263 | −29.67 | 8.08 | 15.109 |
| ATOM | 2526 | CB | CYS | 263 | −30.89 | 7.095 | 17.849 |
| ATOM | 2527 | SG | CYS | 263 | −31.975 | 7.135 | 19.301 |
| ATOM | 2528 | HA | CYS | 263 | −30.084 | 9.143 | 18.04 |
| ATOM | 2529 | HB1 | CYS | 263 | −31.41 | 6.533 | 17.061 |
| ATOM | 2530 | HB2 | CYS | 263 | −29.967 | 6.584 | 18.161 |
| ATOM | 2531 | LPG1 | CYS | 263 | −32.17 | 7.769 | 19.447 |
| ATOM | 2532 | LPG2 | CYS | 263 | −32.523 | 6.745 | 19.21 |
| ATOM | 2533 | H | CYS | 263 | −31.869 | 8.932 | 15.707 |
| ATOM | 2534 | N | GLY | 264 | −28.109 | 8.03 | 16.764 |
| ATOM | 2535 | CA | GLY | 264 | −26.981 | 7.764 | 15.865 |
| ATOM | 2536 | C | GLY | 264 | −25.877 | 6.946 | 16.505 |
| ATOM | 2537 | O | GLY | 264 | −25.924 | 6.676 | 17.691 |
| ATOM | 2538 | HA2 | GLY | 264 | −26.531 | 8.712 | 15.539 |
| ATOM | 2539 | HA1 | GLY | 264 | −27.313 | 7.202 | 14.977 |
| ATOM | 2540 | H | GLY | 264 | −27.904 | 8.112 | 17.745 |
| ATOM | 2541 | N | ILE | 265 | −24.838 | 6.541 | 15.743 |
| ATOM | 2542 | CA | ILE | 265 | −23.909 | 5.507 | 16.221 |
| ATOM | 2543 | C | ILE | 265 | −22.695 | 6.041 | 16.968 |
| ATOM | 2544 | O | ILE | 265 | −21.96 | 6.797 | 16.355 |
| ATOM | 2545 | CB | ILE | 265 | −23.537 | 4.56 | 15.024 |
| ATOM | 2546 | CG1 | ILE | 265 | −24.823 | 4.055 | 14.29 |
| ATOM | 2547 | CG2 | ILE | 265 | −22.554 | 5.185 | 13.999 |
| ATOM | 2548 | CD1 | ILE | 265 | −24.617 | 2.805 | 13.387 |
| ATOM | 2549 | HA | ILE | 265 | −24.442 | 4.861 | 16.936 |
| ATOM | 2550 | HB | ILE | 265 | −22.988 | 3.711 | 15.453 |
| ATOM | 2551 | HG11 | ILE | 265 | −25.582 | 3.793 | 15.037 |
| ATOM | 2552 | HG12 | ILE | 265 | −25.25 | 4.859 | 13.669 |
| ATOM | 2553 | HD11 | ILE | 265 | −24.105 | 2.001 | 13.934 |
| ATOM | 2554 | HD12 | ILE | 265 | −25.592 | 2.419 | 13.05 |
| ATOM | 2555 | HD13 | ILE | 265 | −24.032 | 3.041 | 12.487 |
| ATOM | 2556 | HG21 | ILE | 265 | −22.361 | 4.504 | 13.163 |
| ATOM | 2557 | HG22 | ILE | 265 | −22.969 | 6.104 | 13.573 |
| ATOM | 2558 | HG23 | ILE | 265 | −21.583 | 5.395 | 14.469 |
| ATOM | 2559 | H | ILE | 265 | −24.728 | 6.916 | 14.825 |
| ATOM | 2560 | N | ARG | 266 | −22.394 | 5.665 | 18.246 |
| ATOM | 2561 | CA | ARG | 266 | −21.137 | 6.086 | 18.884 |
| ATOM | 2562 | C | ARG | 266 | −20.476 | 4.966 | 19.685 |
| ATOM | 2563 | O | ARG | 266 | −21.151 | 4.306 | 20.458 |
| ATOM | 2564 | CB | ARG | 266 | −21.438 | 7.316 | 19.782 |
| ATOM | 2565 | CG | ARG | 266 | −20.133 | 8.016 | 20.248 |
| ATOM | 2566 | CD | ARG | 266 | −20.406 | 9.299 | 21.079 |
| ATOM | 2567 | NE | ARG | 266 | −19.188 | 10.098 | 21.237 |
| ATOM | 2568 | CZ | ARG | 266 | −19.155 | 11.317 | 21.729 |
| ATOM | 2569 | NH1 | ARG | 266 | −20.223 | 11.934 | 22.172 |
| ATOM | 2570 | NH2 | ARG | 266 | −18.003 | 11.942 | 21.786 |
| ATOM | 2571 | HA | ARG | 266 | −20.425 | 6.393 | 18.103 |
| ATOM | 2572 | H | ARG | 266 | −22.981 | 5.096 | 18.825 |
| ATOM | 2573 | HB1 | ARG | 266 | −22.042 | 8.025 | 19.191 |
| ATOM | 2574 | HB2 | ARG | 266 | −22.033 | 7.007 | 20.654 |
| ATOM | 2575 | HG1 | ARG | 266 | −19.5 | 7.323 | 20.828 |
| ATOM | 2576 | HG2 | ARG | 266 | −19.589 | 8.311 | 19.342 |
| ATOM | 2577 | HD1 | ARG | 266 | −21.161 | 9.877 | 20.528 |
| ATOM | 2578 | HD2 | ARG | 266 | −20.802 | 9.013 | 22.066 |
| ATOM | 2579 | HE | ARG | 266 | −18.306 | 9.671 | 20.922 |
| ATOM | 2580 | HH12 | ARG | 266 | −20.16 | 12.89 | 22.548 |
| ATOM | 2581 | HH11 | ARG | 266 | −21.145 | 11.484 | 22.171 |
| ATOM | 2582 | HH22 | ARG | 266 | −17.128 | 11.505 | 21.456 |
| ATOM | 2583 | HH21 | ARG | 266 | −17.916 | 12.893 | 22.173 |
| ATOM | 2584 | N | LEU | 267 | −19.148 | 4.706 | 19.533 |
| ATOM | 2585 | CA | LEU | 267 | −18.505 | 3.536 | 20.17 |
| ATOM | 2586 | C | LEU | 267 | −18.369 | 3.72 | 21.677 |
| ATOM | 2587 | O | LEU | 267 | −18.978 | 4.657 | 22.169 |
| ATOM | 2588 | CB | LEU | 267 | −19.172 | 2.199 | 19.711 |
| ATOM | 2589 | CG | LEU | 267 | −18.951 | 1.79 | 18.223 |
| ATOM | 2590 | CD1 | LEU | 267 | −19.432 | 2.868 | 17.214 |
| ATOM | 2591 | CD2 | LEU | 267 | −17.484 | 1.363 | 17.939 |
| ATOM | 2592 | HA | LEU | 267 | −17.456 | 3.517 | 19.837 |
| ATOM | 2593 | H | LEU | 267 | −18.571 | 5.308 | 18.975 |
| ATOM | 2594 | HB1 | LEU | 267 | −18.81 | 1.35 | 20.296 |
| ATOM | 2595 | HB2 | LEU | 267 | −20.246 | 2.239 | 19.905 |
| ATOM | 2596 | HG | LEU | 267 | −19.568 | 0.888 | 18.052 |
| ATOM | 2597 | HD21 | LEU | 267 | −17.414 | 0.929 | 16.932 |
| ATOM | 2598 | HD22 | LEU | 267 | −17.152 | 0.6 | 18.66 |
| ATOM | 2599 | HD23 | LEU | 267 | −16.794 | 2.217 | 17.993 |
| ATOM | 2600 | HD11 | LEU | 267 | −19.508 | 2.42 | 16.213 |
| ATOM | 2601 | HD12 | LEU | 267 | −20.424 | 3.255 | 17.492 |
| ATOM | 2602 | HD13 | LEU | 267 | −18.723 | 3.705 | 17.158 |
| ATOM | 2603 | N | PRO | 268 | −17.584 | 2.938 | 22.48 |
| ATOM | 2604 | CA | PRO | 268 | −17.3 | 3.369 | 23.849 |
| ATOM | 2605 | C | PRO | 268 | −16.26 | 4.459 | 23.748 |
| ATOM | 2606 | O | PRO | 268 | −16.058 | 4.93 | 22.641 |
| ATOM | 2607 | CB | PRO | 268 | −16.627 | 2.082 | 24.397 |
| ATOM | 2608 | CG | PRO | 268 | −15.855 | 1.558 | 23.166 |
| ATOM | 2609 | CD | PRO | 268 | −16.881 | 1.739 | 22.024 |
| ATOM | 2610 | HA | PRO | 268 | −18.185 | 3.684 | 24.424 |
| ATOM | 2611 | HD2 | PRO | 268 | −17.544 | 0.86 | 22.06 |
| ATOM | 2612 | HD1 | PRO | 268 | −16.376 | 1.813 | 21.049 |
| ATOM | 2613 | HG2 | PRO | 268 | −15.51 | 0.517 | 23.282 |
| ATOM | 2614 | HG1 | PRO | 268 | −14.978 | 2.202 | 22.987 |
| ATOM | 2615 | HB1 | PRO | 268 | −15.967 | 2.234 | 25.264 |
| ATOM | 2616 | HB2 | PRO | 268 | −17.393 | 1.346 | 24.683 |
| ATOM | 2617 | N | ASN | 269 | −15.559 | 4.856 | 24.839 |
| ATOM | 2618 | CA | ASN | 269 | −14.345 | 5.663 | 24.658 |
| ATOM | 2619 | C | ASN | 269 | −14.725 | 6.818 | 23.756 |
| ATOM | 2620 | O | ASN | 269 | −14.208 | 6.929 | 22.655 |
| ATOM | 2621 | CB | ASN | 269 | −13.308 | 4.681 | 24.043 |
| ATOM | 2622 | CG | ASN | 269 | −11.857 | 5.025 | 24.254 |
| ATOM | 2623 | OD1 | ASN | 269 | −11.546 | 5.969 | 24.963 |
| ATOM | 2624 | ND2 | ASN | 269 | −10.936 | 4.238 | 23.659 |
| ATOM | 2625 | HA | ASN | 269 | −13.961 | 6.017 | 25.627 |
| ATOM | 2626 | HB1 | ASN | 269 | −13.427 | 3.702 | 24.534 |
| ATOM | 2627 | HB2 | ASN | 269 | −13.516 | 4.545 | 22.975 |
| ATOM | 2628 | HD22 | ASN | 269 | −9.963 | 4.391 | 23.829 |
| ATOM | 2629 | HD21 | ASN | 269 | −11.213 | 3.506 | 23.029 |
| ATOM | 2630 | H | ASN | 269 | −15.785 | 4.53 | 25.758 |
| ATOM | 2631 | N | PRO | 270 | −15.711 | 7.652 | 24.166 |
| ATOM | 2632 | CA | PRO | 270 | −16.597 | 8.264 | 23.185 |
| ATOM | 2633 | C | PRO | 270 | −15.979 | 9.108 | 22.101 |
| ATOM | 2634 | O | PRO | 270 | −16.693 | 9.362 | 21.145 |
| ATOM | 2635 | CB | PRO | 270 | −17.538 | 9.072 | 24.117 |
| ATOM | 2636 | CG | PRO | 270 | −16.753 | 9.197 | 25.442 |
| ATOM | 2637 | CD | PRO | 270 | −16.046 | 7.83 | 25.57 |
| ATOM | 2638 | HA | PRO | 270 | −17.161 | 7.46 | 22.68 |
| ATOM | 2639 | HD2 | PRO | 270 | −16.737 | 7.045 | 25.914 |
| ATOM | 2640 | HD1 | PRO | 270 | −15.181 | 7.912 | 26.247 |
| ATOM | 2641 | HG2 | PRO | 270 | −17.393 | 9.433 | 26.308 |
| ATOM | 2642 | HG1 | PRO | 270 | −15.994 | 9.991 | 25.331 |
| ATOM | 2643 | HB1 | PRO | 270 | −17.814 | 10.059 | 23.719 |
| ATOM | 2644 | HB2 | PRO | 270 | −18.461 | 8.499 | 24.309 |
| ATOM | 2645 | N | ASP | 271 | −14.714 | 9.563 | 22.189 |
| ATOM | 2646 | CA | ASP | 271 | −14.149 | 10.389 | 21.118 |
| ATOM | 2647 | C | ASP | 271 | −13.151 | 9.641 | 20.255 |
| ATOM | 2648 | O | ASP | 271 | −13.116 | 9.897 | 19.061 |
| ATOM | 2649 | CB | ASP | 271 | −13.576 | 11.668 | 21.773 |
| ATOM | 2650 | CG | ASP | 271 | −14.719 | 12.323 | 22.508 |
| ATOM | 2651 | OD1 | ASP | 271 | −15.677 | 12.781 | 21.826 |
| ATOM | 2652 | OD2 | ASP | 271 | −14.689 | 12.367 | 23.765 |
| ATOM | 2653 | HA | ASP | 271 | −14.926 | 10.746 | 20.422 |
| ATOM | 2654 | HB1 | ASP | 271 | −12.76 | 11.408 | 22.465 |
| ATOM | 2655 | HB2 | ASP | 271 | −13.184 | 12.356 | 21.008 |
| ATOM | 2656 | H | ASP | 271 | −14.162 | 9.367 | 23.001 |
| ATOM | 2657 | N | THR | 272 | −12.326 | 8.714 | 20.791 |
| ATOM | 2658 | CA | THR | 272 | −11.335 | 8.043 | 19.949 |
| ATOM | 2659 | C | THR | 272 | −11.98 | 6.911 | 19.18 |
| ATOM | 2660 | O | THR | 272 | −11.983 | 6.962 | 17.959 |
| ATOM | 2661 | CB | THR | 272 | −10.179 | 7.512 | 20.835 |
| ATOM | 2662 | OG1 | THR | 272 | −10.793 | 6.777 | 21.904 |
| ATOM | 2663 | CG2 | THR | 272 | −9.337 | 8.68 | 21.417 |
| ATOM | 2664 | HA | THR | 272 | −10.9 | 8.751 | 19.223 |
| ATOM | 2665 | HB | THR | 272 | −9.528 | 6.852 | 20.234 |
| ATOM | 2666 | HG1 | THR | 272 | −10.152 | 6.426 | 22.511 |
| ATOM | 2667 | HG23 | THR | 272 | −8.881 | 9.271 | 20.609 |
| ATOM | 2668 | HG21 | THR | 272 | −9.97 | 9.342 | 22.028 |
| ATOM | 2669 | HG22 | THR | 272 | −8.53 | 8.284 | 22.054 |
| ATOM | 2670 | H | THR | 272 | −12.389 | 8.436 | 21.751 |
| ATOM | 2671 | N | ASP | 273 | −12.535 | 5.878 | 19.856 |
| ATOM | 2672 | CA | ASP | 273 | −13.207 | 4.808 | 19.114 |
| ATOM | 2673 | C | ASP | 273 | −14.22 | 5.412 | 18.167 |
| ATOM | 2674 | O | ASP | 273 | −14.333 | 4.935 | 17.049 |
| ATOM | 2675 | CB | ASP | 273 | −13.937 | 3.782 | 20.018 |
| ATOM | 2676 | CG | ASP | 273 | −12.973 | 2.988 | 20.858 |
| ATOM | 2677 | OD1 | ASP | 273 | −13.257 | 1.788 | 21.117 |
| ATOM | 2678 | OD2 | ASP | 273 | −11.925 | 3.552 | 21.27 |
| ATOM | 2679 | HA | ASP | 273 | −12.448 | 4.264 | 18.527 |
| ATOM | 2680 | H | ASP | 273 | −12.484 | 5.818 | 20.857 |
| ATOM | 2681 | HB1 | ASP | 273 | −14.507 | 3.081 | 19.386 |
| ATOM | 2682 | HB2 | ASP | 273 | −14.647 | 4.306 | 20.674 |
| ATOM | 2683 | N | LEU | 274 | −14.957 | 6.468 | 18.577 |
| ATOM | 2684 | CA | LEU | 274 | −15.831 | 7.159 | 17.626 |
| ATOM | 2685 | C | LEU | 274 | −15.031 | 7.58 | 16.417 |
| ATOM | 2686 | O | LEU | 274 | −15.4 | 7.227 | 15.307 |
| ATOM | 2687 | CB | LEU | 274 | −16.476 | 8.431 | 18.25 |
| ATOM | 2688 | CG | LEU | 274 | −17.069 | 9.454 | 17.231 |
| ATOM | 2689 | CD1 | LEU | 274 | −18.169 | 8.831 | 16.33 |
| ATOM | 2690 | CD2 | LEU | 274 | −17.634 | 10.701 | 17.97 |
| ATOM | 2691 | HA | LEU | 274 | −16.627 | 6.463 | 17.31 |
| ATOM | 2692 | H | LEU | 274 | −14.884 | 6.807 | 19.519 |
| ATOM | 2693 | HB1 | LEU | 274 | −15.695 | 8.965 | 18.81 |
| ATOM | 2694 | HB2 | LEU | 274 | −17.241 | 8.104 | 18.968 |
| ATOM | 2695 | HG | LEU | 274 | −16.261 | 9.828 | 16.579 |
| ATOM | 2696 | HD21 | LEU | 274 | −16.874 | 11.145 | 18.631 |
| ATOM | 2697 | HD22 | LEU | 274 | −17.943 | 11.47 | 17.244 |
| ATOM | 2698 | HD23 | LEU | 274 | −18.511 | 10.431 | 18.573 |
| ATOM | 2699 | HD11 | LEU | 274 | −19.007 | 8.459 | 16.937 |
| ATOM | 2700 | HD12 | LEU | 274 | −18.551 | 9.599 | 15.64 |
| ATOM | 2701 | HD13 | LEU | 274 | −17.784 | 7.998 | 15.725 |
| ATOM | 2702 | N | TYR | 275 | −13.944 | 8.359 | 16.615 |
| ATOM | 2703 | CA | TYR | 275 | −13.236 | 8.908 | 15.462 |
| ATOM | 2704 | C | TYR | 275 | −12.873 | 7.793 | 14.508 |
| ATOM | 2705 | O | TYR | 275 | −13.114 | 7.907 | 13.316 |
| ATOM | 2706 | CB | TYR | 275 | −11.962 | 9.693 | 15.877 |
| ATOM | 2707 | CG | TYR | 275 | −11.399 | 10.437 | 14.659 |
| ATOM | 2708 | CD1 | TYR | 275 | −11.689 | 11.792 | 14.458 |
| ATOM | 2709 | CD2 | TYR | 275 | −10.598 | 9.764 | 13.73 |
| ATOM | 2710 | CE1 | TYR | 275 | −11.248 | 12.448 | 13.305 |
| ATOM | 2711 | CE2 | TYR | 275 | −10.156 | 10.421 | 12.578 |
| ATOM | 2712 | CZ | TYR | 275 | −10.489 | 11.759 | 12.351 |
| ATOM | 2713 | OH | TYR | 275 | −10.054 | 12.381 | 11.177 |
| ATOM | 2714 | HA | TYR | 275 | −13.923 | 9.606 | 14.96 |
| ATOM | 2715 | H | TYR | 275 | −13.63 | 8.598 | 17.537 |
| ATOM | 2716 | HB2 | TYR | 275 | −11.203 | 9.011 | 16.291 |
| ATOM | 2717 | HB1 | TYR | 275 | −12.217 | 10.422 | 16.662 |
| ATOM | 2718 | HD2 | TYR | 275 | −10.318 | 8.729 | 13.897 |
| ATOM | 2719 | HE2 | TYR | 275 | −9.551 | 9.89 | 11.85 |
| ATOM | 2720 | HE1 | TYR | 275 | −11.505 | 13.493 | 13.158 |
| ATOM | 2721 | HD1 | TYR | 275 | −12.265 | 12.344 | 15.194 |
| ATOM | 2722 | HH | TYR | 275 | −10.35 | 13.279 | 11.079 |
| ATOM | 2723 | N | TRP | 276 | −12.294 | 6.691 | 15.033 |
| ATOM | 2724 | CA | TRP | 276 | −11.946 | 5.575 | 14.157 |
| ATOM | 2725 | C | TRP | 276 | −13.19 | 4.932 | 13.583 |
| ATOM | 2726 | O | TRP | 276 | −13.185 | 4.565 | 12.417 |
| ATOM | 2727 | CB | TRP | 276 | −11.129 | 4.506 | 14.93 |
| ATOM | 2728 | CG | TRP | 276 | −10.814 | 3.347 | 14.02 |
| ATOM | 2729 | CD1 | TRP | 276 | −9.764 | 3.229 | 13.192 |
| ATOM | 2730 | CD2 | TRP | 276 | −11.64 | 2.095 | 13.868 |
| ATOM | 2731 | NE1 | TRP | 276 | −9.84 | 2.09 | 12.554 |
| ATOM | 2732 | CE2 | TRP | 276 | −10.933 | 1.388 | 12.913 |
| ATOM | 2733 | CE3 | TRP | 276 | −12.811 | 1.601 | 14.441 |
| ATOM | 2734 | CZ2 | TRP | 276 | −11.365 | 0.144 | 12.453 |
| ATOM | 2735 | CZ3 | TRP | 276 | −13.265 | 0.359 | 13.974 |
| ATOM | 2736 | CH2 | TRP | 276 | −12.557 | −0.355 | 12.994 |
| ATOM | 2737 | HA | TRP | 276 | −11.324 | 5.948 | 13.327 |
| ATOM | 2738 | H | TRP | 276 | −12.129 | 6.617 | 16.022 |
| ATOM | 2739 | HB1 | TRP | 276 | −11.691 | 4.139 | 15.803 |
| ATOM | 2740 | HB2 | TRP | 276 | −10.191 | 4.958 | 15.291 |
| ATOM | 2741 | HE1 | TRP | 276 | −9.15 | 1.769 | 11.858 |
| ATOM | 2742 | HD1 | TRP | 276 | −8.977 | 3.976 | 13.075 |
| ATOM | 2743 | HZ2 | TRP | 276 | −10.802 | −0.407 | 11.706 |
| ATOM | 2744 | HH2 | TRP | 276 | −12.939 | −1.311 | 12.65 |
| ATOM | 2745 | HZ3 | TRP | 276 | −14.179 | −0.06 | 14.38 |
| ATOM | 2746 | HE3 | TRP | 276 | −13.338 | 2.152 | 15.211 |
| ATOM | 2747 | N | PHE | 277 | −14.266 | 4.765 | 14.384 |
| ATOM | 2748 | CA | PHE | 277 | −15.424 | 4.032 | 13.89 |
| ATOM | 2749 | C | PHE | 277 | −16.072 | 4.732 | 12.726 |
| ATOM | 2750 | O | PHE | 277 | −16.412 | 4.06 | 11.766 |
| ATOM | 2751 | CB | PHE | 277 | −16.518 | 3.779 | 14.952 |
| ATOM | 2752 | CG | PHE | 277 | −17.578 | 2.862 | 14.322 |
| ATOM | 2753 | CD1 | PHE | 277 | −18.769 | 3.379 | 13.797 |
| ATOM | 2754 | CD2 | PHE | 277 | −17.344 | 1.484 | 14.26 |
| ATOM | 2755 | CE1 | PHE | 277 | −19.721 | 2.518 | 13.243 |
| ATOM | 2756 | CE2 | PHE | 277 | −18.299 | 0.622 | 13.716 |
| ATOM | 2757 | CZ | PHE | 277 | −19.495 | 1.139 | 13.212 |
| ATOM | 2758 | HA | PHE | 277 | −15.064 | 3.049 | 13.558 |
| ATOM | 2759 | H | PHE | 277 | −14.274 | 5.099 | 15.327 |
| ATOM | 2760 | HB1 | PHE | 277 | −16.071 | 3.285 | 15.828 |
| ATOM | 2761 | HB2 | PHE | 277 | −16.955 | 4.733 | 15.284 |
| ATOM | 2762 | HD1 | PHE | 277 | −18.958 | 4.448 | 13.814 |
| ATOM | 2763 | HE1 | PHE | 277 | −20.64 | 2.914 | 12.829 |
| ATOM | 2764 | HZ | PHE | 277 | −20.246 | 0.472 | 12.796 |
| ATOM | 2765 | HE2 | PHE | 277 | −18.112 | −0.448 | 13.683 |
| ATOM | 2766 | HD2 | PHE | 277 | −16.413 | 1.076 | 14.633 |
| ATOM | 2767 | N | THR | 278 | −16.277 | 6.066 | 12.765 |
| ATOM | 2768 | CA | THR | 278 | −16.88 | 6.699 | 11.597 |
| ATOM | 2769 | C | THR | 278 | −16.01 | 6.439 | 10.392 |
| ATOM | 2770 | O | THR | 278 | −16.543 | 6.077 | 9.355 |
| ATOM | 2771 | CB | THR | 278 | −17.058 | 8.234 | 11.726 |
| ATOM | 2772 | OG1 | THR | 278 | −17.881 | 8.533 | 12.864 |
| ATOM | 2773 | CG2 | THR | 278 | −17.638 | 8.822 | 10.407 |
| ATOM | 2774 | HA | THR | 278 | −17.878 | 6.255 | 11.449 |
| ATOM | 2775 | H | THR | 278 | −16.025 | 6.629 | 13.56 |
| ATOM | 2776 | HB | THR | 278 | −16.079 | 8.694 | 11.92 |
| ATOM | 2777 | HG1 | THR | 278 | −18.759 | 8.178 | 12.785 |
| ATOM | 2778 | HG23 | THR | 278 | −16.903 | 8.807 | 9.588 |
| ATOM | 2779 | HG21 | THR | 278 | −17.953 | 9.862 | 10.562 |
| ATOM | 2780 | HG22 | THR | 278 | −18.506 | 8.235 | 10.084 |
| ATOM | 2781 | N | LEU | 279 | −14.676 | 6.634 | 10.499 |
| ATOM | 2782 | CA | LEU | 279 | −13.831 | 6.394 | 9.332 |
| ATOM | 2783 | C | LEU | 279 | −14.053 | 4.974 | 8.862 |
| ATOM | 2784 | O | LEU | 279 | −14.311 | 4.766 | 7.687 |
| ATOM | 2785 | CB | LEU | 279 | −12.332 | 6.644 | 9.653 |
| ATOM | 2786 | CG | LEU | 279 | −11.351 | 6.238 | 8.511 |
| ATOM | 2787 | CD1 | LEU | 279 | −11.634 | 7.003 | 7.187 |
| ATOM | 2788 | CD2 | LEU | 279 | −9.886 | 6.499 | 8.968 |
| ATOM | 2789 | HA | LEU | 279 | −14.154 | 7.091 | 8.543 |
| ATOM | 2790 | H | LEU | 279 | −14.261 | 6.929 | 11.363 |
| ATOM | 2791 | HB1 | LEU | 279 | −12.2 | 7.711 | 9.894 |
| ATOM | 2792 | HB2 | LEU | 279 | −12.068 | 6.064 | 10.551 |
| ATOM | 2793 | HG | LEU | 279 | −11.449 | 5.156 | 8.313 |
| ATOM | 2794 | HD21 | LEU | 279 | −9.661 | 5.938 | 9.889 |
| ATOM | 2795 | HD22 | LEU | 279 | −9.174 | 6.18 | 8.192 |
| ATOM | 2796 | HD23 | LEU | 279 | −9.731 | 7.572 | 9.165 |
| ATOM | 2797 | HD11 | LEU | 279 | −11.622 | 8.09 | 7.364 |
| ATOM | 2798 | HD12 | LEU | 279 | −10.865 | 6.763 | 6.436 |
| ATOM | 2799 | HD13 | LEU | 279 | −12.609 | 6.724 | 6.762 |
| ATOM | 2800 | N | TYR | 280 | −13.965 | 3.984 | 9.777 |
| ATOM | 2801 | CA | TYR | 280 | −14.202 | 2.595 | 9.385 |
| ATOM | 2802 | C | TYR | 280 | −15.549 | 2.438 | 8.714 |
| ATOM | 2803 | O | TYR | 280 | −15.613 | 1.86 | 7.64 |
| ATOM | 2804 | CB | TYR | 280 | −14.142 | 1.675 | 10.634 |
| ATOM | 2805 | CG | TYR | 280 | −14.699 | 0.27 | 10.363 |
| ATOM | 2806 | CD1 | TYR | 280 | −16.031 | −0.028 | 10.675 |
| ATOM | 2807 | CD2 | TYR | 280 | −13.882 | −0.726 | 9.819 |
| ATOM | 2808 | CE1 | TYR | 280 | −16.521 | −1.324 | 10.501 |
| ATOM | 2809 | CE2 | TYR | 280 | −14.372 | −2.024 | 9.649 |
| ATOM | 2810 | CZ | TYR | 280 | −15.689 | −2.334 | 10.012 |
| ATOM | 2811 | OH | TYR | 280 | −16.193 | −3.634 | 9.895 |
| ATOM | 2812 | HA | TYR | 280 | −13.417 | 2.28 | 8.677 |
| ATOM | 2813 | H | TYR | 280 | −13.747 | 4.198 | 10.734 |
| ATOM | 2814 | HB2 | TYR | 280 | −13.105 | 1.624 | 10.998 |
| ATOM | 2815 | HB1 | TYR | 280 | −14.745 | 2.112 | 11.44 |
| ATOM | 2816 | HD2 | TYR | 280 | −12.863 | −0.492 | 9.531 |
| ATOM | 2817 | HE2 | TYR | 280 | −13.722 | −2.786 | 9.234 |
| ATOM | 2818 | HE1 | TYR | 280 | −17.553 | −1.552 | 10.748 |
| ATOM | 2819 | HD1 | TYR | 280 | −16.694 | 0.742 | 11.056 |
| ATOM | 2820 | HH | TYR | 280 | −15.546 | −4.285 | 9.646 |
| ATOM | 2821 | N | GLN | 281 | −16.644 | 2.929 | 9.332 |
| ATOM | 2822 | CA | GLN | 281 | −17.968 | 2.718 | 8.753 |
| ATOM | 2823 | C | GLN | 281 | −18.071 | 3.407 | 7.416 |
| ATOM | 2824 | O | GLN | 281 | −18.487 | 2.779 | 6.457 |
| ATOM | 2825 | CB | GLN | 281 | −19.091 | 3.272 | 9.672 |
| ATOM | 2826 | CG | GLN | 281 | −20.498 | 3.103 | 9.036 |
| ATOM | 2827 | CD | GLN | 281 | −21.561 | 3.703 | 9.921 |
| ATOM | 2828 | OE1 | GLN | 281 | −22.321 | 2.968 | 10.53 |
| ATOM | 2829 | NE2 | GLN | 281 | −21.644 | 5.046 | 10.015 |
| ATOM | 2830 | HA | GLN | 281 | −18.126 | 1.635 | 8.62 |
| ATOM | 2831 | H | GLN | 281 | −16.561 | 3.434 | 10.193 |
| ATOM | 2832 | HB1 | GLN | 281 | −19.059 | 2.73 | 10.629 |
| ATOM | 2833 | HB2 | GLN | 281 | −18.907 | 4.337 | 9.879 |
| ATOM | 2834 | HG1 | GLN | 281 | −20.562 | 3.609 | 8.06 |
| ATOM | 2835 | HG2 | GLN | 281 | −20.704 | 2.034 | 8.874 |
| ATOM | 2836 | HE22 | GLN | 281 | −22.344 | 5.454 | 10.604 |
| ATOM | 2837 | HE21 | GLN | 281 | −21.023 | 5.652 | 9.516 |
| ATOM | 2838 | N | PHE | 282 | −17.731 | 4.709 | 7.328 |
| ATOM | 2839 | CA | PHE | 282 | −17.908 | 5.406 | 6.058 |
| ATOM | 2840 | C | PHE | 282 | −17.029 | 4.759 | 5.014 |
| ATOM | 2841 | O | PHE | 282 | −17.486 | 4.521 | 3.907 |
| ATOM | 2842 | CB | PHE | 282 | −17.567 | 6.913 | 6.185 |
| ATOM | 2843 | CG | PHE | 282 | −17.868 | 7.616 | 4.854 |
| ATOM | 2844 | CD1 | PHE | 282 | −19.193 | 7.907 | 4.512 |
| ATOM | 2845 | CD2 | PHE | 282 | −16.837 | 7.967 | 3.975 |
| ATOM | 2846 | CE1 | PHE | 282 | −19.488 | 8.55 | 3.307 |
| ATOM | 2847 | CE2 | PHE | 282 | −17.128 | 8.635 | 2.782 |
| ATOM | 2848 | CZ | PHE | 282 | −18.452 | 8.938 | 2.453 |
| ATOM | 2849 | HA | PHE | 282 | −18.963 | 5.306 | 5.752 |
| ATOM | 2850 | H | PHE | 282 | −17.361 | 5.203 | 8.116 |
| ATOM | 2851 | HB1 | PHE | 282 | −18.184 | 7.368 | 6.977 |
| ATOM | 2852 | HB2 | PHE | 282 | −16.511 | 7.04 | 6.471 |
| ATOM | 2853 | HD2 | PHE | 282 | −15.807 | 7.722 | 4.213 |
| ATOM | 2854 | HE2 | PHE | 282 | −16.325 | 8.919 | 2.11 |
| ATOM | 2855 | HZ | PHE | 282 | −18.677 | 9.471 | 1.535 |
| ATOM | 2856 | HE1 | PHE | 282 | −20.519 | 8.746 | 3.034 |
| ATOM | 2857 | HD1 | PHE | 282 | −19.997 | 7.629 | 5.183 |
| ATOM | 2858 | N | PHE | 283 | −15.757 | 4.456 | 5.35 |
| ATOM | 2859 | CA | PHE | 283 | −14.893 | 3.803 | 4.372 |
| ATOM | 2860 | C | PHE | 283 | −15.482 | 2.476 | 3.943 |
| ATOM | 2861 | O | PHE | 283 | −15.698 | 2.263 | 2.76 |
| ATOM | 2862 | CB | PHE | 283 | −13.466 | 3.567 | 4.937 |
| ATOM | 2863 | CG | PHE | 283 | −12.657 | 2.677 | 3.985 |
| ATOM | 2864 | CD1 | PHE | 283 | −12.443 | 1.326 | 4.287 |
| ATOM | 2865 | CD2 | PHE | 283 | −12.125 | 3.211 | 2.806 |
| ATOM | 2866 | CE1 | PHE | 283 | −11.648 | 0.535 | 3.453 |
| ATOM | 2867 | CE2 | PHE | 283 | −11.348 | 2.415 | 1.96 |
| ATOM | 2868 | CZ | PHE | 283 | −11.088 | 1.083 | 2.296 |
| ATOM | 2869 | HA | PHE | 283 | −14.805 | 4.462 | 3.494 |
| ATOM | 2870 | H | PHE | 283 | −15.398 | 4.657 | 6.265 |
| ATOM | 2871 | HB1 | PHE | 283 | −12.951 | 4.531 | 5.074 |
| ATOM | 2872 | HB2 | PHE | 283 | −13.52 | 3.083 | 5.923 |
| ATOM | 2873 | HD2 | PHE | 283 | −12.314 | 4.247 | 2.544 |
| ATOM | 2874 | HE2 | PHE | 283 | −10.943 | 2.831 | 1.042 |
| ATOM | 2875 | HZ | PHE | 283 | −10.452 | 0.474 | 1.661 |
| ATOM | 2876 | HE1 | PHE | 283 | −11.465 | −0.505 | 3.706 |
| ATOM | 2877 | HD1 | PHE | 283 | −12.891 | 0.882 | 5.17 |
| ATOM | 2878 | N | LEU | 284 | −15.731 | 1.56 | 4.903 |
| ATOM | 2879 | CA | LEU | 284 | −16.166 | 0.213 | 4.537 |
| ATOM | 2880 | C | LEU | 284 | −17.569 | 0.231 | 3.982 |
| ATOM | 2881 | O | LEU | 284 | −17.791 | −0.281 | 2.897 |
| ATOM | 2882 | CB | LEU | 284 | −16.131 | −0.743 | 5.767 |
| ATOM | 2883 | CG | LEU | 284 | −16.772 | −2.146 | 5.524 |
| ATOM | 2884 | CD1 | LEU | 284 | −16.076 | −2.918 | 4.37 |
| ATOM | 2885 | CD2 | LEU | 284 | −16.715 | −2.982 | 6.833 |
| ATOM | 2886 | HA | LEU | 284 | −15.475 | −0.172 | 3.769 |
| ATOM | 2887 | H | LEU | 284 | −15.608 | 1.786 | 5.873 |
| ATOM | 2888 | HB1 | LEU | 284 | −15.084 | −0.86 | 6.091 |
| ATOM | 2889 | HB2 | LEU | 284 | −16.685 | −0.274 | 6.596 |
| ATOM | 2890 | HG | LEU | 284 | −17.839 | −2.026 | 5.266 |
| ATOM | 2891 | HD21 | LEU | 284 | −17.244 | −2.459 | 7.644 |
| ATOM | 2892 | HD22 | LEU | 284 | −17.195 | −3.964 | 6.693 |
| ATOM | 2893 | HD23 | LEU | 284 | −15.67 | −3.144 | 7.134 |
| ATOM | 2894 | HD11 | LEU | 284 | −14.991 | −2.975 | 4.545 |
| ATOM | 2895 | HD12 | LEU | 284 | −16.474 | −3.942 | 4.299 |
| ATOM | 2896 | HD13 | LEU | 284 | −16.257 | −2.427 | 3.404 |
| ATOM | 2897 | N | ALA | 285 | −18.539 | 0.782 | 4.742 |
| ATOM | 2898 | CA | ALA | 285 | −19.945 | 0.616 | 4.381 |
| ATOM | 2899 | C | ALA | 285 | −20.47 | 1.682 | 3.445 |
| ATOM | 2900 | O | ALA | 285 | −21.619 | 1.55 | 3.055 |
| ATOM | 2901 | CB | ALA | 285 | −20.791 | 0.505 | 5.677 |
| ATOM | 2902 | HA | ALA | 285 | −20.068 | −0.337 | 3.842 |
| ATOM | 2903 | H | ALA | 285 | −18.317 | 1.286 | 5.579 |
| ATOM | 2904 | HB1 | ALA | 285 | −21.808 | 0.156 | 5.451 |
| ATOM | 2905 | HB2 | ALA | 285 | −20.329 | −0.218 | 6.367 |
| ATOM | 2906 | HB3 | ALA | 285 | −20.848 | 1.482 | 6.179 |
| ATOM | 2907 | N | PHE | 286 | −19.687 | 2.717 | 3.052 |
| ATOM | 2908 | CA | PHE | 286 | −20.159 | 3.664 | 2.036 |
| ATOM | 2909 | C | PHE | 286 | −19.142 | 3.847 | 0.928 |
| ATOM | 2910 | O | PHE | 286 | −19.489 | 3.612 | −0.22 |
| ATOM | 2911 | CB | PHE | 286 | −20.54 | 5.045 | 2.623 |
| ATOM | 2912 | CG | PHE | 286 | −21.015 | 5.938 | 1.465 |
| ATOM | 2913 | CD1 | PHE | 286 | −22.359 | 5.94 | 1.077 |
| ATOM | 2914 | CD2 | PHE | 286 | −20.103 | 6.742 | 0.772 |
| ATOM | 2915 | CE1 | PHE | 286 | −22.796 | 6.775 | 0.043 |
| ATOM | 2916 | CE2 | PHE | 286 | −20.532 | 7.562 | −0.275 |
| ATOM | 2917 | CZ | PHE | 286 | −21.882 | 7.587 | −0.634 |
| ATOM | 2918 | HA | PHE | 286 | −21.078 | 3.295 | 1.553 |
| ATOM | 2919 | H | PHE | 286 | −18.769 | 2.849 | 3.432 |
| ATOM | 2920 | HB1 | PHE | 286 | −21.338 | 4.926 | 3.373 |
| ATOM | 2921 | HB2 | PHE | 286 | −19.679 | 5.506 | 3.126 |
| ATOM | 2922 | HD2 | PHE | 286 | −19.052 | 6.728 | 1.041 |
| ATOM | 2923 | HE2 | PHE | 286 | −19.817 | 8.179 | −0.81 |
| ATOM | 2924 | HZ | PHE | 286 | −22.22 | 8.234 | −1.438 |
| ATOM | 2925 | HE1 | PHE | 286 | −23.844 | 6.791 | −0.237 |
| ATOM | 2926 | HD1 | PHE | 286 | −23.068 | 5.287 | 1.572 |
| ATOM | 2927 | N | ALA | 287 | −17.892 | 4.281 | 1.204 |
| ATOM | 2928 | CA | ALA | 287 | −16.974 | 4.552 | 0.099 |
| ATOM | 2929 | C | ALA | 287 | −16.807 | 3.319 | −0.757 |
| ATOM | 2930 | O | ALA | 287 | −17.003 | 3.408 | −1.958 |
| ATOM | 2931 | CB | ALA | 287 | −15.578 | 5.039 | 0.565 |
| ATOM | 2932 | HA | ALA | 287 | −17.414 | 5.357 | −0.513 |
| ATOM | 2933 | H | ALA | 287 | −17.587 | 4.467 | 2.14 |
| ATOM | 2934 | HB1 | ALA | 287 | −14.968 | 5.323 | −0.307 |
| ATOM | 2935 | HB2 | ALA | 287 | −15.681 | 5.915 | 1.224 |
| ATOM | 2936 | HB3 | ALA | 287 | −15.049 | 4.243 | 1.106 |
| ATOM | 2937 | N | LEU | 288 | −16.442 | 2.154 | −0.178 |
| ATOM | 2938 | CA | LEU | 288 | −16.23 | 0.981 | −1.024 |
| ATOM | 2939 | C | LEU | 288 | −17.459 | 0.635 | −1.851 |
| ATOM | 2940 | O | LEU | 288 | −17.316 | 0.632 | −3.066 |
| ATOM | 2941 | CB | LEU | 288 | −15.646 | −0.248 | −0.271 |
| ATOM | 2942 | CG | LEU | 288 | −14.229 | −0.037 | 0.356 |
| ATOM | 2943 | CD1 | LEU | 288 | −13.762 | −1.365 | 1.018 |
| ATOM | 2944 | CD2 | LEU | 288 | −13.166 | 0.424 | −0.683 |
| ATOM | 2945 | HA | LEU | 288 | −15.472 | 1.271 | −1.768 |
| ATOM | 2946 | H | LEU | 288 | −16.299 | 2.087 | 0.814 |
| ATOM | 2947 | HB1 | LEU | 288 | −16.335 | −0.531 | 0.536 |
| ATOM | 2948 | HB2 | LEU | 288 | −15.594 | −1.087 | −0.983 |
| ATOM | 2949 | HG | LEU | 288 | −14.28 | 0.727 | 1.15 |
| ATOM | 2950 | HD21 | LEU | 288 | −13.32 | 1.473 | −0.978 |
| ATOM | 2951 | HD22 | LEU | 288 | −12.153 | 0.359 | −0.259 |
| ATOM | 2952 | HD23 | LEU | 288 | −13.204 | −0.213 | −1.579 |
| ATOM | 2953 | HD11 | LEU | 288 | −13.665 | −2.158 | 0.262 |
| ATOM | 2954 | HD12 | LEU | 288 | −12.788 | −1.238 | 1.512 |
| ATOM | 2955 | HD13 | LEU | 288 | −14.486 | −1.693 | 1.778 |
| ATOM | 2956 | N | PRO | 289 | −18.684 | 0.342 | −1.341 |
| ATOM | 2957 | CA | PRO | 289 | −19.775 | 0.046 | −2.258 |
| ATOM | 2958 | C | PRO | 289 | −20.067 | 1.222 | −3.163 |
| ATOM | 2959 | O | PRO | 289 | −20.403 | 0.988 | −4.312 |
| ATOM | 2960 | CB | PRO | 289 | −20.921 | −0.285 | −1.27 |
| ATOM | 2961 | CG | PRO | 289 | −20.52 | 0.409 | 0.051 |
| ATOM | 2962 | CD | PRO | 289 | −18.978 | 0.286 | 0.085 |
| ATOM | 2963 | HA | PRO | 289 | −19.534 | −0.836 | −2.876 |
| ATOM | 2964 | HD2 | PRO | 289 | −18.703 | −0.704 | 0.48 |
| ATOM | 2965 | HD1 | PRO | 289 | −18.531 | 1.083 | 0.694 |
| ATOM | 2966 | HG2 | PRO | 289 | −21.008 | −0.033 | 0.936 |
| ATOM | 2967 | HG1 | PRO | 289 | −20.819 | 1.464 | −0.018 |
| ATOM | 2968 | HB1 | PRO | 289 | −21.904 | 0.045 | −1.64 |
| ATOM | 2969 | HB2 | PRO | 289 | −20.951 | −1.373 | −1.09 |
| ATOM | 2970 | N | PHE | 290 | −19.938 | 2.49 | −2.711 |
| ATOM | 2971 | CA | PHE | 290 | −20.169 | 3.607 | −3.629 |
| ATOM | 2972 | C | PHE | 290 | −19.21 | 3.515 | −4.796 |
| ATOM | 2973 | O | PHE | 290 | −19.617 | 3.696 | −5.934 |
| ATOM | 2974 | CB | PHE | 290 | −20.002 | 4.981 | −2.927 |
| ATOM | 2975 | CG | PHE | 290 | −20.306 | 6.104 | −3.927 |
| ATOM | 2976 | CD1 | PHE | 290 | −21.6 | 6.629 | −4.028 |
| ATOM | 2977 | CD2 | PHE | 290 | −19.293 | 6.608 | −4.75 |
| ATOM | 2978 | CE1 | PHE | 290 | −21.886 | 7.627 | −4.963 |
| ATOM | 2979 | CE2 | PHE | 290 | −19.578 | 7.606 | −5.687 |
| ATOM | 2980 | CZ | PHE | 290 | −20.876 | 8.111 | −5.799 |
| ATOM | 2981 | HA | PHE | 290 | −21.201 | 3.552 | −4.011 |
| ATOM | 2982 | H | PHE | 290 | −19.642 | 2.686 | −1.773 |
| ATOM | 2983 | HB1 | PHE | 290 | −20.69 | 5.047 | −2.069 |
| ATOM | 2984 | HB2 | PHE | 290 | −18.977 | 5.092 | −2.544 |
| ATOM | 2985 | HD2 | PHE | 290 | −18.281 | 6.224 | −4.669 |
| ATOM | 2986 | HE2 | PHE | 290 | −18.79 | 7.985 | −6.331 |
| ATOM | 2987 | HZ | PHE | 290 | −21.099 | 8.878 | −6.534 |
| ATOM | 2988 | HE1 | PHE | 290 | −22.893 | 8.027 | −5.042 |
| ATOM | 2989 | HD1 | PHE | 290 | −22.389 | 6.261 | −3.38 |
| ATOM | 2990 | N | VAL | 291 | −17.916 | 3.23 | −4.527 |
| ATOM | 2991 | CA | VAL | 291 | −16.95 | 3.117 | −5.617 |
| ATOM | 2992 | C | VAL | 291 | −17.307 | 1.934 | −6.486 |
| ATOM | 2993 | O | VAL | 291 | −17.27 | 2.077 | −7.698 |
| ATOM | 2994 | CB | VAL | 291 | −15.484 | 2.992 | −5.101 |
| ATOM | 2995 | CG1 | VAL | 291 | −14.507 | 2.585 | −6.242 |
| ATOM | 2996 | CG2 | VAL | 291 | −15.013 | 4.334 | −4.472 |
| ATOM | 2997 | HA | VAL | 291 | −17.012 | 4.026 | −6.237 |
| ATOM | 2998 | H | VAL | 291 | −17.613 | 3.062 | −3.586 |
| ATOM | 2999 | HB | VAL | 291 | −15.446 | 2.202 | −4.332 |
| ATOM | 3000 | HG11 | VAL | 291 | −14.573 | 3.3 | −7.076 |
| ATOM | 3001 | HG12 | VAL | 291 | −13.47 | 2.573 | −5.871 |
| ATOM | 3002 | HG13 | VAL | 291 | −14.735 | 1.578 | −6.624 |
| ATOM | 3003 | HG21 | VAL | 291 | −15.708 | 4.683 | −3.696 |
| ATOM | 3004 | HG22 | VAL | 291 | −14.02 | 4.213 | −4.012 |
| ATOM | 3005 | HG23 | VAL | 291 | −14.945 | 5.116 | −5.244 |
| ATOM | 3006 | N | VAL | 292 | −17.647 | 0.753 | −5.922 |
| ATOM | 3007 | CA | VAL | 292 | −17.964 | −0.384 | −6.79 |
| ATOM | 3008 | C | VAL | 292 | −19.182 | −0.042 | −7.619 |
| ATOM | 3009 | O | VAL | 292 | −19.177 | −0.295 | −8.814 |
| ATOM | 3010 | CB | VAL | 292 | −18.201 | −1.721 | −6.025 |
| ATOM | 3011 | CG1 | VAL | 292 | −18.628 | −2.852 | −7.006 |
| ATOM | 3012 | CG2 | VAL | 292 | −16.922 | −2.166 | −5.261 |
| ATOM | 3013 | HA | VAL | 292 | −17.114 | −0.54 | −7.476 |
| ATOM | 3014 | H | VAL | 292 | −17.707 | 0.66 | −4.924 |
| ATOM | 3015 | HB | VAL | 292 | −19.015 | −1.566 | −5.296 |
| ATOM | 3016 | HG11 | VAL | 292 | −17.865 | −2.987 | −7.789 |
| ATOM | 3017 | HG12 | VAL | 292 | −18.743 | −3.807 | −6.47 |
| ATOM | 3018 | HG13 | VAL | 292 | −19.587 | −2.623 | −7.494 |
| ATOM | 3019 | HG21 | VAL | 292 | −16.588 | −1.407 | −4.54 |
| ATOM | 3020 | HG22 | VAL | 292 | −17.117 | −3.095 | −4.702 |
| ATOM | 3021 | HG23 | VAL | 292 | −16.099 | −2.353 | −5.969 |
| ATOM | 3022 | N | ILE | 293 | −20.239 | 0.536 | −7.008 |
| ATOM | 3023 | CA | ILE | 293 | −21.428 | 0.906 | −7.779 |
| ATOM | 3024 | C | ILE | 293 | −21.011 | 1.851 | −8.884 |
| ATOM | 3025 | O | ILE | 293 | −21.452 | 1.693 | −10.012 |
| ATOM | 3026 | CB | ILE | 293 | −22.513 | 1.578 | −6.878 |
| ATOM | 3027 | CG1 | ILE | 293 | −23.107 | 0.553 | −5.863 |
| ATOM | 3028 | CG2 | ILE | 293 | −23.649 | 2.214 | −7.733 |
| ATOM | 3029 | CD1 | ILE | 293 | −23.686 | 1.222 | −4.587 |
| ATOM | 3030 | HA | ILE | 293 | −21.859 | −0.002 | −8.232 |
| ATOM | 3031 | H | ILE | 293 | −20.197 | 0.751 | −6.03 |
| ATOM | 3032 | HB | ILE | 293 | −22.018 | 2.393 | −6.323 |
| ATOM | 3033 | HG11 | ILE | 293 | −23.896 | −0.036 | −6.356 |
| ATOM | 3034 | HG12 | ILE | 293 | −22.331 | −0.154 | −5.531 |
| ATOM | 3035 | HD11 | ILE | 293 | −24.559 | 1.85 | −4.816 |
| ATOM | 3036 | HD12 | ILE | 293 | −23.987 | 0.446 | −3.869 |
| ATOM | 3037 | HD13 | ILE | 293 | −22.929 | 1.845 | −4.092 |
| ATOM | 3038 | HG21 | ILE | 293 | −24.088 | 1.46 | −8.403 |
| ATOM | 3039 | HG22 | ILE | 293 | −24.448 | 2.614 | −7.093 |
| ATOM | 3040 | HG23 | ILE | 293 | −23.268 | 3.046 | −8.346 |
| ATOM | 3041 | N | THR | 294 | −20.158 | 2.853 | −8.577 |
| ATOM | 3042 | CA | THR | 294 | −19.786 | 3.827 | −9.601 |
| ATOM | 3043 | C | THR | 294 | −18.961 | 3.155 | −10.676 |
| ATOM | 3044 | O | THR | 294 | −19.277 | 3.29 | −11.848 |
| ATOM | 3045 | CB | THR | 294 | −19.018 | 5.04 | −9 |
| ATOM | 3046 | OG1 | THR | 294 | −19.843 | 5.762 | −8.065 |
| ATOM | 3047 | CG2 | THR | 294 | −18.603 | 6.045 | −10.106 |
| ATOM | 3048 | HA | THR | 294 | −20.71 | 4.226 | −10.051 |
| ATOM | 3049 | H | THR | 294 | −19.773 | 2.936 | −7.654 |
| ATOM | 3050 | HB | THR | 294 | −18.102 | 4.69 | −8.492 |
| ATOM | 3051 | HG1 | THR | 294 | −20.095 | 5.254 | −7.299 |
| ATOM | 3052 | HG23 | THR | 294 | −17.901 | 5.587 | −10.821 |
| ATOM | 3053 | HG21 | THR | 294 | −19.491 | 6.395 | −10.654 |
| ATOM | 3054 | HG22 | THR | 294 | −18.11 | 6.916 | −9.647 |
| ATOM | 3055 | N | ALA | 295 | −17.889 | 2.421 | −10.313 |
| ATOM | 3056 | CA | ALA | 295 | −17.066 | 1.774 | −11.334 |
| ATOM | 3057 | C | ALA | 295 | −17.917 | 0.835 | −12.159 |
| ATOM | 3058 | O | ALA | 295 | −17.821 | 0.837 | −13.376 |
| ATOM | 3059 | CB | ALA | 295 | −15.902 | 0.975 | −10.693 |
| ATOM | 3060 | HA | ALA | 295 | −16.642 | 2.553 | −11.99 |
| ATOM | 3061 | H | ALA | 295 | −17.652 | 2.304 | −9.347 |
| ATOM | 3062 | HB1 | ALA | 295 | −15.291 | 0.499 | −11.476 |
| ATOM | 3063 | HB2 | ALA | 295 | −15.26 | 1.649 | −10.105 |
| ATOM | 3064 | HB3 | ALA | 295 | −16.294 | 0.192 | −10.025 |
| ATOM | 3065 | N | ALA | 296 | −18.773 | 0.022 | −11.504 |
| ATOM | 3066 | CA | ALA | 296 | −19.664 | −0.853 | −12.26 |
| ATOM | 3067 | C | ALA | 296 | −20.524 | −0.018 | −13.18 |
| ATOM | 3068 | O | ALA | 296 | −20.684 | −0.4 | −14.329 |
| ATOM | 3069 | CB | ALA | 296 | −20.56 | −1.684 | −11.311 |
| ATOM | 3070 | HA | ALA | 296 | −19.055 | −1.546 | −12.866 |
| ATOM | 3071 | H | ALA | 296 | −18.832 | 0.034 | −10.502 |
| ATOM | 3072 | HB1 | ALA | 296 | −21.285 | −2.284 | −11.883 |
| ATOM | 3073 | HB2 | ALA | 296 | −19.945 | −2.359 | −10.695 |
| ATOM | 3074 | HB3 | ALA | 296 | −21.098 | −0.997 | −10.644 |
| ATOM | 3075 | N | TYR | 297 | −21.078 | 1.13 | −12.722 |
| ATOM | 3076 | CA | TYR | 297 | −21.82 | 1.975 | −13.653 |
| ATOM | 3077 | C | TYR | 297 | −20.929 | 2.351 | −14.816 |
| ATOM | 3078 | O | TYR | 297 | −21.373 | 2.258 | −15.949 |
| ATOM | 3079 | CB | TYR | 297 | −22.415 | 3.283 | −13.067 |
| ATOM | 3080 | CG | TYR | 297 | −22.918 | 4.142 | −14.241 |
| ATOM | 3081 | CD1 | TYR | 297 | −24.025 | 3.732 | −14.994 |
| ATOM | 3082 | CD2 | TYR | 297 | −22.257 | 5.325 | −14.593 |
| ATOM | 3083 | CE1 | TYR | 297 | −24.4 | 4.432 | −16.146 |
| ATOM | 3084 | CE2 | TYR | 297 | −22.664 | 6.052 | −15.715 |
| ATOM | 3085 | CZ | TYR | 297 | −23.719 | 5.6 | −16.511 |
| ATOM | 3086 | OH | TYR | 297 | −24.07 | 6.323 | −17.655 |
| ATOM | 3087 | HA | TYR | 297 | −22.669 | 1.388 | −14.024 |
| ATOM | 3088 | H | TYR | 297 | −20.966 | 1.433 | −11.771 |
| ATOM | 3089 | HB2 | TYR | 297 | −23.242 | 3.057 | −12.374 |
| ATOM | 3090 | HB1 | TYR | 297 | −21.642 | 3.821 | −12.502 |
| ATOM | 3091 | HD2 | TYR | 297 | −21.419 | 5.684 | −14.004 |
| ATOM | 3092 | HE2 | TYR | 297 | −22.158 | 6.976 | −15.973 |
| ATOM | 3093 | HE1 | TYR | 297 | −25.22 | 4.063 | −16.754 |
| ATOM | 3094 | HD1 | TYR | 297 | −24.594 | 2.86 | −14.692 |
| ATOM | 3095 | HH | TYR | 297 | −24.903 | 6.069 | −18.04 |
| ATOM | 3096 | N | VAL | 298 | −19.675 | 2.786 | −14.571 |
| ATOM | 3097 | CA | VAL | 298 | −18.817 | 3.176 | −15.691 |
| ATOM | 3098 | C | VAL | 298 | −18.662 | 2.009 | −16.644 |
| ATOM | 3099 | O | VAL | 298 | −18.623 | 2.232 | −17.843 |
| ATOM | 3100 | CB | VAL | 298 | −17.435 | 3.726 | −15.228 |
| ATOM | 3101 | CG1 | VAL | 298 | −16.477 | 3.945 | −16.434 |
| ATOM | 3102 | CG2 | VAL | 298 | −17.601 | 5.067 | −14.455 |
| ATOM | 3103 | HA | VAL | 298 | −19.327 | 3.983 | −16.243 |
| ATOM | 3104 | H | VAL | 298 | −19.326 | 2.826 | −13.632 |
| ATOM | 3105 | HB | VAL | 298 | −16.968 | 2.984 | −14.559 |
| ATOM | 3106 | HG11 | VAL | 298 | −16.939 | 4.619 | −17.172 |
| ATOM | 3107 | HG12 | VAL | 298 | −15.529 | 4.392 | −16.096 |
| ATOM | 3108 | HG13 | VAL | 298 | −16.237 | 2.991 | −16.93 |
| ATOM | 3109 | HG21 | VAL | 298 | −18.268 | 4.962 | −13.589 |
| ATOM | 3110 | HG22 | VAL | 298 | −16.624 | 5.419 | −14.086 |
| ATOM | 3111 | HG23 | VAL | 298 | −18.019 | 5.841 | −15.118 |
| ATOM | 3112 | N | ARG | 299 | −18.579 | 0.747 | −16.17 |
| ATOM | 3113 | CA | ARG | 299 | −18.527 | −0.363 | −17.123 |
| ATOM | 3114 | C | ARG | 299 | −19.861 | −0.479 | −17.835 |
| ATOM | 3115 | O | ARG | 299 | −19.889 | −0.597 | −19.049 |
| ATOM | 3116 | CB | ARG | 299 | −18.197 | −1.724 | −16.451 |
| ATOM | 3117 | CG | ARG | 299 | −16.806 | −1.733 | −15.758 |
| ATOM | 3118 | CD | ARG | 299 | −16.452 | −3.154 | −15.238 |
| ATOM | 3119 | NE | ARG | 299 | −15.201 | −3.188 | −14.477 |
| ATOM | 3120 | CZ | ARG | 299 | −14.696 | −4.289 | −13.961 |
| ATOM | 3121 | NH1 | ARG | 299 | −15.274 | −5.461 | −14.081 |
| ATOM | 3122 | NH2 | ARG | 299 | −13.567 | −4.217 | −13.297 |
| ATOM | 3123 | HA | ARG | 299 | −17.744 | −0.161 | −17.874 |
| ATOM | 3124 | H | ARG | 299 | −18.61 | 0.565 | −15.184 |
| ATOM | 3125 | HB1 | ARG | 299 | −18.976 | −1.96 | −15.71 |
| ATOM | 3126 | HB2 | ARG | 299 | −18.213 | −2.503 | −17.231 |
| ATOM | 3127 | HG1 | ARG | 299 | −16.03 | −1.407 | −16.471 |
| ATOM | 3128 | HG2 | ARG | 299 | −16.807 | −1.034 | −14.908 |
| ATOM | 3129 | HD1 | ARG | 299 | −17.284 | −3.503 | −14.603 |
| ATOM | 3130 | HD2 | ARG | 299 | −16.349 | −3.808 | −16.119 |
| ATOM | 3131 | HE | ARG | 299 | −14.701 | −2.294 | −14.351 |
| ATOM | 3132 | HH12 | ARG | 299 | −14.853 | −6.306 | −13.667 |
| ATOM | 3133 | HH11 | ARG | 299 | −16.159 | −5.581 | −14.589 |
| ATOM | 3134 | HH22 | ARG | 299 | −13.138 | −5.055 | −12.878 |
| ATOM | 3135 | HH21 | ARG | 299 | −13.074 | −3.32 | −13.175 |
| ATOM | 3136 | N | ILE | 300 | −20.993 | −0.446 | −17.101 |
| ATOM | 3137 | CA | ILE | 300 | −22.31 | −0.54 | −17.748 |
| ATOM | 3138 | C | ILE | 300 | −22.476 | 0.547 | −18.789 |
| ATOM | 3139 | O | ILE | 300 | −23.066 | 0.285 | −19.828 |
| ATOM | 3140 | CB | ILE | 300 | −23.429 | −0.486 | −16.666 |
| ATOM | 3141 | CG1 | ILE | 300 | −23.42 | −1.806 | −15.832 |
| ATOM | 3142 | CG2 | ILE | 300 | −24.831 | −0.197 | −17.269 |
| ATOM | 3143 | CD1 | ILE | 300 | −24.034 | −1.643 | −14.418 |
| ATOM | 3144 | HA | ILE | 300 | −22.393 | −1.502 | −18.275 |
| ATOM | 3145 | H | ILE | 300 | −20.931 | −0.317 | −16.108 |
| ATOM | 3146 | HB | ILE | 300 | −23.206 | 0.372 | −16.02 |
| ATOM | 3147 | HG11 | ILE | 300 | −23.965 | −2.589 | −16.384 |
| ATOM | 3148 | HG12 | ILE | 300 | −22.389 | −2.168 | −15.686 |
| ATOM | 3149 | HD11 | ILE | 300 | −25.091 | −1.345 | −14.469 |
| ATOM | 3150 | HD12 | ILE | 300 | −23.96 | −2.597 | −13.875 |
| ATOM | 3151 | HD13 | ILE | 300 | −23.484 | −0.89 | −13.837 |
| ATOM | 3152 | HG21 | ILE | 300 | −25.114 | −0.991 | −17.976 |
| ATOM | 3153 | HG22 | ILE | 300 | −25.587 | −0.145 | −16.472 |
| ATOM | 3154 | HG23 | ILE | 300 | −24.846 | 0.771 | −17.795 |
| ATOM | 3155 | N | LEU | 301 | −21.95 | 1.765 | −18.519 |
| ATOM | 3156 | CA | LEU | 301 | −22.023 | 2.862 | −19.489 |
| ATOM | 3157 | C | LEU | 301 | −21.674 | 2.312 | −20.857 |
| ATOM | 3158 | O | LEU | 301 | −22.399 | 2.593 | −21.797 |
| ATOM | 3159 | CB | LEU | 301 | −21.068 | 4.018 | −19.049 |
| ATOM | 3160 | CG | LEU | 301 | −21.218 | 5.403 | −19.747 |
| ATOM | 3161 | CD1 | LEU | 301 | −20.335 | 6.453 | −19.011 |
| ATOM | 3162 | CD2 | LEU | 301 | −20.812 | 5.387 | −21.247 |
| ATOM | 3163 | HA | LEU | 301 | −23.06 | 3.232 | −19.487 |
| ATOM | 3164 | H | LEU | 301 | −21.509 | 1.924 | −17.634 |
| ATOM | 3165 | HB1 | LEU | 301 | −21.257 | 4.199 | −17.979 |
| ATOM | 3166 | HB2 | LEU | 301 | −20.027 | 3.688 | −19.165 |
| ATOM | 3167 | HG | LEU | 301 | −22.266 | 5.73 | −19.655 |
| ATOM | 3168 | HD21 | LEU | 301 | −21.512 | 4.795 | −21.844 |
| ATOM | 3169 | HD22 | LEU | 301 | −20.82 | 6.407 | −21.661 |
| ATOM | 3170 | HD23 | LEU | 301 | −19.802 | 4.969 | −21.37 |
| ATOM | 3171 | HD11 | LEU | 301 | −19.272 | 6.176 | −19.081 |
| ATOM | 3172 | HD12 | LEU | 301 | −20.466 | 7.453 | −19.455 |
| ATOM | 3173 | HD13 | LEU | 301 | −20.605 | 6.522 | −17.946 |
| ATOM | 3174 | N | GLN | 302 | −20.591 | 1.5 | −20.965 |
| ATOM | 3175 | CA | GLN | 302 | −20.178 | 0.924 | −22.252 |
| ATOM | 3176 | C | GLN | 302 | −21.353 | 0.655 | −23.171 |
| ATOM | 3177 | O | GLN | 302 | −21.285 | 1.015 | −24.335 |
| ATOM | 3178 | CB | GLN | 302 | −19.436 | −0.439 | −22.087 |
| ATOM | 3179 | CG | GLN | 302 | −18.001 | −0.315 | −21.499 |
| ATOM | 3180 | CD | GLN | 302 | −17.453 | −1.67 | −21.108 |
| ATOM | 3181 | OE1 | GLN | 302 | −17.229 | −1.907 | −19.931 |
| ATOM | 3182 | NE2 | GLN | 302 | −17.22 | −2.595 | −22.064 |
| ATOM | 3183 | HA | GLN | 302 | −19.506 | 1.642 | −22.749 |
| ATOM | 3184 | H | GLN | 302 | −20.049 | 1.291 | −20.149 |
| ATOM | 3185 | HB1 | GLN | 302 | −20.047 | −1.101 | −21.455 |
| ATOM | 3186 | HB2 | GLN | 302 | −19.35 | −0.92 | −23.075 |
| ATOM | 3187 | HG1 | GLN | 302 | −17.33 | 0.142 | −22.243 |
| ATOM | 3188 | HG2 | GLN | 302 | −17.987 | 0.33 | −20.609 |
| ATOM | 3189 | HE22 | GLN | 302 | −16.865 | −3.493 | −21.799 |
| ATOM | 3190 | HE21 | GLN | 302 | −17.387 | −2.408 | −23.031 |
| ATOM | 3191 | N | ARG | 303 | −22.428 | 0.012 | −22.659 |
| ATOM | 3192 | CA | ARG | 303 | −23.606 | −0.273 | −23.483 |
| ATOM | 3193 | C | ARG | 303 | −24.819 | 0.562 | −23.122 |
| ATOM | 3194 | O | ARG | 303 | −25.538 | 0.943 | −24.034 |
| ATOM | 3195 | CB | ARG | 303 | −24.004 | −1.765 | −23.315 |
| ATOM | 3196 | CG | ARG | 303 | −23.053 | −2.714 | −24.094 |
| ATOM | 3197 | CD | ARG | 303 | −23.43 | −4.205 | −23.858 |
| ATOM | 3198 | NE | ARG | 303 | −22.83 | −5.102 | −24.851 |
| ATOM | 3199 | CZ | ARG | 303 | −23.106 | −6.386 | −24.93 |
| ATOM | 3200 | NH1 | ARG | 303 | −23.879 | −7.002 | −24.067 |
| ATOM | 3201 | NH2 | ARG | 303 | −22.59 | −7.084 | −25.915 |
| ATOM | 3202 | HA | ARG | 303 | −23.407 | −0.094 | −24.553 |
| ATOM | 3203 | H | ARG | 303 | −22.427 | −0.261 | −21.694 |
| ATOM | 3204 | HB1 | ARG | 303 | −24.007 | −2.021 | −22.246 |
| ATOM | 3205 | HB2 | ARG | 303 | −25.026 | −1.914 | −23.704 |
| ATOM | 3206 | HG1 | ARG | 303 | −23.146 | −2.483 | −25.168 |
| ATOM | 3207 | HG2 | ARG | 303 | −22.006 | −2.541 | −23.793 |
| ATOM | 3208 | HD1 | ARG | 303 | −23.112 | −4.476 | −22.84 |
| ATOM | 3209 | HD2 | ARG | 303 | −24.526 | −4.292 | −23.936 |
| ATOM | 3210 | HE | ARG | 303 | −22.19 | −4.681 | −25.544 |
| ATOM | 3211 | HH12 | ARG | 303 | −24.086 | −8.007 | −24.161 |
| ATOM | 3212 | HH11 | ARG | 303 | −24.279 | −6.516 | −23.252 |
| ATOM | 3213 | HH22 | ARG | 303 | −22.794 | −8.089 | −26.022 |
| ATOM | 3214 | HH21 | ARG | 303 | −21.973 | −6.647 | −26.616 |
| ATOM | 3215 | N | MET | 304 | −25.107 | 0.834 | −21.829 |
| ATOM | 3216 | CA | MET | 304 | −26.367 | 1.483 | −21.469 |
| ATOM | 3217 | C | MET | 304 | −26.061 | 2.842 | −20.893 |
| ATOM | 3218 | O | MET | 304 | −26.211 | 3.032 | −19.697 |
| ATOM | 3219 | CB | MET | 304 | −27.138 | 0.542 | −20.505 |
| ATOM | 3220 | CG | MET | 304 | −27.379 | −0.836 | −21.177 |
| ATOM | 3221 | SD | MET | 304 | −28.293 | −1.912 | −20.03 |
| ATOM | 3222 | CE | MET | 304 | −28.665 | −3.338 | −21.1 |
| ATOM | 3223 | HA | MET | 304 | −27.029 | 1.655 | −22.334 |
| ATOM | 3224 | H | MET | 304 | −24.493 | 0.578 | −21.078 |
| ATOM | 3225 | HB1 | MET | 304 | −26.558 | 0.395 | −19.583 |
| ATOM | 3226 | HB2 | MET | 304 | −28.104 | 1 | −20.233 |
| ATOM | 3227 | HG1 | MET | 304 | −27.96 | −0.704 | −22.102 |
| ATOM | 3228 | HG2 | MET | 304 | −26.43 | −1.332 | −21.425 |
| ATOM | 3229 | LPD1 | MET | 304 | −27.91 | −2.093 | −19.499 |
| ATOM | 3230 | LPD2 | MET | 304 | −28.861 | −1.613 | −19.808 |
| ATOM | 3231 | HE1 | MET | 304 | −27.785 | −3.989 | −21.186 |
| ATOM | 3232 | HE2 | MET | 304 | −29.486 | −3.913 | −20.654 |
| ATOM | 3233 | HE3 | MET | 304 | −28.964 | −3.005 | −22.104 |
| ATOM | 3234 | N | THR | 305 | −25.622 | 3.794 | −21.75 |
| ATOM | 3235 | CA | THR | 305 | −25.248 | 5.119 | −21.261 |
| ATOM | 3236 | C | THR | 305 | −26.333 | 5.76 | −20.416 |
| ATOM | 3237 | O | THR | 305 | −26.213 | 5.709 | −19.202 |
| ATOM | 3238 | CB | THR | 305 | −24.747 | 6.028 | −22.423 |
| ATOM | 3239 | OG1 | THR | 305 | −23.668 | 5.336 | −23.075 |
| ATOM | 3240 | CG2 | THR | 305 | −24.276 | 7.416 | −21.911 |
| ATOM | 3241 | HA | THR | 305 | −24.373 | 4.975 | −20.61 |
| ATOM | 3242 | H | THR | 305 | −25.476 | 3.584 | −22.72 |
| ATOM | 3243 | HB | THR | 305 | −25.548 | 6.164 | −23.168 |
| ATOM | 3244 | HG1 | THR | 305 | −23.301 | 5.808 | −23.816 |
| ATOM | 3245 | HG23 | THR | 305 | −25.097 | 7.951 | −21.412 |
| ATOM | 3246 | HG21 | THR | 305 | −23.447 | 7.3 | −21.198 |
| ATOM | 3247 | HG22 | THR | 305 | −23.924 | 8.03 | −22.754 |
| ATOM | 3248 | N | SER | 306 | −27.389 | 6.38 | −20.996 |
| ATOM | 3249 | CA | SER | 306 | −28.354 | 7.12 | −20.176 |
| ATOM | 3250 | C | SER | 306 | −29.596 | 7.406 | −20.998 |
| ATOM | 3251 | O | SER | 306 | −29.965 | 8.56 | −21.149 |
| ATOM | 3252 | CB | SER | 306 | −27.723 | 8.457 | −19.689 |
| ATOM | 3253 | OG | SER | 306 | −26.567 | 8.222 | −18.865 |
| ATOM | 3254 | HA | SER | 306 | −28.655 | 6.523 | −19.3 |
| ATOM | 3255 | H | SER | 306 | −27.509 | 6.392 | −21.988 |
| ATOM | 3256 | HB1 | SER | 306 | −27.433 | 9.054 | −20.569 |
| ATOM | 3257 | HB2 | SER | 306 | −28.468 | 9.023 | −19.105 |
| ATOM | 3258 | HG | SER | 306 | −26.15 | 9.021 | −18.557 |
| ATOM | 3259 | N | SER | 307 | −30.255 | 6.374 | −21.572 |
| ATOM | 3260 | CA | SER | 307 | −31.311 | 6.647 | −22.548 |
| ATOM | 3261 | C | SER | 307 | −32.22 | 5.45 | −22.735 |
| ATOM | 3262 | O | SER | 307 | −31.899 | 4.606 | −23.557 |
| ATOM | 3263 | CB | SER | 307 | −30.594 | 7.004 | −23.878 |
| ATOM | 3264 | OG | SER | 307 | −29.645 | 5.972 | −24.215 |
| ATOM | 3265 | HA | SER | 307 | −31.932 | 7.505 | −22.241 |
| ATOM | 3266 | H | SER | 307 | −29.959 | 5.426 | −21.436 |
| ATOM | 3267 | HB1 | SER | 307 | −31.343 | 7.122 | −24.68 |
| ATOM | 3268 | HB2 | SER | 307 | −30.059 | 7.962 | −23.756 |
| ATOM | 3269 | HG | SER | 307 | −29.174 | 6.157 | −25.023 |
| ATOM | 3270 | N | VAL | 308 | −33.357 | 5.349 | −21.999 |
| ATOM | 3271 | CA | VAL | 308 | −34.25 | 4.2 | −22.162 |
| ATOM | 3272 | C | VAL | 308 | −35.69 | 4.675 | −22.233 |
| ATOM | 3273 | O | VAL | 308 | −36.389 | 4.585 | −21.235 |
| ATOM | 3274 | CB | VAL | 308 | −34.027 | 3.152 | −21.025 |
| ATOM | 3275 | CG1 | VAL | 308 | −34.529 | 1.751 | −21.478 |
| ATOM | 3276 | CG2 | VAL | 308 | −32.549 | 3.132 | −20.541 |
| ATOM | 3277 | HA | VAL | 308 | −34.053 | 3.696 | −23.122 |
| ATOM | 3278 | H | VAL | 308 | −33.6 | 6.033 | −21.308 |
| ATOM | 3279 | HB | VAL | 308 | −34.614 | 3.42 | −20.133 |
| ATOM | 3280 | HG11 | VAL | 308 | −33.989 | 1.404 | −22.371 |
| ATOM | 3281 | HG12 | VAL | 308 | −34.391 | 1.01 | −20.677 |
| ATOM | 3282 | HG13 | VAL | 308 | −35.602 | 1.797 | −21.724 |
| ATOM | 3283 | HG21 | VAL | 308 | −32.296 | 4.066 | −20.017 |
| ATOM | 3284 | HG22 | VAL | 308 | −32.376 | 2.297 | −19.848 |
| ATOM | 3285 | HG23 | VAL | 308 | −31.857 | 3.026 | −21.38 |
| ATOM | 3286 | N | ALA | 309 | −36.15 | 5.186 | −23.403 |
| ATOM | 3287 | CA | ALA | 309 | −37.571 | 5.511 | −23.572 |
| ATOM | 3288 | C | ALA | 309 | −38.352 | 4.335 | −24.145 |
| ATOM | 3289 | O | ALA | 309 | −39.34 | 4.002 | −23.507 |
| ATOM | 3290 | CB | ALA | 309 | −37.806 | 6.857 | −24.314 |
| ATOM | 3291 | HA | ALA | 309 | −38.008 | 5.707 | −22.576 |
| ATOM | 3292 | H | ALA | 309 | −35.536 | 5.313 | −24.181 |
| ATOM | 3293 | HB1 | ALA | 309 | −38.887 | 7.065 | −24.377 |
| ATOM | 3294 | HB2 | ALA | 309 | −37.326 | 7.673 | −23.751 |
| ATOM | 3295 | HB3 | ALA | 309 | −37.399 | 6.876 | −25.331 |
| ATOM | 3296 | N | PRO | 310 | −38.046 | 3.637 | −25.275 |
| ATOM | 3297 | CA | PRO | 310 | −38.853 | 2.473 | −25.634 |
| ATOM | 3298 | C | PRO | 310 | −38.653 | 1.347 | −24.644 |
| ATOM | 3299 | O | PRO | 310 | −37.848 | 1.498 | −23.738 |
| ATOM | 3300 | CB | PRO | 310 | −38.271 | 2.11 | −27.024 |
| ATOM | 3301 | CG | PRO | 310 | −37.534 | 3.393 | −27.47 |
| ATOM | 3302 | CD | PRO | 310 | −36.929 | 3.942 | −26.156 |
| ATOM | 3303 | HA | PRO | 310 | −39.916 | 2.756 | −25.711 |
| ATOM | 3304 | HD2 | PRO | 310 | −36.664 | 4.996 | −26.288 |
| ATOM | 3305 | HD1 | PRO | 310 | −36.043 | 3.361 | −25.852 |
| ATOM | 3306 | HG2 | PRO | 310 | −38.277 | 4.112 | −27.856 |
| ATOM | 3307 | HG1 | PRO | 310 | −36.777 | 3.21 | −28.25 |
| ATOM | 3308 | HB1 | PRO | 310 | −37.521 | 1.312 | −26.928 |
| ATOM | 3309 | HB2 | PRO | 310 | −39.039 | 1.78 | −27.743 |
| ATOM | 3310 | N | ALA | 311 | −39.36 | 0.203 | −24.786 |
| ATOM | 3311 | CA | ALA | 311 | −39.231 | −0.845 | −23.775 |
| ATOM | 3312 | C | ALA | 311 | −39.84 | −2.168 | −24.188 |
| ATOM | 3313 | O | ALA | 311 | −40.556 | −2.201 | −25.174 |
| ATOM | 3314 | CB | ALA | 311 | −39.985 | −0.372 | −22.509 |
| ATOM | 3315 | HA | ALA | 311 | −38.167 | −1.015 | −23.544 |
| ATOM | 3316 | HB1 | ALA | 311 | −39.955 | −1.163 | −21.751 |
| ATOM | 3317 | HB2 | ALA | 311 | −39.534 | 0.538 | −22.086 |
| ATOM | 3318 | HB3 | ALA | 311 | −41.038 | −0.163 | −22.756 |
| ATOM | 3319 | H | ALA | 311 | −40.013 | 0.069 | −25.535 |
| ATOM | 3320 | N | SER | 312 | −39.577 | −3.256 | −23.421 |
| ATOM | 3321 | CA | SER | 312 | −40.21 | −4.547 | −23.696 |
| ATOM | 3322 | C | SER | 312 | −40.432 | −5.266 | −22.374 |
| ATOM | 3323 | O | SER | 312 | −39.445 | −5.627 | −21.752 |
| ATOM | 3324 | CB | SER | 312 | −39.281 | −5.373 | −24.618 |
| ATOM | 3325 | OG | SER | 312 | −39.078 | −4.678 | −25.86 |
| ATOM | 3326 | HA | SER | 312 | −41.172 | −4.413 | −24.219 |
| ATOM | 3327 | HB1 | SER | 312 | −38.313 | −5.527 | −24.121 |
| ATOM | 3328 | HB2 | SER | 312 | −39.744 | −6.353 | −24.804 |
| ATOM | 3329 | HG | SER | 312 | −38.525 | −5.151 | −26.474 |
| ATOM | 3330 | H | SER | 312 | −38.931 | −3.211 | −22.656 |
| ATOM | 3331 | N | GLN | 313 | −41.684 | −5.477 | −21.897 |
| ATOM | 3332 | CA | GLN | 313 | −41.876 | −6.097 | −20.58 |
| ATOM | 3333 | C | GLN | 313 | −41.413 | −7.546 | −20.561 |
| ATOM | 3334 | O | GLN | 313 | −42.215 | −8.443 | −20.754 |
| ATOM | 3335 | CB | GLN | 313 | −43.321 | −5.946 | −20.011 |
| ATOM | 3336 | CG | GLN | 313 | −44.476 | −6.428 | −20.937 |
| ATOM | 3337 | CD | GLN | 313 | −45.695 | −6.716 | −20.092 |
| ATOM | 3338 | OE1 | GLN | 313 | −46.601 | −5.899 | −20.048 |
| ATOM | 3339 | NE2 | GLN | 313 | −45.743 | −7.869 | −19.391 |
| ATOM | 3340 | HA | GLN | 313 | −41.244 | −5.546 | −19.861 |
| ATOM | 3341 | HB1 | GLN | 313 | −43.347 | −6.516 | −19.069 |
| ATOM | 3342 | HB2 | GLN | 313 | −43.517 | −4.889 | −19.76 |
| ATOM | 3343 | HG1 | GLN | 313 | −44.726 | −5.655 | −21.681 |
| ATOM | 3344 | HG2 | GLN | 313 | −44.214 | −7.341 | −21.49 |
| ATOM | 3345 | HE22 | GLN | 313 | −46.545 | −8.065 | −18.826 |
| ATOM | 3346 | HE21 | GLN | 313 | −44.998 | −8.536 | −19.433 |
| ATOM | 3347 | H | GLN | 313 | −42.488 | −5.196 | −22.422 |
| ATOM | 3348 | N | ARG | 314 | −40.108 | −7.808 | −20.317 |
| ATOM | 3349 | CA | ARG | 314 | −39.604 | −9.187 | −20.278 |
| ATOM | 3350 | C | ARG | 314 | −39.717 | −9.938 | −21.589 |
| ATOM | 3351 | O | ARG | 314 | −39.477 | −11.135 | −21.578 |
| ATOM | 3352 | CB | ARG | 314 | −40.136 | −9.993 | −19.057 |
| ATOM | 3353 | CG | ARG | 314 | −39.696 | −9.325 | −17.723 |
| ATOM | 3354 | CD | ARG | 314 | −40.109 | −10.157 | −16.474 |
| ATOM | 3355 | NE | ARG | 314 | −39.675 | −9.487 | −15.244 |
| ATOM | 3356 | CZ | ARG | 314 | −40.067 | −9.824 | −14.036 |
| ATOM | 3357 | NH1 | ARG | 314 | −40.861 | −10.84 | −13.802 |
| ATOM | 3358 | NH2 | ARG | 314 | −39.641 | −9.117 | −13.018 |
| ATOM | 3359 | HA | ARG | 314 | −38.519 | −9.164 | −20.124 |
| ATOM | 3360 | H | ARG | 314 | −39.468 | −7.054 | −20.157 |
| ATOM | 3361 | HB1 | ARG | 314 | −41.234 | −10.068 | −19.101 |
| ATOM | 3362 | HB2 | ARG | 314 | −39.726 | −11.017 | −19.076 |
| ATOM | 3363 | HG1 | ARG | 314 | −38.598 | −9.212 | −17.715 |
| ATOM | 3364 | HG2 | ARG | 314 | −40.148 | −8.32 | −17.657 |
| ATOM | 3365 | HD1 | ARG | 314 | −41.204 | −10.276 | −16.523 |
| ATOM | 3366 | HD2 | ARG | 314 | −39.628 | −11.147 | −16.522 |
| ATOM | 3367 | HE | ARG | 314 | −39.008 | −8.701 | −15.347 |
| ATOM | 3368 | HH12 | ARG | 314 | −41.15 | −11.066 | −12.84 |
| ATOM | 3369 | HH11 | ARG | 314 | −41.203 | −11.445 | −14.556 |
| ATOM | 3370 | HH22 | ARG | 314 | −39.894 | −9.368 | −12.051 |
| ATOM | 3371 | HH21 | ARG | 314 | −39.01 | −8.313 | −13.14 |
| ATOM | 3372 | N | SER | 315 | −40.038 | −9.302 | −22.739 |
| ATOM | 3373 | CA | SER | 315 | −40.243 | −10.083 | −23.961 |
| ATOM | 3374 | C | SER | 315 | −38.95 | −10.448 | −24.668 |
| ATOM | 3375 | O | SER | 315 | −38.767 | −11.629 | −24.921 |
| ATOM | 3376 | CB | SER | 315 | −41.207 | −9.354 | −24.935 |
| ATOM | 3377 | OG | SER | 315 | −42.377 | −8.946 | −24.201 |
| ATOM | 3378 | HA | SER | 315 | −40.772 | −11.014 | −23.691 |
| ATOM | 3379 | H | SER | 315 | −40.168 | −8.31 | −22.778 |
| ATOM | 3380 | HB1 | SER | 315 | −40.716 | −8.467 | −25.363 |
| ATOM | 3381 | HB2 | SER | 315 | −41.485 | −10.032 | −25.762 |
| ATOM | 3382 | HG | SER | 315 | −43.022 | −8.505 | −24.746 |
| ATOM | 3383 | N | ILE | 316 | −38.054 | −9.49 | −25.028 |
| ATOM | 3384 | CA | ILE | 316 | −36.915 | −9.821 | −25.901 |
| ATOM | 3385 | C | ILE | 316 | −35.593 | −9.22 | −25.446 |
| ATOM | 3386 | O | ILE | 316 | −34.682 | −9.987 | −25.172 |
| ATOM | 3387 | CB | ILE | 316 | −37.21 | −9.415 | −27.382 |
| ATOM | 3388 | CG1 | ILE | 316 | −38.597 | −9.962 | −27.84 |
| ATOM | 3389 | CG2 | ILE | 316 | −36.076 | −9.913 | −28.326 |
| ATOM | 3390 | CD1 | ILE | 316 | −38.965 | −9.606 | −29.306 |
| ATOM | 3391 | HA | ILE | 316 | −36.758 | −10.912 | −25.922 |
| ATOM | 3392 | H | ILE | 316 | −38.202 | −8.532 | −24.78 |
| ATOM | 3393 | HB | ILE | 316 | −37.252 | −8.315 | −27.442 |
| ATOM | 3394 | HG11 | ILE | 316 | −38.631 | −11.058 | −27.717 |
| ATOM | 3395 | HG12 | ILE | 316 | −39.378 | −9.521 | −27.203 |
| ATOM | 3396 | HD11 | ILE | 316 | −38.337 | −10.155 | −30.024 |
| ATOM | 3397 | HD12 | ILE | 316 | −40.014 | −9.88 | −29.505 |
| ATOM | 3398 | HD13 | ILE | 316 | −38.85 | −8.524 | −29.481 |
| ATOM | 3399 | HG21 | ILE | 316 | −36.105 | −11.009 | −28.413 |
| ATOM | 3400 | HG22 | ILE | 316 | −36.177 | −9.476 | −29.33 |
| ATOM | 3401 | HG23 | ILE | 316 | −35.084 | −9.626 | −27.951 |
| ATOM | 3402 | N | ARG | 317 | −35.425 | −7.875 | −25.382 |
| ATOM | 3403 | CA | ARG | 317 | −34.08 | −7.299 | −25.233 |
| ATOM | 3404 | C | ARG | 317 | −34.045 | −6.084 | −24.328 |
| ATOM | 3405 | O | ARG | 317 | −33.327 | −6.11 | −23.34 |
| ATOM | 3406 | CB | ARG | 317 | −33.566 | −6.938 | −26.655 |
| ATOM | 3407 | CG | ARG | 317 | −32.153 | −6.288 | −26.654 |
| ATOM | 3408 | CD | ARG | 317 | −31.696 | −5.943 | −28.1 |
| ATOM | 3409 | NE | ARG | 317 | −30.355 | −5.351 | −28.14 |
| ATOM | 3410 | CZ | ARG | 317 | −29.758 | −4.971 | −29.25 |
| ATOM | 3411 | NH1 | ARG | 317 | −30.315 | −5.087 | −30.432 |
| ATOM | 3412 | NH2 | ARG | 317 | −28.554 | −4.453 | −29.178 |
| ATOM | 3413 | HA | ARG | 317 | −33.375 | −8.028 | −24.797 |
| ATOM | 3414 | H | ARG | 317 | −36.191 | −7.257 | −25.554 |
| ATOM | 3415 | HB1 | ARG | 317 | −33.527 | −7.862 | −27.252 |
| ATOM | 3416 | HB2 | ARG | 317 | −34.281 | −6.255 | −27.14 |
| ATOM | 3417 | HG1 | ARG | 317 | −32.16 | −5.362 | −26.055 |
| ATOM | 3418 | HG2 | ARG | 317 | −31.432 | −6.987 | −26.198 |
| ATOM | 3419 | HD1 | ARG | 317 | −31.703 | −6.876 | −28.69 |
| ATOM | 3420 | HD2 | ARG | 317 | −32.427 | −5.233 | −28.522 |
| ATOM | 3421 | HE | ARG | 317 | −29.864 | −5.235 | −27.239 |
| ATOM | 3422 | HH12 | ARG | 317 | −29.822 | −4.78 | −31.284 |
| ATOM | 3423 | HH11 | ARG | 317 | −31.254 | −5.485 | −30.552 |
| ATOM | 3424 | HH22 | ARG | 317 | −28.054 | −4.144 | −30.025 |
| ATOM | 3425 | HH21 | ARG | 317 | −28.072 | −4.341 | −28.274 |
| ATOM | 3426 | N | LEU | 318 | −34.793 | −4.995 | −24.623 |
| ATOM | 3427 | CA | LEU | 318 | −34.72 | −3.821 | −23.751 |
| ATOM | 3428 | C | LEU | 318 | −35.107 | −4.213 | −22.342 |
| ATOM | 3429 | O | LEU | 318 | −34.692 | −3.527 | −21.424 |
| ATOM | 3430 | CB | LEU | 318 | −35.582 | −2.619 | −24.227 |
| ATOM | 3431 | CG | LEU | 318 | −34.908 | −1.777 | −25.353 |
| ATOM | 3432 | CD1 | LEU | 318 | −34.621 | −2.599 | −26.641 |
| ATOM | 3433 | CD2 | LEU | 318 | −35.802 | −0.556 | −25.697 |
| ATOM | 3434 | HA | LEU | 318 | −33.668 | −3.494 | −23.705 |
| ATOM | 3435 | H | LEU | 318 | −35.415 | −4.98 | −25.405 |
| ATOM | 3436 | HB1 | LEU | 318 | −36.575 | −2.979 | −24.54 |
| ATOM | 3437 | HB2 | LEU | 318 | −35.727 | −1.943 | −23.367 |
| ATOM | 3438 | HG | LEU | 318 | −33.945 | −1.387 | −24.979 |
| ATOM | 3439 | HD21 | LEU | 318 | −35.943 | 0.08 | −24.81 |
| ATOM | 3440 | HD22 | LEU | 318 | −35.335 | 0.058 | −26.483 |
| ATOM | 3441 | HD23 | LEU | 318 | −36.786 | −0.899 | −26.049 |
| ATOM | 3442 | HD11 | LEU | 318 | −35.549 | −3.054 | −27.021 |
| ATOM | 3443 | HD12 | LEU | 318 | −34.209 | −1.948 | −27.427 |
| ATOM | 3444 | HD13 | LEU | 318 | −33.88 | −3.389 | −26.449 |
| ATOM | 3445 | N | ARG | 319 | −35.868 | −5.309 | −22.137 |
| ATOM | 3446 | CA | ARG | 319 | −36.099 | −5.824 | −20.789 |
| ATOM | 3447 | C | ARG | 319 | −34.894 | −5.694 | −19.882 |
| ATOM | 3448 | O | ARG | 319 | −35.047 | −5.272 | −18.745 |
| ATOM | 3449 | CB | ARG | 319 | −36.546 | −7.314 | −20.853 |
| ATOM | 3450 | CG | ARG | 319 | −35.583 | −8.306 | −21.575 |
| ATOM | 3451 | CD | ARG | 319 | −36.277 | −9.675 | −21.843 |
| ATOM | 3452 | NE | ARG | 319 | −35.543 | −10.55 | −22.756 |
| ATOM | 3453 | CZ | ARG | 319 | −36.028 | −11.685 | −23.218 |
| ATOM | 3454 | NH1 | ARG | 319 | −37.186 | −12.176 | −22.843 |
| ATOM | 3455 | NH2 | ARG | 319 | −35.326 | −12.354 | −24.102 |
| ATOM | 3456 | HA | ARG | 319 | −36.916 | −5.229 | −20.347 |
| ATOM | 3457 | H | ARG | 319 | −36.293 | −5.783 | −22.905 |
| ATOM | 3458 | HB1 | ARG | 319 | −36.73 | −7.664 | −19.824 |
| ATOM | 3459 | HB2 | ARG | 319 | −37.494 | −7.316 | −21.41 |
| ATOM | 3460 | HG1 | ARG | 319 | −35.281 | −7.894 | −22.548 |
| ATOM | 3461 | HG2 | ARG | 319 | −34.677 | −8.479 | −20.974 |
| ATOM | 3462 | HD1 | ARG | 319 | −36.444 | −10.187 | −20.881 |
| ATOM | 3463 | HD2 | ARG | 319 | −37.235 | −9.457 | −22.337 |
| ATOM | 3464 | HE | ARG | 319 | −34.618 | −10.226 | −23.085 |
| ATOM | 3465 | HH12 | ARG | 319 | −37.546 | −13.051 | −23.251 |
| ATOM | 3466 | HH11 | ARG | 319 | −37.784 | −11.707 | −22.151 |
| ATOM | 3467 | HH22 | ARG | 319 | −35.666 | −13.245 | −24.492 |
| ATOM | 3468 | HH21 | ARG | 319 | −34.426 | −11.991 | −24.452 |
| ATOM | 3469 | N | THR | 320 | −33.68 | −6.042 | −20.358 |
| ATOM | 3470 | CA | THR | 320 | −32.505 | −5.919 | −19.496 |
| ATOM | 3471 | C | THR | 320 | −32.203 | −4.459 | −19.23 |
| ATOM | 3472 | O | THR | 320 | −31.839 | −4.102 | −18.119 |
| ATOM | 3473 | CB | THR | 320 | −31.285 | −6.602 | −20.175 |
| ATOM | 3474 | OG1 | THR | 320 | −31.694 | −7.934 | −20.537 |
| ATOM | 3475 | CG2 | THR | 320 | −30.052 | −6.663 | −19.233 |
| ATOM | 3476 | HA | THR | 320 | −32.72 | −6.444 | −18.553 |
| ATOM | 3477 | H | THR | 320 | −33.557 | −6.348 | −21.306 |
| ATOM | 3478 | HB | THR | 320 | −31.024 | −6.04 | −21.089 |
| ATOM | 3479 | HG1 | THR | 320 | −31.014 | −8.424 | −20.989 |
| ATOM | 3480 | HG23 | THR | 320 | −29.741 | −5.661 | −18.905 |
| ATOM | 3481 | HG21 | THR | 320 | −30.303 | −7.264 | −18.349 |
| ATOM | 3482 | HG22 | THR | 320 | −29.201 | −7.136 | −19.748 |
| ATOM | 3483 | N | LYS | 321 | −32.349 | −3.616 | −20.277 |
| ATOM | 3484 | CA | LYS | 321 | −32.026 | −2.191 | −20.182 |
| ATOM | 3485 | C | LYS | 321 | −33.029 | −1.457 | −19.312 |
| ATOM | 3486 | O | LYS | 321 | −32.638 | −0.559 | −18.581 |
| ATOM | 3487 | CB | LYS | 321 | −31.939 | −1.641 | −21.639 |
| ATOM | 3488 | CG | LYS | 321 | −31.273 | −0.248 | −21.783 |
| ATOM | 3489 | CD | LYS | 321 | −30.932 | 0.05 | −23.275 |
| ATOM | 3490 | CE | LYS | 321 | −30.535 | 1.534 | −23.507 |
| ATOM | 3491 | NZ | LYS | 321 | −29.917 | 1.78 | −24.832 |
| ATOM | 3492 | HA | LYS | 321 | −31.039 | −2.098 | −19.711 |
| ATOM | 3493 | H | LYS | 321 | −32.709 | −3.962 | −21.145 |
| ATOM | 3494 | HB1 | LYS | 321 | −31.338 | −2.347 | −22.233 |
| ATOM | 3495 | HB2 | LYS | 321 | −32.95 | −1.605 | −22.077 |
| ATOM | 3496 | HG1 | LYS | 321 | −31.968 | 0.499 | −21.386 |
| ATOM | 3497 | HG2 | LYS | 321 | −30.352 | −0.196 | −21.184 |
| ATOM | 3498 | HD1 | LYS | 321 | −30.099 | −0.602 | −23.583 |
| ATOM | 3499 | HD2 | LYS | 321 | −31.799 | −0.185 | −23.914 |
| ATOM | 3500 | HE1 | LYS | 321 | −31.456 | 2.133 | −23.444 |
| ATOM | 3501 | HE2 | LYS | 321 | −29.843 | 1.864 | −22.712 |
| ATOM | 3502 | HZ1 | LYS | 321 | −30.563 | 1.468 | −25.642 |
| ATOM | 3503 | HZ2 | LYS | 321 | −29.713 | 2.834 | −24.982 |
| ATOM | 3504 | HZ3 | LYS | 321 | −28.975 | 1.261 | −24.944 |
| ATOM | 3505 | N | ARG | 322 | −34.329 | −1.829 | −19.359 |
| ATOM | 3506 | CA | ARG | 322 | −35.317 | −1.202 | −18.474 |
| ATOM | 3507 | C | ARG | 322 | −34.89 | −1.402 | −17.037 |
| ATOM | 3508 | O | ARG | 322 | −34.783 | −0.443 | −16.29 |
| ATOM | 3509 | CB | ARG | 322 | −36.763 | −1.786 | −18.621 |
| ATOM | 3510 | CG | ARG | 322 | −37.833 | −0.699 | −18.923 |
| ATOM | 3511 | CD | ARG | 322 | −39.258 | −1.321 | −18.948 |
| ATOM | 3512 | NE | ARG | 322 | −40.286 | −0.35 | −19.338 |
| ATOM | 3513 | CZ | ARG | 322 | −41.521 | −0.685 | −19.647 |
| ATOM | 3514 | NH1 | ARG | 322 | −41.96 | −1.919 | −19.581 |
| ATOM | 3515 | NH2 | ARG | 322 | −42.354 | 0.246 | −20.045 |
| ATOM | 3516 | HA | ARG | 322 | −35.324 | −0.12 | −18.681 |
| ATOM | 3517 | H | ARG | 322 | −34.611 | −2.554 | −19.988 |
| ATOM | 3518 | HB1 | ARG | 322 | −36.805 | −2.535 | −19.426 |
| ATOM | 3519 | HB2 | ARG | 322 | −37.073 | −2.304 | −17.697 |
| ATOM | 3520 | HG1 | ARG | 322 | −37.797 | 0.091 | −18.154 |
| ATOM | 3521 | HG2 | ARG | 322 | −37.615 | −0.243 | −19.904 |
| ATOM | 3522 | HD1 | ARG | 322 | −39.214 | −2.155 | −19.667 |
| ATOM | 3523 | HD2 | ARG | 322 | −39.501 | −1.715 | −17.947 |
| ATOM | 3524 | HE | ARG | 322 | −40.004 | 0.641 | −19.401 |
| ATOM | 3525 | HH12 | ARG | 322 | −42.929 | −2.144 | −19.859 |
| ATOM | 3526 | HH11 | ARG | 322 | −41.374 | −2.692 | −19.251 |
| ATOM | 3527 | HH22 | ARG | 322 | −43.333 | 0.011 | −20.292 |
| ATOM | 3528 | HH21 | ARG | 322 | −42.064 | 1.231 | −20.125 |
| ATOM | 3529 | N | VAL | 323 | −34.658 | −2.669 | −16.63 |
| ATOM | 3530 | CA | VAL | 323 | −34.31 | −2.919 | −15.232 |
| ATOM | 3531 | C | VAL | 323 | −33.052 | −2.15 | −14.909 |
| ATOM | 3532 | O | VAL | 323 | −32.969 | −1.546 | −13.852 |
| ATOM | 3533 | CB | VAL | 323 | −34.127 | −4.432 | −14.926 |
| ATOM | 3534 | CG1 | VAL | 323 | −33.519 | −4.638 | −13.512 |
| ATOM | 3535 | CG2 | VAL | 323 | −35.474 | −5.202 | −15.034 |
| ATOM | 3536 | HA | VAL | 323 | −35.119 | −2.543 | −14.587 |
| ATOM | 3537 | H | VAL | 323 | −34.71 | −3.434 | −17.277 |
| ATOM | 3538 | HB | VAL | 323 | −33.43 | −4.844 | −15.673 |
| ATOM | 3539 | HG11 | VAL | 323 | −34.175 | −4.212 | −12.739 |
| ATOM | 3540 | HG12 | VAL | 323 | −33.405 | −5.711 | −13.324 |
| ATOM | 3541 | HG13 | VAL | 323 | −32.525 | −4.177 | −13.422 |
| ATOM | 3542 | HG21 | VAL | 323 | −35.927 | −5.086 | −16.029 |
| ATOM | 3543 | HG22 | VAL | 323 | −35.313 | −6.278 | −14.86 |
| ATOM | 3544 | HG23 | VAL | 323 | −36.188 | −4.832 | −14.283 |
| ATOM | 3545 | N | THR | 324 | −32.043 | −2.143 | −15.802 |
| ATOM | 3546 | CA | THR | 324 | −30.842 | −1.371 | −15.499 |
| ATOM | 3547 | C | THR | 324 | −31.18 | 0.079 | −15.226 |
| ATOM | 3548 | O | THR | 324 | −30.623 | 0.649 | −14.3 |
| ATOM | 3549 | CB | THR | 324 | −29.803 | −1.573 | −16.629 |
| ATOM | 3550 | OG1 | THR | 324 | −29.569 | −2.995 | −16.706 |
| ATOM | 3551 | CG2 | THR | 324 | −28.482 | −0.81 | −16.359 |
| ATOM | 3552 | HA | THR | 324 | −30.418 | −1.778 | −14.572 |
| ATOM | 3553 | H | THR | 324 | −32.102 | −2.654 | −16.666 |
| ATOM | 3554 | HB | THR | 324 | −30.243 | −1.197 | −17.568 |
| ATOM | 3555 | HG1 | THR | 324 | −29.009 | −3.232 | −17.438 |
| ATOM | 3556 | HG23 | THR | 324 | −28.66 | 0.275 | −16.324 |
| ATOM | 3557 | HG21 | THR | 324 | −28.037 | −1.129 | −15.405 |
| ATOM | 3558 | HG22 | THR | 324 | −27.771 | −1.021 | −17.172 |
| ATOM | 3559 | N | ARG | 325 | −32.102 | 0.704 | −15.991 |
| ATOM | 3560 | CA | ARG | 325 | −32.516 | 2.063 | −15.636 |
| ATOM | 3561 | C | ARG | 325 | −33.137 | 2.083 | −14.258 |
| ATOM | 3562 | O | ARG | 325 | −32.883 | 3.028 | −13.527 |
| ATOM | 3563 | CB | ARG | 325 | −33.52 | 2.658 | −16.661 |
| ATOM | 3564 | CG | ARG | 325 | −34.029 | 4.076 | −16.272 |
| ATOM | 3565 | CD | ARG | 325 | −34.859 | 4.707 | −17.427 |
| ATOM | 3566 | NE | ARG | 325 | −35.452 | 5.998 | −17.071 |
| ATOM | 3567 | CZ | ARG | 325 | −36.207 | 6.701 | −17.889 |
| ATOM | 3568 | NH1 | ARG | 325 | −36.505 | 6.306 | −19.106 |
| ATOM | 3569 | NH2 | ARG | 325 | −36.687 | 7.849 | −17.473 |
| ATOM | 3570 | HA | ARG | 325 | −31.619 | 2.706 | −15.625 |
| ATOM | 3571 | H | ARG | 325 | −32.541 | 0.236 | −16.763 |
| ATOM | 3572 | HB1 | ARG | 325 | −33.006 | 2.712 | −17.63 |
| ATOM | 3573 | HB2 | ARG | 325 | −34.387 | 1.988 | −16.769 |
| ATOM | 3574 | HG1 | ARG | 325 | −34.663 | 4.005 | −15.372 |
| ATOM | 3575 | HG2 | ARG | 325 | −33.173 | 4.732 | −16.042 |
| ATOM | 3576 | HD1 | ARG | 325 | −34.178 | 4.86 | −18.278 |
| ATOM | 3577 | HD2 | ARG | 325 | −35.655 | 3.994 | −17.698 |
| ATOM | 3578 | HE | ARG | 325 | −35.254 | 6.362 | −16.125 |
| ATOM | 3579 | HH12 | ARG | 325 | −37.099 | 6.882 | −19.719 |
| ATOM | 3580 | HH11 | ARG | 325 | −36.165 | 5.416 | −19.493 |
| ATOM | 3581 | HH22 | ARG | 325 | −37.282 | 8.431 | −18.08 |
| ATOM | 3582 | HH21 | ARG | 325 | −36.483 | 8.204 | −16.527 |
| ATOM | 3583 | N | THR | 326 | −33.957 | 1.08 | −13.87 |
| ATOM | 3584 | CA | THR | 326 | −34.552 | 1.126 | −12.534 |
| ATOM | 3585 | C | THR | 326 | −33.442 | 1.08 | −11.51 |
| ATOM | 3586 | O | THR | 326 | −33.4 | 1.93 | −10.636 |
| ATOM | 3587 | CB | THR | 326 | −35.568 | −0.006 | −12.198 |
| ATOM | 3588 | OG1 | THR | 326 | −34.89 | −1.271 | −12.099 |
| ATOM | 3589 | CG2 | THR | 326 | −36.718 | −0.098 | −13.238 |
| ATOM | 3590 | HA | THR | 326 | −35.085 | 2.087 | −12.44 |
| ATOM | 3591 | H | THR | 326 | −34.138 | 0.298 | −14.468 |
| ATOM | 3592 | HB | THR | 326 | −36.005 | 0.235 | −11.21 |
| ATOM | 3593 | HG1 | THR | 326 | −35.464 | −1.984 | −11.839 |
| ATOM | 3594 | HG23 | THR | 326 | −37.147 | 0.895 | −13.416 |
| ATOM | 3595 | HG21 | THR | 326 | −36.364 | −0.489 | −14.2 |
| ATOM | 3596 | HG22 | THR | 326 | −37.514 | −0.766 | −12.87 |
| ATOM | 3597 | N | ALA | 327 | −32.527 | 0.09 | −11.603 |
| ATOM | 3598 | CA | ALA | 327 | −31.43 | 0.015 | −10.639 |
| ATOM | 3599 | C | ALA | 327 | −30.728 | 1.35 | −10.518 |
| ATOM | 3600 | O | ALA | 327 | −30.489 | 1.807 | −9.41 |
| ATOM | 3601 | CB | ALA | 327 | −30.377 | −1.042 | −11.066 |
| ATOM | 3602 | HA | ALA | 327 | −31.843 | −0.264 | −9.656 |
| ATOM | 3603 | H | ALA | 327 | −32.607 | −0.606 | −12.323 |
| ATOM | 3604 | HB1 | ALA | 327 | −29.564 | −1.087 | −10.324 |
| ATOM | 3605 | HB2 | ALA | 327 | −30.84 | −2.037 | −11.148 |
| ATOM | 3606 | HB3 | ALA | 327 | −29.945 | −0.778 | −12.043 |
| ATOM | 3607 | N | ILE | 328 | −30.389 | 1.994 | −11.658 |
| ATOM | 3608 | CA | ILE | 328 | −29.706 | 3.287 | −11.581 |
| ATOM | 3609 | C | ILE | 328 | −30.634 | 4.27 | −10.902 |
| ATOM | 3610 | O | ILE | 328 | −30.197 | 4.987 | −10.015 |
| ATOM | 3611 | CB | ILE | 328 | −29.242 | 3.791 | −12.983 |
| ATOM | 3612 | CG1 | ILE | 328 | −28.12 | 2.859 | −13.545 |
| ATOM | 3613 | CG2 | ILE | 328 | −28.759 | 5.269 | −12.901 |
| ATOM | 3614 | CD1 | ILE | 328 | −27.842 | 3.054 | −15.061 |
| ATOM | 3615 | HA | ILE | 328 | −28.809 | 3.176 | −10.95 |
| ATOM | 3616 | H | ILE | 328 | −30.638 | 1.615 | −12.554 |
| ATOM | 3617 | HB | ILE | 328 | −30.113 | 3.759 | −13.658 |
| ATOM | 3618 | HG11 | ILE | 328 | −27.187 | 3.021 | −12.982 |
| ATOM | 3619 | HG12 | ILE | 328 | −28.405 | 1.804 | −13.408 |
| ATOM | 3620 | HD11 | ILE | 328 | −27.423 | 4.048 | −15.273 |
| ATOM | 3621 | HD12 | ILE | 328 | −27.114 | 2.302 | −15.408 |
| ATOM | 3622 | HD13 | ILE | 328 | −28.77 | 2.929 | −15.64 |
| ATOM | 3623 | HG21 | ILE | 328 | −27.924 | 5.359 | −12.188 |
| ATOM | 3624 | HG22 | ILE | 328 | −28.424 | 5.64 | −13.88 |
| ATOM | 3625 | HG23 | ILE | 328 | −29.572 | 5.933 | −12.57 |
| ATOM | 3626 | N | ALA | 329 | −31.927 | 4.328 | −11.288 |
| ATOM | 3627 | CA | ALA | 329 | −32.831 | 5.274 | −10.637 |
| ATOM | 3628 | C | ALA | 329 | −32.82 | 5.074 | −9.138 |
| ATOM | 3629 | O | ALA | 329 | −32.783 | 6.059 | −8.416 |
| ATOM | 3630 | CB | ALA | 329 | −34.289 | 5.136 | −11.149 |
| ATOM | 3631 | HA | ALA | 329 | −32.477 | 6.294 | −10.864 |
| ATOM | 3632 | H | ALA | 329 | −32.28 | 3.732 | −12.014 |
| ATOM | 3633 | HB1 | ALA | 329 | −34.929 | 5.898 | −10.677 |
| ATOM | 3634 | HB2 | ALA | 329 | −34.324 | 5.273 | −12.241 |
| ATOM | 3635 | HB3 | ALA | 329 | −34.694 | 4.144 | −10.904 |
| ATOM | 3636 | N | ILE | 330 | −32.844 | 3.817 | −8.637 |
| ATOM | 3637 | CA | ILE | 330 | −32.819 | 3.632 | −7.186 |
| ATOM | 3638 | C | ILE | 330 | −31.492 | 4.173 | −6.695 |
| ATOM | 3639 | O | ILE | 330 | −31.489 | 4.888 | −5.706 |
| ATOM | 3640 | CB | ILE | 330 | −33.052 | 2.176 | −6.668 |
| ATOM | 3641 | CG1 | ILE | 330 | −34.386 | 1.577 | −7.217 |
| ATOM | 3642 | CG2 | ILE | 330 | −33.084 | 2.163 | −5.108 |
| ATOM | 3643 | CD1 | ILE | 330 | −34.658 | 0.12 | −6.752 |
| ATOM | 3644 | HA | ILE | 330 | −33.631 | 4.245 | −6.762 |
| ATOM | 3645 | H | ILE | 330 | −32.821 | 3.02 | −9.246 |
| ATOM | 3646 | HB | ILE | 330 | −32.208 | 1.559 | −7.019 |
| ATOM | 3647 | HG11 | ILE | 330 | −35.231 | 2.21 | −6.901 |
| ATOM | 3648 | HG12 | ILE | 330 | −34.37 | 1.561 | −8.316 |
| ATOM | 3649 | HD11 | ILE | 330 | −34.907 | 0.076 | −5.681 |
| ATOM | 3650 | HD12 | ILE | 330 | −35.513 | −0.298 | −7.308 |
| ATOM | 3651 | HD13 | ILE | 330 | −33.777 | −0.511 | −6.941 |
| ATOM | 3652 | HG21 | ILE | 330 | −34.016 | 2.618 | −4.739 |
| ATOM | 3653 | HG22 | ILE | 330 | −33.018 | 1.135 | −4.721 |
| ATOM | 3654 | HG23 | ILE | 330 | −32.25 | 2.724 | −4.668 |
| ATOM | 3655 | N | CYS | 331 | −30.351 | 3.871 | −7.36 |
| ATOM | 3656 | CA | CYS | 331 | −29.078 | 4.421 | −6.888 |
| ATOM | 3657 | C | CYS | 331 | −29.175 | 5.92 | −6.709 |
| ATOM | 3658 | O | CYS | 331 | −28.721 | 6.439 | −5.7 |
| ATOM | 3659 | CB | CYS | 331 | −27.875 | 4.161 | −7.838 |
| ATOM | 3660 | SG | CYS | 331 | −27.599 | 2.381 | −8.118 |
| ATOM | 3661 | HA | CYS | 331 | −28.865 | 3.967 | −5.911 |
| ATOM | 3662 | H | CYS | 331 | −30.358 | 3.276 | −8.168 |
| ATOM | 3663 | HB1 | CYS | 331 | −28.034 | 4.653 | −8.809 |
| ATOM | 3664 | HB2 | CYS | 331 | −26.961 | 4.576 | −7.387 |
| ATOM | 3665 | HG | CYS | 331 | −26.81 | 2.447 | −8.743 |
| ATOM | 3666 | LPG1 | CYS | 331 | −27.443 | 2.053 | −7.544 |
| ATOM | 3667 | LPG2 | CYS | 331 | −28.133 | 2.083 | −8.411 |
| ATOM | 3668 | N | LEU | 332 | −29.767 | 6.636 | −7.69 |
| ATOM | 3669 | CA | LEU | 332 | −29.861 | 8.089 | −7.564 |
| ATOM | 3670 | C | LEU | 332 | −30.753 | 8.44 | −6.394 |
| ATOM | 3671 | O | LEU | 332 | −30.376 | 9.276 | −5.587 |
| ATOM | 3672 | CB | LEU | 332 | −30.404 | 8.777 | −8.849 |
| ATOM | 3673 | CG | LEU | 332 | −29.567 | 8.519 | −10.141 |
| ATOM | 3674 | CD1 | LEU | 332 | −30.204 | 9.282 | −11.336 |
| ATOM | 3675 | CD2 | LEU | 332 | −28.075 | 8.927 | −9.982 |
| ATOM | 3676 | HA | LEU | 332 | −28.864 | 8.496 | −7.338 |
| ATOM | 3677 | H | LEU | 332 | −30.169 | 6.178 | −8.487 |
| ATOM | 3678 | HB1 | LEU | 332 | −31.434 | 8.424 | −9.021 |
| ATOM | 3679 | HB2 | LEU | 332 | −30.447 | 9.863 | −8.664 |
| ATOM | 3680 | HG | LEU | 332 | −29.592 | 7.448 | −10.397 |
| ATOM | 3681 | HD21 | LEU | 332 | −27.555 | 8.261 | −9.278 |
| ATOM | 3682 | HD22 | LEU | 332 | −27.552 | 8.855 | −10.948 |
| ATOM | 3683 | HD23 | LEU | 332 | −27.997 | 9.964 | −9.619 |
| ATOM | 3684 | HD11 | LEU | 332 | −30.173 | 10.368 | −11.159 |
| ATOM | 3685 | HD12 | LEU | 332 | −29.662 | 9.064 | −12.27 |
| ATOM | 3686 | HD13 | LEU | 332 | −31.254 | 8.978 | −11.473 |
| ATOM | 3687 | N | VAL | 333 | −31.943 | 7.811 | −6.275 |
| ATOM | 3688 | CA | VAL | 333 | −32.82 | 8.138 | −5.15 |
| ATOM | 3689 | C | VAL | 333 | −32.073 | 7.901 | −3.857 |
| ATOM | 3690 | O | VAL | 333 | −32.135 | 8.734 | −2.965 |
| ATOM | 3691 | CB | VAL | 333 | −34.142 | 7.311 | −5.163 |
| ATOM | 3692 | CG1 | VAL | 333 | −34.924 | 7.469 | −3.829 |
| ATOM | 3693 | CG2 | VAL | 333 | −35.053 | 7.74 | −6.349 |
| ATOM | 3694 | HA | VAL | 333 | −33.077 | 9.209 | −5.204 |
| ATOM | 3695 | H | VAL | 333 | −32.217 | 7.103 | −6.932 |
| ATOM | 3696 | HB | VAL | 333 | −33.887 | 6.244 | −5.273 |
| ATOM | 3697 | HG11 | VAL | 333 | −35.126 | 8.534 | −3.635 |
| ATOM | 3698 | HG12 | VAL | 333 | −35.882 | 6.932 | −3.887 |
| ATOM | 3699 | HG13 | VAL | 333 | −34.36 | 7.056 | −2.979 |
| ATOM | 3700 | HG21 | VAL | 333 | −34.539 | 7.634 | −7.315 |
| ATOM | 3701 | HG22 | VAL | 333 | −35.96 | 7.118 | −6.379 |
| ATOM | 3702 | HG23 | VAL | 333 | −35.358 | 8.791 | −6.235 |
| ATOM | 3703 | N | PHE | 334 | −31.361 | 6.763 | −3.732 |
| ATOM | 3704 | CA | PHE | 334 | −30.632 | 6.493 | −2.496 |
| ATOM | 3705 | C | PHE | 334 | −29.634 | 7.598 | −2.234 |
| ATOM | 3706 | O | PHE | 334 | −29.608 | 8.126 | −1.133 |
| ATOM | 3707 | CB | PHE | 334 | −29.903 | 5.127 | −2.575 |
| ATOM | 3708 | CG | PHE | 334 | −29.133 | 4.862 | −1.274 |
| ATOM | 3709 | CD1 | PHE | 334 | −29.697 | 4.104 | −0.24 |
| ATOM | 3710 | CD2 | PHE | 334 | −27.852 | 5.397 | −1.108 |
| ATOM | 3711 | CE1 | PHE | 334 | −29.052 | 4.02 | 0.998 |
| ATOM | 3712 | CE2 | PHE | 334 | −27.204 | 5.311 | 0.127 |
| ATOM | 3713 | CZ | PHE | 334 | −27.835 | 4.677 | 1.199 |
| ATOM | 3714 | HA | PHE | 334 | −31.355 | 6.451 | −1.663 |
| ATOM | 3715 | H | PHE | 334 | −31.322 | 6.098 | −4.481 |
| ATOM | 3716 | HB1 | PHE | 334 | −30.641 | 4.329 | −2.757 |
| ATOM | 3717 | HB2 | PHE | 334 | −29.204 | 5.145 | −3.425 |
| ATOM | 3718 | HD2 | PHE | 334 | −27.356 | 5.888 | −1.937 |
| ATOM | 3719 | HE2 | PHE | 334 | −26.216 | 5.74 | 0.251 |
| ATOM | 3720 | HZ | PHE | 334 | −27.38 | 4.698 | 2.184 |
| ATOM | 3721 | HE1 | PHE | 334 | −29.503 | 3.456 | 1.807 |
| ATOM | 3722 | HD1 | PHE | 334 | −30.64 | 3.589 | −0.389 |
| ATOM | 3723 | N | PHE | 335 | −28.796 | 7.972 | −3.229 |
| ATOM | 3724 | CA | PHE | 335 | −27.827 | 9.038 | −2.98 |
| ATOM | 3725 | C | PHE | 335 | −28.551 | 10.297 | −2.555 |
| ATOM | 3726 | O | PHE | 335 | −28.109 | 10.947 | −1.621 |
| ATOM | 3727 | CB | PHE | 335 | −26.956 | 9.375 | −4.223 |
| ATOM | 3728 | CG | PHE | 335 | −26.199 | 8.182 | −4.839 |
| ATOM | 3729 | CD1 | PHE | 335 | −26.069 | 8.094 | −6.23 |
| ATOM | 3730 | CD2 | PHE | 335 | −25.623 | 7.178 | −4.049 |
| ATOM | 3731 | CE1 | PHE | 335 | −25.449 | 6.993 | −6.826 |
| ATOM | 3732 | CE2 | PHE | 335 | −25.043 | 6.05 | −4.64 |
| ATOM | 3733 | CZ | PHE | 335 | −24.934 | 5.967 | −6.03 |
| ATOM | 3734 | HA | PHE | 335 | −27.181 | 8.737 | −2.139 |
| ATOM | 3735 | H | PHE | 335 | −28.832 | 7.544 | −4.138 |
| ATOM | 3736 | HB1 | PHE | 335 | −27.625 | 9.808 | −4.983 |
| ATOM | 3737 | HB2 | PHE | 335 | −26.209 | 10.138 | −3.952 |
| ATOM | 3738 | HD2 | PHE | 335 | −25.606 | 7.261 | −2.971 |
| ATOM | 3739 | HE2 | PHE | 335 | −24.673 | 5.24 | −4.02 |
| ATOM | 3740 | HZ | PHE | 335 | −24.448 | 5.112 | −6.488 |
| ATOM | 3741 | HE1 | PHE | 335 | −25.366 | 6.934 | −7.907 |
| ATOM | 3742 | HD1 | PHE | 335 | −26.445 | 8.89 | −6.865 |
| ATOM | 3743 | N | VAL | 336 | −29.666 | 10.67 | −3.219 |
| ATOM | 3744 | CA | VAL | 336 | −30.373 | 11.888 | −2.818 |
| ATOM | 3745 | C | VAL | 336 | −30.892 | 11.739 | −1.404 |
| ATOM | 3746 | O | VAL | 336 | −30.784 | 12.674 | −0.627 |
| ATOM | 3747 | CB | VAL | 336 | −31.547 | 12.226 | −3.786 |
| ATOM | 3748 | CG1 | VAL | 336 | −32.449 | 13.362 | −3.222 |
| ATOM | 3749 | CG2 | VAL | 336 | −31.005 | 12.641 | −5.184 |
| ATOM | 3750 | HA | VAL | 336 | −29.663 | 12.731 | −2.828 |
| ATOM | 3751 | H | VAL | 336 | −30.031 | 10.104 | −3.965 |
| ATOM | 3752 | HB | VAL | 336 | −32.177 | 11.328 | −3.901 |
| ATOM | 3753 | HG11 | VAL | 336 | −31.846 | 14.259 | −3.012 |
| ATOM | 3754 | HG12 | VAL | 336 | −33.231 | 13.629 | −3.951 |
| ATOM | 3755 | HG13 | VAL | 336 | −32.953 | 13.051 | −2.294 |
| ATOM | 3756 | HG21 | VAL | 336 | −30.348 | 11.871 | −5.61 |
| ATOM | 3757 | HG22 | VAL | 336 | −31.839 | 12.801 | −5.885 |
| ATOM | 3758 | HG23 | VAL | 336 | −30.432 | 13.578 | −5.108 |
| ATOM | 3759 | N | CYS | 337 | −31.48 | 10.579 | −1.045 |
| ATOM | 3760 | CA | CYS | 337 | −32.057 | 10.444 | 0.292 |
| ATOM | 3761 | C | CYS | 337 | −30.991 | 10.482 | 1.366 |
| ATOM | 3762 | O | CYS | 337 | −31.207 | 11.122 | 2.384 |
| ATOM | 3763 | CB | CYS | 337 | −32.861 | 9.122 | 0.408 |
| ATOM | 3764 | SG | CYS | 337 | −33.699 | 9.051 | 2.027 |
| ATOM | 3765 | HA | CYS | 337 | −32.761 | 11.278 | 0.455 |
| ATOM | 3766 | H | CYS | 337 | −31.54 | 9.811 | −1.688 |
| ATOM | 3767 | HB1 | CYS | 337 | −33.622 | 9.079 | −0.388 |
| ATOM | 3768 | HB2 | CYS | 337 | −32.19 | 8.255 | 0.303 |
| ATOM | 3769 | HG | CYS | 337 | −34.217 | 8.199 | 1.881 |
| ATOM | 3770 | LPG1 | CYS | 337 | −33.26 | 9 | 2.542 |
| ATOM | 3771 | LPG2 | CYS | 337 | −34.106 | 9.585 | 2.129 |
| ATOM | 3772 | N | TRP | 338 | −29.845 | 9.788 | 1.172 |
| ATOM | 3773 | CA | TRP | 338 | −28.853 | 9.657 | 2.247 |
| ATOM | 3774 | C | TRP | 338 | −27.654 | 10.573 | 2.147 |
| ATOM | 3775 | O | TRP | 338 | −27.08 | 10.867 | 3.186 |
| ATOM | 3776 | CB | TRP | 338 | −28.351 | 8.194 | 2.264 |
| ATOM | 3777 | CG | TRP | 338 | −29.496 | 7.31 | 2.671 |
| ATOM | 3778 | CD1 | TRP | 338 | −30.281 | 6.56 | 1.885 |
| ATOM | 3779 | CD2 | TRP | 338 | −29.974 | 7.115 | 4.086 |
| ATOM | 3780 | NE1 | TRP | 338 | −31.14 | 5.904 | 2.623 |
| ATOM | 3781 | CE2 | TRP | 338 | −30.989 | 6.189 | 3.933 |
| ATOM | 3782 | CE3 | TRP | 338 | −29.614 | 7.635 | 5.327 |
| ATOM | 3783 | CZ2 | TRP | 338 | −31.684 | 5.689 | 5.032 |
| ATOM | 3784 | CZ3 | TRP | 338 | −30.329 | 7.165 | 6.435 |
| ATOM | 3785 | CH2 | TRP | 338 | −31.352 | 6.215 | 6.287 |
| ATOM | 3786 | HA | TRP | 338 | −29.316 | 9.855 | 3.229 |
| ATOM | 3787 | H | TRP | 338 | −29.684 | 9.316 | 0.3 |
| ATOM | 3788 | HB1 | TRP | 338 | −27.958 | 7.908 | 1.276 |
| ATOM | 3789 | HB2 | TRP | 338 | −27.542 | 8.074 | 2.999 |
| ATOM | 3790 | HE1 | TRP | 338 | −31.843 | 5.246 | 2.249 |
| ATOM | 3791 | HD1 | TRP | 338 | −30.201 | 6.512 | 0.801 |
| ATOM | 3792 | HZ2 | TRP | 338 | −32.443 | 4.923 | 4.922 |
| ATOM | 3793 | HH2 | TRP | 338 | −31.895 | 5.875 | 7.163 |
| ATOM | 3794 | HZ3 | TRP | 338 | −30.086 | 7.539 | 7.424 |
| ATOM | 3795 | HE3 | TRP | 338 | −28.82 | 8.367 | 5.426 |
| ATOM | 3796 | N | ALA | 339 | −27.234 | 11.04 | 0.949 |
| ATOM | 3797 | CA | ALA | 339 | −26.059 | 11.908 | 0.891 |
| ATOM | 3798 | C | ALA | 339 | −26.173 | 13.063 | 1.865 |
| ATOM | 3799 | O | ALA | 339 | −25.174 | 13.334 | 2.515 |
| ATOM | 3800 | CB | ALA | 339 | −25.691 | 12.405 | −0.533 |
| ATOM | 3801 | HA | ALA | 339 | −25.203 | 11.294 | 1.213 |
| ATOM | 3802 | H | ALA | 339 | −27.698 | 10.799 | 0.094 |
| ATOM | 3803 | HB1 | ALA | 339 | −24.759 | 12.992 | −0.492 |
| ATOM | 3804 | HB2 | ALA | 339 | −25.529 | 11.545 | −1.201 |
| ATOM | 3805 | HB3 | ALA | 339 | −26.473 | 13.04 | −0.968 |
| ATOM | 3806 | N | PRO | 340 | −27.318 | 13.773 | 2.04 |
| ATOM | 3807 | CA | PRO | 340 | −27.347 | 14.843 | 3.025 |
| ATOM | 3808 | C | PRO | 340 | −26.896 | 14.389 | 4.393 |
| ATOM | 3809 | O | PRO | 340 | −26.103 | 15.086 | 5.007 |
| ATOM | 3810 | CB | PRO | 340 | −28.85 | 15.221 | 2.995 |
| ATOM | 3811 | CG | PRO | 340 | −29.273 | 14.861 | 1.552 |
| ATOM | 3812 | CD | PRO | 340 | −28.548 | 13.519 | 1.3 |
| ATOM | 3813 | HA | PRO | 340 | −26.728 | 15.679 | 2.659 |
| ATOM | 3814 | HD2 | PRO | 340 | −28.427 | 13.331 | 0.225 |
| ATOM | 3815 | HD1 | PRO | 340 | −29.121 | 12.704 | 1.766 |
| ATOM | 3816 | HG2 | PRO | 340 | −28.88 | 15.625 | 0.861 |
| ATOM | 3817 | HG1 | PRO | 340 | −30.366 | 14.79 | 1.421 |
| ATOM | 3818 | HB1 | PRO | 340 | −29.423 | 14.588 | 3.693 |
| ATOM | 3819 | HB2 | PRO | 340 | −29.03 | 16.278 | 3.249 |
| ATOM | 3820 | N | TYR | 341 | −27.377 | 13.231 | 4.903 |
| ATOM | 3821 | CA | TYR | 341 | −26.961 | 12.836 | 6.244 |
| ATOM | 3822 | C | TYR | 341 | −25.511 | 12.418 | 6.225 |
| ATOM | 3823 | O | TYR | 341 | −24.817 | 12.75 | 7.171 |
| ATOM | 3824 | CB | TYR | 341 | −27.803 | 11.72 | 6.921 |
| ATOM | 3825 | CG | TYR | 341 | −27.276 | 11.54 | 8.357 |
| ATOM | 3826 | CD1 | TYR | 341 | −27.327 | 12.608 | 9.26 |
| ATOM | 3827 | CD2 | TYR | 341 | −26.71 | 10.333 | 8.781 |
| ATOM | 3828 | CE1 | TYR | 341 | −26.538 | 12.592 | 10.414 |
| ATOM | 3829 | CE2 | TYR | 341 | −26.004 | 10.283 | 9.988 |
| ATOM | 3830 | CZ | TYR | 341 | −25.798 | 11.448 | 10.732 |
| ATOM | 3831 | OH | TYR | 341 | −24.861 | 11.448 | 11.769 |
| ATOM | 3832 | HA | TYR | 341 | −27.046 | 13.737 | 6.873 |
| ATOM | 3833 | H | TYR | 341 | −27.979 | 12.625 | 4.379 |
| ATOM | 3834 | HB2 | TYR | 341 | −28.867 | 11.998 | 6.954 |
| ATOM | 3835 | HB1 | TYR | 341 | −27.708 | 10.793 | 6.334 |
| ATOM | 3836 | HD2 | TYR | 341 | −26.805 | 9.436 | 8.177 |
| ATOM | 3837 | HE2 | TYR | 341 | −25.606 | 9.342 | 10.348 |
| ATOM | 3838 | HE1 | TYR | 341 | −26.501 | 13.469 | 11.051 |
| ATOM | 3839 | HD1 | TYR | 341 | −27.968 | 13.462 | 9.068 |
| ATOM | 3840 | HH | TYR | 341 | −24.808 | 12.263 | 12.262 |
| ATOM | 3841 | N | TYR | 342 | −25.007 | 11.705 | 5.191 |
| ATOM | 3842 | CA | TYR | 342 | −23.573 | 11.408 | 5.184 |
| ATOM | 3843 | C | TYR | 342 | −22.788 | 12.702 | 5.252 |
| ATOM | 3844 | O | TYR | 342 | −21.801 | 12.76 | 5.968 |
| ATOM | 3845 | CB | TYR | 342 | −23.08 | 10.622 | 3.939 |
| ATOM | 3846 | CG | TYR | 342 | −23.456 | 9.132 | 3.991 |
| ATOM | 3847 | CD1 | TYR | 342 | −24.364 | 8.577 | 3.083 |
| ATOM | 3848 | CD2 | TYR | 342 | −22.865 | 8.3 | 4.95 |
| ATOM | 3849 | CE1 | TYR | 342 | −24.629 | 7.204 | 3.09 |
| ATOM | 3850 | CE2 | TYR | 342 | −23.138 | 6.929 | 4.966 |
| ATOM | 3851 | CZ | TYR | 342 | −24.022 | 6.373 | 4.039 |
| ATOM | 3852 | OH | TYR | 342 | −24.289 | 5.001 | 4.082 |
| ATOM | 3853 | HA | TYR | 342 | −23.344 | 10.82 | 6.086 |
| ATOM | 3854 | H | TYR | 342 | −25.58 | 11.409 | 4.421 |
| ATOM | 3855 | HB2 | TYR | 342 | −23.454 | 11.109 | 3.026 |
| ATOM | 3856 | HB1 | TYR | 342 | −21.979 | 10.661 | 3.904 |
| ATOM | 3857 | HD2 | TYR | 342 | −22.185 | 8.714 | 5.687 |
| ATOM | 3858 | HE2 | TYR | 342 | −22.661 | 6.29 | 5.701 |
| ATOM | 3859 | HE1 | TYR | 342 | −25.31 | 6.792 | 2.355 |
| ATOM | 3860 | HD1 | TYR | 342 | −24.87 | 9.205 | 2.36 |
| ATOM | 3861 | HH | TYR | 342 | −24.843 | 4.677 | 3.38 |
| ATOM | 3862 | N | VAL | 343 | −23.189 | 13.765 | 4.522 |
| ATOM | 3863 | CA | VAL | 343 | −22.43 | 15.013 | 4.618 |
| ATOM | 3864 | C | VAL | 343 | −22.476 | 15.502 | 6.049 |
| ATOM | 3865 | O | VAL | 343 | −21.431 | 15.755 | 6.629 |
| ATOM | 3866 | CB | VAL | 343 | −22.945 | 16.108 | 3.635 |
| ATOM | 3867 | CG1 | VAL | 343 | −22.311 | 17.494 | 3.946 |
| ATOM | 3868 | CG2 | VAL | 343 | −22.638 | 15.71 | 2.162 |
| ATOM | 3869 | HA | VAL | 343 | −21.376 | 14.805 | 4.368 |
| ATOM | 3870 | H | VAL | 343 | −24.008 | 13.718 | 3.944 |
| ATOM | 3871 | HB | VAL | 343 | −24.038 | 16.206 | 3.751 |
| ATOM | 3872 | HG11 | VAL | 343 | −21.212 | 17.427 | 3.916 |
| ATOM | 3873 | HG12 | VAL | 343 | −22.637 | 18.239 | 3.204 |
| ATOM | 3874 | HG13 | VAL | 343 | −22.613 | 17.857 | 4.94 |
| ATOM | 3875 | HG21 | VAL | 343 | −23.06 | 14.725 | 1.916 |
| ATOM | 3876 | HG22 | VAL | 343 | −23.07 | 16.448 | 1.469 |
| ATOM | 3877 | HG23 | VAL | 343 | −21.551 | 15.669 | 1.995 |
| ATOM | 3878 | N | LEU | 344 | −23.68 | 15.648 | 6.646 |
| ATOM | 3879 | CA | LEU | 344 | −23.744 | 16.182 | 8.007 |
| ATOM | 3880 | C | LEU | 344 | −22.959 | 15.3 | 8.957 |
| ATOM | 3881 | O | LEU | 344 | −22.234 | 15.817 | 9.796 |
| ATOM | 3882 | CB | LEU | 344 | −25.193 | 16.299 | 8.55 |
| ATOM | 3883 | CG | LEU | 344 | −26.122 | 17.292 | 7.787 |
| ATOM | 3884 | CD1 | LEU | 344 | −27.535 | 17.236 | 8.426 |
| ATOM | 3885 | CD2 | LEU | 344 | −25.611 | 18.76 | 7.815 |
| ATOM | 3886 | HA | LEU | 344 | −23.28 | 17.178 | 8.003 |
| ATOM | 3887 | H | LEU | 344 | −24.523 | 15.39 | 6.167 |
| ATOM | 3888 | HB1 | LEU | 344 | −25.634 | 15.29 | 8.53 |
| ATOM | 3889 | HB2 | LEU | 344 | −25.149 | 16.625 | 9.598 |
| ATOM | 3890 | HG | LEU | 344 | −26.214 | 16.98 | 6.735 |
| ATOM | 3891 | HD21 | LEU | 344 | −24.675 | 18.878 | 7.251 |
| ATOM | 3892 | HD22 | LEU | 344 | −26.355 | 19.428 | 7.354 |
| ATOM | 3893 | HD23 | LEU | 344 | −25.45 | 19.086 | 8.855 |
| ATOM | 3894 | HD11 | LEU | 344 | −27.526 | 17.691 | 9.428 |
| ATOM | 3895 | HD12 | LEU | 344 | −28.262 | 17.77 | 7.798 |
| ATOM | 3896 | HD13 | LEU | 344 | −27.863 | 16.192 | 8.52 |
| ATOM | 3897 | N | GLN | 345 | −23.099 | 13.962 | 8.834 |
| ATOM | 3898 | CA | GLN | 345 | −22.324 | 13.039 | 9.664 |
| ATOM | 3899 | C | GLN | 345 | −20.859 | 13.403 | 9.588 |
| ATOM | 3900 | O | GLN | 345 | −20.206 | 13.525 | 10.614 |
| ATOM | 3901 | CB | GLN | 345 | −22.485 | 11.586 | 9.125 |
| ATOM | 3902 | CG | GLN | 345 | −21.648 | 10.519 | 9.877 |
| ATOM | 3903 | CD | GLN | 345 | −21.554 | 9.274 | 9.021 |
| ATOM | 3904 | OE1 | GLN | 345 | −20.548 | 9.081 | 8.356 |
| ATOM | 3905 | NE2 | GLN | 345 | −22.586 | 8.408 | 8.991 |
| ATOM | 3906 | HA | GLN | 345 | −22.677 | 13.092 | 10.707 |
| ATOM | 3907 | H | GLN | 345 | −23.726 | 13.586 | 8.152 |
| ATOM | 3908 | HB1 | GLN | 345 | −23.546 | 11.294 | 9.141 |
| ATOM | 3909 | HB2 | GLN | 345 | −22.154 | 11.568 | 8.079 |
| ATOM | 3910 | HG1 | GLN | 345 | −20.624 | 10.875 | 10.069 |
| ATOM | 3911 | HG2 | GLN | 345 | −22.118 | 10.305 | 10.848 |
| ATOM | 3912 | HE22 | GLN | 345 | −22.525 | 7.593 | 8.416 |
| ATOM | 3913 | HE21 | GLN | 345 | −23.402 | 8.556 | 9.545 |
| ATOM | 3914 | N | LEU | 346 | −20.322 | 13.559 | 8.359 |
| ATOM | 3915 | CA | LEU | 346 | −18.885 | 13.776 | 8.223 |
| ATOM | 3916 | C | LEU | 346 | −18.508 | 15.143 | 8.745 |
| ATOM | 3917 | O | LEU | 346 | −17.413 | 15.27 | 9.27 |
| ATOM | 3918 | CB | LEU | 346 | −18.414 | 13.572 | 6.757 |
| ATOM | 3919 | CG | LEU | 346 | −18.583 | 12.101 | 6.256 |
| ATOM | 3920 | CD1 | LEU | 346 | −18.507 | 12.061 | 4.705 |
| ATOM | 3921 | CD2 | LEU | 346 | −17.522 | 11.134 | 6.857 |
| ATOM | 3922 | HA | LEU | 346 | −18.364 | 13.045 | 8.859 |
| ATOM | 3923 | H | LEU | 346 | −20.892 | 13.539 | 7.531 |
| ATOM | 3924 | HB1 | LEU | 346 | −19.004 | 14.261 | 6.131 |
| ATOM | 3925 | HB2 | LEU | 346 | −17.356 | 13.863 | 6.665 |
| ATOM | 3926 | HG | LEU | 346 | −19.575 | 11.716 | 6.547 |
| ATOM | 3927 | HD21 | LEU | 346 | −17.615 | 11.045 | 7.949 |
| ATOM | 3928 | HD22 | LEU | 346 | −17.647 | 10.124 | 6.436 |
| ATOM | 3929 | HD23 | LEU | 346 | −16.505 | 11.482 | 6.62 |
| ATOM | 3930 | HD11 | LEU | 346 | −17.532 | 12.437 | 4.359 |
| ATOM | 3931 | HD12 | LEU | 346 | −18.636 | 11.032 | 4.339 |
| ATOM | 3932 | HD13 | LEU | 346 | −19.303 | 12.68 | 4.26 |
| ATOM | 3933 | N | THR | 347 | −19.367 | 16.184 | 8.65 |
| ATOM | 3934 | CA | THR | 347 | −18.992 | 17.449 | 9.285 |
| ATOM | 3935 | C | THR | 347 | −18.784 | 17.187 | 10.759 |
| ATOM | 3936 | O | THR | 347 | −17.807 | 17.673 | 11.305 |
| ATOM | 3937 | CB | THR | 347 | −19.992 | 18.639 | 9.17 |
| ATOM | 3938 | OG1 | THR | 347 | −21.177 | 18.435 | 9.953 |
| ATOM | 3939 | CG2 | THR | 347 | −20.347 | 18.995 | 7.702 |
| ATOM | 3940 | HA | THR | 347 | −18.037 | 17.776 | 8.839 |
| ATOM | 3941 | H | THR | 347 | −20.258 | 16.091 | 8.2 |
| ATOM | 3942 | HB | THR | 347 | −19.507 | 19.524 | 9.619 |
| ATOM | 3943 | HG1 | THR | 347 | −21.667 | 17.672 | 9.673 |
| ATOM | 3944 | HG23 | THR | 347 | −19.433 | 19.185 | 7.12 |
| ATOM | 3945 | HG21 | THR | 347 | −20.912 | 18.187 | 7.218 |
| ATOM | 3946 | HG22 | THR | 347 | −20.971 | 19.902 | 7.684 |
| ATOM | 3947 | N | GLN | 348 | −19.676 | 16.422 | 11.432 |
| ATOM | 3948 | CA | GLN | 348 | −19.492 | 16.21 | 12.867 |
| ATOM | 3949 | C | GLN | 348 | −18.185 | 15.487 | 13.104 |
| ATOM | 3950 | O | GLN | 348 | −17.487 | 15.836 | 14.042 |
| ATOM | 3951 | CB | GLN | 348 | −20.673 | 15.439 | 13.526 |
| ATOM | 3952 | CG | GLN | 348 | −20.609 | 15.516 | 15.077 |
| ATOM | 3953 | CD | GLN | 348 | −21.761 | 14.852 | 15.787 |
| ATOM | 3954 | OE1 | GLN | 348 | −21.524 | 13.933 | 16.555 |
| ATOM | 3955 | NE2 | GLN | 348 | −23.018 | 15.297 | 15.574 |
| ATOM | 3956 | HA | GLN | 348 | −19.431 | 17.209 | 13.33 |
| ATOM | 3957 | H | GLN | 348 | −20.453 | 15.989 | 10.968 |
| ATOM | 3958 | HB1 | GLN | 348 | −21.611 | 15.902 | 13.19 |
| ATOM | 3959 | HB2 | GLN | 348 | −20.672 | 14.388 | 13.195 |
| ATOM | 3960 | HG1 | GLN | 348 | −19.664 | 15.067 | 15.421 |
| ATOM | 3961 | HG2 | GLN | 348 | −20.636 | 16.565 | 15.398 |
| ATOM | 3962 | HE22 | GLN | 348 | −23.783 | 14.856 | 16.045 |
| ATOM | 3963 | HE21 | GLN | 348 | −23.215 | 16.063 | 14.965 |
| ATOM | 3964 | N | LEU | 349 | −17.818 | 14.483 | 12.274 |
| ATOM | 3965 | CA | LEU | 349 | −16.508 | 13.852 | 12.451 |
| ATOM | 3966 | C | LEU | 349 | −15.428 | 14.903 | 12.332 |
| ATOM | 3967 | O | LEU | 349 | −14.573 | 14.988 | 13.2 |
| ATOM | 3968 | CB | LEU | 349 | −16.224 | 12.749 | 11.391 |
| ATOM | 3969 | CG | LEU | 349 | −14.781 | 12.156 | 11.455 |
| ATOM | 3970 | CD1 | LEU | 349 | −14.526 | 11.417 | 12.797 |
| ATOM | 3971 | CD2 | LEU | 349 | −14.532 | 11.195 | 10.259 |
| ATOM | 3972 | HA | LEU | 349 | −16.48 | 13.406 | 13.458 |
| ATOM | 3973 | H | LEU | 349 | −18.417 | 14.183 | 11.526 |
| ATOM | 3974 | HB1 | LEU | 349 | −16.966 | 11.945 | 11.513 |
| ATOM | 3975 | HB2 | LEU | 349 | −16.365 | 13.182 | 10.39 |
| ATOM | 3976 | HG | LEU | 349 | −14.036 | 12.964 | 11.355 |
| ATOM | 3977 | HD21 | LEU | 349 | −14.694 | 11.712 | 9.3 |
| ATOM | 3978 | HD22 | LEU | 349 | −13.499 | 10.813 | 10.272 |
| ATOM | 3979 | HD23 | LEU | 349 | −15.216 | 10.341 | 10.313 |
| ATOM | 3980 | HD11 | LEU | 349 | −15.345 | 10.718 | 13.019 |
| ATOM | 3981 | HD12 | LEU | 349 | −13.588 | 10.845 | 12.741 |
| ATOM | 3982 | HD13 | LEU | 349 | −14.446 | 12.131 | 13.63 |
| ATOM | 3983 | N | SER | 350 | −15.439 | 15.705 | 11.245 |
| ATOM | 3984 | CA | SER | 350 | −14.368 | 16.679 | 11.05 |
| ATOM | 3985 | C | SER | 350 | −14.322 | 17.682 | 12.18 |
| ATOM | 3986 | O | SER | 350 | −13.236 | 18.053 | 12.597 |
| ATOM | 3987 | CB | SER | 350 | −14.567 | 17.442 | 9.716 |
| ATOM | 3988 | OG | SER | 350 | −14.578 | 16.486 | 8.641 |
| ATOM | 3989 | HA | SER | 350 | −13.407 | 16.14 | 11 |
| ATOM | 3990 | H | SER | 350 | −16.162 | 15.634 | 10.551 |
| ATOM | 3991 | HB1 | SER | 350 | −15.524 | 17.987 | 9.746 |
| ATOM | 3992 | HB2 | SER | 350 | −13.748 | 18.169 | 9.576 |
| ATOM | 3993 | HG | SER | 350 | −14.717 | 16.887 | 7.788 |
| ATOM | 3994 | N | ILE | 351 | −15.492 | 18.139 | 12.677 |
| ATOM | 3995 | CA | ILE | 351 | −15.523 | 19.174 | 13.709 |
| ATOM | 3996 | C | ILE | 351 | −16.68 | 18.877 | 14.644 |
| ATOM | 3997 | O | ILE | 351 | −17.757 | 19.415 | 14.449 |
| ATOM | 3998 | CB | ILE | 351 | −15.615 | 20.593 | 13.057 |
| ATOM | 3999 | CG1 | ILE | 351 | −16.6 | 20.621 | 11.846 |
| ATOM | 4000 | CG2 | ILE | 351 | −14.208 | 21.098 | 12.62 |
| ATOM | 4001 | CD1 | ILE | 351 | −17.067 | 22.048 | 11.448 |
| ATOM | 4002 | HA | ILE | 351 | −14.622 | 19.135 | 14.342 |
| ATOM | 4003 | H | ILE | 351 | −16.368 | 17.806 | 12.319 |
| ATOM | 4004 | HB | ILE | 351 | −15.98 | 21.283 | 13.837 |
| ATOM | 4005 | HG11 | ILE | 351 | −16.137 | 20.136 | 10.972 |
| ATOM | 4006 | HG12 | ILE | 351 | −17.503 | 20.05 | 12.09 |
| ATOM | 4007 | HD11 | ILE | 351 | −16.231 | 22.659 | 11.079 |
| ATOM | 4008 | HD12 | ILE | 351 | −17.819 | 21.983 | 10.646 |
| ATOM | 4009 | HD13 | ILE | 351 | −17.526 | 22.556 | 12.311 |
| ATOM | 4010 | HG21 | ILE | 351 | −13.809 | 20.476 | 11.805 |
| ATOM | 4011 | HG22 | ILE | 351 | −14.25 | 22.139 | 12.27 |
| ATOM | 4012 | HG23 | ILE | 351 | −13.5 | 21.065 | 13.462 |
| ATOM | 4013 | N | SER | 352 | −16.507 | 18.019 | 15.677 |
| ATOM | 4014 | CA | SER | 352 | −17.638 | 17.728 | 16.559 |
| ATOM | 4015 | C | SER | 352 | −18.236 | 19 | 17.121 |
| ATOM | 4016 | O | SER | 352 | −17.529 | 19.99 | 17.206 |
| ATOM | 4017 | CB | SER | 352 | −17.294 | 16.762 | 17.722 |
| ATOM | 4018 | OG | SER | 352 | −18.465 | 16.4 | 18.482 |
| ATOM | 4019 | HA | SER | 352 | −18.398 | 17.242 | 15.935 |
| ATOM | 4020 | H | SER | 352 | −15.635 | 17.552 | 15.831 |
| ATOM | 4021 | HB1 | SER | 352 | −16.802 | 15.855 | 17.331 |
| ATOM | 4022 | HB2 | SER | 352 | −16.589 | 17.268 | 18.401 |
| ATOM | 4023 | HG | SER | 352 | −19.08 | 15.872 | 17.982 |
| ATOM | 4024 | N | ARG | 353 | −19.536 | 18.999 | 17.499 |
| ATOM | 4025 | CA | ARG | 353 | −20.191 | 20.246 | 17.899 |
| ATOM | 4026 | C | ARG | 353 | −20.079 | 21.274 | 16.789 |
| ATOM | 4027 | O | ARG | 353 | −19.596 | 22.363 | 17.056 |
| ATOM | 4028 | CB | ARG | 353 | −19.625 | 20.738 | 19.264 |
| ATOM | 4029 | CG | ARG | 353 | −19.496 | 19.602 | 20.32 |
| ATOM | 4030 | CD | ARG | 353 | −20.844 | 18.895 | 20.638 |
| ATOM | 4031 | NE | ARG | 353 | −20.696 | 17.879 | 21.684 |
| ATOM | 4032 | CZ | ARG | 353 | −21.678 | 17.105 | 22.094 |
| ATOM | 4033 | NH1 | ARG | 353 | −22.892 | 17.176 | 21.602 |
| ATOM | 4034 | NH2 | ARG | 353 | −21.44 | 16.223 | 23.036 |
| ATOM | 4035 | HA | ARG | 353 | −21.274 | 20.09 | 18.033 |
| ATOM | 4036 | H | ARG | 353 | −20.076 | 18.156 | 17.483 |
| ATOM | 4037 | HB1 | ARG | 353 | −18.62 | 21.165 | 19.115 |
| ATOM | 4038 | HB2 | ARG | 353 | −20.268 | 21.543 | 19.656 |
| ATOM | 4039 | HG1 | ARG | 353 | −18.769 | 18.85 | 19.971 |
| ATOM | 4040 | HG2 | ARG | 353 | −19.102 | 20.041 | 21.252 |
| ATOM | 4041 | HD1 | ARG | 353 | −21.57 | 19.662 | 20.958 |
| ATOM | 4042 | HD2 | ARG | 353 | −21.201 | 18.407 | 19.72 |
| ATOM | 4043 | HE | ARG | 353 | −19.759 | 17.772 | 22.11 |
| ATOM | 4044 | HH12 | ARG | 353 | −23.641 | 16.556 | 21.942 |
| ATOM | 4045 | HH11 | ARG | 353 | −23.139 | 17.847 | 20.866 |
| ATOM | 4046 | HH22 | ARG | 353 | −22.184 | 15.602 | 23.386 |
| ATOM | 4047 | HH21 | ARG | 353 | −20.504 | 16.131 | 23.458 |
| ATOM | 4048 | N | PRO | 354 | −20.5 | 20.99 | 15.53 |
| ATOM | 4049 | CA | PRO | 354 | −20.286 | 21.953 | 14.456 |
| ATOM | 4050 | C | PRO | 354 | −21.34 | 23.04 | 14.451 |
| ATOM | 4051 | O | PRO | 354 | −22.253 | 22.988 | 15.259 |
| ATOM | 4052 | CB | PRO | 354 | −20.466 | 20.999 | 13.252 |
| ATOM | 4053 | CG | PRO | 354 | −21.58 | 20.04 | 13.728 |
| ATOM | 4054 | CD | PRO | 354 | −21.126 | 19.724 | 15.171 |
| ATOM | 4055 | HA | PRO | 354 | −19.274 | 22.389 | 14.477 |
| ATOM | 4056 | HD2 | PRO | 354 | −20.364 | 18.934 | 15.135 |
| ATOM | 4057 | HD1 | PRO | 354 | −21.973 | 19.425 | 15.81 |
| ATOM | 4058 | HG2 | PRO | 354 | −21.682 | 19.142 | 13.096 |
| ATOM | 4059 | HG1 | PRO | 354 | −22.543 | 20.576 | 13.749 |
| ATOM | 4060 | HB1 | PRO | 354 | −20.668 | 21.514 | 12.308 |
| ATOM | 4061 | HB2 | PRO | 354 | −19.559 | 20.401 | 13.111 |
| ATOM | 4062 | N | THR | 355 | −21.221 | 24.033 | 13.536 |
| ATOM | 4063 | CA | THR | 355 | −22.145 | 25.171 | 13.517 |
| ATOM | 4064 | C | THR | 355 | −22.791 | 25.268 | 12.15 |
| ATOM | 4065 | O | THR | 355 | −22.122 | 25.71 | 11.231 |
| ATOM | 4066 | CB | THR | 355 | −21.334 | 26.457 | 13.839 |
| ATOM | 4067 | OG1 | THR | 355 | −20.73 | 26.279 | 15.134 |
| ATOM | 4068 | CG2 | THR | 355 | −22.241 | 27.717 | 13.845 |
| ATOM | 4069 | HA | THR | 355 | −22.922 | 25.087 | 14.293 |
| ATOM | 4070 | H | THR | 355 | −20.473 | 24.032 | 12.869 |
| ATOM | 4071 | HB | THR | 355 | −20.539 | 26.581 | 13.082 |
| ATOM | 4072 | HG1 | THR | 355 | −20.18 | 27.011 | 15.397 |
| ATOM | 4073 | HG23 | THR | 355 | −22.719 | 27.865 | 12.864 |
| ATOM | 4074 | HG21 | THR | 355 | −23.024 | 27.615 | 14.61 |
| ATOM | 4075 | HG22 | THR | 355 | −21.642 | 28.612 | 14.074 |
| ATOM | 4076 | N | LEU | 356 | −24.075 | 24.873 | 11.976 |
| ATOM | 4077 | CA | LEU | 356 | −24.703 | 24.953 | 10.655 |
| ATOM | 4078 | C | LEU | 356 | −26.204 | 24.793 | 10.778 |
| ATOM | 4079 | O | LEU | 356 | −26.737 | 23.81 | 10.295 |
| ATOM | 4080 | CB | LEU | 356 | −24.091 | 23.98 | 9.598 |
| ATOM | 4081 | CG | LEU | 356 | −24.039 | 22.458 | 9.954 |
| ATOM | 4082 | CD1 | LEU | 356 | −23.507 | 21.667 | 8.723 |
| ATOM | 4083 | CD2 | LEU | 356 | −23.122 | 22.163 | 11.169 |
| ATOM | 4084 | HA | LEU | 356 | −24.558 | 25.977 | 10.267 |
| ATOM | 4085 | H | LEU | 356 | −24.617 | 24.518 | 12.739 |
| ATOM | 4086 | HB1 | LEU | 356 | −24.676 | 24.099 | 8.671 |
| ATOM | 4087 | HB2 | LEU | 356 | −23.065 | 24.308 | 9.37 |
| ATOM | 4088 | HG | LEU | 356 | −25.04 | 22.068 | 10.195 |
| ATOM | 4089 | HD21 | LEU | 356 | −23.582 | 22.458 | 12.12 |
| ATOM | 4090 | HD22 | LEU | 356 | −22.909 | 21.085 | 11.237 |
| ATOM | 4091 | HD23 | LEU | 356 | −22.177 | 22.708 | 11.037 |
| ATOM | 4092 | HD11 | LEU | 356 | −22.5 | 22.018 | 8.449 |
| ATOM | 4093 | HD12 | LEU | 356 | −23.444 | 20.59 | 8.94 |
| ATOM | 4094 | HD13 | LEU | 356 | −24.171 | 21.8 | 7.855 |
| ATOM | 4095 | N | THR | 357 | −26.921 | 25.745 | 11.421 |
| ATOM | 4096 | CA | THR | 357 | −28.376 | 25.597 | 11.55 |
| ATOM | 4097 | C | THR | 357 | −28.65 | 24.203 | 12.086 |
| ATOM | 4098 | O | THR | 357 | −29.466 | 23.457 | 11.569 |
| ATOM | 4099 | CB | THR | 357 | −28.988 | 25.987 | 10.175 |
| ATOM | 4100 | OG1 | THR | 357 | −28.499 | 27.306 | 9.867 |
| ATOM | 4101 | CG2 | THR | 357 | −30.538 | 26.002 | 10.183 |
| ATOM | 4102 | HA | THR | 357 | −28.764 | 26.306 | 12.3 |
| ATOM | 4103 | HB | THR | 357 | −28.629 | 25.286 | 9.404 |
| ATOM | 4104 | HG1 | THR | 357 | −28.776 | 27.622 | 9.012 |
| ATOM | 4105 | HG23 | THR | 357 | −30.929 | 24.996 | 10.373 |
| ATOM | 4106 | HG21 | THR | 357 | −30.912 | 26.686 | 10.96 |
| ATOM | 4107 | HG22 | THR | 357 | −30.921 | 26.335 | 9.206 |
| ATOM | 4108 | H | THR | 357 | −26.483 | 26.568 | 11.79 |
| ATOM | 4109 | N | PHE | 358 | −27.894 | 23.841 | 13.148 |
| ATOM | 4110 | CA | PHE | 358 | −27.779 | 22.44 | 13.548 |
| ATOM | 4111 | C | PHE | 358 | −29.102 | 21.725 | 13.739 |
| ATOM | 4112 | O | PHE | 358 | −29.359 | 20.764 | 13.031 |
| ATOM | 4113 | CB | PHE | 358 | −26.906 | 22.336 | 14.831 |
| ATOM | 4114 | CG | PHE | 358 | −26.846 | 20.894 | 15.348 |
| ATOM | 4115 | CD1 | PHE | 358 | −25.851 | 20.016 | 14.902 |
| ATOM | 4116 | CD2 | PHE | 358 | −27.788 | 20.447 | 16.279 |
| ATOM | 4117 | CE1 | PHE | 358 | −25.833 | 18.69 | 15.344 |
| ATOM | 4118 | CE2 | PHE | 358 | −27.798 | 19.114 | 16.69 |
| ATOM | 4119 | CZ | PHE | 358 | −26.815 | 18.235 | 16.228 |
| ATOM | 4120 | HA | PHE | 358 | −27.232 | 21.919 | 12.743 |
| ATOM | 4121 | HB1 | PHE | 358 | −25.883 | 22.692 | 14.62 |
| ATOM | 4122 | HB2 | PHE | 358 | −27.318 | 22.986 | 15.619 |
| ATOM | 4123 | HD2 | PHE | 358 | −28.519 | 21.132 | 16.69 |
| ATOM | 4124 | HE2 | PHE | 358 | −28.571 | 18.768 | 17.368 |
| ATOM | 4125 | HZ | PHE | 358 | −26.816 | 17.203 | 16.557 |
| ATOM | 4126 | HE1 | PHE | 358 | −25.058 | 18.012 | 14.999 |
| ATOM | 4127 | HD1 | PHE | 358 | −25.091 | 20.363 | 14.212 |
| ATOM | 4128 | H | PHE | 358 | −27.294 | 24.512 | 13.589 |
| ATOM | 4129 | N | VAL | 359 | −29.959 | 22.135 | 14.701 |
| ATOM | 4130 | CA | VAL | 359 | −31.115 | 21.296 | 15.025 |
| ATOM | 4131 | C | VAL | 359 | −31.951 | 21.017 | 13.798 |
| ATOM | 4132 | O | VAL | 359 | −32.408 | 19.894 | 13.648 |
| ATOM | 4133 | CB | VAL | 359 | −32.042 | 21.822 | 16.166 |
| ATOM | 4134 | CG1 | VAL | 359 | −31.298 | 21.899 | 17.53 |
| ATOM | 4135 | CG2 | VAL | 359 | −32.71 | 23.184 | 15.827 |
| ATOM | 4136 | HA | VAL | 359 | −30.714 | 20.323 | 15.359 |
| ATOM | 4137 | H | VAL | 359 | −29.774 | 22.961 | 15.231 |
| ATOM | 4138 | HB | VAL | 359 | −32.855 | 21.084 | 16.296 |
| ATOM | 4139 | HG11 | VAL | 359 | −30.5 | 22.656 | 17.507 |
| ATOM | 4140 | HG12 | VAL | 359 | −32.002 | 22.17 | 18.332 |
| ATOM | 4141 | HG13 | VAL | 359 | −30.855 | 20.924 | 17.785 |
| ATOM | 4142 | HG21 | VAL | 359 | −33.363 | 23.095 | 14.946 |
| ATOM | 4143 | HG22 | VAL | 359 | −33.336 | 23.519 | 16.669 |
| ATOM | 4144 | HG23 | VAL | 359 | −31.955 | 23.959 | 15.631 |
| ATOM | 4145 | N | TYR | 360 | −32.18 | 22.009 | 12.908 |
| ATOM | 4146 | CA | TYR | 360 | −33.076 | 21.759 | 11.784 |
| ATOM | 4147 | C | TYR | 360 | −32.391 | 20.936 | 10.721 |
| ATOM | 4148 | O | TYR | 360 | −33.032 | 20.033 | 10.208 |
| ATOM | 4149 | CB | TYR | 360 | −33.65 | 23.053 | 11.157 |
| ATOM | 4150 | CG | TYR | 360 | −34.614 | 22.674 | 10.022 |
| ATOM | 4151 | CD1 | TYR | 360 | −35.882 | 22.167 | 10.322 |
| ATOM | 4152 | CD2 | TYR | 360 | −34.236 | 22.819 | 8.683 |
| ATOM | 4153 | CE1 | TYR | 360 | −36.74 | 21.75 | 9.3 |
| ATOM | 4154 | CE2 | TYR | 360 | −35.091 | 22.399 | 7.66 |
| ATOM | 4155 | CZ | TYR | 360 | −36.341 | 21.853 | 7.961 |
| ATOM | 4156 | OH | TYR | 360 | −37.166 | 21.421 | 6.918 |
| ATOM | 4157 | HA | TYR | 360 | −33.945 | 21.192 | 12.158 |
| ATOM | 4158 | H | TYR | 360 | −31.744 | 22.907 | 12.996 |
| ATOM | 4159 | HB2 | TYR | 360 | −34.186 | 23.638 | 11.921 |
| ATOM | 4160 | HB1 | TYR | 360 | −32.826 | 23.669 | 10.776 |
| ATOM | 4161 | HD2 | TYR | 360 | −33.278 | 23.255 | 8.429 |
| ATOM | 4162 | HE2 | TYR | 360 | −34.784 | 22.494 | 6.624 |
| ATOM | 4163 | HE1 | TYR | 360 | −37.714 | 21.346 | 9.555 |
| ATOM | 4164 | HD1 | TYR | 360 | −36.208 | 22.091 | 11.355 |
| ATOM | 4165 | HH | TYR | 360 | −37.977 | 21.014 | 7.202 |
| ATOM | 4166 | N | LEU | 361 | −31.116 | 21.195 | 10.35 |
| ATOM | 4167 | CA | LEU | 361 | −30.508 | 20.356 | 9.315 |
| ATOM | 4168 | C | LEU | 361 | −30.431 | 18.928 | 9.811 |
| ATOM | 4169 | O | LEU | 361 | −30.751 | 18.018 | 9.061 |
| ATOM | 4170 | CB | LEU | 361 | −29.08 | 20.778 | 8.867 |
| ATOM | 4171 | CG | LEU | 361 | −28.94 | 22.228 | 8.317 |
| ATOM | 4172 | CD1 | LEU | 361 | −27.524 | 22.421 | 7.704 |
| ATOM | 4173 | CD2 | LEU | 361 | −30.014 | 22.586 | 7.255 |
| ATOM | 4174 | HA | LEU | 361 | −31.166 | 20.356 | 8.43 |
| ATOM | 4175 | H | LEU | 361 | −30.576 | 21.92 | 10.786 |
| ATOM | 4176 | HB1 | LEU | 361 | −28.386 | 20.647 | 9.714 |
| ATOM | 4177 | HB2 | LEU | 361 | −28.771 | 20.092 | 8.065 |
| ATOM | 4178 | HG | LEU | 361 | −29.049 | 22.926 | 9.159 |
| ATOM | 4179 | HD21 | LEU | 361 | −31.015 | 22.614 | 7.706 |
| ATOM | 4180 | HD22 | LEU | 361 | −29.813 | 23.583 | 6.831 |
| ATOM | 4181 | HD23 | LEU | 361 | −30.009 | 21.849 | 6.439 |
| ATOM | 4182 | HD11 | LEU | 361 | −27.42 | 21.836 | 6.778 |
| ATOM | 4183 | HD12 | LEU | 361 | −27.343 | 23.482 | 7.468 |
| ATOM | 4184 | HD13 | LEU | 361 | −26.749 | 22.086 | 8.407 |
| ATOM | 4185 | N | TYR | 362 | −30.007 | 18.702 | 11.074 |
| ATOM | 4186 | CA | TYR | 362 | −29.919 | 17.328 | 11.566 |
| ATOM | 4187 | C | TYR | 362 | −31.317 | 16.757 | 11.679 |
| ATOM | 4188 | O | TYR | 362 | −31.546 | 15.68 | 11.153 |
| ATOM | 4189 | CB | TYR | 362 | −29.114 | 17.209 | 12.891 |
| ATOM | 4190 | CG | TYR | 362 | −27.597 | 17.243 | 12.616 |
| ATOM | 4191 | CD1 | TYR | 362 | −26.939 | 18.446 | 12.334 |
| ATOM | 4192 | CD2 | TYR | 362 | −26.845 | 16.061 | 12.637 |
| ATOM | 4193 | CE1 | TYR | 362 | −25.567 | 18.47 | 12.064 |
| ATOM | 4194 | CE2 | TYR | 362 | −25.465 | 16.084 | 12.411 |
| ATOM | 4195 | CZ | TYR | 362 | −24.822 | 17.29 | 12.111 |
| ATOM | 4196 | OH | TYR | 362 | −23.452 | 17.332 | 11.852 |
| ATOM | 4197 | HA | TYR | 362 | −29.386 | 16.712 | 10.823 |
| ATOM | 4198 | H | TYR | 362 | −29.798 | 19.465 | 11.692 |
| ATOM | 4199 | HB2 | TYR | 362 | −29.373 | 16.251 | 13.37 |
| ATOM | 4200 | HB1 | TYR | 362 | −29.393 | 18.012 | 13.591 |
| ATOM | 4201 | HD2 | TYR | 362 | −27.331 | 15.112 | 12.829 |
| ATOM | 4202 | HE2 | TYR | 362 | −24.892 | 15.165 | 12.464 |
| ATOM | 4203 | HE1 | TYR | 362 | −25.075 | 19.404 | 11.815 |
| ATOM | 4204 | HD1 | TYR | 362 | −27.483 | 19.377 | 12.321 |
| ATOM | 4205 | HH | TYR | 362 | −23.146 | 16.562 | 11.384 |
| ATOM | 4206 | N | ASN | 363 | −32.285 | 17.436 | 12.334 |
| ATOM | 4207 | CA | ASN | 363 | −33.642 | 16.88 | 12.392 |
| ATOM | 4208 | C | ASN | 363 | −34.145 | 16.572 | 10.997 |
| ATOM | 4209 | O | ASN | 363 | −34.622 | 15.476 | 10.747 |
| ATOM | 4210 | CB | ASN | 363 | −34.645 | 17.862 | 13.057 |
| ATOM | 4211 | CG | ASN | 363 | −36.062 | 17.337 | 13.055 |
| ATOM | 4212 | OD1 | ASN | 363 | −36.261 | 16.138 | 12.95 |
| ATOM | 4213 | ND2 | ASN | 363 | −37.085 | 18.209 | 13.181 |
| ATOM | 4214 | HA | ASN | 363 | −33.621 | 15.949 | 12.982 |
| ATOM | 4215 | H | ASN | 363 | −32.091 | 18.321 | 12.765 |
| ATOM | 4216 | HB1 | ASN | 363 | −34.346 | 18.05 | 14.099 |
| ATOM | 4217 | HB2 | ASN | 363 | −34.621 | 18.817 | 12.513 |
| ATOM | 4218 | HD22 | ASN | 363 | −38.023 | 17.863 | 13.161 |
| ATOM | 4219 | HD21 | ASN | 363 | −36.929 | 19.191 | 13.289 |
| ATOM | 4220 | N | ALA | 364 | −34.051 | 17.544 | 10.063 |
| ATOM | 4221 | CA | ALA | 364 | −34.586 | 17.326 | 8.722 |
| ATOM | 4222 | C | ALA | 364 | −33.891 | 16.159 | 8.064 |
| ATOM | 4223 | O | ALA | 364 | −34.559 | 15.326 | 7.474 |
| ATOM | 4224 | CB | ALA | 364 | −34.414 | 18.585 | 7.833 |
| ATOM | 4225 | HA | ALA | 364 | −35.664 | 17.105 | 8.808 |
| ATOM | 4226 | H | ALA | 364 | −33.616 | 18.421 | 10.274 |
| ATOM | 4227 | HB1 | ALA | 364 | −34.845 | 18.411 | 6.834 |
| ATOM | 4228 | HB2 | ALA | 364 | −34.929 | 19.442 | 8.293 |
| ATOM | 4229 | HB3 | ALA | 364 | −33.348 | 18.836 | 7.718 |
| ATOM | 4230 | N | ALA | 365 | −32.546 | 16.076 | 8.146 |
| ATOM | 4231 | CA | ALA | 365 | −31.852 | 14.953 | 7.516 |
| ATOM | 4232 | C | ALA | 365 | −32.23 | 13.645 | 8.175 |
| ATOM | 4233 | O | ALA | 365 | −32.398 | 12.658 | 7.477 |
| ATOM | 4234 | CB | ALA | 365 | −30.319 | 15.127 | 7.615 |
| ATOM | 4235 | HA | ALA | 365 | −32.122 | 14.926 | 6.448 |
| ATOM | 4236 | H | ALA | 365 | −32.014 | 16.767 | 8.644 |
| ATOM | 4237 | HB1 | ALA | 365 | −29.801 | 14.3 | 7.108 |
| ATOM | 4238 | HB2 | ALA | 365 | −30.024 | 16.072 | 7.136 |
| ATOM | 4239 | HB3 | ALA | 365 | −30.024 | 15.142 | 8.675 |
| ATOM | 4240 | N | ILE | 366 | −32.368 | 13.613 | 9.52 |
| ATOM | 4241 | CA | ILE | 366 | −32.777 | 12.373 | 10.185 |
| ATOM | 4242 | C | ILE | 366 | −34.14 | 11.997 | 9.647 |
| ATOM | 4243 | O | ILE | 366 | −34.34 | 10.864 | 9.235 |
| ATOM | 4244 | CB | ILE | 366 | −32.823 | 12.528 | 11.74 |
| ATOM | 4245 | CG1 | ILE | 366 | −31.387 | 12.728 | 12.32 |
| ATOM | 4246 | CG2 | ILE | 366 | −33.516 | 11.3 | 12.4 |
| ATOM | 4247 | CD1 | ILE | 366 | −31.374 | 13.326 | 13.754 |
| ATOM | 4248 | HA | ILE | 366 | −32.063 | 11.572 | 9.932 |
| ATOM | 4249 | H | ILE | 366 | −32.241 | 14.445 | 10.065 |
| ATOM | 4250 | HB | ILE | 366 | −33.43 | 13.417 | 11.973 |
| ATOM | 4251 | HG11 | ILE | 366 | −30.871 | 11.76 | 12.317 |
| ATOM | 4252 | HG12 | ILE | 366 | −30.797 | 13.398 | 11.678 |
| ATOM | 4253 | HD11 | ILE | 366 | −31.795 | 12.628 | 14.491 |
| ATOM | 4254 | HD12 | ILE | 366 | −30.345 | 13.56 | 14.066 |
| ATOM | 4255 | HD13 | ILE | 366 | −31.953 | 14.26 | 13.781 |
| ATOM | 4256 | HG21 | ILE | 366 | −32.948 | 10.383 | 12.183 |
| ATOM | 4257 | HG22 | ILE | 366 | −33.579 | 11.424 | 13.489 |
| ATOM | 4258 | HG23 | ILE | 366 | −34.544 | 11.17 | 12.03 |
| ATOM | 4259 | N | SER | 367 | −35.102 | 12.945 | 9.649 |
| ATOM | 4260 | CA | SER | 367 | −36.443 | 12.618 | 9.17 |
| ATOM | 4261 | C | SER | 367 | −36.388 | 12.221 | 7.709 |
| ATOM | 4262 | O | SER | 367 | −37.071 | 11.286 | 7.322 |
| ATOM | 4263 | CB | SER | 367 | −37.426 | 13.806 | 9.35 |
| ATOM | 4264 | OG | SER | 367 | −37.668 | 14.111 | 10.738 |
| ATOM | 4265 | HA | SER | 367 | −36.831 | 11.767 | 9.756 |
| ATOM | 4266 | H | SER | 367 | −34.903 | 13.879 | 9.958 |
| ATOM | 4267 | HB1 | SER | 367 | −37.053 | 14.696 | 8.816 |
| ATOM | 4268 | HB2 | SER | 367 | −38.401 | 13.531 | 8.917 |
| ATOM | 4269 | HG | SER | 367 | −36.898 | 14.422 | 11.206 |
| ATOM | 4270 | N | LEU | 368 | −35.584 | 12.911 | 6.87 |
| ATOM | 4271 | CA | LEU | 368 | −35.504 | 12.529 | 5.46 |
| ATOM | 4272 | C | LEU | 368 | −34.986 | 11.114 | 5.355 |
| ATOM | 4273 | O | LEU | 368 | −35.572 | 10.327 | 4.63 |
| ATOM | 4274 | CB | LEU | 368 | −34.577 | 13.476 | 4.648 |
| ATOM | 4275 | CG | LEU | 368 | −34.371 | 13.067 | 3.157 |
| ATOM | 4276 | CD1 | LEU | 368 | −35.705 | 13.058 | 2.358 |
| ATOM | 4277 | CD2 | LEU | 368 | −33.36 | 14.038 | 2.482 |
| ATOM | 4278 | HA | LEU | 368 | −36.525 | 12.58 | 5.05 |
| ATOM | 4279 | H | LEU | 368 | −35.031 | 13.678 | 7.2 |
| ATOM | 4280 | HB1 | LEU | 368 | −34.983 | 14.499 | 4.701 |
| ATOM | 4281 | HB2 | LEU | 368 | −33.591 | 13.477 | 5.137 |
| ATOM | 4282 | HG | LEU | 368 | −33.933 | 12.055 | 3.111 |
| ATOM | 4283 | HD21 | LEU | 368 | −32.398 | 14.026 | 3.017 |
| ATOM | 4284 | HD22 | LEU | 368 | −33.173 | 13.743 | 1.438 |
| ATOM | 4285 | HD23 | LEU | 368 | −33.753 | 15.066 | 2.49 |
| ATOM | 4286 | HD11 | LEU | 368 | −36.21 | 14.032 | 2.444 |
| ATOM | 4287 | HD12 | LEU | 368 | −35.512 | 12.858 | 1.293 |
| ATOM | 4288 | HD13 | LEU | 368 | −36.385 | 12.274 | 2.721 |
| ATOM | 4289 | N | GLY | 369 | −33.894 | 10.76 | 6.07 |
| ATOM | 4290 | CA | GLY | 369 | −33.393 | 9.389 | 5.989 |
| ATOM | 4291 | C | GLY | 369 | −34.505 | 8.395 | 6.225 |
| ATOM | 4292 | O | GLY | 369 | −34.549 | 7.379 | 5.55 |
| ATOM | 4293 | HA2 | GLY | 369 | −32.619 | 9.203 | 6.751 |
| ATOM | 4294 | HA1 | GLY | 369 | −32.945 | 9.224 | 4.996 |
| ATOM | 4295 | H | GLY | 369 | −33.419 | 11.416 | 6.663 |
| ATOM | 4296 | N | TYR | 370 | −35.419 | 8.667 | 7.183 |
| ATOM | 4297 | CA | TYR | 370 | −36.497 | 7.713 | 7.452 |
| ATOM | 4298 | C | TYR | 370 | −37.385 | 7.551 | 6.233 |
| ATOM | 4299 | O | TYR | 370 | −37.885 | 6.458 | 6.015 |
| ATOM | 4300 | CB | TYR | 370 | −37.362 | 8.161 | 8.662 |
| ATOM | 4301 | CG | TYR | 370 | −36.631 | 8.222 | 10.018 |
| ATOM | 4302 | CD1 | TYR | 370 | −37.398 | 8.499 | 11.156 |
| ATOM | 4303 | CD2 | TYR | 370 | −35.256 | 8.005 | 10.18 |
| ATOM | 4304 | CE1 | TYR | 370 | −36.821 | 8.508 | 12.429 |
| ATOM | 4305 | CE2 | TYR | 370 | −34.667 | 8.065 | 11.447 |
| ATOM | 4306 | CZ | TYR | 370 | −35.441 | 8.33 | 12.578 |
| ATOM | 4307 | OH | TYR | 370 | −34.823 | 8.415 | 13.831 |
| ATOM | 4308 | HA | TYR | 370 | −36.062 | 6.725 | 7.68 |
| ATOM | 4309 | H | TYR | 370 | −35.372 | 9.518 | 7.715 |
| ATOM | 4310 | HB2 | TYR | 370 | −37.787 | 9.154 | 8.458 |
| ATOM | 4311 | HB1 | TYR | 370 | −38.199 | 7.453 | 8.767 |
| ATOM | 4312 | HD2 | TYR | 370 | −34.615 | 7.782 | 9.338 |
| ATOM | 4313 | HE2 | TYR | 370 | −33.599 | 7.902 | 11.555 |
| ATOM | 4314 | HE1 | TYR | 370 | −37.455 | 8.651 | 13.298 |
| ATOM | 4315 | HD1 | TYR | 370 | −38.459 | 8.706 | 11.054 |
| ATOM | 4316 | HH | TYR | 370 | −35.421 | 8.636 | 14.535 |
| ATOM | 4317 | N | ALA | 371 | −37.593 | 8.616 | 5.421 |
| ATOM | 4318 | CA | ALA | 371 | −38.404 | 8.469 | 4.209 |
| ATOM | 4319 | C | ALA | 371 | −37.957 | 7.274 | 3.399 |
| ATOM | 4320 | O | ALA | 371 | −38.814 | 6.627 | 2.818 |
| ATOM | 4321 | CB | ALA | 371 | −38.328 | 9.715 | 3.288 |
| ATOM | 4322 | HA | ALA | 371 | −39.453 | 8.319 | 4.512 |
| ATOM | 4323 | H | ALA | 371 | −37.192 | 9.512 | 5.627 |
| ATOM | 4324 | HB1 | ALA | 371 | −39.028 | 9.604 | 2.444 |
| ATOM | 4325 | HB2 | ALA | 371 | −38.596 | 10.624 | 3.849 |
| ATOM | 4326 | HB3 | ALA | 371 | −37.313 | 9.83 | 2.88 |
| ATOM | 4327 | N | ASN | 372 | −36.635 | 6.976 | 3.366 |
| ATOM | 4328 | CA | ASN | 372 | −36.132 | 5.78 | 2.682 |
| ATOM | 4329 | C | ASN | 372 | −37.116 | 4.63 | 2.713 |
| ATOM | 4330 | O | ASN | 372 | −37.372 | 4.018 | 1.687 |
| ATOM | 4331 | CB | ASN | 372 | −34.817 | 5.314 | 3.367 |
| ATOM | 4332 | CG | ASN | 372 | −34.245 | 4.071 | 2.731 |
| ATOM | 4333 | OD1 | ASN | 372 | −33.333 | 4.189 | 1.928 |
| ATOM | 4334 | ND2 | ASN | 372 | −34.75 | 2.866 | 3.069 |
| ATOM | 4335 | HA | ASN | 372 | −35.902 | 6.031 | 1.636 |
| ATOM | 4336 | H | ASN | 372 | −35.974 | 7.58 | 3.82 |
| ATOM | 4337 | HB1 | ASN | 372 | −34.068 | 6.118 | 3.303 |
| ATOM | 4338 | HB2 | ASN | 372 | −35.002 | 5.114 | 4.433 |
| ATOM | 4339 | HD22 | ASN | 372 | −34.387 | 2.041 | 2.643 |
| ATOM | 4340 | HD21 | ASN | 372 | −35.48 | 2.766 | 3.744 |
| ATOM | 4341 | N | SER | 373 | −37.676 | 4.327 | 3.905 |
| ATOM | 4342 | CA | SER | 373 | −38.541 | 3.152 | 4.045 |
| ATOM | 4343 | C | SER | 373 | −39.811 | 3.158 | 3.222 |
| ATOM | 4344 | O | SER | 373 | −40.484 | 2.139 | 3.225 |
| ATOM | 4345 | CB | SER | 373 | −38.892 | 2.903 | 5.537 |
| ATOM | 4346 | OG | SER | 373 | −39.751 | 3.928 | 6.07 |
| ATOM | 4347 | HA | SER | 373 | −37.965 | 2.285 | 3.688 |
| ATOM | 4348 | H | SER | 373 | −37.468 | 4.884 | 4.712 |
| ATOM | 4349 | HB1 | SER | 373 | −39.433 | 1.951 | 5.649 |
| ATOM | 4350 | HB2 | SER | 373 | −37.956 | 2.83 | 6.115 |
| ATOM | 4351 | HG | SER | 373 | −39.35 | 4.791 | 6.074 |
| ATOM | 4352 | N | CYS | 374 | −40.178 | 4.247 | 2.508 |
| ATOM | 4353 | CA | CYS | 374 | −41.358 | 4.209 | 1.642 |
| ATOM | 4354 | C | CYS | 374 | −41.009 | 4.572 | 0.211 |
| ATOM | 4355 | O | CYS | 374 | −41.895 | 5.004 | −0.508 |
| ATOM | 4356 | CB | CYS | 374 | −42.395 | 5.183 | 2.256 |
| ATOM | 4357 | SG | CYS | 374 | −44.008 | 5.021 | 1.42 |
| ATOM | 4358 | HA | CYS | 374 | −41.817 | 3.208 | 1.588 |
| ATOM | 4359 | H | CYS | 374 | −39.662 | 5.104 | 2.55 |
| ATOM | 4360 | HB1 | CYS | 374 | −42.54 | 4.951 | 3.323 |
| ATOM | 4361 | HB2 | CYS | 374 | −42.018 | 6.215 | 2.166 |
| ATOM | 4362 | HG | CYS | 374 | −44.402 | 5.899 | 1.717 |
| ATOM | 4363 | LPG1 | CYS | 374 | −43.946 | 4.981 | 0.745 |
| ATOM | 4364 | LPG2 | CYS | 374 | −44.363 | 4.489 | 1.646 |
| ATOM | 4365 | N | LEU | 375 | −39.741 | 4.415 | −0.241 |
| ATOM | 4366 | CA | LEU | 375 | −39.382 | 4.809 | −1.608 |
| ATOM | 4367 | C | LEU | 375 | −39.246 | 3.611 | −2.527 |
| ATOM | 4368 | O | LEU | 375 | −39.733 | 3.667 | −3.646 |
| ATOM | 4369 | CB | LEU | 375 | −38.059 | 5.626 | −1.611 |
| ATOM | 4370 | CG | LEU | 375 | −38.042 | 6.796 | −0.58 |
| ATOM | 4371 | CD1 | LEU | 375 | −36.724 | 7.612 | −0.702 |
| ATOM | 4372 | CD2 | LEU | 375 | −39.26 | 7.75 | −0.725 |
| ATOM | 4373 | HA | LEU | 375 | −40.153 | 5.456 | −2.057 |
| ATOM | 4374 | H | LEU | 375 | −39.024 | 4.038 | 0.351 |
| ATOM | 4375 | HB1 | LEU | 375 | −37.224 | 4.945 | −1.378 |
| ATOM | 4376 | HB2 | LEU | 375 | −37.895 | 6.026 | −2.625 |
| ATOM | 4377 | HG | LEU | 375 | −38.076 | 6.362 | 0.431 |
| ATOM | 4378 | HD21 | LEU | 375 | −40.201 | 7.246 | −0.458 |
| ATOM | 4379 | HD22 | LEU | 375 | −39.148 | 8.614 | −0.053 |
| ATOM | 4380 | HD23 | LEU | 375 | −39.335 | 8.119 | −1.76 |
| ATOM | 4381 | HD11 | LEU | 375 | −36.717 | 8.173 | −1.648 |
| ATOM | 4382 | HD12 | LEU | 375 | −36.637 | 8.331 | 0.128 |
| ATOM | 4383 | HD13 | LEU | 375 | −35.844 | 6.952 | −0.681 |
| ATOM | 4384 | N | ASN | 376 | −38.584 | 2.509 | −2.104 |
| ATOM | 4385 | CA | ASN | 376 | −38.366 | 1.403 | −3.035 |
| ATOM | 4386 | C | ASN | 376 | −39.666 | 0.89 | −3.623 |
| ATOM | 4387 | O | ASN | 376 | −39.682 | 0.68 | −4.827 |
| ATOM | 4388 | CB | ASN | 376 | −37.519 | 0.233 | −2.46 |
| ATOM | 4389 | CG | ASN | 376 | −36.294 | 0.784 | −1.769 |
| ATOM | 4390 | OD1 | ASN | 376 | −35.418 | 1.273 | −2.464 |
| ATOM | 4391 | ND2 | ASN | 376 | −36.189 | 0.741 | −0.421 |
| ATOM | 4392 | HA | ASN | 376 | −37.771 | 1.828 | −3.863 |
| ATOM | 4393 | H | ASN | 376 | −38.196 | 2.453 | −1.184 |
| ATOM | 4394 | HB1 | ASN | 376 | −38.094 | −0.397 | −1.769 |
| ATOM | 4395 | HB2 | ASN | 376 | −37.207 | −0.417 | −3.294 |
| ATOM | 4396 | HD22 | ASN | 376 | −35.369 | 1.12 | 0.01 |
| ATOM | 4397 | HD21 | ASN | 376 | −36.896 | 0.347 | 0.175 |
| ATOM | 4398 | N | PRO | 377 | −40.78 | 0.677 | −2.873 |
| ATOM | 4399 | CA | PRO | 377 | −42.013 | 0.261 | −3.53 |
| ATOM | 4400 | C | PRO | 377 | −42.438 | 1.216 | −4.62 |
| ATOM | 4401 | O | PRO | 377 | −42.905 | 0.773 | −5.657 |
| ATOM | 4402 | CB | PRO | 377 | −43.003 | 0.321 | −2.337 |
| ATOM | 4403 | CG | PRO | 377 | −42.101 | 0.051 | −1.114 |
| ATOM | 4404 | CD | PRO | 377 | −40.834 | 0.883 | −1.428 |
| ATOM | 4405 | HA | PRO | 377 | −41.896 | −0.766 | −3.911 |
| ATOM | 4406 | HD2 | PRO | 377 | −39.979 | 0.515 | −0.846 |
| ATOM | 4407 | HD1 | PRO | 377 | −41.013 | 1.945 | −1.197 |
| ATOM | 4408 | HG2 | PRO | 377 | −41.844 | −1.022 | −1.095 |
| ATOM | 4409 | HG1 | PRO | 377 | −42.574 | 0.326 | −0.156 |
| ATOM | 4410 | HB1 | PRO | 377 | −43.423 | 1.335 | −2.23 |
| ATOM | 4411 | HB2 | PRO | 377 | −43.84 | −0.388 | −2.431 |
| ATOM | 4412 | N | PHE | 378 | −42.288 | 2.539 | −4.392 |
| ATOM | 4413 | CA | PHE | 378 | −42.748 | 3.508 | −5.383 |
| ATOM | 4414 | C | PHE | 378 | −41.952 | 3.358 | −6.657 |
| ATOM | 4415 | O | PHE | 378 | −42.544 | 3.289 | −7.721 |
| ATOM | 4416 | CB | PHE | 378 | −42.611 | 4.953 | −4.835 |
| ATOM | 4417 | CG | PHE | 378 | −43.155 | 5.989 | −5.826 |
| ATOM | 4418 | CD1 | PHE | 378 | −44.527 | 6.267 | −5.864 |
| ATOM | 4419 | CD2 | PHE | 378 | −42.291 | 6.676 | −6.685 |
| ATOM | 4420 | CE1 | PHE | 378 | −45.015 | 7.293 | −6.678 |
| ATOM | 4421 | CE2 | PHE | 378 | −42.775 | 7.711 | −7.491 |
| ATOM | 4422 | CZ | PHE | 378 | −44.135 | 8.035 | −7.471 |
| ATOM | 4423 | HA | PHE | 378 | −43.813 | 3.317 | −5.598 |
| ATOM | 4424 | H | PHE | 378 | −41.854 | 2.873 | −3.552 |
| ATOM | 4425 | HB1 | PHE | 378 | −43.17 | 5.038 | −3.889 |
| ATOM | 4426 | HB2 | PHE | 378 | −41.557 | 5.18 | −4.613 |
| ATOM | 4427 | HD2 | PHE | 378 | −41.238 | 6.413 | −6.728 |
| ATOM | 4428 | HE2 | PHE | 378 | −42.096 | 8.263 | −8.132 |
| ATOM | 4429 | HZ | PHE | 378 | −44.508 | 8.855 | −8.076 |
| ATOM | 4430 | HE1 | PHE | 378 | −46.078 | 7.514 | −6.694 |
| ATOM | 4431 | HD1 | PHE | 378 | −45.217 | 5.69 | −5.258 |
| ATOM | 4432 | N | VAL | 379 | −40.604 | 3.302 | −6.583 |
| ATOM | 4433 | CA | VAL | 379 | −39.83 | 3.192 | −7.82 |
| ATOM | 4434 | C | VAL | 379 | −40.18 | 1.876 | −8.484 |
| ATOM | 4435 | O | VAL | 379 | −40.449 | 1.852 | −9.677 |
| ATOM | 4436 | CB | VAL | 379 | −38.289 | 3.291 | −7.596 |
| ATOM | 4437 | CG1 | VAL | 379 | −37.531 | 3.116 | −8.944 |
| ATOM | 4438 | CG2 | VAL | 379 | −37.887 | 4.654 | −6.962 |
| ATOM | 4439 | HA | VAL | 379 | −40.13 | 4.014 | −8.492 |
| ATOM | 4440 | H | VAL | 379 | −40.14 | 3.295 | −5.694 |
| ATOM | 4441 | HB | VAL | 379 | −37.976 | 2.484 | −6.91 |
| ATOM | 4442 | HG11 | VAL | 379 | −37.877 | 3.874 | −9.662 |
| ATOM | 4443 | HG12 | VAL | 379 | −36.446 | 3.243 | −8.812 |
| ATOM | 4444 | HG13 | VAL | 379 | −37.703 | 2.116 | −9.37 |
| ATOM | 4445 | HG21 | VAL | 379 | −38.362 | 4.81 | −5.983 |
| ATOM | 4446 | HG22 | VAL | 379 | −36.796 | 4.691 | −6.812 |
| ATOM | 4447 | HG23 | VAL | 379 | −38.176 | 5.485 | −7.623 |
| ATOM | 4448 | N | TYR | 380 | −40.173 | 0.764 | −7.715 |
| ATOM | 4449 | CA | TYR | 380 | −40.447 | −0.544 | −8.312 |
| ATOM | 4450 | C | TYR | 380 | −41.803 | −0.562 | −8.978 |
| ATOM | 4451 | O | TYR | 380 | −41.905 | −0.973 | −10.123 |
| ATOM | 4452 | CB | TYR | 380 | −40.401 | −1.647 | −7.217 |
| ATOM | 4453 | CG | TYR | 380 | −40.696 | −3.086 | −7.684 |
| ATOM | 4454 | CD1 | TYR | 380 | −40.091 | −3.625 | −8.826 |
| ATOM | 4455 | CD2 | TYR | 380 | −41.554 | −3.903 | −6.934 |
| ATOM | 4456 | CE1 | TYR | 380 | −40.194 | −4.992 | −9.108 |
| ATOM | 4457 | CE2 | TYR | 380 | −41.642 | −5.272 | −7.203 |
| ATOM | 4458 | CZ | TYR | 380 | −40.955 | −5.827 | −8.285 |
| ATOM | 4459 | OH | TYR | 380 | −41.045 | −7.201 | −8.524 |
| ATOM | 4460 | HA | TYR | 380 | −39.668 | −0.723 | −9.069 |
| ATOM | 4461 | H | TYR | 380 | −39.964 | 0.823 | −6.734 |
| ATOM | 4462 | HB2 | TYR | 380 | −39.392 | −1.658 | −6.774 |
| ATOM | 4463 | HB1 | TYR | 380 | −41.105 | −1.367 | −6.418 |
| ATOM | 4464 | HD2 | TYR | 380 | −42.146 | −3.488 | −6.126 |
| ATOM | 4465 | HE2 | TYR | 380 | −42.244 | −5.915 | −6.57 |
| ATOM | 4466 | HE1 | TYR | 380 | −39.671 | −5.4 | −9.965 |
| ATOM | 4467 | HD1 | TYR | 380 | −39.514 | −3.001 | −9.496 |
| ATOM | 4468 | HH | TYR | 380 | −40.353 | −7.537 | −9.089 |
| ATOM | 4469 | N | ILE | 381 | −42.863 | −0.121 | −8.268 |
| ATOM | 4470 | CA | ILE | 381 | −44.208 | −0.188 | −8.839 |
| ATOM | 4471 | C | ILE | 381 | −44.322 | 0.781 | −9.99 |
| ATOM | 4472 | O | ILE | 381 | −44.859 | 0.41 | −11.021 |
| ATOM | 4473 | CB | ILE | 381 | −45.302 | 0.096 | −7.763 |
| ATOM | 4474 | CG1 | ILE | 381 | −45.334 | −1.073 | −6.731 |
| ATOM | 4475 | CG2 | ILE | 381 | −46.698 | 0.287 | −8.427 |
| ATOM | 4476 | CD1 | ILE | 381 | −46.045 | −0.72 | −5.397 |
| ATOM | 4477 | HA | ILE | 381 | −44.378 | −1.203 | −9.236 |
| ATOM | 4478 | H | ILE | 381 | −42.736 | 0.276 | −7.355 |
| ATOM | 4479 | HB | ILE | 381 | −45.046 | 1.038 | −7.249 |
| ATOM | 4480 | HG11 | ILE | 381 | −45.839 | −1.932 | −7.199 |
| ATOM | 4481 | HG12 | ILE | 381 | −44.309 | −1.387 | −6.475 |
| ATOM | 4482 | HD11 | ILE | 381 | −47.105 | −0.473 | −5.548 |
| ATOM | 4483 | HD12 | ILE | 381 | −45.995 | −1.579 | −4.709 |
| ATOM | 4484 | HD13 | ILE | 381 | −45.548 | 0.137 | −4.915 |
| ATOM | 4485 | HG21 | ILE | 381 | −46.957 | −0.601 | −9.023 |
| ATOM | 4486 | HG22 | ILE | 381 | −47.485 | 0.443 | −7.676 |
| ATOM | 4487 | HG23 | ILE | 381 | −46.705 | 1.167 | −9.089 |
| ATOM | 4488 | N | VAL | 382 | −43.847 | 2.036 | −9.858 |
| ATOM | 4489 | CA | VAL | 382 | −44.057 | 2.989 | −10.947 |
| ATOM | 4490 | C | VAL | 382 | −43.367 | 2.513 | −12.206 |
| ATOM | 4491 | O | VAL | 382 | −43.981 | 2.56 | −13.261 |
| ATOM | 4492 | CB | VAL | 382 | −43.606 | 4.43 | −10.563 |
| ATOM | 4493 | CG1 | VAL | 382 | −43.591 | 5.376 | −11.797 |
| ATOM | 4494 | CG2 | VAL | 382 | −44.556 | 5.021 | −9.483 |
| ATOM | 4495 | HA | VAL | 382 | −45.138 | 3.032 | −11.163 |
| ATOM | 4496 | H | VAL | 382 | −43.364 | 2.323 | −9.026 |
| ATOM | 4497 | HB | VAL | 382 | −42.577 | 4.383 | −10.164 |
| ATOM | 4498 | HG11 | VAL | 382 | −44.583 | 5.401 | −12.273 |
| ATOM | 4499 | HG12 | VAL | 382 | −43.322 | 6.4 | −11.493 |
| ATOM | 4500 | HG13 | VAL | 382 | −42.854 | 5.045 | −12.542 |
| ATOM | 4501 | HG21 | VAL | 382 | −44.628 | 4.381 | −8.593 |
| ATOM | 4502 | HG22 | VAL | 382 | −44.182 | 6.004 | −9.171 |
| ATOM | 4503 | HG23 | VAL | 382 | −45.57 | 5.152 | −9.89 |
| ATOM | 4504 | N | LEU | 383 | −42.097 | 2.057 | −12.146 |
| ATOM | 4505 | CA | LEU | 383 | −41.395 | 1.712 | −13.385 |
| ATOM | 4506 | C | LEU | 383 | −41.633 | 0.281 | −13.829 |
| ATOM | 4507 | O | LEU | 383 | −41.836 | 0.063 | −15.015 |
| ATOM | 4508 | CB | LEU | 383 | −39.867 | 1.963 | −13.239 |
| ATOM | 4509 | CG | LEU | 383 | −39.481 | 3.434 | −12.881 |
| ATOM | 4510 | CD1 | LEU | 383 | −37.945 | 3.548 | −12.659 |
| ATOM | 4511 | CD2 | LEU | 383 | −39.9 | 4.453 | −13.98 |
| ATOM | 4512 | HA | LEU | 383 | −41.749 | 2.346 | −14.213 |
| ATOM | 4513 | H | LEU | 383 | −41.626 | 1.961 | −11.263 |
| ATOM | 4514 | HB1 | LEU | 383 | −39.496 | 1.289 | −12.45 |
| ATOM | 4515 | HB2 | LEU | 383 | −39.375 | 1.687 | −14.186 |
| ATOM | 4516 | HG | LEU | 383 | −39.978 | 3.715 | −11.936 |
| ATOM | 4517 | HD21 | LEU | 383 | −40.991 | 4.526 | −14.08 |
| ATOM | 4518 | HD22 | LEU | 383 | −39.527 | 5.458 | −13.729 |
| ATOM | 4519 | HD23 | LEU | 383 | −39.477 | 4.16 | −14.954 |
| ATOM | 4520 | HD11 | LEU | 383 | −37.404 | 3.362 | −13.599 |
| ATOM | 4521 | HD12 | LEU | 383 | −37.677 | 4.555 | −12.302 |
| ATOM | 4522 | HD13 | LEU | 383 | −37.605 | 2.82 | −11.908 |
| ATOM | 4523 | N | CYS | 384 | −41.594 | −0.732 | −12.935 |
| ATOM | 4524 | CA | CYS | 384 | −41.614 | −2.117 | −13.416 |
| ATOM | 4525 | C | CYS | 384 | −43.001 | −2.552 | −13.854 |
| ATOM | 4526 | O | CYS | 384 | −43.678 | −3.225 | −13.093 |
| ATOM | 4527 | CB | CYS | 384 | −40.999 | −3.07 | −12.357 |
| ATOM | 4528 | SG | CYS | 384 | −40.82 | −4.739 | −13.076 |
| ATOM | 4529 | HA | CYS | 384 | −40.943 | −2.198 | −14.288 |
| ATOM | 4530 | H | CYS | 384 | −41.519 | −0.557 | −11.948 |
| ATOM | 4531 | HB1 | CYS | 384 | −40 | −2.706 | −12.066 |
| ATOM | 4532 | HB2 | CYS | 384 | −41.631 | −3.11 | −11.459 |
| ATOM | 4533 | HG | CYS | 384 | −40.455 | −5.218 | −12.269 |
| ATOM | 4534 | LPG1 | CYS | 384 | −41.416 | −4.995 | −13.277 |
| ATOM | 4535 | LPG2 | CYS | 384 | −40.383 | −4.747 | −13.596 |
| ATOM | 4536 | N | GLU | 385 | −43.432 | −2.192 | −15.091 |
| ATOM | 4537 | CA | GLU | 385 | −44.742 | −2.619 | −15.595 |
| ATOM | 4538 | C | GLU | 385 | −45.127 | −4.016 | −15.159 |
| ATOM | 4539 | O | GLU | 385 | −46.136 | −4.162 | −14.491 |
| ATOM | 4540 | CB | GLU | 385 | −44.822 | −2.658 | −17.151 |
| ATOM | 4541 | CG | GLU | 385 | −45.185 | −1.304 | −17.808 |
| ATOM | 4542 | CD | GLU | 385 | −45.37 | −1.548 | −19.283 |
| ATOM | 4543 | OE1 | GLU | 385 | −46.474 | −2.001 | −19.684 |
| ATOM | 4544 | OE2 | GLU | 385 | −44.401 | −1.311 | −20.052 |
| ATOM | 4545 | HA | GLU | 385 | −45.503 | −1.922 | −15.209 |
| ATOM | 4546 | H | GLU | 385 | −42.875 | −1.585 | −15.664 |
| ATOM | 4547 | HB1 | GLU | 385 | −43.869 | −3.022 | −17.566 |
| ATOM | 4548 | HB2 | GLU | 385 | −45.611 | −3.363 | −17.461 |
| ATOM | 4549 | HG1 | GLU | 385 | −46.134 | −0.923 | −17.404 |
| ATOM | 4550 | HG2 | GLU | 385 | −44.394 | −0.559 | −17.627 |
| ATOM | 4551 | N | THR | 386 | −44.371 | −5.064 | −15.557 |
| ATOM | 4552 | CA | THR | 386 | −44.827 | −6.424 | −15.273 |
| ATOM | 4553 | C | THR | 386 | −45.144 | −6.593 | −13.81 |
| ATOM | 4554 | O | THR | 386 | −46.14 | −7.222 | −13.488 |
| ATOM | 4555 | CB | THR | 386 | −43.83 | −7.526 | −15.723 |
| ATOM | 4556 | OG1 | THR | 386 | −44.44 | −8.814 | −15.508 |
| ATOM | 4557 | CG2 | THR | 386 | −42.479 | −7.45 | −14.968 |
| ATOM | 4558 | HA | THR | 386 | −45.756 | −6.586 | −15.844 |
| ATOM | 4559 | H | THR | 386 | −43.539 | −4.923 | −16.092 |
| ATOM | 4560 | HB | THR | 386 | −43.649 | −7.391 | −16.803 |
| ATOM | 4561 | HG1 | THR | 386 | −43.902 | −9.538 | −15.812 |
| ATOM | 4562 | HG23 | THR | 386 | −42.032 | −6.449 | −15.034 |
| ATOM | 4563 | HG21 | THR | 386 | −42.598 | −7.727 | −13.909 |
| ATOM | 4564 | HG22 | THR | 386 | −41.781 | −8.155 | −15.43 |
| ATOM | 4565 | N | PHE | 387 | −44.325 | −6.035 | −12.893 |
| ATOM | 4566 | CA | PHE | 387 | −44.639 | −6.204 | −11.482 |
| ATOM | 4567 | C | PHE | 387 | −45.892 | −5.423 | −11.176 |
| ATOM | 4568 | O | PHE | 387 | −46.768 | −5.954 | −10.514 |
| ATOM | 4569 | CB | PHE | 387 | −43.519 | −5.745 | −10.519 |
| ATOM | 4570 | CG | PHE | 387 | −44.061 | −5.88 | −9.091 |
| ATOM | 4571 | CD1 | PHE | 387 | −44.321 | −4.748 | −8.311 |
| ATOM | 4572 | CD2 | PHE | 387 | −44.324 | −7.154 | −8.572 |
| ATOM | 4573 | CE1 | PHE | 387 | −44.855 | −4.892 | −7.028 |
| ATOM | 4574 | CE2 | PHE | 387 | −44.956 | −7.29 | −7.335 |
| ATOM | 4575 | CZ | PHE | 387 | −45.177 | −6.161 | −6.546 |
| ATOM | 4576 | HA | PHE | 387 | −44.822 | −7.275 | −11.303 |
| ATOM | 4577 | H | PHE | 387 | −43.548 | −5.461 | −13.152 |
| ATOM | 4578 | HB1 | PHE | 387 | −42.618 | −6.364 | −10.651 |
| ATOM | 4579 | HB2 | PHE | 387 | −43.256 | −4.7 | −10.733 |
| ATOM | 4580 | HD2 | PHE | 387 | −44.044 | −8.043 | −9.127 |
| ATOM | 4581 | HE2 | PHE | 387 | −45.272 | −8.267 | −6.986 |
| ATOM | 4582 | HZ | PHE | 387 | −45.593 | −6.267 | −5.556 |
| ATOM | 4583 | HE1 | PHE | 387 | −45.02 | −4.025 | −6.4 |
| ATOM | 4584 | HD1 | PHE | 387 | −44.109 | −3.754 | −8.691 |
| ATOM | 4585 | N | ARG | 388 | −46.006 | −4.163 | −11.646 |
| ATOM | 4586 | CA | ARG | 388 | −47.235 | −3.414 | −11.392 |
| ATOM | 4587 | C | ARG | 388 | −48.435 | −4.167 | −11.926 |
| ATOM | 4588 | O | ARG | 388 | −49.471 | −4.187 | −11.281 |
| ATOM | 4589 | CB | ARG | 388 | −47.165 | −1.997 | −12.02 |
| ATOM | 4590 | CG | ARG | 388 | −48.47 | −1.188 | −11.798 |
| ATOM | 4591 | CD | ARG | 388 | −48.324 | 0.306 | −12.198 |
| ATOM | 4592 | NE | ARG | 388 | −48.046 | 0.466 | −13.634 |
| ATOM | 4593 | CZ | ARG | 388 | −46.864 | 0.711 | −14.166 |
| ATOM | 4594 | NH1 | ARG | 388 | −45.742 | 0.759 | −13.49 |
| ATOM | 4595 | NH2 | ARG | 388 | −46.794 | 0.928 | −15.46 |
| ATOM | 4596 | HA | ARG | 388 | −47.331 | −3.294 | −10.302 |
| ATOM | 4597 | H | ARG | 388 | −45.27 | −3.741 | −12.185 |
| ATOM | 4598 | HB1 | ARG | 388 | −46.321 | −1.491 | −11.534 |
| ATOM | 4599 | HB2 | ARG | 388 | −46.96 | −2.067 | −13.099 |
| ATOM | 4600 | HG1 | ARG | 388 | −49.281 | −1.637 | −12.396 |
| ATOM | 4601 | HG2 | ARG | 388 | −48.762 | −1.245 | −10.736 |
| ATOM | 4602 | HD1 | ARG | 388 | −49.305 | 0.776 | −12.008 |
| ATOM | 4603 | HD2 | ARG | 388 | −47.638 | 0.833 | −11.521 |
| ATOM | 4604 | HE | ARG | 388 | −48.87 | 0.435 | −14.256 |
| ATOM | 4605 | HH12 | ARG | 388 | −44.846 | 0.986 | −13.949 |
| ATOM | 4606 | HH11 | ARG | 388 | −45.703 | 0.586 | −12.483 |
| ATOM | 4607 | HH22 | ARG | 388 | −45.895 | 1.14 | −15.917 |
| ATOM | 4608 | HH21 | ARG | 388 | −47.635 | 0.91 | −16.055 |
| ATOM | 4609 | N | LYS | 389 | −48.326 | −4.804 | −13.109 |
| ATOM | 4610 | CA | LYS | 389 | −49.458 | −5.556 | −13.648 |
| ATOM | 4611 | C | LYS | 389 | −49.718 | −6.76 | −12.765 |
| ATOM | 4612 | O | LYS | 389 | −50.87 | −7.047 | −12.477 |
| ATOM | 4613 | CB | LYS | 389 | −49.185 | −5.966 | −15.124 |
| ATOM | 4614 | CG | LYS | 389 | −49.164 | −4.709 | −16.044 |
| ATOM | 4615 | CD | LYS | 389 | −48.572 | −4.998 | −17.452 |
| ATOM | 4616 | CE | LYS | 389 | −48.612 | −3.72 | −18.337 |
| ATOM | 4617 | NZ | LYS | 389 | −47.904 | −3.896 | −19.628 |
| ATOM | 4618 | HA | LYS | 389 | −50.354 | −4.913 | −13.63 |
| ATOM | 4619 | H | LYS | 389 | −47.455 | −4.799 | −13.603 |
| ATOM | 4620 | HB1 | LYS | 389 | −48.224 | −6.5 | −15.172 |
| ATOM | 4621 | HB2 | LYS | 389 | −49.968 | −6.656 | −15.479 |
| ATOM | 4622 | HG1 | LYS | 389 | −50.194 | −4.329 | −16.147 |
| ATOM | 4623 | HG2 | LYS | 389 | −48.56 | −3.914 | −15.58 |
| ATOM | 4624 | HD1 | LYS | 389 | −47.527 | −5.329 | −17.34 |
| ATOM | 4625 | HD2 | LYS | 389 | −49.14 | −5.807 | −17.94 |
| ATOM | 4626 | HE1 | LYS | 389 | −49.663 | −3.446 | −18.53 |
| ATOM | 4627 | HE2 | LYS | 389 | −48.144 | −2.884 | −17.788 |
| ATOM | 4628 | HZ1 | LYS | 389 | −48.304 | −4.692 | −20.241 |
| ATOM | 4629 | HZ2 | LYS | 389 | −47.86 | −2.975 | −20.199 |
| ATOM | 4630 | HZ3 | LYS | 389 | −46.858 | −4.138 | −19.499 |
| ATOM | 4631 | N | ARG | 390 | −48.664 | −7.472 | −12.307 |
| ATOM | 4632 | CA | ARG | 390 | −48.879 | −8.571 | −11.364 |
| ATOM | 4633 | C | ARG | 390 | −49.477 | −8.067 | −10.069 |
| ATOM | 4634 | O | ARG | 390 | −50.291 | −8.765 | −9.481 |
| ATOM | 4635 | CB | ARG | 390 | −47.564 | −9.333 | −11.043 |
| ATOM | 4636 | CG | ARG | 390 | −47.108 | −10.194 | −12.254 |
| ATOM | 4637 | CD | ARG | 390 | −45.809 | −10.989 | −11.951 |
| ATOM | 4638 | NE | ARG | 390 | −45.38 | −11.77 | −13.117 |
| ATOM | 4639 | CZ | ARG | 390 | −44.435 | −12.683 | −13.076 |
| ATOM | 4640 | NH1 | ARG | 390 | −43.763 | −12.961 | −11.986 |
| ATOM | 4641 | NH2 | ARG | 390 | −44.143 | −13.35 | −14.168 |
| ATOM | 4642 | HA | ARG | 390 | −49.594 | −9.278 | −11.807 |
| ATOM | 4643 | H | ARG | 390 | −47.727 | −7.224 | −12.567 |
| ATOM | 4644 | HB1 | ARG | 390 | −46.782 | −8.615 | −10.758 |
| ATOM | 4645 | HB2 | ARG | 390 | −47.73 | −9.988 | −10.175 |
| ATOM | 4646 | HG1 | ARG | 390 | −47.902 | −10.915 | −12.513 |
| ATOM | 4647 | HG2 | ARG | 390 | −46.942 | −9.549 | −13.129 |
| ATOM | 4648 | HD1 | ARG | 390 | −45.01 | −10.283 | −11.668 |
| ATOM | 4649 | HD2 | ARG | 390 | −46.039 | −11.648 | −11.098 |
| ATOM | 4650 | HE | ARG | 390 | −45.869 | −11.595 | −14.01 |
| ATOM | 4651 | HH12 | ARG | 390 | −43.049 | −13.7 | −11.991 |
| ATOM | 4652 | HH11 | ARG | 390 | −43.919 | −12.461 | −11.103 |
| ATOM | 4653 | HH22 | ARG | 390 | −43.399 | −14.069 | −14.17 |
| ATOM | 4654 | HH21 | ARG | 390 | −44.642 | −13.176 | −15.052 |
| ATOM | 4655 | N | LEU | 391 | −49.077 | −6.86 | −9.612 |
| ATOM | 4656 | CA | LEU | 391 | −49.686 | −6.279 | −8.42 |
| ATOM | 4657 | C | LEU | 391 | −51.144 | −6.025 | −8.709 |
| ATOM | 4658 | O | LEU | 391 | −51.977 | −6.403 | −7.901 |
| ATOM | 4659 | CB | LEU | 391 | −48.971 | −4.966 | −7.987 |
| ATOM | 4660 | CG | LEU | 391 | −49.726 | −4.124 | −6.918 |
| ATOM | 4661 | CD1 | LEU | 391 | −49.976 | −4.928 | −5.617 |
| ATOM | 4662 | CD2 | LEU | 391 | −48.912 | −2.841 | −6.585 |
| ATOM | 4663 | HA | LEU | 391 | −49.603 | −7.014 | −7.607 |
| ATOM | 4664 | H | LEU | 391 | −48.382 | −6.333 | −10.102 |
| ATOM | 4665 | HB1 | LEU | 391 | −47.954 | −5.212 | −7.639 |
| ATOM | 4666 | HB2 | LEU | 391 | −48.882 | −4.322 | −8.869 |
| ATOM | 4667 | HG | LEU | 391 | −50.703 | −3.809 | −7.326 |
| ATOM | 4668 | HD21 | LEU | 391 | −48.738 | −2.241 | −7.492 |
| ATOM | 4669 | HD22 | LEU | 391 | −49.453 | −2.216 | −5.858 |
| ATOM | 4670 | HD23 | LEU | 391 | −47.936 | −3.111 | −6.152 |
| ATOM | 4671 | HD11 | LEU | 391 | −49.013 | −5.228 | −5.18 |
| ATOM | 4672 | HD12 | LEU | 391 | −50.525 | −4.299 | −4.903 |
| ATOM | 4673 | HD13 | LEU | 391 | −50.58 | −5.826 | −5.791 |
| ATOM | 4674 | N | VAL | 392 | −51.505 | −5.406 | −9.853 |
| ATOM | 4675 | CA | VAL | 392 | −52.926 | −5.189 | −10.117 |
| ATOM | 4676 | C | VAL | 392 | −53.631 | −6.532 | −10.155 |
| ATOM | 4677 | O | VAL | 392 | −54.698 | −6.632 | −9.572 |
| ATOM | 4678 | CB | VAL | 392 | −53.19 | −4.32 | −11.381 |
| ATOM | 4679 | CG1 | VAL | 392 | −54.708 | −4.279 | −11.717 |
| ATOM | 4680 | CG2 | VAL | 392 | −52.673 | −2.866 | −11.177 |
| ATOM | 4681 | HA | VAL | 392 | −53.339 | −4.628 | −9.263 |
| ATOM | 4682 | H | VAL | 392 | −50.819 | −5.122 | −10.527 |
| ATOM | 4683 | HB | VAL | 392 | −52.644 | −4.764 | −12.229 |
| ATOM | 4684 | HG11 | VAL | 392 | −55.27 | −3.863 | −10.868 |
| ATOM | 4685 | HG12 | VAL | 392 | −54.89 | −3.647 | −12.601 |
| ATOM | 4686 | HG13 | VAL | 392 | −55.101 | −5.283 | −11.932 |
| ATOM | 4687 | HG21 | VAL | 392 | −51.606 | −2.847 | −10.914 |
| ATOM | 4688 | HG22 | VAL | 392 | −52.804 | −2.282 | −12.102 |
| ATOM | 4689 | HG23 | VAL | 392 | −53.231 | −2.367 | −10.37 |
| ATOM | 4690 | N | LEU | 393 | −53.075 | −7.589 | −10.794 |
| ATOM | 4691 | CA | LEU | 393 | −53.712 | −8.911 | −10.701 |
| ATOM | 4692 | C | LEU | 393 | −53.816 | −9.364 | −9.257 |
| ATOM | 4693 | O | LEU | 393 | −54.722 | −10.119 | −8.94 |
| ATOM | 4694 | CB | LEU | 393 | −52.925 | −10.027 | −11.452 |
| ATOM | 4695 | CG | LEU | 393 | −53.066 | −10.019 | −13.003 |
| ATOM | 4696 | CD1 | LEU | 393 | −51.96 | −10.897 | −13.656 |
| ATOM | 4697 | CD2 | LEU | 393 | −54.455 | −10.545 | −13.464 |
| ATOM | 4698 | HA | LEU | 393 | −54.742 | −8.832 | −11.082 |
| ATOM | 4699 | H | LEU | 393 | −52.209 | −7.486 | −11.289 |
| ATOM | 4700 | HB1 | LEU | 393 | −51.869 | −9.906 | −11.176 |
| ATOM | 4701 | HB2 | LEU | 393 | −53.242 | −11.022 | −11.104 |
| ATOM | 4702 | HG | LEU | 393 | −52.931 | −8.989 | −13.363 |
| ATOM | 4703 | HD21 | LEU | 393 | −55.271 | −9.957 | −13.031 |
| ATOM | 4704 | HD22 | LEU | 393 | −54.544 | −10.484 | −14.56 |
| ATOM | 4705 | HD23 | LEU | 393 | −54.594 | −11.595 | −13.163 |
| ATOM | 4706 | HD11 | LEU | 393 | −52.032 | −11.935 | −13.298 |
| ATOM | 4707 | HD12 | LEU | 393 | −52.065 | −10.896 | −14.752 |
| ATOM | 4708 | HD13 | LEU | 393 | −50.958 | −10.513 | −13.415 |
| ATOM | 4709 | N | SER | 394 | −52.902 | −8.961 | −8.348 |
| ATOM | 4710 | CA | SER | 394 | −53.068 | −9.325 | −6.94 |
| ATOM | 4711 | C | SER | 394 | −54.137 | −8.477 | −6.283 |
| ATOM | 4712 | O | SER | 394 | −54.874 | −9.015 | −5.471 |
| ATOM | 4713 | CB | SER | 394 | −51.73 | −9.193 | −6.172 |
| ATOM | 4714 | OG | SER | 394 | −50.757 | −10.03 | −6.826 |
| ATOM | 4715 | HA | SER | 394 | −53.364 | −10.384 | −6.874 |
| ATOM | 4716 | H | SER | 394 | −52.124 | −8.39 | −8.612 |
| ATOM | 4717 | HB1 | SER | 394 | −51.409 | −8.141 | −6.176 |
| ATOM | 4718 | HB2 | SER | 394 | −51.865 | −9.502 | −5.121 |
| ATOM | 4719 | HG | SER | 394 | −49.892 | −9.945 | −6.436 |
| ATOM | 4720 | N | VAL | 395 | −54.238 | −7.166 | −6.609 |
| ATOM | 4721 | CA | VAL | 395 | −55.274 | −6.325 | −6.003 |
| ATOM | 4722 | C | VAL | 395 | −56.628 | −6.757 | −6.521 |
| ATOM | 4723 | O | VAL | 395 | −57.52 | −6.991 | −5.722 |
| ATOM | 4724 | CB | VAL | 395 | −55.072 | −4.805 | −6.301 |
| ATOM | 4725 | CG1 | VAL | 395 | −56.276 | −3.954 | −5.803 |
| ATOM | 4726 | CG2 | VAL | 395 | −53.761 | −4.279 | −5.652 |
| ATOM | 4727 | HA | VAL | 395 | −55.256 | −6.462 | −4.909 |
| ATOM | 4728 | H | VAL | 395 | −53.629 | −6.766 | −7.296 |
| ATOM | 4729 | HB | VAL | 395 | −55 | −4.664 | −7.393 |
| ATOM | 4730 | HG11 | VAL | 395 | −56.428 | −4.103 | −4.723 |
| ATOM | 4731 | HG12 | VAL | 395 | −56.09 | −2.884 | −5.985 |
| ATOM | 4732 | HG13 | VAL | 395 | −57.204 | −4.225 | −6.329 |
| ATOM | 4733 | HG21 | VAL | 395 | −52.894 | −4.849 | −6.007 |
| ATOM | 4734 | HG22 | VAL | 395 | −53.599 | −3.221 | −5.911 |
| ATOM | 4735 | HG23 | VAL | 395 | −53.81 | −4.367 | −4.556 |
| ATOM | 4736 | N | LYS | 396 | −56.803 | −6.85 | −7.857 |
| ATOM | 4737 | CA | LYS | 396 | −58.077 | −7.299 | −8.416 |
| ATOM | 4738 | C | LYS | 396 | −58.027 | −8.813 | −8.41 |
| ATOM | 4739 | O | LYS | 396 | −57.198 | −9.297 | −9.162 |
| ATOM | 4740 | CB | LYS | 396 | −58.236 | −6.79 | −9.876 |
| ATOM | 4741 | CG | LYS | 396 | −58.377 | −5.244 | −9.958 |
| ATOM | 4742 | CD | LYS | 396 | −58.514 | −4.78 | −11.435 |
| ATOM | 4743 | CE | LYS | 396 | −58.565 | −3.233 | −11.595 |
| ATOM | 4744 | NZ | LYS | 396 | −59.764 | −2.612 | −10.98 |
| ATOM | 4745 | HA | LYS | 396 | −58.92 | −6.888 | −7.843 |
| ATOM | 4746 | H | LYS | 396 | −56.033 | −6.696 | −8.478 |
| ATOM | 4747 | HB1 | LYS | 396 | −57.349 | −7.09 | −10.453 |
| ATOM | 4748 | HB2 | LYS | 396 | −59.121 | −7.265 | −10.331 |
| ATOM | 4749 | HG1 | LYS | 396 | −59.267 | −4.944 | −9.384 |
| ATOM | 4750 | HG2 | LYS | 396 | −57.492 | −4.771 | −9.501 |
| ATOM | 4751 | HD1 | LYS | 396 | −57.647 | −5.157 | −12.003 |
| ATOM | 4752 | HD2 | LYS | 396 | −59.42 | −5.225 | −11.878 |
| ATOM | 4753 | HE1 | LYS | 396 | −57.652 | −2.791 | −11.162 |
| ATOM | 4754 | HE2 | LYS | 396 | −58.562 | −3.006 | −12.677 |
| ATOM | 4755 | HZ1 | LYS | 396 | −59.76 | −2.658 | −9.9 |
| ATOM | 4756 | HZ2 | LYS | 396 | −59.834 | −1.559 | −11.222 |
| ATOM | 4757 | HZ3 | LYS | 396 | −60.682 | −3.07 | −11.325 |
| ATOM | 4758 | N | PRO | 397 | −58.822 | −9.599 | −7.635 |
| ATOM | 4759 | CA | PRO | 397 | −58.615 | −11.045 | −7.57 |
| ATOM | 4760 | C | PRO | 397 | −58.211 | −11.777 | −8.833 |
| ATOM | 4761 | O | PRO | 397 | −59.055 | −12.385 | −9.472 |
| ATOM | 4762 | CB | PRO | 397 | −60.009 | −11.462 | −7.041 |
| ATOM | 4763 | CG | PRO | 397 | −60.363 | −10.323 | −6.06 |
| ATOM | 4764 | CD | PRO | 397 | −59.901 | −9.054 | −6.817 |
| ATOM | 4765 | HA | PRO | 397 | −57.862 | −11.246 | −6.79 |
| ATOM | 4766 | HD2 | PRO | 397 | −59.593 | −8.283 | −6.096 |
| ATOM | 4767 | HD1 | PRO | 397 | −60.705 | −8.672 | −7.467 |
| ATOM | 4768 | HG2 | PRO | 397 | −59.764 | −10.45 | −5.142 |
| ATOM | 4769 | HG1 | PRO | 397 | −61.431 | −10.299 | −5.787 |
| ATOM | 4770 | HB1 | PRO | 397 | −60.746 | −11.454 | −7.861 |
| ATOM | 4771 | HB2 | PRO | 397 | −60.008 | −12.458 | −6.568 |
| ATOM | 4772 | N | ALA | 398 | −56.909 | −11.747 | −9.197 |
| ATOM | 4773 | CA | ALA | 398 | −56.448 | −12.47 | −10.38 |
| ATOM | 4774 | C | ALA | 398 | −57.235 | −12.074 | −11.607 |
| ATOM | 4775 | O | ALA | 398 | −57.574 | −12.936 | −12.402 |
| ATOM | 4776 | CB | ALA | 398 | −56.462 | −13.997 | −10.11 |
| ATOM | 4777 | HA | ALA | 398 | −55.406 | −12.188 | −10.603 |
| ATOM | 4778 | H | ALA | 398 | −56.245 | −11.192 | −8.685 |
| ATOM | 4779 | HB1 | ALA | 398 | −56.024 | −14.545 | −10.959 |
| ATOM | 4780 | HB2 | ALA | 398 | −55.875 | −14.219 | −9.206 |
| ATOM | 4781 | HB3 | ALA | 398 | −57.491 | −14.355 | −9.957 |
| ATOM | 4782 | N | ALA | 399 | −57.526 | −10.765 | −11.784 |
| ATOM | 4783 | CA | ALA | 399 | −58.278 | −10.326 | −12.96 |
| ATOM | 4784 | C | ALA | 399 | −57.801 | −8.958 | −13.392 |
| ATOM | 4785 | O | ALA | 399 | −57.246 | −8.259 | −12.562 |
| ATOM | 4786 | CB | ALA | 399 | −59.782 | −10.285 | −12.603 |
| ATOM | 4787 | HA | ALA | 399 | −58.117 | −11.013 | −13.808 |
| ATOM | 4788 | H | ALA | 399 | −57.266 | −10.072 | −11.104 |
| ATOM | 4789 | HB1 | ALA | 399 | −60.377 | −9.955 | −13.469 |
| ATOM | 4790 | HB2 | ALA | 399 | −60.12 | −11.287 | −12.298 |
| ATOM | 4791 | HB3 | ALA | 399 | −59.948 | −9.587 | −11.768 |
| ATOM | 4792 | N | GLN | 400 | −57.975 | −8.541 | −14.667 |
| ATOM | 4793 | CA | GLN | 400 | −57.433 | −7.246 | −15.081 |
| ATOM | 4794 | C | GLN | 400 | −58.057 | −6.792 | −16.38 |
| ATOM | 4795 | O | GLN | 400 | −58.449 | −7.645 | −17.159 |
| ATOM | 4796 | CB | GLN | 400 | −55.893 | −7.38 | −15.241 |
| ATOM | 4797 | CG | GLN | 400 | −55.175 | −6.023 | −15.475 |
| ATOM | 4798 | CD | GLN | 400 | −53.675 | −6.191 | −15.393 |
| ATOM | 4799 | OE1 | GLN | 400 | −52.992 | −6.029 | −16.391 |
| ATOM | 4800 | NE2 | GLN | 400 | −53.125 | −6.523 | −14.207 |
| ATOM | 4801 | HA | GLN | 400 | −57.669 | −6.499 | −14.305 |
| ATOM | 4802 | H | GLN | 400 | −58.425 | −9.113 | −15.356 |
| ATOM | 4803 | HB1 | GLN | 400 | −55.496 | −7.831 | −14.322 |
| ATOM | 4804 | HB2 | GLN | 400 | −55.669 | −8.068 | −16.072 |
| ATOM | 4805 | HG1 | GLN | 400 | −55.449 | −5.618 | −16.463 |
| ATOM | 4806 | HG2 | GLN | 400 | −55.477 | −5.294 | −14.708 |
| ATOM | 4807 | HE22 | GLN | 400 | −52.138 | −6.658 | −14.141 |
| ATOM | 4808 | HE21 | GLN | 400 | −53.681 | −6.645 | −13.388 |
| ATOM | 4809 | N | GLY | 401 | −58.168 | −5.471 | −16.645 |
| ATOM | 4810 | CA | GLY | 401 | −58.754 | −5.037 | −17.91 |
| ATOM | 4811 | C | GLY | 401 | −58.833 | −3.534 | −17.983 |
| ATOM | 4812 | O | GLY | 401 | −58.48 | −2.864 | −17.026 |
| ATOM | 4813 | HA2 | GLY | 401 | −59.772 | −5.446 | −18.012 |
| ATOM | 4814 | HA1 | GLY | 401 | −58.139 | −5.401 | −18.748 |
| ATOM | 4815 | H | GLY | 401 | −57.859 | −4.768 | −16 |
| ATOM | 4816 | H13 | GLY | 401 | −59.204 | −3.036 | −18.879 |

The data shown in Table 1 are expressed based on the Protein Data Bank (PDB) format: The PDB format is a format containing coordinates (X, Y, Z), etc. of individual atoms constituting a protein, and is one of the standard formats in handling coordinates of biopolymers. In Table 1, the “ATOM” appearing in the utmost left column (1st column) denotes each atom of the atomic coordinates. The numbers (1, 2, 3, . . . 4816) appearing in the next column (2nd column) are serial numbers of individual atoms. Subsequently, in the left to right direction in this Table, there are denoted the type of each atom and its position in the amino acid to which it belongs (e.g., “CB”, “CG”, “SD”) (in the 3rd column); the amino acid residue to which each atom belongs (three-letter abbreviations for amino acids, e.g. “MET”, “ASN”) (in the 4th column); the sequence number of the residue counted from the N-terminal (in the 5th column); X-coordinate (in angstrom unit) (in the 6th column); Y-coordinate (in angstrom unit) (in the 7th column); Z-coordinate (in angstrom unit)(in the 8th column).

In addition to the above-described atomic coordinates, the present invention also includes derivatives therefrom, i.e., derivatives from the above-mentioned atomic coordinates (related receptors, etc.) obtained by techniques such as homology modeling on computer. The term “derivatives” means proteins of the GPCR family that have 15% or more, preferably 30% or more, homology to native human MCH-R1 in the amino acid sequence; or atomic coordinates (derived coordinates) in which a part of the amino acid sequence of native human MCH-R1 (preferably one or several (e.g., one to ten) amino acids) is deleted, substituted or added and which exhibit a three-dimensional structure of a protein having activity functionally equivalent to native human MCH-R1 (i.e., the signal transduction activity of GPCRs). Further, in the case of a GPCR family protein that is too low in homology to make an alignment of amino acid sequences with human MCH-R1 in all regions, modeling may be performed on the seven helix sites alone that can be determined using hydrophobicity or the like as an indicator. Whether a given set of atomic coordinates constituting a mutated GPCR amino acid sequence is included as a derivative of the present invention can be analyzed by comparing the three-dimensional structure of the GPCR in question and native human MCH-R1 (as represented by Table 1) by using as a parameter the discrepancies between the positions of α carbon atoms of specific amino acid residues.

Specifically, an image of the three-dimensional structure composed of the atomic coordinates of native human MCH-R1 (designated “three-dimensional structure I”) and an image of the three-dimensional structure composed of the derived coordinates in question (designated “three-dimensional structure II”) are created on computer based on the two sets of atomic coordinates. Subsequently, one image is superimposed on the other image on a computer display. The image of three-dimensional structure I contains seven helix sites of human MCH-R1. These regions are superimposed on the regions of the corresponding helix sites in the image of three-dimensional structure II, or vice versa. This superimposition may be processed automatically based on a computer program, or may be carried out manually on a computer display in a manner similar to the one employed in comparing fingerprints. A suitable program includes COMPOSER or MODELER.

The seven helix sites (regions), the three extra-cellular sites (regions), the three intra-cellular sites (regions) and the carboxy terminus of human MCH-R1 include the first helix (expressed as “HI (106-135)”) consisting of an amino acid sequence from position 106 to position 135 (SEQ ID NO: 2) of the amino acid sequence as shown in SEQ ID NO: 1 (hereinafter, all the position numbers mentioned in this paragraph refer to position numbers in SEQ ID NO: 1), the second helix (expressed as “HII (146-175)”) consisting of an amino acid sequence from position 146 to position 175 (SEQ ID NO: 3), the third helix (expressed as “HIII (183-213)”) consisting of an amino acid sequence from position 183 to position 213 (SEQ ID NO: 4), the fourth helix (expressed as “HIV (226-249)”) consisting of an amino acid sequence from position 226 to position 249 (SEQ ID NO: 5), the fifth helix (expressed as “HV (274-302)”) consisting of an amino acid sequence from position 274 to position 302 (SEQ ID NO: 6), the sixth helix (expressed as “HVI (319-350)”) consisting of an amino acid sequence from position 319 to position 350 (SEQ ID NO: 7), the seventh helix (expressed as “HVII (360-383)”) consisting of an amino acid sequence from position 360 to position 383 (SEQ ID NO: 8); the first extra-cellular region (expressed as “ECI (176-182)”) consisting of an amino acid sequence from position 176 to position 182 (SEQ ID NO: 9), the second extra-cellular region (expressed as “ECII (250-273)”) consisting of an amino acid sequence from position 250 to position 273 (SEQ ID NO: 10), the third extra-cellular region (expressed as “ECIII (351-359)”) consisting of an amino acid sequence from position 351 to position 359 (SEQ ID NO: 11), the first intra-cellular region (expressed as “ICI (136-145)”) consisting of an amino acid sequence from position 136 to position 145 (SEQ ID NO: 12), the second intra-cellular region (expressed as “ICII (214-225)”) consisting of an amino acid sequence from position 214 to position 225 (SEQ ID NO: 13), the third intra-cellular region (expressed as “ICIII (303-318)”) consisting of an amino acid sequence from position 303 to position 318 (SEQ ID NO: 14), and the carboxy terminal region (expressed as “CTERM (384-401)”) consisting of an amino acid sequence from position 384 to position 401 (SEQ ID NO: 15). Then, these regions are superimposed on the corresponding regions of the three-dimensional structure II or vice versa, to calculate the discrepancies between the positions of the α carbon atoms in the amino acid residues of those helices in the three-dimensional structure I and the positions of the corresponding α carbon atoms in the amino acid residues of the corresponding helices in the three-dimensional structure II. When the mean residual of the discrepancies is 1.5 Å, preferably 1.0 , more preferably 0.5, or less, the three-dimensional structure II is regarded as a derivative of the atomic coordinates as shown in Table 1 (derived coordinates). Discrepancies between the positions of corresponding α carbon atoms can be calculated by the so-called least squares method. Generally, such discrepancies can be calculated using a computer software (e.g., LSQKAB, Quanta). The mean residual is calculated as follows. Briefly, individual distances between two corresponding α carbon atoms are squared; the mean value of these squares is calculated; and then the square root of the mean value is calculated to obtain the mean residual.

An active site or protein-binding site is formed by at least one helix selected from the above-described seven helices. Accordingly, a G protein-coupled receptor that has a three-dimensional structure of that active site or protein-binding site, preferably at least one helix consists of HIII, HV, HVI and HVII, is also included in the G protein-coupled receptor of the invention.

In addition to the GPCR (human MCH-R1) having the three-dimensional structure consisting of the atomic coordinates as shown in Table 1, the present invention also includes a three-dimensional structure consisting of such derived coordinates as the GPCRs of the invention.

In Table 1, atomic coordinates for the amino acid residues of individual helices and extra-cellular regions corresponds to amino acid residues 106-135 for HI, 146-175 for HII, 176-182 for ECI, 183-213 for HIII, 226-249 for HIV, 250-273 for ECII, 274-302 for HV, 319-350 for HVI, 351-359 for ECIII and 360-383 for HVII (residue numbers are entered in the 5th column).

II. Uses of the 3D Coordinates of MCH-R1

For the first time, the present invention permits the use of virtual design techniques (i.e., computer modeling or “in silico”) to design, select, and synthesize a drug candidate capable of inhibiting/stimulating or binding MCH-R1. In turn, these drug candidates may be effective in the treatment of a MCH-R1 mediated disorder.

The term “MCH-R1 mediated disorder” is a disorder that involves a modulation in MCH-R1 activity in the biological manifestation of the disease, disorder, and/or condition; in the biological cascade leading to the disorder; or as a symptom of the disorder. This “involvement” of MCH-R1 in a MCH-R1 mediated disorder includes, but is not limited to, the following: (1) The modulation of MCH-R1 activity as a “cause” of the disorder or biological manifestation, whether the MCH-R1 is modulated genetically, by infection, by autoimmunity, trauma, biomechanical causes, lifestyle, or by some other causes. (2) The modulated MCH-R1 activity is part of the observable manifestation of the disease or disorder. That is, the disease or disorder is measurable in terms of the modulated MCH-R1 activity. From a clinical standpoint, modulated MCH-R1 activity indicates the disease, however, MCH-R1 activity need not be the “hallmark” of the disease or disorder. (3) The modulated MCH-R1 activity is part of the biochemical or cellular cascade that results in the disease or disorder. In this respect, inhibiting or stimulating of MCH-R1 (per the respective therapeutic goal) interrupts the cascade, and thus controls the disease. “Modulation in MCH-R1 activity,” as used herein, encompasses both unwanted or elevated MCH-R1 activity and desired or reduced MCH-R1 activity.

Non-limiting examples of MCH-R1 mediated disorders that may be treated by agonists or antagonists identified by the present invention include obesity, anorexia, inflammation, mental disorders, cachexia, metabolic and eating disorders, diabetes, insulin resistance, glucose intolerance, Type-2 diabetes mellitus, coronary artery disease, elevated blood pressure, hypertension, dyslipidaeniia, cancer (e.g., endometrial, cervical, ovarian, breast, prostate, gallbladder, colon), menstrual irregularities, hirsutism, infertility, gallbladder disease, restrictive lung disease, sleep apnea, gout, osteoarthritis, and thromboembolic disease, memory (including learning), cardiovascular function, sepsis, cardiogenic and hypovolemic shock, sexual dysfunction, penile erection, muscle atrophy, nerve growth and repair, intrauterine fetal growth, and the like.

In the present invention, it is possible to carry out virtual screening for drugs using the above-described atomic coordinates or derived coordinates therefrom.

Briefly, the atomic coordinates of the three-dimensional structure elucidated by the invention are input into a computer so that images of the structure and various parameters are shown on the display. All the information contained in Table 1 may be input, or atomic coordinates corresponding to at least one helix, or atomic coordinates corresponding to at least one extra-cellular region, or atomic coordinates corresponding to two helices linked by an extra-cellular region, or atomic coordinates corresponding to the top fifteen residues of at least one helix, or atomic coordinates corresponding to the top fifteen residues of two helices linked by an extra-cellular region may be input. For example, atomic coordinates corresponding to HI (106-135), HII (146-175), HIII (183-213), HIV (226-249), HV (274-302), HVI (319-350), HVII (360-383), ECI (176-182), ECII (250-273), ECIII (351-359), THII-ECI-THIII (161-197), THIV-ECII-THV (235-288), THVI-ECIII-THVII (336-374), TH-I (106-120), TH-II (161-175), TH-III (183-197), TH-IV (235-249), TH-V (274-288), TH-VI (336-350), and/or TH-VII (360-374) may be input.

Then, the resultant data are input into a virtual compound library. Since a virtual compound library is contained in a virtual screening software such as DOCK-4 (Kuntz, UCSF), the above-described data may be input into such a software. Candidate drugs may be searched for, using a three-dimensional structure database of virtual or non-virtual drug candidate compounds, such as MDDR (Prous Science, Spain).

The potential stimulating/inhibitory or binding effect (i.e., interaction or association) of a drug candidate compound may be analyzed prior to its actual synthesis and testing by the use of computer modeling techniques. If the theoretical structure of the given compound suggests insufficient interaction and association between it and MCH-R1, synthesis and testing of the candidate is obviated. However, if computer modeling indicates a strong interaction, the molecule may then be synthesized and tested for its ability to bind to or stimulate/inhibit MCH-R1 using various methods known in the art. Examples of such methods include the measurement of second messenger responses, in particular cAMP, such as the use of modified cell systems yielding color reaction upon accumulation of second messenger elements such as cAMP, e.g. as described by Chen et al. 1995 (Anal Biochem. 1995, 226, 349-54), Cytosensor Microphysiometer techniques (see Boyfield et al. 1996). In this manner, synthesis of inoperative candidates may be avoided.

Agonist/antagonist or binding drug candidates of MCH-R1 may be computationally evaluated and designed by means of a series of steps in which chemical entities or fragments are screened and selected for their ability to bind with the individual binding sites (e.g., HI, HV, HVI, HVII, ECI, ECII, ECIII) or other areas of MCH-R1.

One skilled in the art may use one of several methods to screen chemical entities or fragments for their ability to associate with MCH-R1 and more particularly with the individual binding sites. This process may begin by visual inspection of, for example, the active site on the computer screen based on the MCH-R1 coordinates in Table I. Selected fragments or chemical entities may then be positioned in a variety of orientations, or docked, within an individual binding site of MCH-R1 as defined supra. Docking may be accomplished using software such as QUANTA, SYBYL, followed by energy minimization and molecular dynamics with standard molecular mechanics forcefields, such as CHARMM and AMBER.

Specialized computer programs may also assist in the process of selecting fragments or chemical entities. These include: (1) GRID (Goodford, P. J., “A Computational Procedure for Determining Energetically Favorable Binding Sites on Biologically Important Macromolecules” J. Med. Chem., 28, pp. 849-857 (1985)), available from Oxford University, Oxford, UK; (2) MCSS (Miranker, A. and M. Karplus, “Functionality Maps of Binding Sites: A Multiple Copy Simultaneous Search Method.” Proteins: Structure, Function and Genetics, 11, pp. 29-34 (1991)), available from Molecular Simulations, Burlington, Mass; (3) AUTODOCK (Goodsell, D. S. and A. J. Olsen, “Automated Docking of Substrates to Proteins by Simulated Annealing” Proteins: Structure. Function, and Genetics, 8, pp. 195-202 (1990)), available from Scripps Research Institute, La Jolla, Calif.; and (4) DOCK (Kuntz, I. D. et al., “A Geometric Approach to Macromolecule-Ligand Interactions” J. Mol. Biol., 161, pp. 269-288 (1982)), available from University of California, San Francisco, Calif; (5) GLIDE available from Schrodinger Inc.; (6) FlexX available from Tripos Inc; (7) GOLD (Jones et al., J. Mol. Biol., 245, 43-53, 1995) available from the Cambridge Crystallographic Data Centre.

Once suitable chemical entities or fragments have been selected, they can be assembled in silico or synthesized into a single drug candidate. In silico assembly may proceed by visual inspection of the relationship of the fragments to each other on the three-dimensional image displayed on a computer screen in relation to the structure coordinates of MCH-R1. This would be followed by manual model building using software such as QUANTA or SYBYL. Chemical syntheses are by those methods well-known in the art.

Useful programs to aid one of skill in the art in connecting the individual chemical entities or fragments include the following: (1) CAVEAT (Bartlett, P. A. et al, “CAVEAT: A Program to Facilitate the Structure-Derived Design of Biologically Active Molecules”. In Molecular Recognition in Chemical and Biological Problems”, Special Pub., Royal Chem. Soc., 78, pp. 182-196 (1989)), available from the University of California, Berkeley, Calif.; (2) 3D Database systems such as MACCS-3D (MDL Information Systems, San Leandro, Calif.). This area is reviewed in Martin, Y. C., “3D Database Searching in Drug Design”, J. Med. Chem., 35, pp. 2145-2154 (1992)); and (3) HOOK (available from Molecular Simulations, Burlington, Mass.).

Instead of proceeding to build an MCH-R1 agonist or antagonist in a step-wise fashion one fragment or chemical entity at a time as described above, drug candidates useful for the treatment of MCH-R1 mediated disorders may be designed as a whole or “de novo” using either an empty active site or optionally including some portion(s) of a known agonist or antagonist. These methods include the following: (1) LUDI (Bohm, H.-J., “The Computer Program LUDI: A New Method for the De Novo Design of Enzyme Inhibitors”, J. ComR. Aid. Molec. Design, 6, pp. 61-78 (1992)), available from Biosym Technologies, San Diego, Calif.; (2) LEGEND (Nishibata, Y. and A. Itai, Tetrahedron, 47, p. 8985 (1991)), available from Molecular Simulations, Burlington, Mass.; (3) LeapFrog (available from Tripos Associates, St. Louis, Mo.).

Once a drug candidate has been designed or selected by the above methods, the efficiency with which that candidate may bind to MCH-R1 may be tested and optimized by computational evaluation. An effective MCH-R1 agonist must preferably demonstrate a relatively small difference in energy between its bound and free states (i.e., a small deformation energy of binding). Thus, the most efficient MCH-R1 agonist should preferably be designed with a deformation energy of binding of not greater than about 10 kcal/mole, preferably, not greater than 7 kcal/mole. MCH-R1 agonists may interact with the enzyme in more than one conformation that is similar in overall binding energy. In those cases, the deformation energy of binding is taken to be the difference between the energy of the free compound and the average energy of the conformations observed when the agonist binds to the enzyme.

A compound designed or selected as binding to MCH-R1 may be further computationally optimized so that in its bound state it would preferably lack repulsive electrostatic interaction with the target enzyme. Such non-complementary (e.g., electrostatic) interactions include repulsive charge-charge, dipole-dipole and charge-dipole interactions. Specifically, the sum of all electrostatic interactions between the compound and the enzyme when the compound is bound to MCH-R1, preferably make a neutral or favorable contribution to the enthalpy of binding.

Specific computer software is available in the art to evaluate compound deformation energy and electrostatic interaction. Examples of programs designed for such uses include: Gaussian 92, revision C [M. J. Frisch, Gaussian, Inc., Pittsburgh, Pa. ©1992]; AMBER, version 4.0 [P. A. Kollman, University of California at San Francisco, ©1994]; QUANTA/CHARMM [Molecular Simulations, Inc., Burlington, Mass. ©1994]; and Insight II/Discover (Biosysm Technologies Inc., San Diego, Calif. ©1994). Other software packages will be known to those skilled in the art.

Once a MCH-R1-binding compound has been optimally selected or designed, as described above, substitutions may then be made in some of its atoms or side groups in order to improve or modify its binding properties. Generally, initial substitutions are conservative, i.e., the replacement group will have approximately the same size, shape, hydrophobicity and charge as the original group. It should, of course, be understood that components known in the art to alter conformation should be avoided. Such substituted chemical compounds may then be analyzed for efficiency of fit to MCH-R1 by the same computer methods described in detail, above.

Except as otherwise noted, all amounts including quantities, percentages, portions, and proportions, are understood to be modified by the word “about”, and amounts are not intended to indicate significant digits.

Except as otherwise noted, the articles “a”, “an”, and “the” mean “one or more”.

All documents cited are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope.

1. A method of identifying a drug candidate useful for the treatment of a melanin concentrating hormone receptor 1 (MCH-R1) mediated disorder comprising the steps of:
(a) using a three-dimensional (3D) structure of MCH-R1 according to Table 1;
(b) employing said 3D structure to design or select in silico said drug candidate.

2. The method according to claim 1, further comprising the step of synthesizing said drug candidate.

3. The method according to claim 2, further comprising contacting said drug candidate in the presence of a substrate to test the ability of said candidate to modulate MCH-R1.

4. The method according to claim 3, wherein said candidate is selected from a database of virtual or non-virtual compounds.

5. The method according to claim 3, wherein said candidate is designed de novo.

6. The method according to claim 3, wherein said candidate is designed from a known agonist or antagonist.

7. The method according to claim 1, wherein said step of employing said 3D structure to design or select said candidate comprises the steps of:
(a) identifying in silico chemical entities or fragments capable of binding with a peptide selected from the group consisting of SEQ ID NOS: 2-11; and
(b) assembling in silico or synthesizing the identified chemical entities or fragments in into a single molecule to provide the structure of said candidate.

8. The method according to claim 1, wherein said step of employing said 3D structure to design or select said candidate comprises the steps of:
(a) identifying in silico chemical entities or fragments capable of binding with a peptide selected from the group consisting of SEQ ID NOS: 16-19; and
(b) assembling in silico or synthesizing the identified chemical entities or fragments into a single molecule to provide the structure of said candidate.

9. The method according to claim 1, wherein said step of employing said 3D structure to design or select said candidate comprises the steps of:
(a) identifying in silico chemical entities or fragments capable of binding with a peptide selected from the group consisting of SEQ ID NOS: 16, 20-25; and
(b) assembling in silico or synthesizing the identified chemical entities or fragments into a single molecule to provide the structure of said candidate.

10. The method according to claim 1, wherein the candidate is an agonist or an antagonist of MCH-R1.

11. A G-protein-coupled receptor selected from the group consisting of the following (a) or (b):
(a) a G protein-coupled receptor having three-dimensional structure I defined by the atomic coordinates as shown in Table 1;
(b) a G protein-coupled receptor having three-dimensional structure II defined by derived coordinates from the atomic coordinates as shown in Table 1, wherein the mean residual of the discrepancies between the positions of the α carbon atoms in the amino acid residues of seven helix sites SEQ ID NOS: 2-8 of the three-dimensional structure I and the positions of the corresponding α carbon atoms in the amino acid residues of the corresponding seven helix sites of the three-dimensional structure II is 1.5 Å or less when an image of the three-dimensional structure I obtained by computer-processing the atomic coordinates of Table 1 and an image of the three-dimensional structure II obtained by computer-processing said derived coordinates are superimposed.

12. The receptor of claim 11, wherein the G protein-coupled receptor is MCH-R1.

13. The receptor of claim 11, wherein the G protein-coupled receptor is human MCH-R1.

14. A method of identifying a drug candidate useful for the treatment of a melanin concentrating hormone receptor 1 (MCH-R1) mediated disorder comprising the steps of:
(a) using a three-dimensional (3D) structure of a G-protein-coupled receptor selected from the group consisting of the following (i) or (ii):
(i) a G protein-coupled receptor having three-dimensional structure I defined by the atomic coordinates as shown in Table 1;
(ii) a G protein-coupled receptor having three-dimensional structure II defined by derived coordinates from the atomic coordinates as shown in Table 1, wherein the mean residual of the discrepancies between the positions of the α carbon atoms in the amino acid residues of seven helix sites SEQ ID NOS: 2-8 of the three-dimensional structure I and the positions of the corresponding α carbon atoms in the amino acid residues of the corresponding seven helix sites of the three-dimensional structure II is 1.5 Å or less when an image of the three-dimensional structure I obtained by computer-processing the atomic coordinates of Table 1 and an image of the three-dimensional structure II obtained by computer-processing said derived coordinates are superimposed.
(b) employing said 3D structure to design or select said drug candidate.

15. The method according to claim 14, further comprising the step of synthesizing said drug candidate.

16. The method according to claim 15, further comprising contacting said drug candidate in the presence of a substrate to test the ability of said candidate to modulate MCH-R1.

17. The method according to claim 16, wherein said candidate is selected from a database of virtual or non-virtual compounds.

18. The method according to claim 16, wherein said candidate is designed de novo.

19. The method according to claim 16, wherein said candidate is designed from a known agonist or antagonist.
